(19) United States
(12) Patent Application Publication
(10) Pub. No.: US 2020/0386742 A1
(43) Pub. Date: Dec. 10, 2020

(54) CELL SYSTEMS USING SPHEROIDS AND METHODS OF MAKING AND USING THE SAME

(71) Applicants: The Administrators of the Tulane Educational Fund, New Orleans, LA (US); AXOSIM INC., New Orleans, LA (US)

(72) Inventors: Michael James Moore, New Orleans, LA (US); Jabe Lowry Curley, New Orleans, LA (US); Anup D. Sharma, New Orleans, LA (US); Devon Bowser, New Orleans, LA (US); Jordan Behn, New Orleans, LA (US)

(73) Assignees: The Administrators of the Tulane Educational Fund, New Orleans, LA (US); AxoSim Technologies LLC, New Orleans, LA (US)

(21) Appl. No.: 16/769,985

(22) PCT Filed: Dec. 4, 2018
(86) PCT No.: PCT/US2018/063861

§ 371 Date: Jun. 4, 2020

Related U.S. Application Data
(60) Provisional application No. 62/594,525, filed on Dec. 4, 2017.

(51) Int. Cl. G01N 33/50; C12N 5/0793; C12N 5/071

(52) U.S. Cl. CPC G01N 33/5026; C12N 5/0619; C12N 5/0697; G01N 33/5058; C12N 2533/54; C12N 2502/1335; C12N 2506/45; C12N 2513/00; C12N 2533/40; C12N 2502/081

(57) ABSTRACT

The present disclosure generally relates to a cell culturing system, and specifically to a three-dimensional cell culturing system for neuronal cells that promotes both structural and functional characteristics that mimic those of in vivo peripheral fibers, including cell myelination. Using a dual hydrogel construct and spheroids comprising neuronal cells, the present disclosure provides methods, devices, and systems for in vitro spatially-controlled, three-dimensional models that permit intra- and extra-cellular electro-physiological measurements and recordings. The three-dimensional hydrogel constructs allow for flexibility in incorporated cell types, geometric fabrication, and electrical manipulation, providing viable systems for culture, perturbation, and testing of biomimetic neural growth with physiologically-relevant results.

### CROSS REFERENCE TO RELATED APPLICATIONS

This application is an international application designating the United States of America and filed under 35 U.S.C. § 120, which claims priority to U.S. Provisional Application No. 62/594,525, filed on Dec. 4, 2017, which is herein incorporated by reference in its entirety.

### STATEMENT REGARDING FEDERALLY SPONSORED RESEARCH

This invention was made with government support under grant number NIH STTR Grant No. R42-TR001270. The United States government has certain rights in this invention.

### FIELD

The present disclosure generally relates to a cell culturing system, and specifically to a three-dimensional cell culturing system for spheroids that promotes both structural and functional characteristics that mimic those of in vivo nerve fibers, including cell myelination and propagation of compound action potentials.

### BACKGROUND

Replicating functional aspects of physiology for bench top assessment is especially challenging for peripheral neuronal tissue, where bioelectrical conduction over long distances is one of the most relevant physiological outcomes. For this reason, three dimensional tissue models of peripheral nerves are lagging behind models of epithelial, metabolic, and tumor tissues, where soluble analytes serve as appropriate metrics. The application of electrophysiological techniques has recently been possible through multi-electrode array technologies for the screening of environmental toxins as well as for disease modeling and therapeutic testing. This application is groundbreaking for the study of both peripheral nervous system (PNS) and central nervous system (CNS) applications, but the dissociated nature of the cultures fails to replicate the population level environment and metrics critical for peripheral tissue. Instead, clinical methods of investigating peripheral neuropathy and neuroprotection include nerve conduction testing through measurement of compound action potentials (CAP) and nerve fiber density (NFD) using morphometric analysis of skin biopsies.

### SUMMARY

The present disclosure relates to microphysiological models of the nervous system that provide 3D architecture as well as specified organization. Other model systems tend to allow only one or the other. Organotypic tissue slices can provide 3D architecture as well as organization specified by nature, but these models are not amenable to very high-throughput analysis.

The present disclosure relates to a composition comprising a spheroid of cells comprising one or a combination of cells and/or tissues chosen from: a neuronal cell, nervous system ganglia, a stem cell, and an immune cell. In some embodiments, the spheroid comprises a tissue chosen from: a dorsal root ganglia and a trigeminal ganglia. In some embodiments, the spheroid comprises one or a plurality of cells chosen from: a glial cell, an embryonic cell, a mesenchymal stem cell, a cell derived from an induced pluripotent stem cell, a sympathetic neuron, a parasympathetic neuron, a spinal motor neurons, a central nervous system neuron, a peripheral nervous system neuron, an enteric nervous system neurons, a motor neuron, a sensory neuron, a cholinergic neuron, a GABAergic neuron, a glutamatergic neuron, a dopaminergic neuron, a serotonergic neuron, an interneuron, an adrenergic neuron, a trigeminal ganglion, astrocytes, oligodendrocytes, Schwann cells, microglia, ependymal cells, radial glia, satellite cells, enteric glial cells, and pituyicytes. In some embodiments, the spheroid comprises one or a plurality of glial cells. In some embodiments, the spheroid comprises one or a plurality of embryonic cells. In some embodiments, the spheroid comprises one or a plurality of mesenchymal stem cells. In some embodiments, the spheroid comprises one or a plurality of cells derived from an induced pluripotent stem cell. In some embodiments, the spheroid comprises one or a plurality of parasympathetic neurons. In some embodiments, the spheroid comprises one or a plurality of spinal motor neurons. In some embodiments, the spheroid comprises one or a plurality of central nervous system neurons. In some embodiments, the spheroid comprises one or a plurality of peripheral nervous system neurons. In some embodiments, the spheroid comprises one or a plurality of enteric nervous system neurons. In some embodiments, the spheroid comprises one or a plurality of motor neurons. In some embodiments, the spheroid comprises one or a plurality of sensory neurons. In some embodiments, the spheroid comprises one or a plurality of interneurons. In some embodiments, the spheroid comprises one or a plurality of cholinergic neurons. In some embodiments, the spheroid comprises one or a plurality of GABAergic neurons. In some embodiments, the spheroid comprises one or a plurality of glutamergic neurons, In some embodiments, the spheroid comprises one or a plurality of dopaminergic neurons. In some embodiments, the spheroid comprises one or a plurality of serotonergic neurons. In some embodiments, the spheroid comprises one or a plurality of trigeminal ganglion cells. In some embodiments, the spheroid comprises one or a plurality of astrocytes. In some embodiments, the spheroid comprises one or a plurality of oligodendrocytes. In some embodiments, the spheroid comprises one or a plurality of Schwann cells. In some embodiments, the spheroid comprises one or a plurality of microglial cells. In some embodiments, the spheroid comprises one or a plurality of ependymal cells. In some embodiments, the spheroid comprises one or a plurality of radial glia. In some embodiments, the spheroid comprises one or a plurality of satellite cells. In some embodiments, the spheroid comprises one or a plurality of enteric glial cells. In some embodiments, the spheroid comprises one or a plurality of pituyicytes.

In some embodiments, the spheroid comprises one or a plurality of one or combination of immune cells chosen from: a T cell, B cell, macrophage and astrocytes. In some embodiments, the spheroid comprises one or a plurality of one or a combination of stem cells chosen from: an embryonic stem cell, a mesenchymal stem cell, and an induced pluripotent stem cell. In some embodiments, the neuronal cell is derived from a stem cell chosen from: an embryonic stem cell, a mesenchymal stem cell, and an induced pluripotent stem cell. Embodiments include each of the above-mentioned cell types with each other individually or in combination.

In some embodiments, the spheroid has a diameter from about 200 microns to about 700 microns. In some embodiments, the spheroid has a diameter from about 150 microns to about 800 microns. In some embodiments, the spheroid has a diameter of about 200 microns. In some embodiments, the spheroid has a diameter of about 300 microns. In some embodiments, the spheroid has a diameter of about 400 microns. In some embodiments, the spheroid has a diameter of about 500 microns. In some embodiments, the spheroid has a diameter of about 600 microns. In some embodiments, the spheroid has a diameter of about 700 microns. In some embodiments, the spheroid has a diameter of about 800 microns. In some embodiments, the spheroid has a diameter of about 900 microns. In some embodiments, the spheroid has a diameter of about 350 microns. In some embodiments, the spheroid has a diameter of about 450 microns. In some embodiments, the spheroid has a diameter of about 550 microns. In some embodiments, the spheroid has a diameter of about 650 microns.

In some embodiments, the spheroid comprises one or a plurality of neuronal cells and one or a plurality of Schwann cells at a ratio of cell types equal to about 4 neuronal cells for every 1 Schwann cell. In some embodiments, the spheroid comprises one or a plurality of neuronal cells and one or a plurality of astrocytes at a ratio of about 4 neuronal cells for every 1 astrocyte. In some embodiments, the spheroid comprises one or a plurality of neuronal cells and one or a plurality of astrocytes at a ratio of about 1 neuronal cell for every 1 astrocyte. In some embodiments, the spheroid comprises one or a plurality of neuronal cells and one or a plurality of Schwann cells at a ratio of about 10 neuronal cells for every 1 Schwann cell. In some embodiments, the spheroid comprises one or a plurality of neuronal cells and one or a plurality of glial cells at a ratio equal to about four neuronal cells for every 1 glial cell.

In some embodiments, any one or plurality of cells described herein are differentiated from induced pluripotent stem cells. In some embodiments, the spheroid are free of induced pluripotent stem cells and/or immune cells. In some embodiments, the spheroid are free of undifferentiated stem cells.

In some embodiments, the spheroid comprises no less than about 30,000, 35,000, 40,000, 45,000, 50,000, 55,000, 60,000, 65,000, 70,000, or 75,000 cells. In some embodiments, the spheroid comprises no less than 75,000 cells. In some embodiments, the spheroid comprises no less than 65,000 cells. In some embodiments, the spheroid comprises no less than 60,000 cells. In some embodiments, the spheroid comprises no less than 100,000 cells. In some embodiments, the spheroid comprises no less than 125,000 cells. In some embodiments, the spheroid comprises no less than 150,000 cells. In some embodiments, the spheroid comprises no less than 175,000 cells. In some embodiments, the spheroid comprises no less than 200,000 cells.

In some embodiments, the spheroid comprises no less than 225,000 cells. In some embodiments, the spheroid comprises no less than 250,000 cells. In some embodiments, the spheroid comprises no less than 12,500 cells. In some embodiments, the spheroid comprises from about 12,500 cells to about 250,000 cells. In some embodiments, the spheroid comprises from about 12,500 cells to about 100,000 cells. In some embodiments, the spheroid comprises from about 12,500 cells to about 75,000 cells.

In some embodiments, the spheroid further comprises one or a plurality of magnetic particles. In some embodiments the magnetic particles comprise one or more hollow interiors. In some embodiments, the magnetic particles comprises one or more layers of polymer onto which the cells form a spheroid.

The present disclosure also relates to a system comprising: (i) a cell culture vessel comprising a hydrogel; (ii) one or a plurality of spheroids comprising one or plurality of neuronal cells and/or isolated tissue explants; (iii) an amplifier comprising a generator for electrical current; (iv) a voltmeter and/or ammeter; and (v) at least a first stimulating electrode and at least a first recording electrode; wherein the amplifier, voltmeter and/or ammeter, and electrodes are electrically connected to the each other via a circuit in which electrical current is fed to the at least one stimulating electrode from the amplifier and electrical current is received at the recording electrode and fed to the voltmeter and/or ammeter; wherein the stimulating electrode is positioned at or proximate to one or a plurality of soma of the neuronal cells and/or isolated tissue explants and the recording electrode is positioned at a predetermined distance distal to the soma, such that an electrical field is established across the cell culture vessel. In some embodiments, the spheroid is any of the spheroids described herein.

In some embodiments, the culture vessel comprises 96, 192, 384 or more interior chambers. In some embodiments, the 96, 192, 384 or more interior chambers comprise one or plurality of isolated Schwann cells and/or one or plurality of oligodendrocytes sufficiently proximate to the one or plurality of isolated tissue explants and/or the one or plurality of neuronal cells such that the Schwann cells or the oligodendrocytes deposit myelin to axon growth from the tissue explants and/or neuronal cells.

In some embodiments, the system further comprises a solid substrate onto which the hydrogel matrix is crosslinked, said solid substrate comprising at least one plastic surface with pores from about 1 micron to about 5 microns in diameter. In some embodiments, the solid substrate comprises a contiguous exterior surface and an interior surface, such solid substrate comprising at least one portion in a cylindrical or substantially cylindrical shape and at least one hollow interior defined at its edge by at least one portion of the interior surface, said interior surface comprising one or a plurality of pores from about 0.1 microns to about 1.0 microns in diameter wherein the hollow interior of the solid substrate is accessible from a point exterior to the solid substrate through at least one opening; wherein the hollow interior portion comprises a first portion proximate to the opening and at least a second portion distal to the opening; wherein the one or plurality of neuronal cells and/or the one or plurality of tissue explants are positioned at or proximate to the first portion of the hollow interior and are in physical contact with the hydrogel matrix, and wherein the second portion of the at least one hollow interior is in fluid communication with the first portion such that axons are capable of growth from the one or plurality of neuronal cells and/or the one or plurality of tissue explants into the second interior portion of the hollow interior.

In some embodiments, the system or composition is free of a sponge. In some embodiments, the hydrogel comprises at least a first cell-impenetrable polymer and a first cell-penetrable polymer. In some embodiments, the at least one cell-impenetrable polymer comprises no greater than about 15% PEG and the at least one cell-penetrable polymer comprises from about 0.05% to about 1.00% of one or a combination of self-assembling peptides chosen from: RAD 16-I, RAD 16-II, EAK 16-I, EAK 16-II, and dEAK 16. In some embodiments, the composition is free of polyethylene glycol (PEG). In some embodiments, the hydrogel comprises a first region and a second region, the first region is formed in the shape of a cylinder or rectangular prism oriented with its longitudinal axis passing through the top and bottom of the cell culture vessel and each of either the cylinder or rectangular prism comprising a space defined by an inner surface of the cylinder or rectangular prism, said space and accessible by one or more openings through the top of the cell culture vessel; wherein the second region comprises a space formed in the shape of its interior walls with an opening on its side adjacent to and in fluid communication with the first region. In some embodiments, the hydrogel comprises at least 1% polyethylene glycol (PEG).

In some embodiments, the system further comprises a cell medium comprising nerve growth factor (NGF) at a concentration from about 5 to about 20 picograms per milliliter and/or ascorbic acid in a concentration ranging from about 0.001% weight by volume to about 0.01% weight by volume.

In some embodiments, the system comprises one or a plurality of spheroids comprising at least one or a combination of cells chosen from: a glial cell, an embryonic cell, a mesenchymal stem cell, a cell derived from an induced pluripotent stem cell, a sympathetic neuron, a parasympathetic neuron, a spinal motor neurons, a central nervous system neuron, a peripheral nervous system neuron, an enteric nervous system neurons, a motor neuron, a sensory neuron, a cholinergic neuron, a GABAergic neuron, a glutamatergic neuron, a dopaminergic neuron, a serotonergic neuron, an interneuron, an adrenergic neuron, a trigeminal ganglion neuron, astrocytes, oligodendrocytes, Schwann cells, microglia, ependymal cells, radial glia, satellite cells, enteric glial cells, and pituyicytes. In some embodiments, the system further comprises one or a plurality of stem cells, pluripotent cells, myoblasts and osteoblasts. In some embodiments, the one or more neuronal cells comprise a primary mammalian cell derived from the peripheral nervous system of the mammal.

In some embodiments, the spheroids are in culture for no less than about 3, 30, 90, or 365 days.

In some embodiments, at least one portion of the solid substrate is cylindrical or substantially cylindrical such that at least one portion of the interior surface of the solid substrate defines a cylindrical or substantially cylindrical hollow interior chamber in which the spheroids are positioned. In some embodiments, the hydrogel comprises a series of two or more cavities in fluid communication with each other by a series of channels, at least one cavity comprising a spheroid and at least a second cavity comprising a second spheroid, suspension of cells, or DRG; wherein the spheroid and the second spheroid, suspension of cells, or DRG is connected by a three-dimensional axon. In some embodiments, the cavities are wells with a U-shaped or rounded wells positioned in a horizontal or substantially horizontal plane of the solid substrate with each channel comprises one or a plurality of axons connecting the one or plurality of spheroids.

In some embodiments, the one or plurality of spheroids comprises one or a plurality of neuronal cells with axonal growth from about 100 microns to about 500 microns in width and from about 0.11 to about 10000 microns in length. In some embodiments, the three-dimensional axon is at least about 10 microns in height at its lowest point or is at least three cellular monolayers in height.

In some embodiments, the system comprises a first spheroid comprising: (i) one or a plurality of neuronal cells; and/or (ii) one or a plurality of Schwann cells or oligodendrocytes; and a second spheroid comprising: (i) one or a plurality of peripheral neurons; wherein each spheroid is positioned in the cavity. In some embodiments, the system comprises a first, second and third cavity each configured to hold a spheroid and at least 50 microliters of cell culture medium, wherein the cavities are aligned such that the first cavity is positioned proximal to the second cavity and distal to the third cavity. In some embodiments, the system comprises at least a fourth cavity into which cavities are positioned in a pattern such that each cavity defines a corner of a square. In some embodiments, the cavities are aligned into a line such that axons originating from the first spheroid in the first cavity extend to the second cavity, and axons from the spheroid in the second cavity extend to the axons in the third cavity.

The present disclosure also relates to a method of manufacturing a three-dimensional culture of one or a plurality of spheroids in a culture vessel. In some embodiments, the method comprises: (a) contacting one or a plurality of neuronal cells with the solid substrate, said substrate comprising at least one exterior surface, at least one interior surface and at least one interior chamber defined by the at least one interior surface and accessible from a point exterior to the solid substrate through at least one opening; (b) positioning one or a plurality of spheroids comprising neuronal cells to the at least one interior chamber; and (c) applying a cell medium into the culture vessel with a volume of cell medium sufficient to cover the at least one spheroid; wherein at least one portion of the interior surface comprises a first cell-impenetrable polymer and a first cell-penetrable polymer. In some embodiments, step (b) comprises positioning spheroids comprising tissue explants selected from one or a combination of: an isolated dorsal root ganglion, a spinal cord explant, a retinal explant, and a cortex explant.

In some embodiments, the spheroids are formed as a suspension of neuronal cells selected from one or a combination of: motor neurons, sensory neurons, sympathetic neurons, parasympathetic neurons, cortical neurons, spinal cord neurons, peripheral neurons, optionally derived from a stem cell. In some embodiments, the spheroids are formed from a suspension of neuronal cells selected from one or a combination of: motor neurons, sensory neurons, sympathetic neurons, parasympathetic neurons, cortical neurons, spinal cord neurons, peripheral neurons, optionally derived from a stem cell. In some embodiments, the spheroids further comprise isolated Schwann cells and/or oligodendrocytes.

In some embodiments, the method further comprises a step of (d) allowing the spheroids to grow neurites and/or axons after step (c) for a period of from about 12 hours to about 1 year. In some embodiments, the method further comprises the step of: isolating one or a plurality of neural cells from a sample prior to step (a); and/or isolating dorsal root ganglion (DRG) from one or a plurality of mammals prior to step (b), if the one or plurality of spheroids comprise a DRG; and/or isolating one or a plurality of Schwann cells and/or one or a plurality of oligodendrocytes, if the one or a plurality of spheroids comprise a Schwann cell or oligodendrocyte.

In some embodiments, the method further comprises positioning at least one stimulating electrode at or proximate to soma of the one or plurality of neuronal cells or tissue explants and positioning at least one recording electrode at or proximate to an axon at a point most distal from the soma, such that, upon introducing a current in the stimulating electrode, the recording electrode is capable of receiving a signal corresponding to one or a plurality of electrophysiological metrics capable of being measured at the recording electrode; wherein the one or plurality of electrophysiological metrics are one or a combination of: electrical conduction velocity, action potential, amplitude of the wave associated with passage of an electrical impulse along a membrane of one or a plurality of neuronal cells, a width of an electrical impulses along a membrane of one or a plurality of neuronal cells, latency of the electrical impulse along a membrane of one or a plurality of neuronal cells, and envelope of the electrical impulse along a membrane of one or a plurality of neuronal cells.

The present disclosure also relates to a method of evaluating the toxicity and/or neuroprotective effects of an agent comprising: (a) culturing one or more spheroids in any of the compositions disclosed herein; (b) exposing at least one agent to the one or more spheroids; (c) measuring and/or observing one or more morphometric changes and/or one or more electrophysiological metrics of the one or more spheroids; and (d) correlating one or more morphometric changes and/or one or more electrophysiological metrics of the one or more spheroids with the toxicity of the agent, such that, if the morphometric changes and/or electrophysiological metrics are indicative of decreased cell viability, the agent is characterized as toxic and, if the morphometric changes and/or electrophysiological metrics are indicative of unchanged or increased cell viability, the agent is characterized as non-toxic and/or neuroprotective.

The present disclosure also relates to a method of measuring myelination or demyelination of one or more axons of one or a plurality of spheroids comprising: (a) culturing one or more spheroids in any of the compositions disclosed herein in the presence or absence of an agent for a time and under conditions sufficient to grow at least one axon; and (b) detecting the amount of myelination on one or a plurality of axons from the one or more spheroids; wherein detecting optionally comprises the steps of: (i) measuring and/or observing one or more morphometric changes and/or one or more electrophysiological metrics of the one or more spheroids in the presence or absence of an agent; and (ii) correlating one or more morphometric changes and/or one or more electrophysiological metrics of the one or more spheroids in the presence or absence of an agent with a quantitative or qualitative change of myelination of the spheroids.

The present disclosure also relates to a method of method of detecting and/or quantifying neuronal cell growth and/or axon degeneration comprising: (a) quantifying one or a plurality of spheroids and/or number or density of axons grown from spheroids; (b) culturing the one or more spheroids in any of the compositions disclosed herein; and (c) calculating the number of cells within the spheroid and/or number or density of axons grown from spheroids in the composition after culturing the spheroids for a time period sufficient to allow growth of the one or plurality axons or of growth of cells in the spheroid. In some embodiments, step (b) optionally comprises contacting the one or more spheroids with one or more agents. In some embodiments, step (c) optionally comprises detecting an internal and/or external recording of such one or more spheroids after culturing one or more spheroids and correlating the recording with a measurement of the same recording corresponding to a known or control number of cells. In some embodiments, step (c) optionally comprises the additional steps of: (i) measuring an intracellular and/or extracellular recording and/or a morphometric change before and after the step of contacting the one or more spheroids to one or more agents; and (ii) correlating the difference in the recordings and/or morphometric changes before contacting the one or more spheroids to the one or more agents to the recordings and/or morphometric changes after contacting the one or more spheroids to the one or more agents to a change in cell number and/or number or density of axons.

The present disclosure also relates to a method of measuring or quantifying a neuromodulatory effect of an agent comprising: (a) culturing one or a plurality of spheroids in any of the compositions disclosed herein in the presence and absence of the agent; (b) applying a voltage potential across the one or a plurality of spheroids in the presence and absence of the agent; (c) measuring one or a plurality of electrophysiological metrics from the one or plurality of spheroids in the presence and absence of the agent; and (d) correlating the difference in one or a plurality of electrophysiological metrics through the one or plurality of spheroids to the neuromodulatory effect of the agent, such that a change in electrophysiological metrics in the presence of the agent as compared to the electrophysiological metrics measured in the absence of the agent is indicative of a neuromodulatory effect, and no change of electrophysiological metrics in the presence of the agent as compared to the electrophysiological metrics measured in the absence of the agent is indicative of the agent not conferring a neuromodulatory effect.

The present disclosure also relates to a method of measuring or quantifying a neuromodulatory effect of an agent comprising: (a) culturing one or a plurality of spheroids in any of the compositions disclosed herein in the presence and absence of the agent; (b) measuring and/or observing one or more morphometric changes of the one or plurality of spheroids in the presence and absence of the agent; and (c) correlating the one or more morphometric changes with the neuromodulatory effect of the agent, such that a change in morphometrics in the presence of the agent as compared to the morphometrics measured and/or observed in the absence of the agent is indicative of a neuromodulatory effect, and no change of morphometrics in the presence of the agent as compared to the morphometrics measured and/or observed in the absence of the agent is indicative of the agent not conferring a neuromodulatory effect.

### BRIEF DESCRIPTION OF THE DRAWINGS

FIG. 1A-FIG. 1B depict the fabricated 3D hydrogel scaffolding for the nerve-on-a-chip design.

FIG. 2 depicts a representative spheroid and axon growth within the hydrogel. The hydrogel construct is able to direct and confine 3D axon growth and cellular positioning in order to mimic the nerve fiber tract.

FIG. 3 depicts a list of the morphological and physiological measurements that can be taken at the ganglion, at the proximal tract, at the midpoint of the tract, and at the distal tract of a dorsal root ganglion.

FIG. 4 depicts confocal image stacks of unmyelinated neural fiber tracts proximal to the dorsal root ganglion, the midpoint, and distal from the ganglion, stained with β-III Tubulin to show neurites, DAPI to show nuclei, and S100 to show Schwann cells.

FIG. 5 depicts a confocal depth map demonstrating 3D neurite density.

FIG. 6A-FIG. 6C depict transmission electron microscopy (TEM) of neural culture cross-sections. FIG. 6A depicts high density of parallel, fasciculated unmyelinated neurites in channel approximately 1.875 mm from ganglion. FIG. 6B depicts a focus centered on an axon (Ax) encapsulated by a Schwann cell (SC) approximately 1 mm from the ganglion. FIG. 6C depicts myelin sheaths around individual nerve fibers in 25 day cultures.

FIG. 7A-FIG. 7B depict three-dimensional renderings of confocal images. FIG. 7A depicts the immunohistochemistry for MBP protein. FIG. 7B depicts the immunohistochemistry for MAG. The culture thickness for both is 190 μm, confirming three dimensional myelin formation ability of the in vitro system.

FIG. 8 depicts neurite outgrowth from spheroids formed from human neurons derived from induced pluripotent stem cells. Neurites extend on 2D surfaces from human motor neuron spheroids co-cultured with astrocytes (left) and Schwann cells (right).

FIG. 9 depicts spheroids of motor neurons and astrocytes grown in the 3D hydrogel system. These spheroids displayed robust 3D neurite outgrowth (approximately 5 mm).

FIG. 10 depicts neurites extending in 3D from human motor neuron/Schwann cell spheroids (top) and human sensory neurons (bottom).

FIG. 11 depicts a 96-well spheroid printing drive. A 96-well plate sits on top of the drive, effectively placing one magnet in the center of each well. The magnetized cells are then attracted to the magnet, causing aggregation and allowing for spheroid formation.

FIG. 12 depicts a protocol for making rat spinal cord spheroids. Spheroids may be formed by adding magnetic nanoparticles to cultured cells and culturing in non-adherent plates above a magnet. (Not shown: Spheroids may also be formed by spinning in non-adherent round bottom plates with magnetic nanoparticles present.) One or more magnetic cell spheroids may be held in place with magnets while hydrogel growth matrices are added.

FIG. 13 depicts the device for placing magnetic spheroids into hydrogel voids. The outer portion of this design has the magnet in the center. The dark gray portion allows movement in the y-direction, and the inner/top-most piece houses a glass slide, aligns the insert, and allows movement in the x-direction. The single magnet in the center allows control of placement for constructs requiring a single magnet, without regard to construct shape. Other device designs contain multiple magnets for placing spheroids in connected wells.

FIG. 14A-FIG. 14B depict placement of spheroids in hydrogel constructs. Neurite outgrowth from rat embryonic spinal cord spheroids, as indicated by β-III tubulin staining, conforms to the hydrogel pattern of the outer mold. FIG. 14A depicts incorrect placement of the spheroid when no magnet was used. FIG. 14B depicts proper placement with the device shown in FIG. 13.

FIG. 15 depicts a bar graph showing the reproducibility of spheroid production. The same spheroid formation method yielded batch-to-batch consistency. Spheroids of 27,000 cells had an average diameter of 0.47±0.03 mm and 0.72±0.15 circularity in the xy direction.

FIG. 16A-FIG. 16B depicts representative phase images and viability of cell spheroids. These show examples of reproducible, well-formed spheroid formation from cells of primary embryonic rat spinal cords demonstrating consistent size and shape.

FIG. 17 depicts neurite outgrowth from spheroids in 1:20 diluted Matrigel. Neurites stay confined to the growth permissive channel within the 3D environment. Neurites grew to approximately 100-150 μm thick.

FIG. 18 depicts a defined circuit created with a dorsal root ganglion (DRG) explant combined with Schwann cell spheroids in a gelatin methacrylate hydrogel. This configuration allowed for unidirectional DRG neurite outgrowth.

FIG. 19 depicts a defined circuit created with multiple Schwann cell spheroids in 1:20 diluted Matrigel arranged in a rectangular formation.

FIG. 20 depicts a schematic of method for producing neural microphysiological system using magnetic spheroids. Digital projection lithography may be used to cure a hydrogel “mold” into which a growth-permissive hydrogel will be contained.

FIG. 21 depicts 3D spheroid seeding using nanoshuttle to place the spheroids within the micropatterned hydrogels. Spheroids may be formed by adding magnetic nanoparticles to cultured cells and culturing in non-adherent plates above a magnet. One or more magnetic cell spheroids may be held in place with magnets while hydrogel growth matrices are added.

FIG. 22 depicts a bar graph showing that the number of cells seeded affects the diameter of the spheroid.

FIG. 23A-FIG. 23B depict the viability and 3D structure of spheroids. FIG. 23A: Calcein stain indicating high viability of cells in spheroids formed from primary rat embryonic spinal cord tissue. FIG. 23B: Cross-sectional views of β-III tubulin-stained spheroids in hydrogel constructs obtained with fluorescent light-sheet microscopy demonstrate 3D structure of spheroids.

FIG. 24 depicts spheroids with different cell types incorporated. Neurons β-III), oligodendrocyte-lineage cells (Olig2), astrocytes (GFAP), and microglia and macrophages (CDIIb) are present in the spheroids, as indicated by antibody staining.

FIG. 25 depicts formation of 3D neural networks using spheroids and micropatterned hydrogels. Spheroids formed from rat embryonic DRG cells (left) extend neurites, as shown by calcein staining, toward spinal cord spheroids (right) in methacrylated gelatin, but not vice-versa.

FIG. 26 depicts placement of multiple spinal cord spheroids in fixed locations and neural networks that conform to the geometry of outer mold.

FIG. 27 depicts confocal images (10× on top, 20× bottom) of cocultured iPSC derived motor neurons grown on top of human muscle myocytes and myotubes. Myocytes were allowed to differentiate for 3 days in growth media, then motor neurons were added and media was switched to motor neuron media.

FIG. 28 depicts a confocal image (10×) of 2D culture of mature myotubes forming a 3D sheath expressing heavy chain myosin. Myotubes were cultured for 3 weeks: 3 days in growth media, 7 days in differentiation media, and the remainder in growth media.

FIG. 29 depicts a 10× phase contrast image of 3D muscle cells encapsulated in 5% GelMA after 3 weeks in culture.

FIG. 30 depicts a 255 μM 10× max intensity projection Z-stack of Desmin-expressing human skeletal myotubes after 3 weeks in culture.

FIG. 31 depicts a 96 μM 10× max intensity projection Z-stack of heavy chain myosin-expressing human skeletal myotubes after 3 weeks in GelMA culture.

FIG. 32 depicts 3D spheroid generation in a hanging drop at different seeing densities of induced neurons.

FIG. 33 depicts 3D spheroid generation in U-bottom wells at different seeing densities of induced neurons.

FIG. 34 depicts 3D spheroid generation in a hanging drop at different seeing densities of motor neurons.

FIG. 35 depicts 3D spheroid generation in U-bottom wells at different seeing densities of motor neurons.

FIG. 36 depicts 3D spheroid generation in a hanging drop at different seeing densities of motor neurons over a 24 or 48 hour period.

FIG. 37 depicts 3D spheroid generation in U-bottom wells at different seeing densities of motor neurons over a 24 or 48 hour period.

FIG. 38 depicts growth of a hanging drop spheroid seeded at 25,000 iPSC-derived motor neurons and 25,000 astrocytes after 4 days.

FIG. 39 depicts growth of a U-bottom well spheroid seeded at 25,000 iPSC-derived motor neurons and 25,000 astrocytes after 4 days.

FIG. 40 depicts growth of a hanging drop spheroid seeded at 40,000 iPSC-derived motor neurons and 10,000 astrocytes after 4 days.

FIG. 41 depicts different combinations of neurons and glia over a 24 hour period.

FIG. 42 depicts spheroids of oligodendrocyte precursor cells in 4× (top) and 10× (bottom).

FIG. 43A-43J depict fabrication of spheroids consisting of human neurons (hNs) and/or human Schwann cells (hSCs) after 2 days in vitro. Presence of hSCs expedited spheroid formation in the co-culture system while the hN-only condition did not form spheroids in 2 days. hSC spheroids were fabricated with three different cellular densities: 25,000 (a), 50,000 (b) and 75,000 (c). Co-culture spheroids were created with a constant hN density (75,000) but a changing hSC density: 25,000 (d), 50,000 (e), and 75,000 (f). In parallel, hN spheroids were fabricated at three different densities: 50,000 (g), 75,000 (h) and 100,000 (i). (j) Comparison of diameters of different spheroids revealed that co-culture spheroids were more compact as compared to mono-culture spheroids, showing that the affinity of hSCs for hNs resulted in a more tightly packed cluster of cells. K (multiple of thousand). N=4 and error bars represent standard error of the mean (SEM). Scale bar: 100 μm. ****(p-value≤0.0001), ***(p-value≤0.001), **(p-value≤0.01), *(p-value≤0.05).

FIG. 44 depicts the formation of spheroids consisting of human neurons (hNs) alone. Qualitative inspection at >2 days after initiating the hN-only culture showed a consistent significant increase in spheroid sizes as the total number of cells increase (25K, 50K, 75K and 100K). Graph comparing the individual spheroid diameters supported the qualitative inspection. K (multiple of thousand). N=4 and error bars represent standard error of the mean (SEM). Scale bar: 100 μm. ****(p-value≤0.0001), **(p-value≤0.01).

FIGS. 45A and 45B depict Schwann cells migrated out of the spheroid and elongated along the axons. (45A) Image showing how human Schwann cells (hSCs) stained for the hSC marker S100 (light grey) migrated out of the spheroid along with growing axons stained for βIII-tubulin (grey) over a period of 4 weeks. Nuclei were labeled with DAPI (dark grey). Scale bar: 1000 μm. (45B) High-magnification image of inset from image A. Scale bar: 25 μm.

FIG. 46A through FIG. 46C depict recordings of an axon grown from a spheroid in culture. FIG. 46B′ and FIG. 46B″ depict a graphical representation of the recordings that were collected. The graphs depict nerve conductance velocity (NCV) measured in meters per second of two spheroids over time. FIG. 46C depicts the same NCV experiments with bar graphs depicting NCV at two time points: the onset and peak of electrical current onset.

FIG. 47A-47F. Various stages of myelin formation observed in in vitro reconstituted human nerve. (47A) Non-compact myelin. (47B) Compact myelin. (47C) Myelin in the process of compaction. (47D) Myelin formation without axons. (47E) Intracytoplasmic lamellar bodies. (47F) Naked (unmyelinated) axons.

FIG. 48A-48D. Dorsal Horn Spheroid Characterization. (48A) Photomicrograph shows a typical DRG to DH Synapse culture at 14 DIV. (48B) Fluorescent image showing β-tubulin staining of the synapse culture at 28 DIV. (48C) Photomicrograph shows the synapse culture on the E-phys rig. Blue arrow shows the distance between the stimulating and recording electrodes, which was 3.1 mm. This image shows the stimulating electrode at the DRG axons and the recording electrode at the DH spheroid. (48D) Electrical activity was recorded at the DH spheroid in response to a 20V stimulation at the DRG axons. X-axis is in milliseconds; y-axis is in millivolts.

### DETAILED DESCRIPTION

Various terms relating to the methods and other aspects of the present disclosure are used throughout the specification and claims. Such terms are to be given their ordinary meaning in the art unless otherwise indicated. Other specifically defined terms are to be construed in a manner consistent with the definition provided herein.

As used in this specification and the appended claims, the singular forms “a,” “an,” and “the” include plural referents unless the content clearly dictates otherwise.

The term “more than 2” as used herein is defined as any whole integer greater than the number two, e.g. 3, 4, or 5.

The term “about” as used herein when referring to a measurable value such as an amount, a temporal duration, and the like, is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value, as such variations are appropriate to perform the disclosed methods.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

The term “plurality” as used herein is defined as any amount or number greater or more than 1.

As used herein, “substantially equal” can be, for example, within a range known to be correlated to an abnormal or normal range at a given measured metric. For example, if a control sample is from a diseased patient, substantially equal is within an abnormal range. If a control sample is from a patient known not to have the condition being tested, substantially equal is within a normal range for that given metric.

The disclosure generally relates to a system capable of housing and culturing one or a plurality of spheroids in three dimensional culture. In some embodiments, the system uses a solid substrate, such as plastic or similar polymer, comprising pores upon which a hydrogel can rest in any shape or size. The hydrogel of the system, in some embodiments, acts as a support for the cells of the disclosure to grow and propagate neurites and/or form axons under conditions sufficient for mature cells of the nervous system to grow, divide, and/or propagate axons, whether in spheroid form or in suspension. In some embodiments, the system comprises a hydrogel that forms a cavity with at least two regions: a first region, resembling a well with a flat or curved bottom and a diameter across a longitudinal plane of the sold support, the first region also comprising an opening at the top of the region with access outside of the system and an opening on at least one lateral side of the first region that is in fluid communication with a second region. In some embodiments the first region is about 1 mm in diameter or less. The second region is the form of a channel extending laterally from the first region with sides defining a height of the channel. In some embodiments, the width of the channel is from about 10 to about 750 microns. In some embodiments, the channel is from about 100 to about 10,000 microns in length. After growing spheroids from any one or combination of cells identified in the disclosure, the spheroids can be placed in the first region with cell culture medium. Following the placement, neurites may grow spontaneously or are encouraged to grow by exposure to one or more growth stimulating molecules. Neurite and/or axon growth can occur in the second region emanating in the first region of the system and passing through the at least one opening on the lateral side of the hydrogel and into the second region. After growth of the neurites or axons to a desired length, an agent may be exposed to culture of cells to determine how that agent affects the growth, morphology, or action potentials of the axon or neurites.

In some embodiments, the cavities or wells that hold the spheroid and define the first region may be in a pattern or network connected by the corresponding second regions such that spheroids are connected by channels of axons growing from one or more of the spheroids. In some embodiments, the spheroids are in a square or rectangular pattern connected by a channel positioned between each spheroid. In some embodiments, the pattern is in the shape of an “L” with spheroids defining the end and corner of the “L” configuration. In some embodiments, the spheroid may be positioned in a triangular or angular pattern with three channels between each of three cavities comprising a spheroid. At one end of the hydrogel network, a first cavity may comprise a spheroid with a central nervous system character. In these embodiments, cells that typically populate the central nervous system make up the spheroid. Such cells may be selected from any combination or composition comprising individual neuronal cells and may also include astrocytes or immune cells. In the same embodiments, the cavity most distal to the first cavity may hold a spheroid with a sensory character, such as those spheroids comprising sensory neurons. The axonal connection between the first spheroid and the spheroid most distal from the first spheroid therefore models a sensory nerve fiber where the axon runs from the spheroid with a central nervous system character to the spheroid comprising peripheral sensory neurons. Electrophysiological measurements between such spheroids can be taken by placing electrodes at either end of the circuit and measuring recordings.

In some embodiments, the spheroids comprise a mixture of neuronal and non-neuronal cells. Non-neuronal cells include skeletal muscle cells, cardiac muscle cells, and smooth muscle cells. Non-neuronal cells also include cells from organ tissues such as kidney cells, liver cells, and pancreatic cells. Examples of non-neuronal cells also include endothelial cells, epithelial cells of the skin and corneal cells of the eye. In some embodiments, the cells are mammalian cells, non-human animal cells, or human cells. In some embodiments, any one or plurality of the cells of the spheroid are a primary human cell. In some embodiments, the cells are taken from a human subject. In some embodiments, the cells are rat or murine cells. In some embodiments, the cells are non-human primate cells, porcine cells, dog cells, or bovine cells. In some embodiments, any of the disclosed systems may comprise a spheroid of neuronal cells mixed or not mixed with non-neuronal cells.

Methods of the disclosure include a method of culturing a spheroid disclosed herein and methods of measuring toxicity or biological effect of a toxin, drug, therapeutic, biomolecule or pollutant when such molecules, drugs, or therapeutics are exposed to the culture of spheroid and axons or neurites sprouting from such spheroid in the system. In some embodiments, the methods include a method of causing unidirectional growth of axons and/or neurites in culture from a first spheroid to a second spheroid. In some embodiments, any of the disclosed systems comprises an agent that stimulates, accelerates, slows or stops the growth of the neurites and/or axons in culture. In some embodiments, any methods of the disclosure comprise stimulating direction growth of an axon in culture. In some embodiments, agents are used to either attract guidance of the axon and/or neurites or repulse growth of axons and/or neurons. In some embodiments, attractive guidance proteins are added to the system chosen from: netrins, neurotrophins, adhesive extracellular matrix proteins, cell adhesion receptors (such as cadherins, Ig-CAMs, or integrins); one could also use peptides that mimic the putative binding sites of these proteins. In some embodiments, proteins that repulse axon and/or neurite growth are components of the system. Repulsive proteins include: Ephrins (sometimes), Semaphorins (most of the time), Slits; chondroitin sulfate proteoglycans and the like.

Method of manufacturing spheroids with and without magnetic particles or beads are also disclosed. If magnetic particles are components of the spheroids, any device comprising a magnet may be used to place one or plurality of spheroids in a position within one of the cavities of the disclosed formed by the walls of the hydrogel. The disclosure generally relates to a device comprising a movable frame, said movable frame is movable in any lateral direction parallel to a horizontal on which the device is operating. The frame is attached to one or a plurality of magnets with a magnetic force sufficient to attract a spheroid comprising magnetic particles. In some embodiments, the frame movable in the x and y direction of a longitudinal plane of the device is mechanically attached to a magnet by a glue, polymer, or fastener such that movement of the frame causes movement of a magnet with a magnetic force sufficient to move a spheroid in any direction if the spheroid is within a magnetic field of the magnet. In some embodiments, the device comprises a first frame and a second frame, at least one of the first or second frames movable in the lateral direction parallel to a longitudinal plane of the device and a horizontal or substantially horizontal on the device.

The term “bioreactor” refers to an enclosure or partial enclosure in which cells are cultured, optionally in suspension. In some embodiments, the bioreactor refers to an enclosure or partial enclosure in which cells are cultured where said cells may be in liquid suspension, or alternatively may be growing in contact with, on, or within another non-liquid substrate including but not limited to a solid growth support material. In some embodiments, the solid growth support material, or solid substrate, comprises at least one or a combination of: silica, plastic, metal, hydrocarbon, or gel. The disclosure relates to a system comprising a bioreactor comprising one or a plurality of culture vessels into which neuronal cells may be cultured in the presence or cellular growth media.

The term “culture vessel” as used herein can be any vessel suitable for growing, culturing, cultivating, proliferating, propagating, or otherwise similarly manipulating cells. A culture vessel may also be referred to herein as a “culture insert”. In some embodiments, the culture vessel is made out of biocompatible plastic and/or glass. In some embodiments, the plastic is a thin layer of plastic comprising one or a plurality of pores that allow diffusion of protein, nucleic acid, nutrients (such as heavy metals and hormones) antibiotics, and other cell culture medium components through the pores. in some embodiments, the pores are not more than about 0.1, 0.5 1.0, 2, 3, 4, 5, 6, 7, 8, 9, 10, 15, 20, 25, 30, 40, 50 microns wide. In some embodiments, the culture vessel in a hydrogel matrix and free of a base or any other structure. In some embodiments, the culture vessel is designed to contain a hydrogel or hydrogel matrix and various culture mediums. In some embodiments, the culture vessel consists of or consists essentially of a hydrogel or hydrogel matrix. In some embodiments, the only plastic component of the culture vessel is the components of the culture vessel that make up the side walls and/or bottom of the culture vessel that separate the volume of a well or zone of cellular growth from a point exterior to the culture vessel. In some embodiments, the culture vessel comprises a hydrogel and one or a plurality of isolated glial cells. In some embodiments, the culture vessel comprises a hydrogel and one or a plurality of isolated glial cells, to which one or a plurality of neuronal cells are seeded.

The term “electrical stimulation” refers to a process in which the cells are being exposed to an electrical current of either alternating current (AC) or direct current (DC). The current may be introduced into the solid substrate or applied via the cell culture media or other suitable components of the cell culture system. In some embodiments, the electrical stimulation is provided to the device or system by positioning one or a plurality of electrodes at different positions within the device or system to create a voltage potential across the cell culture vessel. The electrodes are in operable connection with one or a plurality of amplifiers, voltmeters, ammeters, and/or electrochemical systems (such as batteries or electrical generators) by one or a plurality of wires. Such devices and wires create a circuit through which an electrical current is produced and by which an electrical potential is produced across the tissue culture system.

The term “hydrogel” as used herein can be, for example, any water-insoluble, crosslinked, three-dimensional network of polymer chains with the voids between polymer chains filled with or capable of being filled with water. The term “hydrogel matrix” as used herein refers to, for example, any three-dimensional hydrogel construct, system, device, or similar structure. Hydrogels and hydrogel matrices are known in the art and various types have been described, for example, in U.S. Pat. Nos. 5,700,289, and 6,129,761; and in Curley and Moore, 2011; Curley et al., 2011; Irons et al., 2008; and Tibbitt and Anseth, 2009; each of which are incorporated by reference in their entireties. In some embodiments, the hydrogel or hydrogel matrix can be solidified by subjecting the liquefied pregel solution to ultraviolet light, visible light or ay light above about 300 nm, 400 nm, 450 nm or 500 nm in wavelength. In some embodiments, the hydrogel or hydrogel matrix can be solidified into various shapes, for example, a bifurcating shape designed to mimic a neuronal tract. In some embodiments, the hydrogel or hydrogel matrix comprises poly (ethylene glycol) dimethacrylate (PEG). In some embodiments, the hydrogel or hydrogel matrix comprises Puramatrix. In some embodiments, the hydrogel or hydrogel matrix comprises glycidyl methacrylate-dextran (MeDex). In some embodiments, neuronal cells are incorporated in the hydrogel or hydrogel matrices. In some embodiments, cells from nervous system are incorporated into the hydrogel or hydrogel matrices. In some embodiments, the cells from nervous system are Schwann cells and/or oligodendrocytes. In some embodiments, the hydrogel or hydrogel matrix comprises tissue explants from the nervous system of an animal, (such as a mammal) and a supplemental population of cells derived from the nervous system but isolated and cultured to enrich its population in the culture. In some embodiments, the hydrogel or hydrogel matrix comprises a tissue explant such as a retinal tissue explant, DRG, or spinal cord tissue explant and a population of isolated and cultured Schwann cells, oligodendrocytes, and/or microglial cells. In some embodiments, two or more hydrogels or hydrogel matrixes are used simultaneously cell culture vessel. In some embodiments, two or more hydrogels or hydrogel matrixes are used simultaneously in the same cell culture vessel but the hydrogels are separated by a wall that create independently addressable microenvironments in the tissue culture vessel such as wells. In a multiplexed tissue culture vessel it is possible for some embodiments to include any number of aforementioned wells or independently addressable location within the cell culture vessel such that a hydrogel matrix in one well or location is different or the same as the hydrogel matrix in another well or location of the cell culture vessel.

The term “immune cell” as used herein can be any cell, for example, that participates in the immune activity of as subject, including defending a subject from infection or the symptom of infection or attacking, clearing or otherwise eliminating a dysfunction cell or pathogen from a cell in a subject, or improving the a symptoms of a disease caused by a pathogen. In some embodiments, immune cells comprise one or a plurality of B cells, T cells, antigen presenting cells such as astrocytes, dendritic cells and macrophages, stellate cells, granulocytes, monocytes, basophils, eosinophils, and/or mast cells. In some embodiments, the immune cell expresses CD4 or CD8 and one or more immunomodulatory molecule. In some embodiments, the immunomodulatory molecule is chosen from one of the following: IL-28, MHC, CD80, CD86, IL-1, IL-2, IL-4, IL-5, IL-6, IL-10, IL-18, MCP-1, MIP-Iα, MIP-Iβ, IL-8, L-selectin, P-selectin, E-selectin, CD34, GlyCAM-1, MadCAM-1, LFA-1, VLA-1, Mac-1, pl50.95, PECAM, ICAM-1, ICAM-2, ICAM-3, CD2, LFA-3, M-CSF, G-CSF, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAPl, TAP2 and functional fragments thereof, or a combination thereof. Immunomodulatory proteins are exemplified in U.S. Pat. No. 8,008,265.

The term “immunomodulatory” refers to a substance that has a modulatory effect on the immune system. Such substances can be readily identified using standard assays which indicate various aspects of the immune response, such as cytokine secretion, antibody production, NK cell activation and T cell proliferation. See, e.g., WO 97/28259; WO 98/16247; WO 99/11275; Krieg et al. (1995) Nature 374:546-549; Yamamoto et al. (1992) J. Immunol. 148:4072-76; Ballas et al. (1996) J. Immunol. 157:1840-45; Klinman et al. (1997) J. Immunol. 158:3635-39; Sato et al. (1996) Science 273:352-354; Pisetsky (1996) J. Immunol. 156:421-423; Shimada et al. (1986) Jpn. J. Cancer Res. 77:808-816; Cowdery et al. (1996) J. Immunol. 156:4570-75; Roman et al. (1997) Nat. Med. 3:849-854; Lipford et al. (1997a) Eur. J. Immunol. 27:2340-44; WO 98/55495 and WO 00/61151. Accordingly, these and other methods can be used to identify, test and/or confirm immunostimulatory substances, such as immunostimulatory nucleotides, immunostimulatory isolated nucleic acids.

In some embodiments, the two or more hydrogels may comprise different amount of PEG and/or Puramatrix. In some embodiments, the two or more hydrogels may have various densities. In some embodiments, the two or more hydrogels may have various permeabilities that are capable of allowing cells to grow within the hydrogel. In some embodiments, the two or more hydrogels may have various flexibilities. In some embodiments, the bioreactor, cell culture device or composition disclosed herein comprises a hydrogel comprising two layers of polymers: a cell-penetrable polymer and a cell-impenetrable polymer. In some embodiments, the cell-penetrable layer is layered at least in one region on top of the cell-impenetrable layer.

The term “cell-penetrable polymer” refers to a hydrophilic polymer, with identical or mixed monomer subunits, at a concentration and/or density sufficient to create spaces upon crosslinking in a solid or semisolid state on a solid substrate, such space are sufficiently biocompatible such that a cell or part of a cell can grow in culture.

The term “cell-impenetrable polymer” refers to a hydrophilic polymer, with identical or mixed monomer subunits, at a concentration and/or density sufficient to, upon crosslinking in a solid or semisolid state on a solid substrate, not create biocompatible spaces or compartments. In other words, an cell-impenetrable polymer is a polymer that, after crosslinking at a particular concentration and/or density, cannot support growth of a cell or part of a cell in culture.

The term “functional fragment” can be any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which the sequence is derived. In some embodiments, the functional fragment may retain 87%, 85%, 80%, 75%, 70%, 65%, or 60% sequence homology to the wild-type sequence upon which the sequence is derived.

One of ordinary skill can appreciate that a cell-impenetrable polymer and a cell-penetrable polymer may comprise the same or substantially the same polymers but the difference in concentration or density after crosslinking creates a hydrogel matrix with some portions conducive to grow a cell or part of cell in culture.

In some embodiments, the hydrogel or hydrogel matrixes can have various thicknesses. In some embodiments, the thickness of the hydrogel or hydrogel matrix is from about 100 μm to about 800 μm. In some embodiments, the thickness of the hydrogel or hydrogel matrix is from about 150 μm to about 800 μm. In some embodiments, the thickness of the hydrogel or hydrogel matrix is from about 200 μm to about 800 μm. In some embodiments, the thickness of the hydrogel or hydrogel matrix is from about 250 μm to about 800 μm. In some embodiments, the thickness of the hydrogel or hydrogel matrix is from about 300 μm to about 800 μm. In some embodiments, the thickness of the hydrogel or hydrogel matrix is from about 350 μm to about 800 μm. In some embodiments, the thickness of the hydrogel or hydrogel matrix is from about 400 μm to about 800 μm. In some embodiments, the thickness of the hydrogel or hydrogel matrix is from about 450 μm to about 800 μm. In some embodiments, the thickness of the hydrogel or hydrogel matrix is from about 500 μm to about 800 μm. In some embodiments, the thickness of the hydrogel or hydrogel matrix is from about 550 μm to about 800 μm. In some embodiments, the thickness of the hydrogel or hydrogel matrix is from about 600 μm to about 800 μm. In some embodiments, the thickness of the hydrogel or hydrogel matrix is from about 650 μm to about 800 μm. In some embodiments, the thickness of the hydrogel or hydrogel matrix is from about 700 μm to about 800 μm. In some embodiments, the thickness of the hydrogel or hydrogel matrix is from about 750 μm to about 800 μm. In some embodiments, the thickness of the hydrogel or hydrogel matrix is from about 100 μm to about 750 μm. In some embodiments, the thickness of the hydrogel or hydrogel matrix is from about 100 μm to about 700 μm. In some embodiments, the thickness of the hydrogel or hydrogel matrix is from about 100 μm to about 650 μm. In some embodiments, the thickness of the hydrogel or hydrogel matrix is from about 100 μm to about 600 μm. In some embodiments, the thickness of the hydrogel or hydrogel matrix is from about 100 μm to about 550 μm. In some embodiments, the thickness of the hydrogel or hydrogel matrix is from about 100 μm to about 500 μm. In some embodiments, the thickness of the hydrogel or hydrogel matrix is from about 100 μm to about 450 μm. In some embodiments, the thickness of the hydrogel or hydrogel matrix is from about 100 μm to about 400 μm. In some embodiments, the thickness of the hydrogel or hydrogel matrix is from about 100 μm to about 350 μm. In some embodiments, the thickness of the hydrogel or hydrogel matrix is from about 100 μm to about 300 μm. In some embodiments, the thickness of the hydrogel or hydrogel matrix is from about 100 μm to about 250 μm. In some embodiments, the thickness of the hydrogel or hydrogel matrix is from about 100 μm to about 200 μm. In some embodiments, the thickness of the hydrogel or hydrogel matrix is from about 100 μm to about 150 μm. In some embodiments, the thickness of the hydrogel or hydrogel matrix is from about 300 μm to about 600 μm. In some embodiments, the thickness of the hydrogel or hydrogel matrix is from about 400 μm to about 500 μm.

In some embodiments, the hydrogel or hydrogel matrixes can have various thicknesses. In some embodiments, the thickness of the hydrogel or hydrogel matrix is from about 10 μm to about 3000 μm. In some embodiments, the thickness of the hydrogel or hydrogel matrix is from about 150 μm to about 3000 μm. In some embodiments, the thickness of the hydrogel or hydrogel matrix is from about 200 μm to about 3000 μm. In some embodiments, the thickness of the hydrogel or hydrogel matrix is from about 250 μm to about 3000 μm. In some embodiments, the thickness of the hydrogel or hydrogel matrix is from about 300 μm to about 3000 μm. In some embodiments, the thickness of the hydrogel or hydrogel matrix is from about 350 μm to about 3000 μm. In some embodiments, the thickness of the hydrogel or hydrogel matrix is from about 400 μm to about 3000 μm. In some embodiments, the thickness of the hydrogel or hydrogel matrix is from about 450 μm to about 3000 μm. In some embodiments, the thickness of the hydrogel or hydrogel matrix is from about 500 μm to about 3000 μm. In some embodiments, the thickness of the hydrogel or hydrogel matrix is from about 550 μm to about 3000 μm. In some embodiments, the thickness of the hydrogel or hydrogel matrix is from about 600 μm to about 3000 μm. In some embodiments, the thickness of the hydrogel or hydrogel matrix is from about 650 μm to about 3000 μm. In some embodiments, the thickness of the hydrogel or hydrogel matrix is from about 700 μm to about 3000 μm. In some embodiments, the thickness of the hydrogel or hydrogel matrix is from about 750 μm to about 3000 μm. In some embodiments, the thickness of the hydrogel or hydrogel matrix is from about 800 μm to about 3000 μm. In some embodiments, the thickness of the hydrogel or hydrogel matrix is from about 850 μm to about 3000 μm. In some embodiments, the thickness of the hydrogel or hydrogel matrix is from about 900 μm to about 3000 μm. In some embodiments, the thickness of the hydrogel or hydrogel matrix is from about 950 μm to about 3000 μm. In some embodiments, the thickness of the hydrogel or hydrogel matrix is from about 1000 μm to about 3000 μm. In some embodiments, the thickness of the hydrogel or hydrogel matrix is from about 1500 μm to about 3000 μm. In some embodiments, the thickness of the hydrogel or hydrogel matrix is from about 2000 μm to about 3000 μm. In some embodiments, the thickness of the hydrogel or hydrogel matrix is from about 2500 μm to about 3000 μm. In some embodiments, the thickness of the hydrogel or hydrogel matrix is from about 100 μm to about 2500 μm. In some embodiments, the thickness of the hydrogel or hydrogel matrix is from about 100 μm to about 2000 μm. In some embodiments, the thickness of the hydrogel or hydrogel matrix is from about 100 μm to about 1500 μm. In some embodiments, the thickness of the hydrogel or hydrogel matrix is from about 100 μm to about 1000 μm. In some embodiments, the thickness of the hydrogel or hydrogel matrix is from about 100 μm to about 950 μm. In some embodiments, the thickness of the hydrogel or hydrogel matrix is from about 100 μm to about 900 μm. In some embodiments, the thickness of the hydrogel or hydrogel matrix is from about 100 μm to about 850 μm. In some embodiments, the thickness of the hydrogel or hydrogel matrix is from about 100 μm to about 800 μm. In some embodiments, the thickness of the hydrogel or hydrogel matrix is from about 100 μm to about 750 μm. In some embodiments, the thickness of the hydrogel or hydrogel matrix is from about 100 μm to about 700 μm. In some embodiments, the thickness of the hydrogel or hydrogel matrix is from about 100 μm to about 650 μm. In some embodiments, the thickness of the hydrogel or hydrogel matrix is from about 100 μm to about 600 μm. In some embodiments, the thickness of the hydrogel or hydrogel matrix is from about 100 μm to about 550 μm. In some embodiments, the thickness of the hydrogel or hydrogel matrix is from about 100 μm to about 500 μm. In some embodiments, the thickness of the hydrogel or hydrogel matrix is from about 100 μm to about 450 μm. In some embodiments, the thickness of the hydrogel or hydrogel matrix is from about 100 μm to about 400 μm. In some embodiments, the thickness of the hydrogel or hydrogel matrix is from about 100 μm to about 350 μm. In some embodiments, the thickness of the hydrogel or hydrogel matrix is from about 100 μm to about 300 μm. In some embodiments, the thickness of the hydrogel or hydrogel matrix is from about 100 μm to about 250 μm. In some embodiments, the thickness of the hydrogel or hydrogel matrix is from about 100 μm to about 200 μm. In some embodiments, the thickness of the hydrogel or hydrogel matrix is from about 100 μm to about 150 μm. In some embodiments, the thickness of the hydrogel or hydrogel matrix is from about 300 μm to about 600 μm. In some embodiments, the thickness of the hydrogel or hydrogel matrix is from about 400 μm to about 500 μm.

In some embodiments, the hydrogel or hydrogel matrix comprises one or more synthetic polymers. In some embodiments, the hydrogel or hydrogel matrix comprises one or more of the following synthetic polymers: polyethylene glycol (polyethylene oxide), polyvinyl alcohol, poly-2-hydroxyethyl methacrylate, polyacrylamide, silicones, and any derivatives or combinations thereof.

In some embodiments, the hydrogel or hydrogel matrix comprises one or more synthetic and/or natural polysaccharides. In some embodiments, the hydrogel or hydrogel matrix comprises one or more of the following polysaccharides: hyaluronic acid, heparin sulfate, heparin, dextran, agarose, chitosan, alginate, and any derivatives or combinations thereof.

In some embodiments, the hydrogel or hydrogel matrix comprises one or more proteins and/or glycoproteins. In some embodiments, the hydrogel or hydrogel matrix comprises one or more of the following proteins: collagen, gelatin, elastin, titin, laminin, fibronectin, fibrin, keratin, silk fibroin, and any derivatives or combinations thereof.

In some embodiments, the hydrogel or hydrogel matrix comprises one or more synthetic and/or natural polypeptides. In some embodiments, the hydrogel or hydrogel matrix comprises one or more of the following polypeptides: polylysine, polyglutamate or polyglycine.

In some embodiments, the hydrogel comprises one or a combination of polymers sletec from those published in Khoshakhlagh et al., “Photoreactive interpenetrating network of hyaluronic acid and Puramatrix as a selectively tunable scaffold for neurite growth” Acta Biomaterialia, Jan. 21, 2015.

Any hydrogel suitable for cell growth can be formed by placing any one or combination of polymers disclosed herein at a concentration and under conditions and for a sufficient time period sufficient to create two distinct densities of crosslinked polymers: one cell-penetrable and one cell-impenetrable. The polymers may be synthetic polymers, polysaccharides, natural proteins or glycoproteins and/or polypeptides such as those selected from below.

### Synthetic Polymers

Such as polyethylene glycol (polyethylene oxide), polyvinyl alcohol, poly-2-hydroxyethyl methacrylate, polyacrylamide, silicones, their combinations, and their derivatives.
Polysaccharides (Whether Synthetic or Derived from Natural Sources)
Such as hyaluronic acid, heparan sulfate, heparin, dextran, agarose, chitosan, alginate, their combinations, and their derivatives.

### Natural Proteins or Glycoproteins

Such as collagen, gelatin, elastin, titin, laminin, fibronectin, fibrin, keratin, silk fibroin, their combinations, and their derivatives.

### Polypeptides (Whether Synthetic or Natural Sources)

Such as polylysine, and all of the RAD and EAK peptides already listed.

The term “three-dimensional” or “3D” as used herein means, for example, a thickness of culture of cells such that there are at least three layers of cells growing adjacent to one another. In some embodiments, the term three-dimensional means that, in context of the disclosed systems, the neurites and/or axons are from about 10 to about 1000 microns in thickness or height. In some embodiments, the term three-dimensional means that, in context of the disclosed systems, the neurites and/or axons are from about 10 to about 100 microns in thickness or height.

The term “isolated neurons” refers to neuronal cells that have been removed or disassociated from an organism or culture from which they originally grow. In some embodiments isolated neurons are neurons in suspension. In some embodiments, isolated neurons are a component of a larger mixture of cells including a tissue sample or a suspension with non-neuronal cells. In some embodiments, neuronal cells have become isolated when they are removed from the animal from which they are derived, such as in the case of a tissue explant. In some embodiments isolated neurons are those neurons in a DRG excised from an animal. In some embodiments, the isolated neurons comprise at least one or a plurality cells that are from one species or a combination of the species chosen from: sheep cells, goat cells, horse cells, cow cells, human cells, monkey cells, mouse cells, rat cells, rabbit cells, canine cells, feline cells, porcine cells, or other non-human mammals. In some embodiments, the isolated neurons are human cells. In some embodiments, the isolated neurons are stem cells that are pre-conditioned to have a differentiated phenotype similar to or substantially similar to a human neuronal cell. In some embodiments, the isolated neurons are human cells. In some embodiments, the isolated neurons are stem cells that are pre-conditioned to have a differentiated phenotype similar to or substantially similar to a non-human neuronal cell. In some embodiments, the stem cells are selected from: mesenchymal stem cells, induced pluripotent stem cells, embryonic stem cells, hematopoietic stem cells, epidermal stem cells, stem cells isolated from the umbilical cord of a mammal, or endodermal stem cells.

The term “neurodegenerative disease” is used throughout the specification to describe a disease which is caused by damage to the central nervous system ad or peripheral nervous system. Exemplary neurodegenerative diseases which may be examples of diseases that could be studied using the disclosed model, system or device include for example, Parkinson's disease, Huntington's disease, amyotrophic lateral sclerosis (Lou Gehrig's disease), Alzheimer's disease, lysosomal storage disease (“white matter disease” or glial/demyelination disease, as described, for example by Folkerth, J. Neuropath. Exp. Neuro., 58, 9, September, 1999), Tay Sachs disease (beta hexosamimidase deficiency), other genetic diseases, multiple sclerosis, brain injury or trauma caused by ischemia, accidents, environmental insult, etc., spinal cord damage, ataxia and alcoholism. In addition, the present invention may be used to test the efficacy, toxicity, or neurodegenerative effect of agents on neuronal cells in culture for the study of treatments for neurodegenerative diseases. The term neurodegenerative diseases also includes neurodevelopmental disorders including for example, autism and related neurological diseases such as schizophrenia, among numerous others.

The term “neuronal cells” as used herein refers to, for example, cells that comprise at least one or a combination of dendrites, axons, and somata, or, alternatively, any cell or group of cells isolated from nervous system tissue. In some embodiments, neuronal cells are any cell that comprises or is capable of forming an axon. In some embodiments, the neuronal cell is a Schwann cell, glial cell, neuroglia, cortical neuron, embryonic cell isolated from or derived from neuronal tissue or that has differentiated into a cell with a neuronal phenotype or a phenotype which is substantially similar to a phenotype of a neuronal cell, induced pluripotent stem cells (iPS) that have differentiated into a neuronal phenotype, or mesenchymal stem cells that are derived from neuronal tissue or differentiated into a neuronal phenotype. In some embodiments, neuronal cells are neurons from dorsal root ganglia (DRG) tissue, retinal tissue, spinal cord tissue, or brain tissue from an adult, adolescent, child or fetal subject. In some embodiments, neuronal cells are any one or plurality of cells isolated from the neuronal tissue of a subject. In some embodiments, the neuronal cells are mammalian cells. In some embodiments, the cells are human cells and/or rat cells. In some embodiments, the cells are non-human mammalian cells or derived from cells that are isolated from non-human mammals. If isolated or disassociated from the original animal from which the cells are derived, the neuronal cells may comprise isolated neurons from more than one species. In some embodiments, the spheroid are free of a DRG tissue.

In some embodiments, neuronal cells are one or more of the following: central nervous system neurons, peripheral nervous system neurons, sympathetic neurons, parasympathetic neurons, enteric nervous system neurons, spinal motor neurons, motor neurons, sensory neurons, autonomic neurons, somatic neurons, dorsal root ganglia, cholinergic neurons, GABAergic neurons, glutamatergic neurons, dopaminergic neurons, serotonergic neurons, interneurons, adrenergic neurons, and trigeminal ganglia. In some embodiments, glial cells are one or more of the following: astrocytes, oligodendrocytes, Schwann cells, microglia, ependymal cells, radial glia, satellite cells, enteric glial cells, and pituyicytes. In some embodiments, immune cells are one or more of the following: macrophages, T cells, B cells, leukocytes, lymphocytes, monocytes, mast cells, neutrophils, natural killer cells, and basophils. In some embodiments, stem cells are one or more of the following: hematopoietic stem cells, neural stem cells, embryonic stem cells, adipose derived stem cells, bone marrow derived stem cells, induced pluripotent stem cells, astrocyte derived induced pluripotent stem cells, fibroblast derived induced pluripotent stem cells, renal epithelial derived induced pluripotent stem cells, keratinocyte derived induced pluripotent stem cells, peripheral blood derived induced pluripotent stem cells, hepatocyte derived induced pluripotent stem cells, mesenchymal derived induced pluripotent stem cells, neural stem cell derived induced pluripotent stem cells, adipose stem cell derived induced pluripotent stem cells, preadipocyte derived induced pluripotent stem cells, chondrocyte derived induced pluripotent stem cells, and skeletal muscle derived induced pluripotent stem cells. In some embodiments, spheroids may also include other cell types such as keratinocytes or endothelial cells.

The terms “neuronal cell culture medium” or simply “culture medium” as used herein can be any nutritive substance suitable for supporting the growth, culture, cultivating, proliferating, propagating, or otherwise manipulating neuronal cells. In some embodiments, the medium comprises neurobasal medium supplemented with nerve growth factor (NGF). In some embodiments, the medium comprises fetal bovine serum (FBS). In some embodiments, the medium comprises L-glutamine. In some embodiments, the medium comprises ascorbic acid in a concentration ranging from about 0.001% weight by volume to about 0.01% weight by volume. In some embodiments, the medium comprises ascorbic acid in a concentration ranging from about 0.001% weight by volume to about 0.008% weight by volume. In some embodiments, the medium comprises ascorbic acid in a concentration ranging from about 0.001% weight by volume to about 0.006% weight by volume. In some embodiments, the medium comprises ascorbic acid in a concentration ranging from about 0.001% weight by volume to about 0.004% weight by volume. In some embodiments, the medium comprises ascorbic acid in a concentration ranging from about 0.002% weight by volume to about 0.01% weight by volume. In some embodiments, the medium comprises ascorbic acid in a concentration ranging from about 0.003% weight by volume to about 0.01% weight by volume. In some embodiments, the medium comprises ascorbic acid in a concentration ranging from about 0.004% weight by volume to about 0.01% weight by volume. In some embodiments, the medium comprises ascorbic acid in a concentration ranging from about 0.006% weight by volume to about 0.01% weight by volume. In some embodiments, the medium comprises ascorbic acid in a concentration ranging from about 0.008% weight by volume to about 0.01% weight by volume. In some embodiments, the medium comprises ascorbic acid in a concentration ranging from about 0.002% weight by volume to about 0.006% weight by volume. In some embodiments, the medium comprises ascorbic acid in a concentration ranging from about 0.003% weight by volume to about 0.005% weight by volume.

In some embodiments, the hydrogel, hydrogel matrix, and/or neuronal cell culture medium comprises any one or more of the following components: artemin, ascorbic acid, ATP, μ-endorphin, BDNF, bovine calf serum, bovine serum albumin, calcitonin gene-related peptide, capsaicin, carageenan, CCL2, ciliary neurotrophic factor, CX3CL1, CXCL1, CXCL2, D-serine, fetal bovine serum, fluorocitrate. formalin, glial cell line-derived neurotrophic factor, glial fibrillary acid protein, glutamate, IL-1, IL-1α, IL-1β, IL-6, IL-10, IL-12, IL-17, IL-18, insulin, laminin, lipoxins, mac-1-saporin, methionine sulfoximine, minocycline, neuregulin-1, neuroprotectins, neurturin, NGF, nitric oxide, NT-3, NT-4, persephin, platelet lysate, PMX53, Poly-D-lysine (PLL), Poly-L-lysine (PLL), propentofylline, resolvins, S100 calcium-binding protein B, selenium, substance P, TNF-α, type I-V collagen, and zymosan.

As described herein, the term “optogenetics” refers to a biological technique which involves the use of light to control cells in living tissue, typically neurons, that have been genetically modified to express light-sensitive ion channels. It is a neuromodulation method employed in neuroscience that uses a combination of techniques from optics and genetics to control and monitor the activities of individual neurons in living tissue—even within freely-moving animals—and to precisely measure the effects of those manipulations in real-time. The key reagents used in optogenetics are light-sensitive proteins. Spatially-precise neuronal control is achieved using optogenetic actuators like channelrhodopsin, halorhodopsin, and archaerhodopsin, while temporally-precise recordings can be made with the help of optogenetic sensors for calcium (Aequorin, Cameleon, GCaMP), chloride (Clomeleon) or membrane voltage (Mermaid). In some embodiments, neural cells modified with optogenetic actuators and/or sensors are used in the culture systems described herein.

The term “plastic” refers to biocompatible polymers comprising hydrocarbons. In some embodiments, the plastic is selected from the group consisting of: Polystyrene (PS), Poly acrylo nitrile (PAN), Poly carbonate (PC), polyvinylpyrrolidone, polybutadiene (PVP), Polyvinyl butyral (PVB), Poly vinyl chloride (PVC), Poly vinyl methyl ether (PVME), poly lactic-co-glycolic acid (PLGA), poly(l-lactic acid), polyester, polycaprolactone (PCL), poly ethylene oxide (PEO), polyaniline (PANI), polyflourenes, polypyrroles (PPY), poly ethylene dioxythiophene (PEDOT), and a mixture of two or any two or more of the foregoing polymers. In some embodiments, the plastic is a mixture of three, four, five, six, seven, eight or more polymers.

The term “seeding” as used herein refers to, for example, transferring an amount of cells into a new culture vessel. The amount may be defined and may use volume or number of cells as the basis of the defined amount. The cells may be part of a suspension.

The terms “sequence identity” as used herein refers to, in the context of two or more nucleic acids or polypeptide sequences, the specified percentage of residues that are the same over a specified region. The term is synonymous with “sequence homology” or sequences being “homologous to” another sequence. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

The term “solid substrate” as used herein refers to any substance that is a solid support that is free of or substantially free of cellular toxins. In some embodiments, the solid substrate comprise one or a combination of silica, plastic, and metal. In some embodiments, the solid substrate comprises pores of a size and shape sufficient to allow diffusion or non-active transport of proteins, nutrients, and gas through the solid substrate in the presence of a cell culture medium. In some embodiments, the pore size is no more than about 10, 9, 8, 7, 6, 5, 4, 3, 2 or 1 micron in diameter. One of ordinary skill could determine how big of a pore size is necessary based upon the contents of the cell culture medium and exposure of cells growing on the solid substrate in a particular microenvironment. For instance, one of ordinary skill in the art can observe whether any cultured cells in the system or device are viable under conditions with a solid substrate comprises pores of various diameters. In some embodiments, the solid substrate comprises a base with a predetermined shape that defines the shape of the exterior and interior surface. In some embodiments, the base comprises one or a combination of silica, plastic, ceramic, or metal and wherein the base is in a shape of a cylinder or in a shape substantially similar to a cylinder, such that the first cell-impenetrable polymer and a first cell-penetrable polymer coat the interior surface of the base and define a cylindrical or substantially cylindrical interior chamber; and wherein the opening is positioned at one end of the cylinder. In some embodiments, the base comprises one or a plurality of pores of a size and shape sufficient to allow diffusion of protein, nutrients, and oxygen through the solid substrate in the presence of the cell culture medium. In some embodiments, the solid substrate comprises a plastic base with a pore size of no more than 1 micron in diameter and comprises at least one layer of hydrogel matrix; wherein the hydrogel matrix comprises at least a first cell-impenetrable polymer and at least a first cell-penetrable polymer; the base comprises a predetermined shape around which the first cell-impenetrable polymer and at least a first cell-penetrable polymer physically adhere or chemically bond; wherein the solid substrate comprises at least one compartment defined at least in part by the shape of an interior surface of the solid substrate and accessible from a point outside of the solid substrate by an opening, optionally positioned at one end of the solid substrate. In some embodiments, where the solid substrate comprises a hollow interior portion defined by at least one interior surface, the cells in suspension or tissue explants may be seeded by placement of cells at or proximate to the opening such that the cells may adhere to at least a portion the interior surface of the solid substrate for prior to growth. The at least one compartment or hollow interior of the solid substrate allows a containment of the cells in a particular three-dimensional shape defined by the shape of the interior surface solid substrate and encourages directional growth of the cells away from the opening. In the case of neuronal cells, the degree of containment and shape of the at least one compartment are conducive to axon growth from soma positioned within the at least one compartment and at or proximate to the opening. In some embodiments, the solid substrate is cylindrical, tubular or substantially tubular or cylindrical such that the interior compartment is cylindrical or partially cylindrical in shape. In some embodiments, the solid substrate comprises one or a plurality of branched tubular interior compartments. In some embodiments, the bifurcating or multiply bifurcating shape of the hollow interior portion of the solids is configured for or allows axons to grow in multiple branched patterns. When and if electrodes are placed at to near the distal end of an axon and at or proximate to a neuronal cell soma, electrophysiological metrics, such as intracellular action potential can be measured within the device or system. In some embodiments, the electrodes are operably linked to a voltmeter, ammeter and/or a device capable of generating a current on a length of wire physically connecting the electrodes to the voltmeter, ammeter and/or device.

The disclosure relates to properly stuff hydrogel that comprises a mixture of both cell penetrable and cell impenetrable polymers. In some embodiments, the hydrogel comprises from about 10% to about 20% PEG and has a total modulus from about 0.1 to about 200 Pa. In some embodiments, the hydrogel has a modulus of about 0.5 Pa. In some embodiments, the hydrogel has a modulus of about 10 Pa. In some embodiments, the hydrogel has a modulus of about 50 Pa. In some embodiments, the hydrogel has a modulus of about 75 Pa. In some embodiments, the hydrogel has a modulus of about 90 Pa. In some embodiments, the hydrogel has a modulus of about 100 Pa. In some embodiments, the hydrogel has a modulus of about 125 Pa. In some embodiments, the hydrogel has a modulus of about 150 Pa. In some embodiments, the hydrogel has a modulus of about 175 Pa. In some embodiments, the hydrogel has a modulus of about 200 Pa. In some embodiments, the hydrogel has a modulus of no more than about 230 Pa.

### Spheroids

As used herein, a “spheroid” or “cell spheroid” can be, for example, any grouping of cells in a three-dimensional shape that generally corresponds to an oval or circle or convex or concave arc rotated about one of its principal axes, major or minor, and includes three-dimensional egg shapes, oblate and prolate spheroids, spheres, lens-shaped or substantially equivalent shapes.

A spheroid of the present invention can have any suitable width, length, thickness, and/or diameter. In some embodiments, a spheroid may have a width, length, thickness, and/or diameter in a range from about 10 μm to about 50,000 μm, or any range therein, such as, but not limited to, from about 10 μm to about 900 μm, about 100 μm to about 700 μm, about 300 μm to about 600 μm, about 400 μm to about 500 μm, about 500 μm to about 1,000 μm, about 600 μm to about 1,000 μm, about 700 μm to about 1,000 μm, about 800 μm to about 1,000 μm, about 900 μm to about 1,000 μm, about 750 μm to about 1,500 μm, about 1,000 μm to about 5,000 μm, about 1,000 μm to about 10,000 μm, about 2,000 to about 50,000 μm, about 25,000 μm to about 40,000 μm, or about 3,000 μm to about 15,000 μm. In some embodiments, a spheroid may have a width, length, thickness, and/or diameter of about 50 μm, 100 μm, 200 μm, 300 μm, 400 μm, 500 μm, 600 μm, 700 μm, 800 m, 900 μm, 1,000 μm, 5,000 μm, 10,000 μm, 20,000 μm, 30,000 μm, 40,000 μm, or 50,000 μm. In some embodiments, a plurality of spheroids are generated, and each of the spheroids of the plurality may have a width, length, thickness, and/or diameter that varies by less than about 20%, such as, for example, less than about 15%, 10%, or 5%. In some embodiments, each of the spheroids of the plurality may have a different width, length, thickness, and/or diameter within any of the ranges set forth above.

The cells in a spheroid may have a particular orientation. In some embodiments, the spheroid may comprise an interior core and an exterior surface. In some embodiments, the spheroid may be hollow (i.e., may not comprise cells in the interior). In some embodiments, the interior core cells and the exterior surface cells are different types of cell. In some embodiments, the interior core comprises a magnetic nanoparticle.

The spheroids may vary in their stiffness, e.g., as measured by elastic modulus (Pascals; Pa). In certain embodiments, the elastic moduli of the spheroids are in a range from about 100 Pa to about 10,000 Pa, e.g., from about 100 Pa to about 12,000 Pa or from about 100 Pa to about, 4800 Pa. In some embodiments, the elastic moduli of the spheroids may be about 1200 Pa. As another example, the spheroid modulus may vary from about at least 10 Pa, at least about 100 Pa., at least about 150 Pa, at least about 200 Pa, or at least about 450 Pa. In some embodiments, the composition or device of the disclosure comprises one or a plurality of wells and each well comprises one or a plurality of different spheroids, a first, second, third, fourth or fifth or more population of spheroids. In one embodiment, the first spheroid comprises an elastic modulus from about 100 Pa to about 300 Pa, and the second spheroid comprises an elastic modulus from about 400 Pa to about 800 Pa. In another example, the first spheroid is characterized by an elastic modulus from about 50 to about 200 Pa, and a second spheroid is characterized by an elastic modulus from about 250 Pa to about 500 Pa.

In some embodiments, spheroids may be made up of one, two, three or more different cell types, including one or a plurality of neuronal cell types and/or one or a plurality of stem cell types. In some embodiments, the interior core cells may be made up of one, two, three, or more different cell types. In some embodiments, the exterior surface cells may be made up of one, two, three, or more different cell types.

In some embodiments, the spheroids comprise at least two types of cells. In some embodiments the spheroids comprise neuronal cells and non-neuronal cells. In some embodiments, the spheroids comprise neuronal cells and astrocytes at a ratio of about 5:1, 4:1, 3:1, 2:1 or 1:1 of neuronal cells to astrocytes. In some embodiments, the spheroids comprise neuronal cells and non-neuronal cells at a ratio of about 5:1, 4:1, 3:1, 2:1 or 1:1. In some embodiments, the spheroids comprise neuronal cells and non-neuronal cells at a ratio of about 1:5: 1:4, 1:3, or 1:2. Any combination of cell types disclosed herein may be used in the above-identified ratios within the spheroids of the disclosure.

Depending on the particular embodiment, groups of cells may be placed according to any suitable shape, geometry, and/or pattern. In some embodiments, the cells are arranged in a sphere across the surface area of a bead or nanoparticle with a solid or hollow core. For example, independent groups of cells may be deposited as spheroids, and the spheroids may be arranged within a three dimensional grid, or any other suitable three dimensional pattern. The independent spheroids may all comprise approximately the same number of cells and be approximately the same size, or alternatively, different spheroids may have different numbers of cells and different sizes. In some embodiments, multiple spheroids may be arranged in shapes such as an L or T shape, radially from a single point or multiple points, sequential spheroids in a single line or parallel lines, tubes, cylinders, toroids, hierarchically branched vessel networks, high aspect ratio objects, thin closed shells, organoids, or other complex shapes which may correspond to geometries of tissues, vessels or other biological structures.

Any suitable physiological response of the spheroid may be determined, evaluated, measured, and/or identified in a method of the present disclosure. In some embodiments, 1, 2, 3, 4, or more physiological response(s) of the spheroid may be determined, evaluated, measured, and/or identified in a method of the present disclosure. In some embodiments, the physiological response of the spheroid may be a change in morphology for the spheroid. The method may comprise determining a change in morphology for the spheroid, which may include estimating at least one morphology parameter prior to contacting the spheroid with an agent, such as a chemical and/or biological compound, estimating the at least one morphology parameter after contacting the spheroid with the agent, and calculating the difference between the at least one morphology parameter prior to and after contacting the spheroid with the agent to provide the change in morphology for the spheroid. In some embodiments, the physiological response of the spheroid may be the spheroid shrinking or swelling in response to contact with an agent. Morphology of the spheroid may be determined using any methods known to those of skill in the art, such as, but not limited to, quantifying eccentricity and/or cross sectional area.

In some embodiments, the physiological response of the spheroid may be a change in volume for the spheroid. The method may comprise determining a change in volume for the spheroid, which may include estimating a first volume prior to contacting the spheroid with an agent, estimating a second volume after contacting the spheroid with the agent, and calculating the difference between the first volume and the second volume to provide the change in volume for the spheroid. In some embodiments, the physiological response of the spheroid may be the spheroid shrinking or swelling in response to contact with an agent.

The agent may be any suitable compound, such as, for example, an organic compound, a small molecule compound (e.g., a small molecule organic compound), a protein, an antibody, an oligonucleotide (e.g., DNA and/or RNA), a gene therapy vehicle (e.g., a viral vector) and any combination thereof. One or more (e.g., 1, 2, 3, 4, 5, or more) agents may be used in a method of the present invention. For example, a method of the present invention may comprise contacting a spheroid of the present invention with two or more different agents. In some embodiments, a method of the present invention may modulate an activity in a spheroid indirectly, such as, for example, by contacting a spheroid of the present invention with a gene therapy vehicle (e.g., a viral vector).

A method of the present invention may comprise culturing cells and/or a spheroid. Culturing may be carried out using methods known to those knowledgeable in the field. In some embodiments, cells and/or a spheroid may be cultured for any desired period of time, such as, but not limited, hours, days, weeks, or months. In some embodiments, cells and/or a spheroid may be cultured for about 1, 2, 3, 4, 5, 6, or 7 days, or about 1, 2, 3, 4, 5, 6, 7, 8, 9, 10, or 11 or more weeks.

Cell culture media suitable for the methods of the present invention are known in the art and include, but are not limited to, BEGM™ Bronchial Epithelial Cell Growth medium, Dulbecco's Modified Eagle's Medium (DMEM), Dulbecco's Modified Eagle's Medium high glucose (DMEM-H), McCoy's 5A Modified Medium, RPMI, Ham's media, Medium 199, mTeSR, and so on. The cell culture medium may be supplemented with additional components such as, but not limited to, vitamins, minerals, salts, growth factors, carbohydrates, proteins, serums, amino acids, attachment factors, cytokines, growth factors, hormones, antibiotics, therapeutic agents, buffers, etc. The cell culture components and/or conditions may be selected and/or changed during the methods of the present invention to enhance and/or stimulate certain cellular characteristics and/or properties. Examples of seeding methods and cell culturing methods are described in U.S. Pat. Nos. 5,266,480, 5,770,417, 6,537,567, and 6,962,814 and Oberpenning et al. “De novo reconstitution of a functional mammalian urinary bladder by tissue engineering” Nature Biotechnology 17:149-155 (1999), which are incorporated herein by reference in their entirety. In some embodiments, the cell culture medium is changed in a stepwise fashion to encourage myelination of axons in culture. Pre-myelination and myelination media includes the following components:

TABLE 1
Components of myelination inducing media regimen.

| Myelination | Neural | Premyelination | Myelination |
|---|---|---|---|
| media regimen | growth media | media | media |
| Neurobasal | X | | |
| media | | | |
| Basal medium | | X | X |
| eagle | | | |
| NGF | X | X | X |
| B-27 | X | | |
| supplement | | | |
| ITS supplement | | X | X |
| D-glucose | | X. | X |
| BSA | | X | |
| AA | | | X |
| Anti-anti | X | X | X |
| Glutamax | X | X | X |
| FBS | | | X |

In some embodiments, the solid substrate, cell culture device, or nanoparticles comprise a spheroid comprising one or plurality of cell types disclosed herein. Any of the particles may comprise any one or combination of 1, 2, 3, 4, 5, 6, 7, 8 or more cell types in the application.

The terms “nanoparticles” or “nanoshuttles,” as each term may be used interchangeably, are particles comprising at least one region. Magnetic particles ranging from about 0.7 to about 1.5 microns have been described in the patent literature, including, by way of example, U.S. Pat. Nos. 3,970,518; 4,018,886; 4,230,685; 4,267,234; 4,452,773; 4,554,088; 4,659,678; 6,623,982, 6,645,731 and US. Application Number 20110250146, each of which is incorporated by reference in their respective entireties. The nanaoparticles may be used to magnetize, i.e. make responsive to a magnetic field, any cell or spheroid described herein. Some compositions and/or systems of the disclosure include cells in contact with or spheroids comprising a magnetic responsive element. In some embodiments, compositions and/or systems of the disclosure include cells in contact with or spheroids comprising one or a plurality of magnetic nanoparticles. As used herein a “magnetically responsive element” can be any element or molecule that will respond to a magnetic field. One or a plurality of the nanoparticles must contain or be a magnetically responsive element. In some embodiments, nanoparticles may taken up or adsorbed by any of the cells described herein. In some embodiments, a magnetic field can be used to manipulate the location, shape, patterns or motion of a cell or spheroid.

In some embodiments, charged nanoparticles have a nano-scale size. In some embodiments, nanoparticles of the disclosure have a size from about 5 nm to about 1000 nm, or any range therein. In some embodiments, the nanoparticles have a diameter from about 5 nm to about 250 nm, from about 25 nm to about 225 nm, from about 50 nm to about 200 nm, from about 75 nm to about 150 nm in size. In some embodiments, the nanoparticles have a diameter of about 5 nm, about 10 nm, about 20 nm, about 40 nm, about 60 nm, about 80 nm, about 100 nm, about 120 nm, about 140 nm, about 160 nm, about 180 nm, about 200 nm, about 250 nm, about 300 nm, about 400 nm, about 500 nm, about 600 nm, about 700 nm, about 800 nm, about 900 nm, or about 1000 nm. In some embodiments the nanoparticles are no more than about 5 nm, about 10 nm, about 20 nm, about 40 nm, about 60 nm, about 80 nm, about 100 nm, about 120 nm, about 140 nm, about 160 nm, about 180 nm, about 200 nm, about 250 nm, about 300 nm, about 400 nm, about 500 nm, about 600 nm, about 700 nm, about 800 nm, about 900 nm, or about 1000 nm in diameter. In some embodiments, nanoparticles are of a substantially uniform size. In some embodiments, nanoparticles are of different sizes. In some embodiments, the size of the nanoparticle will depend on what type of cell is being used.

The magnetically responsive element can be any element or molecule that will respond to a magnetic field. In some embodiments, the magnetically responsive element is a rare earth magnet such as, for example, samarium cobalt (SmCo) or neodymium iron boron (NdFeB). In some embodiments, the magnetically responsive element is a ceramic magnet material, such as, for example, strontium ferrite. In some embodiments, the magnetically responsive element is a magnetic element, such as, for example, iron, cobalt, nickel, or any alloy or oxide thereof. In some embodiments, the magnetically responsive element comprises gold. In some embodiments, the magnetically responsive element is a paramagnetic material that reacts to a magnetic field, but is not a magnet itself, as this allows for easier assembly of the materials.

In some embodiments, nanoparticles comprise one or a plurality of iron oxides, including, for example, iron(III) oxide, α-Fe₂O₃, γ-Fe₂O₃, β-Fe₂O₃, ε-Fe₂O₃, iron(II) oxide, or iron(II,III) oxide. In some embodiments, nanoparticles comprise one or a plurality of gold, iron oxide, and poly-lysine.

In some aspects of the present disclosure, a coated, magnetic particle is provided which comprises a nanoparticle core of magnetic material, and a base coating material on the magnetic core in an amount sufficient to hinder non-specific binding of biological macromolecules to the magnetic core. These magnetic particles are characterized by extremely low, non-specific binding as well as highly efficient target capture which are essential to achieve a level of enrichment the enrichment required to effectively isolate very rare cells, such as neurons or other cell types disclosed herein. In an alternative embodiment, a coated, magnetic particle is provided which comprises the following: i. a nanoparticle core of magnetic material; ii. a base coating material that forms a discontinuous coating on the magnetic core, providing at least one area of discontinuity which, if accessible, contributes to non-specific binding of the base coated particle to biological macromolecules; and iii. an additional coating material that hinders access to the areas of discontinuity by biological macromolecules. The magnetic core material of the particles described immediately above may comprise at least one transition metal oxide and a suitable base coating material comprises a protein. Proteins suitable for coating magnetic particles include but are not limited to bovine serum albumin and casein. The additional coating material may be the original coating proteins or one member of a specific binding pair which is coupled to the base material on the magnetic core. Exemplary specific binding pairs include biotin-streptavidin, antigen-antibody, receptor-hormone, receptor-ligand, agonist-antagonist, lectin-carbohydrate, Protein A-antibody Fc, and avidin-biotin. In one embodiment, the member of the specific binding pair is coupled to the base coating material through a bifunctional linking compound. Exemplary biofunctional linking compounds include succinimidyl-propiono-dithiopyridine (SPDP), and sulfosuccinimidil-4-[maleimidomethyl]cyclohexane-1-carboxylate (SMCC), however a variety of other such heterobifunctional linker compounds are available from Pierce, Rockford, Ill.

The coated magnetic particles of the invention preferably have between 70-90% magnetic mass. In some embodiments, a major portion of the magnetic particles have a particle size in the range from about 90 to about 150 nm. Particles may be synthesized such that they are more monodisperse, e.g., in the range of from about 90 to about 120 nm or from about 120 to about 150 nm. The particles of the invention are typically suspended in a biologically compatible medium.

In some embodiments, nanoparticles may be combined with a support molecule. The “support molecule” is generally a polymer or other long molecule that serves to hold the nanoparticles and cells together in an intimate admixture. The support molecule can be positively charged, negatively charged, of mixed charge, or neutral, and can be combinations of more than one support molecule. In some embodiments, the support molecule is a natural polymer or cell-derived polymer. Non-limiting examples of such polymers include peptides, polysaccharides, and nucleic acids. In other embodiments, the support molecule is a synthetic polymer. In some embodiments, the polymer is poly-lysine. In some embodiments, the support molecule can be one or more of poly-lysine, fibronectin, collagen, laminin, BSA, hyaluronan, glycosaminoglycan, anionic, non-sulfated glycosaminoglycan, gelatin, nucleic acid, extracellular matrix protein mixtures, matrigel, antibodies, and mixtures and derivatives thereof. In some embodiments, nanoparticles comprise Ferridex, a material composed of dextran-coated superparamagnetic iron oxide nanoparticles (SPIONs).

Nanoparticles may be either positively or negatively charged. In some embodiments, the negatively charged nanoparticles contain charge stabilized metals (e.g. silver, copper, platinum, palladium, gold). In some embodiments, the negatively charged nanoparticle comprises gold.

In some embodiments, the positively charged nanoparticles contain surfactant or polymer stabilized or coated alloys and/or oxides (e.g. elementary iron, iron-cobalt, nickel oxide, iron oxide). In some embodiments, the positively charged nanoparticles contain iron oxide.

The disclosure also relates to a system comprising:

(i) a hydrogel matrix;

(ii) one or a plurality of spheroids;

(iii) a generator for electrical current;

(iv) a voltmeter and/or ammeter;

(v) at least a first stimulating electrode and at least a first recording electrode;

wherein the generator, voltmeter and/or ammeter, and electrodes are electrically connected to the each other via a circuit in which electrical current is fed to the at least one stimulating electrode from the generator and electrical current is received at the recording electrode and fed to the voltmeter and/or ammeter; wherein the stimulating electrode is positioned at or proximate to one or a plurality of soma of the neuronal cells and the recording electrode is positioned at a predetermined distance distal to the soma, such that an electrical potential is established across the cell culture vessel.

In some embodiments, the solid substrate consists of hydrogel or hydrogel matrix. In some embodiments, the solid substrate consists of hydrogel or hydrogel matrix and is free of glass, metal, or ceramic. In some embodiments, the solid substrate is shaped into a form or mold that is predetermined for seeding cells of a particular size suitable for axonal growth. In some embodiments, the solid substrate or at least one base portion is shaped with at least one branched interior tube-like structure with an optional tapering in diameter the more distal the position of the tube is from the position in which the seeding of the tissue explants or neuronal cells takes place. For instance, this disclosure contemplates a focal point at one end of a semi-cylindrical or cylindrical portion of the solid substrate accessible to a point exterior to the solid substrate by an opening or hole at the exterior surface. The opening or hole can be used to place or seed cells (any one or plurality of any of the one or combination of disclosed) at the above focal point. As the cells are allowed to grow in culture over several days, the cells are exposed to culture medium with any of the components disclosed herein at concentrations and for a time period sufficient for axons to grow from the neuronal cells. If the cells are to be myelinated or the myelination is desired for study, glial cells may be introduced through the same hole and seeded prior to addition of the neuronal cells or explants. As the axons grow in the semi-cylindrical or tube-like structure, the axonal process growth can occur more and more distal from the focal point. Access points or opening in the solid substrate at points increasingly distal from the focal point (or seeding point) can be used to address or observe axonal growth of axon status. This disclosure contemplates the structure of the solid substrate to take any form to encourage axonal growth. In some embodiments, the interior chamber or compartment that houses the axonal process comprises a semi-circular or substantially cylindrical diameter. In some embodiments, the solid substrate is branched in two or more interior compartments at a point distal from the focal point. In some embodiments, this branching can resemble a keyhole shape or tree in which there are 2, 3, 4, 5, 6, 7, or 8 or more tube-like or substantially cylindrical interior chambers in fluid communication with each other such that the axonal growth originates from the seeding point of one or a plurality of somata and extends longitudinally along the interior chamber and into any one or plurality of branches. In some embodiments, one or a plurality of electrodes can be placed at or proximate to one or more openings such that recordings can be taken across one or a plurality of positions along an axon length. This can be used to also interrogate one or multiple positions along the length of the axon.

The disclosure relates to a system for accurately measuring recordings between an artificial central nervous system node and a peripheral nervous system node, the system comprising at least a first spheroid and a second spheroid, the first spheroid comprising a dorsal root ganglia or neuronal cells from the central nervous system or mammalian embryonic cells; and the second spheroid comprises at least one neuronal cell from a peripheral nervous system or primary mammalian stem cells. The disclosure relates to manufacturing any system disclosed herein by positioning at least a first or second spheroid comprising a magnetic substance in a well or channel defined by a hydrogel, moving the first or second spheroid by alignment of a magnet at or adjacent to the well or channel. If the system mimics the axons running between a central nervous system node or group of cells and a peripheral nervous system node or group of cells, in some embodiments, the first spheroid is positioned at or near a first well or channel and the second spheroid is positioned at or near a second well or channel at a distance sufficient to allow growth of the axons between the two spheroids after exposure to cell culture medium. The disclosure relates to measuring a recording between a central nervous node or group of cells and a peripheral node or group of cells, the method comprising placing an electrode at or adjacent to a first spheroid, placing an electrode at or near the second spheroid and stimulating the system with electricity using a amplifier comprising a generator or a generator. In some embodiments, the method further comprises measuring an electrophysiological response.

The term “recording” as used herein refers to, for example, measuring the responses of one or more neuronal cells. Such responses may be electro-physiological responses, for example, patch clamp electrophysiological recordings or field potential recordings.

The present disclosure discloses methods and devices to obtain physiological measurements of a microscale organotypic model of in vitro nerve tissue that mimics clinical nerve conduction and NFD tests. The results obtained from the use of these methods and devices are better predictive of clinical outcomes, enabling a more cost-effective approach for selecting promising lead compounds with higher chances of late-stage success. The disclosure includes the fabrication and utilization of a three-dimensional microengineered system that enables the growth of a uniquely dense, highly parallel neural fiber tract. Due to the confined nature of the tract, this in vitro model is capable of measuring both CAPs and intracellular patch clamp recordings. In addition, subsequent confocal and transmission electron microscopy (TEM) analysis allows for quantitative structural analysis, including NFD. Taken together, the in vitro model system has the novel ability to assess tissue morphometry and population electrophysiology, analogous to clinical histopathology and nerve conduction testing.

The present disclosure also provides a method for measuring the myelination of axons created using the in vitro model described herein. Similar to the structure of a human afferent peripheral nerve, dorsal root ganglion (DRG) neurons in these in vitro constructs project long, parallel, fasciculated axons to the periphery. In native tissue, axons of varying diameter and degree of myelination conduct sensory information back to the central nervous system at different velocities. Schwann cells support the sensory relay by myelinating axons and providing insulation for swifter conduction. Similarly, the three-dimensional growth induced by this in vitro construct comprises axons of various diameters in dense, parallel orientation spanning distances up to 3 mm. Schwann cell presence and sheathing was observed in confocal and TEM imaging.

Although neuronal morphology is a useful indicator of phenotypic maturity, a more definitive sign of healthy neurons is their ability to conduct an action potential. Apoptosis alone is not a full measure of the neuronal health, as many pathological changes may occur before cell death manifests. Electrophysiological studies of action potential generation can determine whether the observed structures support predicted function, and the ability to measure clinically relevant endpoints produces more predictive results. Similarly, information gathered from imaging can determine quantitative metrics for the degree of myelination, while CAP measurement demonstrates the overall health of myelin and lends further insight into toxic and neuroprotective mechanisms of various agents or compounds of interest.

In some embodiments, the at least one agent comprises a small chemical compound. In some embodiments, the at least one agent comprises at least one environmental or industrial pollutant. In some embodiments, the at least one agent comprises one or a combination of small chemical compounds chosen from: chemotherapeutics, analgesics, cardiovascular modulators, cholesterol, neuroprotectants, neuromodulators, immunomodulators, anti-inflammatories, and anti-microbial drugs.

In some embodiments, the at least one agent comprises one or a combination of chemotherapeutics chosen from: Actinomycin, Alitretinoin, All-trans retinoic acid, Azacitidine, Azathioprine, Bexarotene, Bleomycin, Bortezomib, Capecitabine, Carboplatin, Chlorambucil, Cisplatin, Cyclophosphamide, Cytarabine, Dacarbazine (DTIC), Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Irinotecan, Mechlorethamine, Melphalan, Mercaptopurine, Methotrexate, Mitoxantrone, Nitrosoureas, Oxaliplatin, Paclitaxel, Pemetrexed, Romidepsin, Tafluposide, Temozolomide (Oral dacarbazine), Teniposide, Tioguanine (formerly Thioguanine), Topotecan, Tretinoin, Valrubicin, Vemurafenib, Vinblastine, Vincristine, Vindesine, Vinorelbine, Vismodegib, and Vorinostat.

In some embodiments, the at least one agent comprises one or a combination of analgesics chosen from: Paracetoamol, Non-steroidal anti-inflammatory drugs (NSAIDs), COX-2 inhibitors, opioids, flupirtine, tricyclic antidepressants, carbamaxepine, gabapentin, and pregabalin.

In some embodiments, the at least one agent comprises one or a combination of cardiovascular modulators chosen from: nepicastat, cholesterol, niacin, scutellaria, prenylamine, dehydroepiandrosterone, monatepil, esketamine, niguldipine, asenapine, atomoxetine, flunarizine, milnacipran, mexiletine, amphetamine, sodium thiopental, flavonoid, bretylium, oxazepam, and honokiol.

In some embodiments, the at least one agent comprises one or a combination of neuroprotectants and/or neuromodulators chosen from: tryptamine, galanin receptor 2, phenylalanine, phenethylamine, N-methylphenethylamine, adenosine, kyptorphin, substance P, 3-methoxytyramine, catecholamine, dopamine, GABA, calcium, acetylcholine, epinephrine, norepinephrine, and serotonin.

In some embodiments, the at least one agent comprises one or a combination of immunomodulators chosen from: clenolizimab, enoticumab, ligelizumab, simtuzumab, vatelizumab, parsatuzumab, Imgatuzumab, tregalizaumb, pateclizumab, namulumab, perakizumab, faralimomab, patritumab, atinumab, ublituximab, futuximab, and duligotumab.

In some embodiments, the at least one agent comprises one or a combination of anti-inflammatories chosen from: ibuprofen, aspirin, ketoprofen, sulindac, naproxen, etodolac, fenoprofen, diclofenac, flurbiprofen, ketorolac, piroxicam, indomethacin, mefenamic acid, meloxicam, nabumetone, oxaprozin, ketoprofen, famotidine, meclofenamate, tolmetin, and salsalate.

In some embodiments, the at least one agent comprises one or a combination of anti-microbials chosen from: antibacterials, antifungals, antivirals, antiparasitics, heat, radiation, and ozone.

The present disclosure additionally discloses a method of measuring both intracellular and extracellular recordings of biomimetic neural tissue in a three-dimensional culture platform. Previously, electrophysiological experiments were undertaken in either dissociated surface-plated cultures or organotypic slice preparations, with limitations inherent to each method. Investigation in dissociated cell cultures is typically limited to single-cell recordings due to a lack of organized, multi-cellular neuritic architecture, as would be found in organotypic preparations. Organotypic preparations have intact neural circuitry and allow both intra- and extracellular studies. However, acute brain slices present a complex, simultaneous array of variables without the means to control individual factors and thus are inherently limited in throughput possibility.

Intracellular recording in in vitro three-dimensional cultures has been previously demonstrated. However, neuronal outgrowth was not spatially confined to an anatomically relevant structure supporting extracellular population investigation. A more biomimetic three dimensional neural culture is needed to allow examination of population-level electrophysiological behavior. The present disclosure supports whole-cell patch clamp techniques and synchronous population-level events in extracellular field recordings resulting from the confined neurite growth in a three dimensional geometry. Prior to the present disclosure, the measurement of these endpoints, directly analogous to clinical nerve conduction testing, had yet to be demonstrated for purely cellular in vitro studies.

Using the methods and devices disclosed herein, field recordings are used to measure the combined extracellular change in potential caused by signal conduction in all recruited fibers. The population response elicited by electrical stimulation is a CAP. Electrically evoked population spikes are graded in nature, comprising the combined effect of action potentials in slow and fast fibers. Spikes are single, cohesive events with swift onsets and short durations that are characteristic of CAPs or responses comprised purely of action potentials with quick signal conduction in the absence of synaptic input. The three-dimensional neural constructs disclosed by the present disclosure also support CAPs stimulated from farther distances along the neurite tract or channel, demonstrating the neural culture's ability to swiftly carry signals from distant stimuli much like an afferent peripheral nerve. The three dimensional neural cultures of the present disclosure support proximal and distal stimulation techniques useful for measuring conduction properties.

The present disclosure may be used with one or more growth factors that induce recruitment of numerous fiber types, as is typical in nerve fiber tracts. In particular, nerve growth factor (NGF) preferentially recruits small diameter fibers, often associated with pain signaling, as demonstrated in the data presented herein. It has been shown that brain derived neurotrophic factor (BDNF) and neurotrophic factor 3 (NT-3) preferentially support the outgrowth of larger-diameter, proprioceptive fibers. Growth-influencing factors like bioactive molecules and pharmacological agents may be incorporated with electrophysiological investigation to allow for a systematic manipulation of conditions for mechanistic studies.

The three-dimensional neural cultures created using the present disclosure may be used as a platform to study the mechanisms underlying myelin-compromising diseases and peripheral neuropathies by investigating the effects of known dysmyelination agents, neuropathy-inducing culture conditions, and toxic neuropathy-inducing compounds on the neural cultures. The present disclosure permits conduction velocity to be used as a functional measure of myelin and nerve fiber integrity under toxic and therapeutic conditions, facilitating studies on drug safety and efficacy. The incorporation of genetic mutations and drugs into neural cultures produced using the techniques disclosed herein may enable the reproduction of disease phenomena in a controlled manner, leading to a better understanding of neural degeneration and possible treatment therapies.

The present disclosure provides devices, methods, and systems involving production, maintenance, and physiological interrogation of neural cells and neural networks in microengineered configurations designed to mimic native nerve tissue anatomy. In some embodiments, the devices and systems comprise one or plurality of cultured or isolated Schwann cells and/or one or a plurality of cultured or isolated oligodendrocytes in contact with one or a plurality of neuronal cells in a cell culture vessel comprising a solid substrate, said substrate comprising at least one exterior surface, at least one interior surface and at least one interior chamber; the shape of the interior chamber defined, at least in part, by the at least one interior surface and accessible from a point exterior to the solid substrate through at least one opening in the exterior surface; wherein soma of the one or plurality of neuronal cells are positioned at one end of the interior chamber and axons are capable of growing within the interior chamber along at least one length of the interior chamber, such that the position of a tip of an axon extends distally from the soma. In some embodiments, the interior surface of the solid substrate is in the shape of a cylinder or is substantially cylindrical, such that the soma from the neuronal cells are positioned proximal to the opening at one end of the cylindrical or substantially cylindrical interior surface and the axons of the neuronal cells comprise a length of cellular matter extending from a point at an edge of the soma to a point distal from the soma along the length of the interior surface. In some embodiments, the interior surface of the solid substrate is in the shape of a cylinder or is substantially cylindrical, such that the soma from the neuronal cells are positioned proximal to the opening at one end of the cylindrical or substantially cylindrical interior surface and the axons of the neuronal cells comprise a length of cellular matter extending from a point at an edge of the soma to a point distal from the soma along the length of the interior surface. In some embodiments, the interior surface of the solid substrate is in the shape of a cylinder or is substantially cylindrical, such that the soma from the neuronal cells are positioned proximal to the opening at one end of the cylindrical or substantially cylindrical interior surface and the axons of the neuronal cells comprise a length of cellular matter extending from a point at an edge of the soma to a point distal from the soma along the length of the interior surface; wherein, if the cell culture vessel comprises a plurality of neuronal cells, a plurality of axons extend from a plurality of somata (or soma) such that the plurality of axons define a bundle of axons capable of growth distally from the soma along the length of the interior surface. In some embodiments, the neuronal cells grow on and within the penetrable polymer. In some embodiments, one or a plurality of electrodes are positioned at or proximate to the tip of at least one axon and one or a plurality of electrodes are positioned at or proximate to the soma such that a voltage potential is established across the length of one or a plurality of neuronal cells.

It is another object of the disclosure to provide a medium to high-throughput assay of neurological function for the screening of pharmacological and/or toxicological properties of chemical and biological agents. In some embodiments, the agents are cells, such as any type of cell disclosed herein, or antibodies, such as antibodies that are used to treat clinical disease. in some embodiments, the agents are any drugs or agents that are used to treat human disease such that toxicities, effects or neuromodulation can be compared among a new agent which is a proposed mammalian treatment and existing treatments from human disease. In some embodiments, new agents for treatment of human disease are treatments for neurodegenerative disease and are compared to existing treatments for neurodegenerative disease. In the case of multiple sclerosis as a non-limiting example, the effects of a new agent (modified cell, antibody, or small chemical compound) may be compared and contrasted to the same effects of an existing treatment for multiple sclerosis such as Copaxone, Rebif, other interferon therapies, Tysabri, dimethyl fumarate, fingolimod, teriflunomide, mitoxantrone, prednisone, tizanidine, baclofen,

It is another object of the disclosure to employ unique assembly of technologies such as two-dimensional and three-dimensional microengineered neural bundles in conjunction with electrophysiological stimulation and recording of neural cell populations.

It is another object of the disclosure to provide a novel approach to evaluate neural physiology in vitro, using the compound action potential (CAP) as a clinically analogous metric to obtain results that are more sensitive and predictive of human physiology than those offered by current methods.

It is another object of this disclosure to provide microengineered neural tissue that mimics native anatomical and physiological features and that is susceptible to evaluation using high-throughput electrophysiological stimulation and recording methods.

It is another object of the present disclosure to provide methods of replicating, manipulating, modifying, and evaluating mechanisms underlying myelin-compromising diseases and peripheral neuropathies.

It is another object of the present disclosure to allow medium to high-throughput assay of neuromodulation in human neural cells for the screening of pharmacological and/or toxicological activities of chemical and biological agents.

It is another object of the present disclosure to employ unique assembly of technologies such as 2D and 3D microengineered neural bundles in conjunction with optical and electrochemical stimulation and recording of human neural cell populations.

It is another object of the present disclosure to quantify evoked post-synaptic potentials in a biomimetic, engineered thalamocortical circuits. Our observation of antidromically-generated population spike in neural tracts suggest that they are capable of population-level physiology, such as the conduction of compound action potentials and postsynaptic potentials.

It is another object of the present disclosure to utilize optogenetic methods, hardware and software control of illumination, and fluorescent imaging to allow for noninvasive stimulation and recording of multi-unit physiological responses to evoked potentials in neural circuits.

It is another object of the present disclosure to use the microengineered circuits in testing selective 5-HT reuptake inhibitors (SSRIs) and second-generation antipsychotic drugs to see if they alter their developmental maturation.

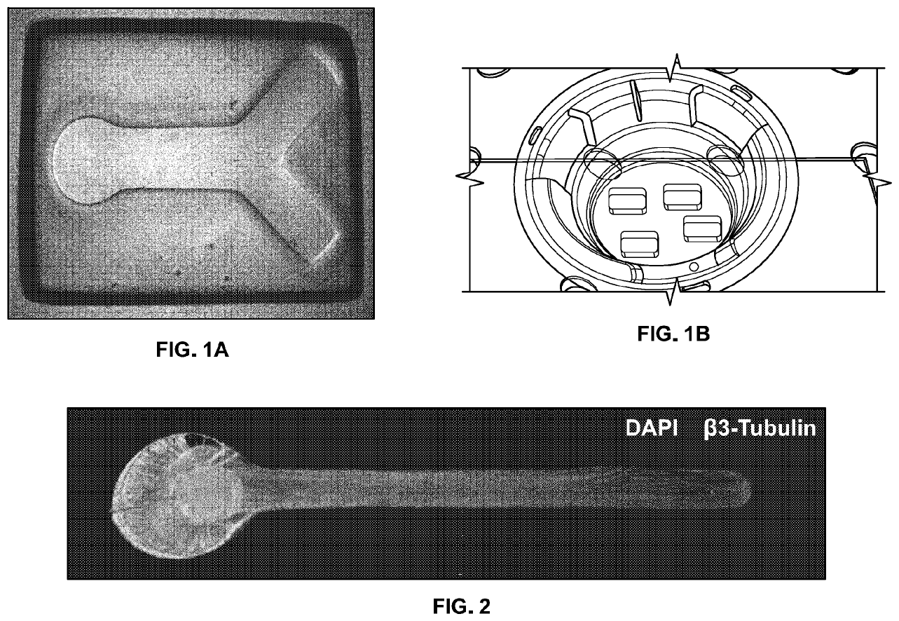

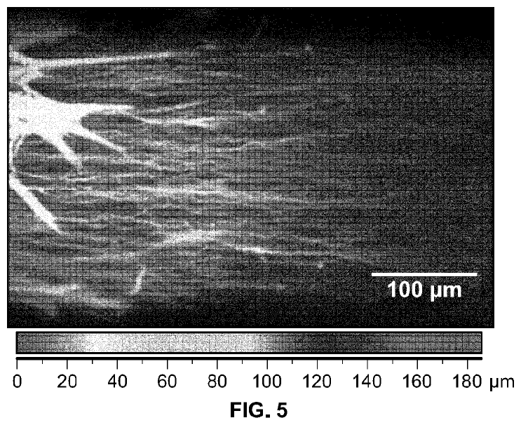

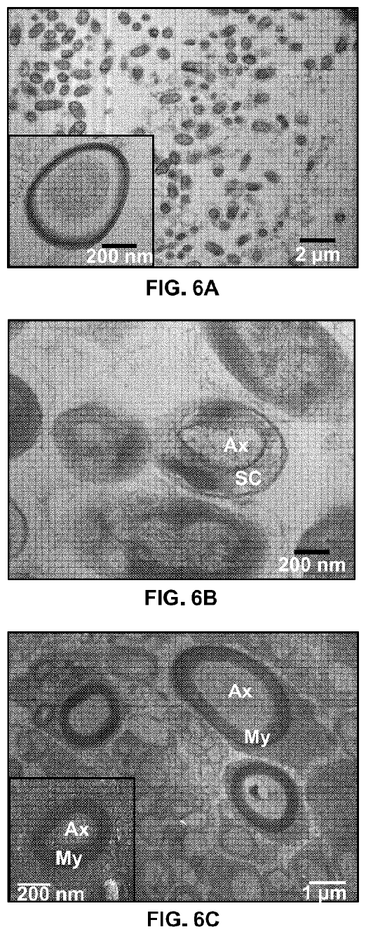

In one embodiment, projection photolithography using a digital micromirror device (DMD) is employed to micro pattern a combination of polyethylene glycol dimethacrylate and Puramatrix hydrogels, as shown in FIG. 1. This method enables rapid micropatterning of one or more hydrogels directly onto conventional cell culture materials. Because the photomask never makes contact with the gel materials, multiple hydrogels can rapidly be cured in succession, enabling fabrication of many dozens of gel constructs in an hour, without automation. This approach enables the use of polyethylene glycol (PEG), a mechanically robust, cell growth-restrictive gel, to constrain neurite growth within a biomimetic, growth conducive gel. In some embodiments, this growth-conducive gel may be Puramatrix, agarose, or methacrylated dextran. When embryonic dorsal root ganglion (DRG) explants are grown in this constrained three dimensional environment, axons grow out from the ganglion with high density and fasciculation, as shown in FIG. 5 and FIG. 6. The majority of axons appear as small diameter, unmyelinated fibers that grow to lengths approaching 1 mm in 2 to 4 weeks. The structure of this culture model with a dense, highly-parallel, three dimensional neural fiber tract extending out from the ganglion is roughly analogous to peripheral nerve architecture. Its morphology may be assessed using neural morphometry, allowing for clinically-analogous assessment unavailable to traditional cellular assays.

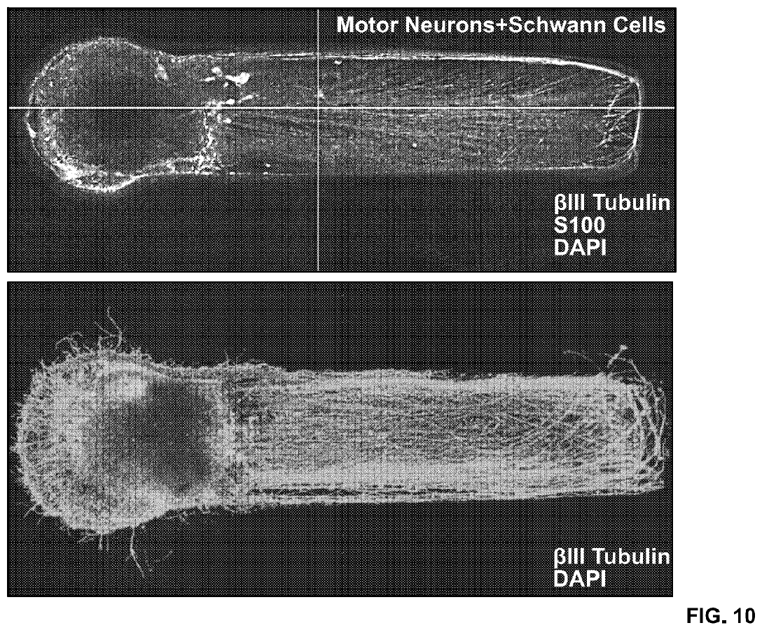

In some embodiments, the culture model provides the ability to record electrically evoked population field potentials resulting from compound action potentials (CAPs). Example traces show the characteristic uniform, fast, short latency, population spike responses, which remain consistent with high frequency (100 Hz) stimulation, as seen in FIG. 8B. The CAPs are reversibly abolished by tetrodotoxin (TTX), as shown in FIGS. 8E and 8F, demonstrating that drugs can be applied and shown to have an effect. There is a measurable increase in delay to onset associated with distal tract stimulation, seen in FIGS. 8C and 8D. The responses are insensitive to neurotransmitter blockers, indicating the evoked responses are primarily CAPs rather than synaptic potentials, shown in FIG. 10. Embryonic DRG cultures have been used effectively as models of peripheral nerve biology for decades. While extremely useful as model systems, conventional DRG cultures are known to be poorly predictive of clinical toxicity when assessed with traditional cell viability assays. While it is possible to perform single-cell patch clamp recording in DRG cultures, there are no reports of recording CAPs, due to the lack of tissue architecture. In a preferred embodiment, the present disclosure provides the ability to assess tissue morphometry and population electrophysiology, analogous to clinical histopathology and nerve conduction testing.

In some embodiments, the present disclosure uses human neural cells to grow nerve tissue in a three dimensional environment in which neuronal cell bodies are bundled together and located in distinct locations from axonal fiber tracts, mimicking native nerve architecture and allowing the measurement of morphometric and electrophysiological data, including CAPs. In some embodiments, the present disclosure uses neuronal cells and glial cells derived from primary human tissue. In other embodiments, neuronal cells and glial cells may be derived from human stem cells, including induced pluripotent stem cells.

In another embodiment. the present disclosure uses conduction velocity as a functional measure of neural tissue condition under toxic and therapeutic conditions. Information on degree of myelination, myelin health, axonal transport, mRNA transcription and neuronal damage may be determined from electrophysiological analysis. Taken in combination with morphometric analysis of nerve density, myelination percentage and nerve fiber type, mechanisms of action can be determined for compounds of interest. In some embodiments, the devices, methods, and systems disclosed herein may incorporate genetic mutations and drugs to reproduce disease phenomena in a controlled manner, leading to a better understanding of neural degeneration and possible treatment therapies.

The disclosure relates to systems comprising any of the disclosed compositions and methods of using those systems for capture of data around that is physiologically more relevant than the data collected using 2D tissue culture systems or systems without the use of multiple cell types. In some embodiments, the systems or compositions disclosed herein comprise one or more cells that comprise any mutation. In some embodiments, at least 1, 100, 500, 1,000 or more cells comprise a mutation that is relevant to a particular model of human disease. Any of the disclosed systems can include cells with the disclosed mutation below in Table B. In some embodiments, the cells of the disclosure comprise or express endogenous mutant proteins disclosed in Table B or those mutant proteins that are at least about 70%, 80%, 85%, 90%, 91%, 92%, 93%, 94%, 95%, 96%, 97%, 98%, 99% of Those model systems may be then be useful to test efficacy or toxicity of a certain drug, biomolecule or other therapeutic agent added to the system. The models may also be useful to understand basic biology in the context of environmental pollutants, pathogens or endogenously expressed protein and how such molecule effect the nervous system based upon such information such as response to agent with axon growth, myelination and demyelination or morphological changes to the cells themselves.

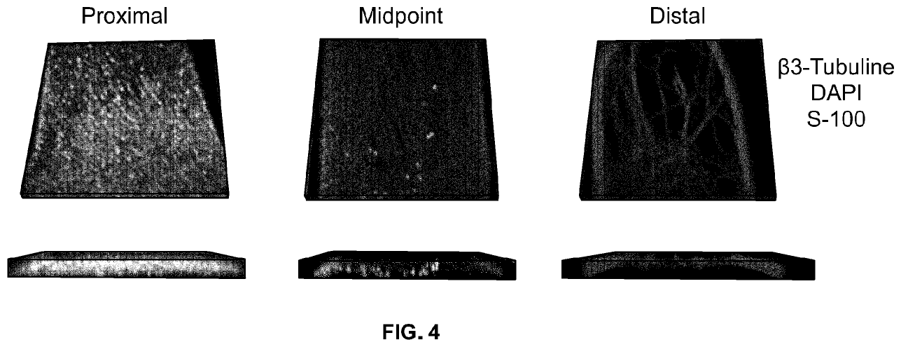

TABLE B
MUTATIONS

| Disease | Mutation | | GenBank Accession Number |
|---|---|---|---|
| 1. Amyotrophic | 1. SOD1 | | 1. KJ174530.1 (SEQ ID NO: 1) |
| lateral | 2. Alsin | | 2. AF391100.1 (SEQ ID NO: 2) |
| sclerosis | 3. SETX | | 3. NM_015046.6 (SEQ ID NO: 3) |
| (ALS) | 4. SPG 11 | | 4. NG_008885.1 (SEQ ID NO: 4) |
| | 5. FUS | | 5. NG_012889.2 (SEQ ID NO: 5) |
| | 6. VAPB | | 6. AY358464.1 (SEQ ID NO: 6) |
| | 7. ANG | | 7. NG_008717.2 (SEQ ID NO: 7) |
| | 8. TARDBP | | 8. CR533534.1 (SEQ ID NO: 8) |
| | 9. FIG. 4 | | 9. NM_014845.5 (SEQ ID NO: 9) |
| | 10. OPTN | | 10. KJ892931.1 (SEQ ID NO: 10) |
| | 11. VCP | | 11. NG_007887.1 (SEQ ID NO: 11) |
| | 12. UBQLN2 | | 12. NM_013444.3 (SEQ ID NO: 12) |
| | 13. C9ORF72 | | 13. BC068445.1 (SEQ ID NO: 13) |
| | 14. DCTN1 | | 14. NG_008735.2 (SEQ ID NO: 14) |
| 2. | HTT gene mutation | | NM_002111.8 (SEQ ID NO: 15) |
| Huntington's | (CAG segment) | | |
| 3. Parkinson's | 1. LRRK2 | | 1. NM_198578.3 (SEQ ID NO: 16) |
| | 2. PARK7 | | 2. NM_007262.4 (SEQ ID NO: 17) |
| | 3. PINK1 | | 3. NM_032409.2 (SEQ ID NO: 18) |
| | 4. PRKN | | 4. NM_004562.2 (SEQ ID NO: 19) |
| | 5. SNCA | | 5. NM_000345.3 (SEQ ID NO: 20) |
| | -Substantia nigra | | |
| | die | | |
| 4. Multiple | 1. HLA-DRB1 | | 1. AB698844.1 (SEQ ID NO: 21) |
| sclerosis (MS) | 2. IL7R | | 2. AH007043.2 (SEQ ID NO: 22) |
| 5. Rett | MECP2 | | NM_004992.3 (SEQ ID NO: 23) |
| Syndrome | | | |
| 6. Friedreich | FXN | | KU178079.1 (SEQ ID NO: 24) |
| ataxia | | | |
| 7. Prion | PRNP | | AF085477.2 (SEQ ID NO: 25) |
| disease | | | |
| 8. Spinal | SMN1. | | NM_001297715.1 (SEQ ID NO: 26) |
| muscular | | | |
| atrophy | | | |
| (SMA) | | | |
| (Kennedy's | | | |
| Disease) | | | |
| 9. CLN3 | CLN3 | | U32680.1 (SEQ ID NO: 27) |
| Disease | | | |
| (Batten | | | |
| Disease) | | | |
| 10. Pick's | Tau | | BC114948.1 (SEQ ID NO: 28) |
| Disease | | | |
| (Frontotemporal | | | |
| Degeneration) | | | |
| | GenBank | SEQ | |
| | Accession | ID | |
| | No. | NO: | Sequence |
| | SOD 1 | 1 | gtttggggccagagtgggcgagacgcggaggtctggcctataaagtagtcgcggagacggggtgctggtttgcgtcg |
| | (KJ174530.1) | | tagtctcctgcagcgtctggggtttccgttgcagtcctcggaaccaggacctcggcgtggcctagcgagttatggcgac |
| | | | gaaggccgtgtgcgtgctgaagggcgacggcccagtgcagggcatcatcaatttcgagcagaag |
| | Alsin | 2 | ggacccactgggttgccaagctcgcgccggatgcggagcgcggtgctgccggtggagcttcaggtcttgatagacttt |
| | (AF391100.1) | | ctgtaaagaaggaatgatttggtgatggagtgttcccactgaccgatggactcaaagaagagaagctcaacagaggc |
| | | | agaaggatccaaggaaagaggcctggtccatatctggcaggcaggatcctttcccataacaccagagagattgccag |
| | | | gctggggaggaaagactgttttgcaggcagccctcggagtgaaacatggagttcttctgactgaagatggtgaggtct |
| | | | acagctttgggactcttctctggagaagtggaccagtggagatttgtccaagtagccccattctagaaaatgccctggt |
| | | | tgggcaatatgttattactgtggcaacaggaagcttccatagtggagcagtgacagacaatggtgtcgcgtacatgtg |
| | | | gggagagaattctgctggccagtgtgcagtagccaaccagcagtatgtgccggaaccaaatcctgtcagcattgctga |
| | | | ttctgaggccagccctttgttagcagtcaggattttacagttggcgtgtggcgaggagcacactctggcattgtcaataa |
| | | | gcagagagatttgggcatggggtaccggttgtcagttgggtctcattaccactgccttcccagtgacaaagccgcaaa |
| | | | aggtagaacatcttgctgggcgagtggtgcttcaagttgcctgtggtgctttccacagcttagcccttgtacaatgcctc |
| | | | ccttcccaggatctgaagccagtcccagaacgatgcaaccagtgcagccagctcttgattactatgactgacaaagaa |
| | | | gaccatgtgattatatcagacagtcattgttgcccattaggtgtgacactgacagaatctcaggcagaaaaccatgcca |
| | | | gcactgctctcagcccctccactgaaacccttgacaggcaggaagaagtatttgagaacactcttgtagcaaatgatc |
| | | | agtctgttgctactgaactgaatgcagtaagtgctcagatcacaagcagcgatgccatgtcctctcaacaaaatgtcat |
| | | | gggaacaactgaaatttcctctgccagaaacataccatcataccctgacacccaagcagtcaatgaatacctacggaa |
| | | | actgtcagatcattcagtaagagaggactcagagcatggtgaaaagccagtgccatctcagcctcttttagaagaagc |
| | | | aattcctaatctccacagcccgcctaccacaagcacctcagccctaaacagcctggtggtctcttgtgcatctgctgttg |
| | | | gtgtgagagtggctgctacttatgaagctggtgccttgtcactgaagaaagttatgaacttttatagtacaaccccttgt |
| | | | gaaactggagctcaggcaggcagtagtgccattggccccgaaggtttgaaagatagcagggaagaacaggttaaac |
| | | | aggaatcaatgcaaggaaagaaaagttcaagtcttgtggatatcagagaagaagaaacagagggaggcagtcgaa |
| | | | gactctccctccctggattgttgtcacaagtttcccccaggctcttaagaaaggctgcacgggtgaaaacgaggacagt |
| | | | ggttctgacccccacatacagtggagaagcagatgcgctcctgccttctctgagaacagaagtgtggacctggggga |
| | | | aagggaaggaagggcagctggggcacggcgatgttctgcctaggcttcaaccgttgtgtgtaaaatgtctggatggc |
| | | | aaagaagtaatccatctggaggcaggtggttaccattctcttgcacttactgcgaaatcccaggtttactcatggggta |
| | | | gcaatacctttggtcaacttgggcattccgattttccaacaacagttcctcgtcttgcaaagataagcagtgaaaatgga |
| | | | gtctggagcatagctgcaggcagggattattccctgtttttagtggatacagaagacttccagcctgggttatattacag |
| | | | tggccgacaggaccctacagaaggtgacaaccttccagagaatcacagtggttctaagactccagtacttctctcctgt |
| | | | agtaagcttggatatataagcagagtgacagcaggaaaagatagctatttagccttggtggataaaaacattatgggg |
| | | | tatattgccagtctccacgagttagctactacagaaagacgattctattcaaaactaagtgatatcaaatctcagattct |
| | | | caggcctcttctcagtttagaaaatttgggcactacaactacagtccagctgttgcaggaggtggctagccgattcagc |
| | | | aagctgtgttacctcattggtcagcatggagcctcattgagcagcttccttcatggggtaaaggaagccaggagtttgg |
| | | | tcatcctgaagcattcaagtctcttcttggatagttatacagagtattgcacatctattacaaatttcctggttatgggag |
| | | | gattccagcttcttgctaagcctgccattgatttcctaaataaaaaccaagagctgttgcaagatttgtcagaagtgaat |
| | | | gacgaaaacactcagttgatggaaatactgaatactttgtttttcttgccaatcagacgacttcataattacgcaaaagt |
| | | | tttgctaaagcttgctacttgttttgaagtggcatctccagaatatcagaaactgcaggattccagttcttgttatgagtgt |
| | | | cttgctctccatctcggcaggaaaaggaaggaagcagaatacacactgggcttctggaagaccttccccggaaaaat |
| | | | gacggattccttgaggaagccagagcgtcgactgctgtgtgagagtagtaaccgagccctgtctctgcagcatgctgg |
| | | | gaggttttccgtgaattggttcattctctttaatgatgccctggtccatgcccagttctccacgcaccatgttttccctctgg |
| | | | ccacgctgtgggcagagccactgtctgaagaagctggtggtgtgaatggcttaaagataactacacctgaggagcag |
| | | | ttcactctcatttcatctacaccccaggaaaagacaaagtggctacgagctataagccaagccgtagatcaggctttga |
| | | | gagggatgtctgatctccccccttatggaagtggtagcagtgttcagagacaggaaccacccatttcacgcagtgcca |
| | | | aatatactttctacaaggatcctcgcctaaaggatgccacctatgatggacgctggctttcagggaagcctcatggcag |
| | | | aggggttttgaagtggcctgatggaaagatgtattctggcatgttcaggaatggcttggaagatgggtatggagaata |
| | | | cagaatcccaaacaaggcaatgaacaaagaagaccattatgtgggccattggaaagaaggaaaaatgtgcggtcaa |
| | | | ggagtctacagctatgcttctggtgaagtatttgagggctgttttcaagataatatgcgtcatggtcatggtcttctacga |
| | | | agtgggaaattgacgtcctcttctcctagtatgttcattggccagtgggtaatggataagaaagcaggatatggtgtctt |
| | | | tgatgatatcactaggggggaaaagtatatgggaatgtggcaagatgatgtgtgtcaagggaatggtgtggtggttac |
| | | | ccagtttggattatactacgagggcaactttcaccttaataaaatgatgggaaatggggttttgctttccgaagatgata |
| | | | ctatctatgaaggagaattttcagatgactggactcttagtggaaagggaacactgactatgccaaatggagactaca |
| | | | ttgaaggttattttagtggagaatggggatctgggataaaaatcactggaacctacttcaaacctagtctatatgagag |
| | | | tgataaagacagacctaaagttttcaggaagctaggaaacctggcagtgccagctgatgagaagtggaaagcggtgt |
| | | | ttgacgaatgttggcgccaactgggctgtgagggcccaggccaaggggaagtttggaaagcatgggacaatattgct |
| | | | gtggccttgaccaccagtcggcgccagcacagagacagtccagaaatactgagtcgttcacagactcagacactaga |
| | | | gagtttggaattcattccacagcatgttggtgccttctctgtggagaaatatgatgacatcaggaaatatttaataaagg |
| | | | cctgtgacactcctctgcaccccctgggcaggcttgtggagacactggttgcagtgtatagaatgacatacgtgggcgt |
| | | | aggagccaaccgcaggttattgcaggaggctgtaaaggagattaagtcctatcttaagcgaattttccagctggtgag |
| | | | gttcttatttcctgagctgcctgaagaaggcagcacaattcctctctctgctcctctgccaaccgaaaggaagtctttttg |
| | | | cactgggaagtcagattcccgatctgaatcaccagagccaggttatgtagtaacgagttctggattattgcttcctgtgc |
| | | | tgctacctcggctctacccaccgctgtttatgctttatgctttggataatgatcgcgaggaagacatttactgggaatgtg |
| | | | tccttcgactaaataagcagccagatattgctctcctgggctttcttggggtgcagaggaaattttggccagcaaccttg |
| | | | tcaatccttggagagagtaaaaaggttttgccaaccacgaaagatgcttgttttgcctcagcagtagaatgtctgcagc |
| | | | agatcagcacaacatttaccccatcagacaaacttaaggtcatccagcagacttttgaggagatctctcagagtgtcct |
| | | | ggcgtcactccacgaagacttcttgtggtccatggatgacttgtttcctgttttcttatatgtggtgctacgggccaggatt |
| | | | aggaatttaggctctgaggtacacctcattgaggatctaatggacccctatcttcagcatggggaacagggtataatgt |
| | | | tcaccaccttgaaggcatgttactaccagattcagcgtgagaagcttaactaggctgcataacagcttgaaaactggat |
| | | | tatctactacagagtgttataacaccatctggagtcttcctgtagtggcaaaaaagaacagtgttgaaattggaaagga |
| | | | ctttgtgttatttaggttgttagaatgagccttaccaataataagagccctgagcccagaaaaaaggactgtatagttta |
| | | | aagggaggattgaaagggaggtaaaaaatcagattagaccagttcttggcctatgataagttccaaaaataccattta |
| | | | tctactatttgaaaaaagaagaggatatcccttcctacagtaaagggtatgtcagctacatgaagttgtaagaaaagct |
| | | | tccagtagagcttcttatattaaagaagttgatggatatttttgaatttctggtttgcctgaatccacctgcagttaccccg |
| | | | atccgtttgcaagaaccagatcgtacttgaaactatagtggccacactctgccttcctgagtcccttccagtcatgtgtg |
| | | | catcatgtctctttgccaagggaggggagaaaggaacttttaaactgcagttttaactttttctaagctgtttcttgatgg |
| | | | gagaggttctgtgcaaaactaccacattctgtccccaaaatgtggaatgcatccaaataggagtcttctgcctcttaact |
| | | | taaaagaacataggaattttgtttttggtttctttatcatgctacagagagtgaatacactggaattcagacaccgactct |
| | | | gagctgctaggaacctcatttgtccatgtgcaaacgctgtattccaaggcctgtgaatggcagcctgaggaagttttgc |
| | | | atgcaggctgtgttttcgagcaggactaacaactgggaaataagcaaaaaactgcatcgatccccagcctggtgttgt |
| | | | tcttccctatacttcacactgaactcaggatgggaagaaaaaggaaacaagctttggctttttccatctcaaaagtattg |
| | | | tggcacctcaacatttcagtgttttgctttttaaaaaatgccctattgtaagttgttggtttatactgtataagtaacacta |
| | | | gtagctgttttgaataacataggtgctcttcctcatctcatctcctacaccgtggtgagcatacagagtgtcctgatttgt |
| | | | gttaagtgactgagaagatgttaattacttttgaaaaaggatcatggtttttgctctactttataatcaagacaagtgttt |
| | | | attaaaatactgttttggaatgttggctgtaatgtaacagcaattttcataataaaaggcattcatcttt |
| | SETX | 3 | acttccgctggtggcctagagcggggcccggtatggaggtgggctagaggccgacgccagccagagagcgaaatgt |
| | (NM_ | | tcttttggggccagagtctgggcatatatgaatgcaaatccgtgtttgttcacaactaagcccagctgagacgatcactt |
| | 015046. | | ttctgtaggccatttgtccaggtacagaatgagcacatgttgttggtgtacgccaggtggtgcttccaccattgacttcct |
| | 6) | | aaagcgctatgcttccaacactccgtccggtgaatttcaaacagccgacgaagacctctgctactgcttggagtgtgtg |
| | | | gctgagtaccacaaagcaagagatgaattgccattcttgcatgaggttttatgggaattagaaaccttacgtctcataa |
| | | | atcactttgaaaaatccatgaaggcagaaattggagatgatgatgagttatatatagtagacaataatggagagatgc |
| | | | cactgtttgacatcactgggcaagactttgaaaataagcttcgagttcctcttcttgaaatactgaaatatccttacttgc |
| | | | ttctacatgaacgtgttaacgagttatgtgttgaagcactttgtcggatggaacaagccaattgctcctttcaggtgtttg |
| | | | ataaacatccagggatctatttgtttttagtccatcccaatgaaatggttcggcgttgggctatcttgactgcaagaaact |
| | | | tggggaaagtggacagagatgattattatgacttacaagaagttttactttgcctttttaaagtcattgagttggggcttt |
| | | | tagagagtccagacatttatacttcttctgtcctagagaagggtaaactgattcttctgccctcacacatgtatgatacta |
| | | | ccaactacaaaagctattggttaggtatttgcatgttgctgaccattcttgaggaacaagccatggattccctgttgttgg |
| | | | gctcagacaaacaaaatgattttatgcaatcgatacttcacactatggagagggaagcagatgatgatagtgtggatc |
| | | | ctttctggccagcgttacactgttttatggtgattctggatcgccttggatctaaggtctggggtcaacttatggatcctat |
| | | | tgtggcatttcaaaccattatcaacaacgcaagctacaatagagagatccgacatatacggaacagctctgtaaggac |
| | | | caagttagaaccggagtcctatttggatgatatggtgacttgcagccagatcgtatacaattataatcctgaaaagacc |
| | | | aaaaaggattctggatggagaacagccatttgcccagattattgtcctaacatgtatgaagaaatggaaacattagcc |
| | | | agtgtacttcagtcagatattggtcaagacatgcgtgttcataacagcacatttctatggttcatcccttttgtccagtccc |
| | | | tcatggatcttaaggatttgggtgtggcttacatagcacaggttgttaatcatctgtactctgaagtcaaagaagtcctc |
| | | | aaccaaacagatgctgtgtgtgacaaagtcactgaattttttcttctaattttggtatcagtgattgaactgcatagaaat |
| | | | aaaaaatgtttgcatttgctgtgggtaagttcccagcaatgggtggaagccgtcgtcaaatgtgccaagcttcctacca |
| | | | ctgcgtttacacggagttctgagaaatcatctggaaattgctccaaaggaacagcaatgatatcttcactgtcattgcat |
| | | | tccatgccatctaactctgtacaacttgcttatgtgcagctgattagaagtctccttaaagaaggttatcagcttgggca |
| | | | gcagtctctttgcaagcgattctgggataagctcaacttattccttagaggaaatttatctctaggttggcagttgactag |
| | | | tcaggaaacccatgagctacaaagttgcttaaagcaaattattagaaacataaaattcaaagcacctccatgtaacac |
| | | | ttttgtggatctgacttctgcatgtaaaatctctcctgcatcttataataaagaagaaagtgaacaaatggggaagacgt |
| | | | ctagaaaagatatgcattgtttggaagcttccagcccaacattttctaaagaaccaatgaaagtgcaagacagtgtatt |
| | | | gatcaaagcagataacactatagaaggtgacaataatgagcaaaattatataaaggatgtgaaactagaggaccatc |
| | | | tcttagctgggtcatgcttaaagcagagtagtaaaaacatttttactgaaagagctgaagatcaaattaaaataagtac |
| | | | aaggaagcagaagtctgtaaaagagatctcttcatatacaccaaaggactgtacttcaagaaatggtccagaaaggg |
| | | | gatgtgacagaggaataatagtatcaacacgtttgttgactgattctagcactgatgctttggaaaaagtgtccacatc |
| | | | gaatgaagatttctctttaaaggatgatgctcttgctaaaacctcaaaacgaaaaactaaggtacagaaagatgaaat |
| | | | ctgtgcaaagttatcacatgtaataaagaagcaacacaggaagagtactttggtcgataatactatcaatttagatgaa |
| | | | aatttgactgtatctaacattgagagtttctattcaaggaaagatacaggagttcagaaaggagatggtttcatacaca |
| | | | atctttctttagaccctagtggtgttctggatgataagaatggagaacaaaaatctcaaaacaatgtattgccaaaaga |
| | | | gaaacaattaaagaatgaagaattagttattttctctttccatgaaaacaattgtaaaatacaggaatttcatgttgatg |
| | | | gtaaagaattgatcccttttacagaaatgaccaatgcttcagagaagaaatcatctccctttaaagatcttatgactgta |
| | | | cctgaatcaagagatgaggagatgagtaatagtaccagtgtgatttattctaacttgacaagagaacaggcccctgac |
| | | | atcagtcctaaatctgacaccttaacggattctcagatagacagagaccttcacaaattatctttactagctcaagccag |
| | | | tgttattacgttcccatccgattcacctcagaactcatcgcagctgcaaaggaaagtaaaagaagataaaagatgtttc |
| | | | acagctaaccaaaataatgttggagatacctcccgtggacaggttattattatttcagattctgatgatgatgatgatga |
| | | | aagaatcctgagtcttgagaaactcactaaacaggacaaaatatgccttgagagggaacatccagagcagcacgttt |
| | | | caacagttaatagtaaggaggaaaagaatccagtaaaggaagaaaagacagagactctttttcagtttgaggaatct |
| | | | gattctcagtgttttgagtttgaaagttcatctgaagtgttttcagtttggcaagatcatccagacgataataattcagttc |
| | | | aagatggtgagaaaaaatgtttggctcctatagccaatactacaaatggtcagggttgtacagattatgtatctgaagt |
| | | | tgttaaaaaaggagcagagggcattgaagaacacacaagaccacggagtatttctgttgaagaattttgtgaaattga |
| | | | agtaaaaaagcctaagagaaaacgatctgaaaaaccaatggctgaagatcctgtgaggccttcatcttctgtcagaa |
| | | | atgagggccagtctgatactaataagagagatcttgtgggaaatgattttaaaagtattgatagaaggacttcaactcc |
| | | | caattcacgtattcagagagccactacggtttcacaaaagaagtcttcaaagctttgtacttgtacagaacccatcagg |
| | | | aaagttccagtttctaagacccctaagaaaactcattcagatgccaaaaaaggacagaatagaagttcaaattaccta |
| | | | agttgtagaacaactcctgctatagtgccgccaaagaaatttcgtcagtgtcctgagccaacttcaacagctgagaaa |
| | | | cttggcctgaaaaagggtcctcgtaaggcatatgagttgtcccagcggtctttggattatgtagctcaattacgtgatca |
| | | | tggcaaaactgttggagtagttgatacccgaaaaaagactaaattaatttctcctcagaacctgtctgtcagaaataat |
| | | | aagaaacttctgactagtcaagaacttcagatgcaaaggcagatcagacccaaatcacaaaaaaatagacgaagac |
| | | | tttctgattgtgaaagtacagatgttaaaagagcagggtcacatacagcacagaattctgacatatttgtaccagaatc |
| | | | tgataggtcagattataattgtacaggaggaactgaggtacttgccaacagtaacagaaaacagttaataaaatgcat |
| | | | gccttctgaaccagaaaccataaaagcaaaacatgggtctccagcaactgatgatgcttgccctttgaaccagtgtga |
| | | | ttctgtagtgttaaatggaacagtaccaacaaatgaagtaattgtctccacttcagaagaccctctgggtggaggtgat |
| | | | ccaacagcacgtcatatagagatggcagctttgaaagaaggagagcctgactccagcagtgatgcagaggaagata |
| | | | acttatttttaacccaaaatgatcctgaagatatggatttatgttcacaaatggagaatgacaattataaactcattgaa |
| | | | ctaattcatggaaaagatacagttgaggttgaagaagattctgtaagtcggcctcagttggaatctttgagtggcacaa |
| | | | agtgtaagtacaaagattgtcttgaaaccacaaaaaaccagggtgaatactgcccaaaacactctgaagtgaaagca |
| | | | gcagatgaagatgtatttcgtaaacctggcttgcctcctcctgcatctaaacctttgagacctaccactaagatttttagc |
| | | | tcaaagagtacttcacgaattgctggtctttctaaatctttggaaacttcttcagcactttcaccgtctctaaaaaataag |
| | | | tcaaaggggatacagtcgattttgaaagtaccacagccagttcccctcatagctcagaagccagttggtgaaatgaag |
| | | | aattcgtgcaatgttcttcatcctcagtctccgaataattccaacaggcaaggttgcaaagttccatttggtgaaagcaa |
| | | | atattttccatcttcctctccagtaaacattcttttgtcatcacagtctgtctctgacaccttcgttaaagaggtcttaaaat |
| | | | ggaaatatgaaatgtttttgaactttggtcagtgtgggccccctgcaagtctttgtcagtccatctcaagacctgtgcctg |
| | | | tcagatttcacaattatggagattattttaatgtttttttccctttgatggtattgaatacttttgaaacagtggcacaaga |
| | | | atggctcaactctccaaatagagagaatttctatcagttgcaagtacgaaaatttcctgccgattatataaaatactggg |
| | | | agtttgcagtttatctggaagaatgtgaactggctaaacagctttatccaaaggaaaacgatttggtgtttttagctcct |
| | | | gagagaataaatgaagagaagaaagatacagagagaaatgacatacaagatctccacgaatatcattctggttatgt |
| | | | tcataaatttcgccgcacgtcagtcatgcgtaatgggaaaactgagtgttacctttccatccagactcaagagaactttc |
| | | | cggccaatttaaacgaacttgtgaattgtattgtaatcagttctctggtaactacacaaaggaagttgaaagccatgtct |
| | | | ctgttgggtagtcggaaccaactggctagagctgttctgaatccaaaccctatggacttctgtacaaaagatttactga |
| | | | ctacaacatctgagagaattattgcgtacttaagagatttcaatgaagatcaaaagaaagcaatagaaactgcatatg |
| | | | ctatggtgaaacactcaccatcagttgccaaaatctgcttgattcatggaccacctggaacaggaaaatcaaaaacta |
| | | | ttgttggcctcctctatcgtctactgacagagaaccagaggaaggggcattcagacgaaaactccaatgccaaaatca |
| | | | aacaaaaccgtgtcctcgtgtgtgcaccttccaatgcagctgttgatgaactcatgaaaaaaattatccttgaattcaaa |
| | | | gaaaaatgtaaagacaagaagaatcctttaggaaactgtggagatataaatttagtacgactgggtccagaaaagtc |
| | | | tattaatagtgaggttctaaagttcagtttggacagccaagtaaaccacagaatgaaaaaagagttaccttctcatgtt |
| | | | caggcgatgcataaaagaaaggaatttctagattatcagctggatgagctttcccggcagcgagctctatgccgaggt |
| | | | ggacgggaaatacagaggcaagaattagatgaaaacatttccaaagtttctaaggaaaggcaggaacttgcttctaa |
| | | | aattaaagaggttcaaggacgcccacagaaaacacagagtatcatcatcttagagtcccatatcatctgctgcacgtt |
| | | | gagcacaagtggtggtttactacttgagtctgctttccgtgggcaagggggtgtccccttcagctgtgtcattgttgatg |
| | | | aggctggacagtcttgtgaaattgagactcttactccactcatccatcgctgcaataagctcatcctagtaggagatcct |
| | | | aagcagctccctccgacagtcatctctatgaaagcacaggagtatggctacgaccagtcaatgatggctcgcttctgc |
| | | | agactgctggaagagaatgtagaacacaacatgatcagcaggctgcccattctacagctcactgttcagtacaggatg |
| | | | catccagacatatgcctcttcccttctaattatgtttataacagaaacttaaaaacaaatagacagacagaagccattc |
| | | | gatgttcatcagattggccatttcagccataccttgtgtttgatgttggagatggttcagaaagacgggataatgactca |
| | | | tatataaatgttcaagaaataaaactggtgatggaaataattaagcttattaaagacaaaagaaaggatgttagttttc |
| | | | gaaacattggcataataactcattacaaggcccagaagacgatgattcagaaggatttggacaaagagttcgataga |
| | | | aaaggaccagcagaagtagacactgtggatgcattccagggtcggcagaaggattgtgttattgttacgtgtgtcaga |
| | | | gcaaatagcatccaaggttcaattggattcctggcaagtttgcagagattgaatgtcaccatcacacgagccaagtac |
| | | | agcctcttcatcctcggacatttgaggaccctgatggaaaaccagcattggaatcagctgattcaggatgctcagaag |
| | | | cgtggtgccattattaagacctgtgacaaaaactatagacatgatgcagtgaagattctgaaactcaagcctgtgctgc |
| | | | agagaagtctcactcaccctcctaccatagccccagaggggtccagaccccagggtggtttgcccagcagcaagcta |
| | | | gacagtggatttgccaagacatctgttgctgcttctctataccacacaccctctgactccaaggaaattactcttactgtt |
| | | | acttcaaaggaccctgaaagacctcctgttcatgaccaacttcaggacccacgactgctgaagaggatgggcattgag |
| | | | gtcaaaggaggaatattcctttgggatccacaaccctcgagcccccagcatcctggagcaacacctcctacgggcga |
| | | | gccgggcttccctgtcgttcaccaggacctgagccatatacagcagcccgctgctgtagtggctgctctgagcagccac |
| | | | aaacctcccgtgcggggcgaacctccagctgccagtcccgaggcttccacgtgtcagagcaaatgtgatgacccgga |
| | | | agaggagctctgtcacaggagagaggccagggctttcagtgaaggggagcaggagaagtgtggttccgagacccat |
| | | | cacaccaggaggaactctaggtgggacaagaggacactggagcaggaggacagcagttccaagaaaagaaagctt |
| | | | ttataggaaagcccagtgacatgggccagcagccacagcatattgtaaactgaagatgaccagctcgtgggaccatc |
| | | | tagataagcttgttttttgtaaggagtttgtgtgctgttggaaaacatggaaaatgcatccttaacacctgagcctctggt |
| | | | catcttcagtattttctgtcatttgcaaaagctttcagagggcattgtgtatccgtaataatgtccttgaagtcagagact |
| | | | ggaaatgttgatctcttagtcttctatagacaaggaggtcagactggagtgaatgttgtaaaagttgaaatgtatatttg |
| | | | tatagcaaataacaaaatccttttaaaatttaatctagtagaccgcttttctttccccctgtttaaaatgttaatcagttttc |
| | | | aacagaacaaactttatattaccaaaaaaaaaaagatgggaggagggcgggtcctttcaaccattgttagagcaaga |
| | | | cagataattatttaacagcctagcttggaataagctgagttagtgctggtgggtcaggtgtctctggctctattaaaaaa |
| | | | agcaaaaacccccccaaaaacctggagtctcctaggggacactttgggcagcacggttatgttaggtaaagtcttgct |
| | | | gacatggtgcatttttagatagagtgcattggcccagggtattatatttctgtgatgagttcatttacctgtttcagtatgc |
| | | | acatagttccctagctaaaattcctaatcttcttgagagagaagaatatggagtgaaagaataattcttgagctatcatt |
| | | | caaatgctcccagcatattgtgagccttgtgtgactggtgaggtacagacatttggttgccaaatgctagattagcgctg |
| | | | ggtcagctgtggaagaatcgcctcagcttacagattgtcagacagatctaactagttttccagaaagcctgggaagct |
| | | | gtgtgttcaacatttcccaagggattctgatcaccaggcagcttgggaaccactggggcaggccaaatagaatattttg |
| | | | ggcgggaaagaagcacccgatttaaaatgaagcgtaaccagaggagttcagaactgggaagagagtggtagacttc |
| | | | ctgtgatcttcagaaatcatctacctggtaaaaatacatgctgtttagaatatctgataggtgtttccagctactattaga |
| | | | ggtgatagtgcttttgtgggggaaaaaattggtcatggtgaatggagatcgaggaagctcgggacaagggaggggtg |
| | | | ggctgcctgattttgtccagttttccaaatatccacgcagtgaactggagtatcctaaacatgagaatgtacagttgaca |
| | | | gttgtaaaaactagggatctgtagtgaatgctgtgcagccccatatctcatttgggggtaggaaaatagctgaagattc |
| | | | atgtgcattatttgacatttcctttgtcatctgctttttaagcaaaaaagggttttgtgttagaaattctacttgagcagatt |
| | | | ataaagagctttaaaaaacaactttcggttgccaaaagtttgagcatttgatttcattacctgtgtctccctcactggtgt |
| | | | ccagacggtcaactgaatactcctgaaacccagggagcaggtgacttcctggagtgctttgtccccagagtcagccac |
| | | | tgcttcctctgtgggggtggagagtttgtctttggccatgcagtgtgcgacagttcaggacgggtagggatgggtccca |
| | | | ttctgtctgggtcaagggctctatcagcttcttccatgtgcctttgggaagaaatctcgttactttaagtttgctttcctgtt |
| | | | atcttgatgaagtgcccattttagcagacacttgtagtgctgaccacttagggaatgtacaaactcctaagcttctaaag |
| | | | ggaggcatggcaaaaacgttggggtcaggatgtctctcacgctgctcatgttaatactattaacacatgatttgagaaa |
| | | | taagttttctctaaaatgcatattttgccgccacacactgaacaatattatttccagtgaagtttgatgcctgttcttacgtt |
| | | | gtgttcacctgttggttcaccactcagcagatctgattctgcaagaattaatggtagaactagatcatcctttctaacag |
| | | | acgagcctgtgtcctgtgacggcctttcacagcggaatgcagttgtacctcacattacttttgaaacttcactcgttccag |
| | | | ttggtacaagtatttgccaaagccatttcctatgttcaccgtggcccctcctgatgtggctgtcagcgcagcgttgttgaa |
| | | | cagggctattctttttacaaggtgtgaagtgtggctcttcgcttcgtctttgccatggcattaaaagaaagttccctgtctt |
| | | | ctttcaatattagttatttcaaatgaatatgtgctacttaaaagcttgttttgtttctttgtatataatttgccttggatttatt |
| | | | gtgcacagtttgttgagttgtatgtttttgtgaattatcaggagtaaatttgacaagtacatgtgaataacctcctgtaaa |
| | | | tgaattttataacaaaaatgtactgaactattttttaaagttgtgcagattagcaattttttgctatagctttgacttttctat |
| | | | gctgtgaattaatagctgcgatttggcaaacagccctgttgtctttgttaaaccctaaattttaagaggaaatggcagaa |
| | | | ttaaaagcagaaacaagaagatggacatggattagaggttatgtattatgaagtaaactacaaggtactaacatcatt |
| | | | tcgtctgccatttggtttgctttatgctgaaattacttggtggggatttgtgcaattcagatataaaaagtttcattatcca |
| | | | aaaa |
| | SPG 11 | 4 | MAAEEGVASAASAGGSWGTAAMGRVLPMLLVPVPAEAMGQLGSRAQLRTQPEALGSL |
| | (NG_008885. | | TAAGSLQVLSLTPGSRGGGRCCLEGPFWHFLWEDSRNSSTPTEKPKLLALGENYELLIY |
| | 1) | | EFNLKDGRCDATILYSCSREALQKLIDDQDISISLLSLRILSFHNNTSLLFINKCVILHIIFP |
| | | | ERDAAIRVLNCFTLPLPAQAVDMIIDTQLCRGILFVLSSLGWIYIFDVVDGTYVAHVDLA |
| | | | LHKEDMCNEQQQEPAKISSFTSLKVSQDLDVAVIVSSSNSAVALNLNLYFRQHPGHLLC |
| | | | ERILEDLPIQGPKGVDEDDPVNSAYNMKLAKFSFQIDRSWKAQLSSLNETIKNSKLEVS |
| | | | CCAPWFQDILHLESPESGNHSTSVQSWAFIPQDIMHGQYNVLQKDHAKTSDPGRSWKI |
| | | | MHISEQEEPIELKCVSVTGFTALFTWEVERMGYTITLWDLETQGMQCFSLGTKCIPVDS |
| | | | SGDQQLCFVLTENGLSLILFGLTQEEFLNRLMIHGSASTVDTLCHLNGWGRCSIPIHALE |
| | | | AGIENRQLDTVNFFLKSKENLFNPSSKSSVSDQFDHLSSHLYLRNVEELIPALDLLCSAIR |
| | | | ESYSEPQSKHFSEQLLNLTLSFLNNQIKELFIHTEELDEHLQKGVNILTSYINELRTFMIK |
| | | | FPWKLTDAIDEYDVHENVPKVKESNIWKKLSFEEVIASAILNNKIPEAQTFFRIDSHSAQ |
| | | | KLEELIGIGLNLVFDNLKKNNIKEASELLKNMGFDVKGQLLKICFYTTNKNIRDFLVEIL |
| | | | KEKNYFSEKEKRTIDFVHQVEKLYLGHFQENMQIQSFPRYWIKEQDFFKHKSVLDSFLK |
| | | | YDCKDEFNKQDHRIVLNWALWWDQLTQESILLPRISPEEYKSYSPEALWRYLTARHD |
| | | | WLNIILWIGEFQTQHSYASLQQNKWPLLTVDVINQNTSCNNYMRNEILDKLARNGVFL |
| | | | ASELEDFECFLLRLSRIGGVIQDTLPVQNYKTKEGWDFHSQFILYCLEHSLQHLLYVYLD |
| | | | CYKLSPENCPFLEKKELHEAHPWFEFLVQCRQVASNLTDPKLIFQASLANAQILIPTNQ |
| | | | ASVSSMLLEGHTLLALATTMYSPGGVSQVVQNEENENCLKKVDPQLLKMALTPYPKLK |
| | | | TALFPQCTPPSVLPSDITIYHLIQSLSPFDPSRLFGWQSANTLAIGDAWSHLPHFSSPDLV |
| | | | NKYAIVERLNFAYYLHNGRPSFAFGTFLVQELIKSKTPKQLIQQVGNEAYVIGLSSFHIPS |
| | | | IGAACVCFLELLGLDSLKLRVDMKVANIILSYKCRNEDAQYSFIRESVAEKLSKLADGEK |
| | | | TTTEELLVLLEEGTWNSIQQQEIKRLSSESSSQWALVVQFCRLHNMKLSISYLRECAKA |
| | | | NDWLQFIIHSQLHNYHPAEVKSLIQYFSPVIQDHLRLAFENLPSVPTSKMDSDQVCNKC |
| | | | PQELQGSKQEMTDLFEILLQCSEEPDSWHWLLVEAVKQQAPILSVLASCLQGASAISCLC |
| | | | VWIITSVEDNVATEAMGHIQDSTEDHTWNLEDLSVIWRTLLTRQKSKTLIRGFQLFFK |
| | | | DSPLLLVMEMYELCMFFRNYKEAEAKLLEFQKSLETLNTAATKVHINIPAMWLEDQV |
| | | | CFLLKLMLQQCKTQYELGKLLQLFVEREHLFSDGPDVKKLCILCQILKDTSIAINHTIITS |
| | | | YSIENLQHECRSILERLQTDGQFALARRVAELAELPVDNLVIKEITQEMQTLKHIEQWSL |
| | | | KQARIDFWKKCHENFKKNSISSKAASSFFSTQAHVACEHPTGWSSMEERHLLLTLAGH |
| | | | WLAQEDVVPLDKLEELEKQIWLCRITQHTLGRNQEETEPRFSRQISTSGELSFDSLASEF |
| | | | SFSKLAALNTSKYLELNSLPSKETCENRLDWKEQESLNFLIGRUDDGCVHEASRVCRY |
| | | | FHFYNPDVALVLHCRALASGEASMEDLHPEIHALLQSAELLEEEAPDIPLRRVHSTSSLD |
| | | | SQKFVTVPSSNEVVTNLEVLTSKCLHGKNYCRQVLCLYDLAKELGCSYTDVAAQDGEA |
| | | | MLRKILASQQPDRCKRAQAFISTQGLKPDTVAELVAEEVTRELLTSSQGTGHKQMFNP |
| | | | TEESQTFLQLTTLCQDRTLVGMKLLDKISSVPHGELSCTTELLILAHHCFTLTCHMEGII |
| | | | RVLQAAHMLTDNHLAPSEEYGLVVRLLTGIGRYNEMTYIFDLLHKKHYFEVLMRKKLD |
| | | | PSGTLKTALLDYIKRCRPGDSEKHNMIALCFSMCREIGENHEAAARIQLKLIESQPWEDS |
| | | | LKDGHQLKQLLLKALTLMLDAAESYAKDSCVRQAQHCQRLTKLITLQIHFLNTGQNTM |
| | | | LINLGRHKLMDCILALPRFYQASIVAEAYDFVPDWAEILYQQVILKGDFNYLEEFKQQRL |
| | | | LKSSIFEEISKKYKQHQPTDMVMENLKKLLTYCEDVYLYYKLAYEHKFYEIVNVLLKDP |
| | | | QTGCCLKDMLAG |
| | FUS | 5 | MASNDYTQQATQSYGAYPTQPGQGYSQQSSQPYGQQSYSGYSQSTDTSGYGQSSYSSYG |
| | (NG_012889. | | QSQNTGYGTQSTPQGYGSTGGYGSSQSSQSSYGQQSSYPGYGQQPAPSSTSGSYGSSSQSS |
| | 2) | | SYGQPQSGSYSQQPSYGGQQQSYGQQQSYNPPQGYGQQNQYNSSSGGGGGGGGGGNYG |
| | | | QDQSSMSSGGGSGGGYGNQDQSGGGGSGGYGQQDRGGRGRGGSGGGGGGGGGGYNRSS |
| | | | GGYEPRGRGGGRGGRGGMGGSDRGGFNKFGGPRDQGSRHDSEQDNSDNNTIFVQGLG |
| | | | ENVTIESVADYFKQIGIIKTNKKTGQPMINLYTDRETGKLKGEATVSFDDPPSAKAAID |
| | | | WFDGKEFSGNPIKVSFATRRADFNRGGGNGRGGRGRGGPMGRGGYGGGGSGGGGRGG |
| | | | FPSGGGGGGGQQRAGDWKCPNPTCENMNFSWRNECNQCKAPKPDGPGGGPGGSHMG |
| | | | GNYGDDRRGGRGGYDRGGYRGRGGDRGGFRGGRGGGDRGGFGPGKMDSRGEHRQDR |
| | | | RERPY |
| | VAPB | 6 | gcgtgccgtcagctcgccgggcaccgcggcctcgccctcgccctccgcccctgcgcctgcaccgcgtagaccgacccc |
| | (AY358464.1) | | cccctccagcgcgcccacccggtagaggacccccgcccgtgccccgaccggtccccgcctttttgtaaaacttaaagc |
| | | | gggcgcagcattaacgcttcccgccccggtgacctctcaggggtctccccgccaaaggtgctccgccgctaaggaaca |
| | | | tggcgaaggtggagcaggtcctgagcctcgagccgcagcacgagctcaaattccgaggtcccttcaccgatgttgtca |
| | | | ccaccaacctaaagcttggcaacccgacagaccgaaatgtgtgttttaaggtgaagactacagcaccacgtaggtact |
| | | | gtgtgaggcccaacagcggaatcatcgatgcaggggcctcaattaatgtatctgtgatgttacagcctttcgattatgat |
| | | | cccaatgagaaaagtaaacacaagtttatggttcagtctatgtttgctccaactgacacttcagatatggaagcagtat |
| | | | ggaaggaggcaaaaccggaagaccttatggattcaaaacttagatgtgtgtttgaattgccagcagagaatgataaa |
| | | | ccacatgatgtagaaataaataaaattatatccacaactgcatcaaagacagaaacaccaatagtgtctaagtctctg |
| | | | agttcttctttggatgacaccgaagttaagaaggttatggaagaatgtaagaggctgcaaggtgaagttcagaggcta |
| | | | cgggaggagaacaagcagttcaaggaagaagatggactgcggatgaggaagacagtgcagagcaacagccccatt |
| | | | tcagcattagccccaactgggaaggaagaaggccttagcacccggctcttggctctggtggttttgttctttatcgttgg |
| | | | tgtaattattgggaagattgccttgtagaggtagcatgcacaggatggtaaattggattggtggatccaccatatcatg |
| | | | ggatttaaatttatcataaccatgtgtaaaaagaaattaatgtatgatgacatctcacaggtcttgcctttaaattacccc |
| | | | tccctgcacacacatacacagatacacacacacaaatataatgtaacgatcttttagaaagttaaaaatgtatagtaac |
| | | | tgattgagggggaaaaagaatgatctttattaatgacaagggaaaccatgagtaatgccacaatggcatattgtaaat |
| | | | gtcattttaaacattggtaggccttggtacatgatgctggattacctctcttaaaatgacacccttcctcgcctgttggtgc |
| | | | tggcccttggggagctggagcccagcatgctggggagtgcggtcagctccacacagtagtccccacgtggcccactc |
| | | | ccggcccaggctgctttccgtgtcttcagttctgtccaagccatcagctccttgggactgatgaacagagtcagaagcc |
| | | | caaaggaattgcactgtggcagcatcagacgtactcgtcataagtgagaggcgtgtgttgactgattgacccagcgct |
| | | | ttggaaataaatggcagtgctttgttcacttaaagggaccaagctaaatttgtattggttcatgtagtgaagtcaaactg |
| | | | ttattcagagatgtttaatgcatatttaacttatttaatgtatttcatctcatgttttcttattgtcacaagagtacagttaat |
| | | | gctgcgtgctgctgaactctgttgggtgaactggtattgctgctggagggctgtgggctcctctgtctctggagagtctg |
| | | | gtcatgtggaggtggggtttattgggatgctggagaagagctgccaggaagtgttttttctgggtcagtaaataacaac |
| | | | tgtcatagggagggaaattctcagtagtgacagtcaactctaggttaccttttttaatgaagagtagtcagtcttctaga |
| | | | ttgttcttataccacctctcaaccattactcacacttccagcgcccaggtccaagtctgagcctgacctccccttgggga |
| | | | cctagcctggagtcaggacaaatggatcgggctgcagagggttagaagcgagggcaccagcagttgtgggtgggga |
| | | | gcaagggaagagagaaactcttcagcgaatccttctagtactagttgagagtttgactgtgaattaattttatgccataa |
| | | | aagaccaacccagttctgtttgactatgtagcatcttgaaaagaaaaattataataaagccccaaaattaagaaaa |
| | ANG | 7 | aaatatatatatatgtatatatatgtgtgtatatatatgtatatatgtgtgtatatatgtatatatatgtgtatatatgtgtgt |
| | (NG_008717. | | atatatatgtatatatatgtgtatatatatgtatatgtatatggaagagaggttaaaataaatggaaaacaaggtagga |
| | 2) | | agctctaatagatgtttaatttcagttccaaaaagagagaaaaaaatagagtaaaaacaatataggaggctggctggt |
| | | | ctgagtgcagtggtgtttacaactaattgatcacaagcagttacaggtctctttgttccttctccactcccactgcttcact |
| | | | tgactagcctaaaaaaaaaaattaaaaaaatatataaaaaaataggagaagataatggctactattagttaaaatgt |
| | | | atctcccccaaatttatatgttgaagccctaatccctagtatcccagaatgtaactgtatttggagatagggcgtttaaa |
| | | | gagatgattaagttaaaatgaggccattaaggtgggccctaatccaacctggtgtccctgtaaggggagtaaatttag |
| | | | acacaaagagaggagctaagggtgtggacccacagaaggacagctatgtgaggcggcagcaagaggccagccatc |
| | | | tctaagtgaaggagagaggcctcagaaggaaccaaacctgccaacaccttggtcttggatttttgacctccaggactg |
| | | | tgagaaaatgaatttcagttgtttaagtcacccagtctgtggtattttattatggaagccctagcaaactaacacaatga |
| | | | ctgagagttttccagaactaaagaaagacagcagtatatggatttaagaaaacccatacccaaaaacttgacactgat |
| | | | accatatttggaaaaacatgggttttccttatggtatcctgtactcctgttttatgtttctaaatggaaagcttcctgggcg |
| | | | atgcatctgattggtggagcccaggtcacatgcccattctcagctgtaagctgggaaagcaagtgttctaggacagact |
| | | | tacttataatatgtgaaatatccacatgtaaggagaatttttaaaagatgttggacacacactaacataacatatgccta |
| | | | taacagggctattcaaaacttatgttctagtctcagctgttttctctgtattcttttccttcaagagtgcataactcgtagag |
| | | | tctctaactcaatggatgacttatgtaatttgattaattcatttatcaattcatgaaactctagacacgagactattctggc |
| | | | tatcatgcaattcccatgaactgctccaccttcacttcatattcagcttacctaaatcctagcgtcacccctccgccctctc |
| | | | cagatacgctgaagaagtaggcttccttttttctgcaggtgttatcttgcctcttcaagacacctttgagagtcccctctct |
| | | | ttcctcttgatttcacttcatacctaggttcagcagaatcgccctgccctgtaacgctctcactcatcccttcctttccagtc |
| | | | ccataacgattacctttttgcagctttatgtttgagcatcttcaccatggccagtcactggtgtttctgactctggttgctcg |
| | | | tcccttcaattaattccgcaaactcatcaatgtttggtgaatttctattttcttctactaatcttctcacctgctgccccagc |
| | | | cagtgttcccttgaatgtctcagtctttctctcggactttgctcgtaattttattctaccagggaccaccctgctcccattttt |
| | | | gtctaagtcttaacctcccttcatagtgcagctcaggccaatgccctcttgaagacacccccggatggttcactttgtaa |
| | | | atgtgtggaccttggcatggccaacgccccttcacacctggcacagctacttttcattgttttccttcatgaaaactaaag |
| | | | ttcttttgttccacaaaactgtgacagtgacttcaggataaagatcatatctggatccctaattcttagtgcagagcctag |
| | | | tacagaactggaatgcagtgtttgttgaatgaaggaatgaattaaactgcaaattaacgaatgcctgctctggcactga |
| | | | gactcgcagctgtgagaaaggtctctggaggcagacagttgctggtacgaagtcctgagtcaagcaggaaacaaaat |
| | | | gcggatgtcagcagtttcaagtgagacctgtggtgagctccttcctctgccggttttcccttcttaattttcagtctgaagt |
| | | | gagggagtggagaatctttctgtgttagactgtgtcgcgttgggcagatttctccaatcttaggatagtagtggtccctct |
| | | | cactggccttcactttgtaccatccccagcaagaacactgagtctgtgaaccgaattctagtagtcgggaaatggattg |
| | | | aaaggacatccaggttttacttagtttctatgctcagatgctggcagaagccctgcaaatcttttccttttctgtgaaagg |
| | | | aagaggcggtttctgcgtgaggccctatagctcaggggttagagcactggtcttgtaaaccaggggtcgcgagttcaa |
| | | | atctcgctggggccttgcgaaactactttcttgattcaggtgttttttgaaaaactccccttctagtctttctgaactaagt |
| | | | cacagaaaatgtggcaaattagagcttctttcctctgttcagagcttccaaacactagctcagcagaacgtttcctttag |
| | | | gttccagtagtaatgctgctttagctcctggaatgcggcggaatccattacacaggcggtggaaatcaccgtcagcctc |
| | | | tgcccacgccctggcttgcggactggcggcgagagccctgctgactgccgggcgggaaatggggacagctaggcga |
| | | | gacaatagccctttaagctattctcaagccccgggtctgggcatatttggagtagagatcaaggttccacttctgaaac |
| | | | gggaagagccagcaatggccgctccggccaggcgtccagaagagagatgcacgttgtgaggcaaggtttcatgtgc |
| | | | cagtggggttttgggaaattaagggcccgactttaggggcgagacttgggtcactaaatttgattttaacgcagagaa |
| | | | atgtggccttccttttgaatgagctgcaaatattatctccaaaagcgaattcacacagaaacattgggtttgcccagatg |
| | | | tcctctatttcaggggttcccaacccttgggccgcgcacctgtccatggcctgttagggacccggctgtacagcaggag |
| | | | gtgaactccgggctagctaccagtaccgcctgagctccgcctcctgtgagatcagtggtggcattagaatctcacagg |
| | | | agcgcgaaccctattgggaactgcgcatgccagggatctaaactgcactccttatgagaatctaagtgatgcctgatg |
| | | | atctgaggtggaacagtttcatcccgaaaccatcccccaactcttccccccgcccccgcggtccgtggaaaaactgtct |
| | | | tccacgaaaccggtccctagtgccaaaaatgttggggaccactgctccattccacaccctctccctcccgtgtctgtgg |
| | | | acgtgtaagcggaagagccgagattgggagggaagcgcaggaagtgaggacatcagggagcccgggcctaagcct |
| | | | ggaggcccgccctctccggcgttcacagttgcgcagtcagaattgtgtgcgtcccaccgcctcagcccccagttctccg |
| | | | acgcccaggtgccggtccccaccctacacacaaacacacgcatccacgctcgcgaggcccctgggtccgcgcccagt |
| | | | gaggccaacagccctcgggcgcccagaggatgagcagcctcggtatgcccgagtttcaggggcgcctgctctgcttg |
| | | | ggagcccctcagtgaggcaggggaatccagatttagaaaggttgtcctcagaacctcggacgcaggactgtcttaca |
| | | | agctggctttgtgactctgcagtaattactttccgggtataaagagatttctttctttcccgtctctggttaaggatgtctc |
| | | | gagtagggatttttaggagagttgtttccgtgcttccttcgatagctcagctggtagagcggaggactgtagtacttaat |
| | | | gtgtggtcatccttaggtcgctggttcgattccggctcgaaggagacgctgctgttttggggtttacggttgctcttcgttt |
| | | | gtgcaaaaaatctctgtatcttgacgtgatcaaatctgtttctgtctctcacccttgtccgaaggaaacagcttagccgc |
| | | | gaggtgggctgcgttaatcgcgtcttgaaaagttggttacctttctgaaagatagtagacccgaatcgtcccaccgttg |
| | | | ggggaaggcactgttgtcttaccgaagtgtgttatattttggcttctacgttaagtccaaaggccggcagatatatacgt |
| | | | atcgcgcgtctgtcctggggctcagtaccctacagccttgcaaccgagctcagctctggccctcgatctctatttcagcc |
| | | | ccctccaggttccctccaggccctagcgggcttaaatcgctccccgggctcagtgagacacaagcatgtgacctaaag |
| | | | tctaaaaagctgccttccttcaccccatcctgcatctggatcgaggagaataagccagagaataagccacacttgaaa |
| | | | cttggtgaagatggaaggaataactaaataaggcctaagggaaatgtcacaacagaggaaaagaaacaaaacaaa |
| | | | aaaccttcagaacagcaggcggaaaagagaaattcagagtgataggaagcaccactcagctacagtggctcgttgg |
| | | | tctaggggtatgattctcgctttgggtgcgagaggtcccgggttcaaatcccggacgagccctcgtggctactgtttttc |
| | | | cacccccttttgtcaactactgaaaaaaagtctccgttaaatttgaaacacaaactgtctcgcgatgggctgtcctgcca |
| | | | aagaaagcagcagcgaataagtacgtggcagatggtgctgtcgaccagtgtcaagaccaagtttcatgggttcctgg |
| | | | acgctcagccaggggtagtctctgaagggccgcctggcaattgggagcagaagccagtttcccgccatctgctctccg |
| | | | tggaggcagtgctctcgcggctgctgcccgttggggtccagctgaggaaggagcgcggatcccaggctcgttctttgc |
| | | | ctgggctgcccctgcggcccctggggcgggtgctgctgcggcgccagctccaagggcggatccaggcgggaggggc |
| | | | ctcctcggagaagcggggcgcggtcccaactacgcagaggctggcacgccgaccctccacacctcaccacgccccca |
| | | | tctccgtccgtgtacacacactcacacaaggacgccaaccccacctagatgcaaagcaggattcaaaagaacatcttt |
| | | | gcgttttctaccggctccccatcatcgtactagggaggaagaagcgggtgagaaacaaaacttctttccattgtcctgc |
| | | | ccgtttctgcggacttgttctgaggccgaggtaggttcacactcctgctcctcctcctcctctctggtagcttcataaggg |
| | | | gcgtttaggccggggagtcctttcctagtagaggcagcttgggactccttggggaggagtttgcgggacggggctgga |
| | | | gagggaggaatggggagggacgtgaaagtcccgtatttgtatgagttaacgactttacattcccactcaggtttacttt |
| | | | gcgcagctgggcacagatgccagcttctattgattagtaggcttggagggggaccacagaatcgccgaacctaaggg |
| | | | aagccctaggaggcctgcacttgcccacaactaagaaagctttgctagttattcaggctaatcaccagctttttgaagc |
| | | | aggccttagttttttgcaagcgtaactaacatttggggatcggtgttttctgtttactctcctctgcctcctcacccttaacc |
| | | | caccctcagccagtgttgtgcaattcttagagacacttgccaacagcctcttctctaagccttcctactggaaggttggg |
| | | | atcctccgttttctcgctctgagacagcacgtcaaatgctaatagcagcacatgagtaaagcgcaacatctttattaaa |
| | | | acaatttcagcaccactcagggggctagggaaatctcagatgagccaagaccctccctcgtgacagtaaaaatcttat |
| | | | aatcaagtcataatgtaatcagtagaaacccaagggacatcattatctgtggcacaggaccaccttcccagaagcagc |
| | | | ctcaggtattcaagacttacttaaccacagagccactaacaagttttgcaagaggataggtttattacatacgcttcaat |
| | | | tcaagcaattcttttactgatgacaagcagagtaaaataatttttgaatttatggtaatagtcaatgtaggtttccttcga |
| | | | acagcaactttcagtggcccctttaaagttagctctgattgtctagaaagtactagtatggaatataaaagttgtgtggg |
| | | | cccggcacggtggctcatgcctgtaatcccagcactttgggaggccgaggtgggtgcatcacaagatcaggagttcga |
| | | | gaccagcctggccaagatggtgaaacctcgtctctactaaaaatacaaaaattagccgggcacggtggtgagcacct |
| | | | gtaaccccagctactcgggaggctgagacaggagactctcttgaacctgggaggcggaggttacagtgaacagagat |
| | | | tgcgtcactgcactctagcctgggtgacagagcaagactccgtcttaaaaaaaaaaaaaaaaaaaattgtgtggtgc |
| | | | attggaaagcttccaggccggcgaatgcaatgagttccttgggcaatacatgtctctttctctgttcctcagttggctctt |
| | | | ctttaatatgaggggtggacagagtctcagaggcccctcccactcttgttctatgactccatggagtggtacgatgttag |
| | | | taacaggacatgaggcactctagtaatgtggagatgaactatcgtttggcagagaggatggaaaaaatcattccagg |
| | | | aaaagaggaatggctttagctattgttgaaaacagataatcaggttataagcaggcaagagtaagaggtttgtcaagt |
| | | | tattgttggaagcattggtcactgtgactcaaattaaagtagctggataaaaaagtggaccaatgaaggtcagtgaaa |
| | | | cctttctttaggagtttttattgaatctgtatttaagcagattctctgtaattctctgtaaacagttattcccggaagaatgc |
| | | | catgaaatacttgggaaagagggaatgggaacaaagagtgagtgggcacaaagcccagagttggttgaaaagcaa |
| | | | atgtttaatttttgtctttgtttggaaatacaggcatgtcagcaggactttgtccccactgtccttcaatcagtagtgttgc |
| | | | ctcttcctggagacgtggttcttcttgccacgtgtgcttttccttctttacctgttacttccgttattgaataagttcatgctt |
| | | | atagacctaaagtgttagaataataaagaaatatagaaattatttagtccattcttcccatcatctaattttataattagt |
| | | | cctaagacagaatgatacatatatactctgtatgaaggaggaaagtgctctaacactagggtccttggaaacaggacc |
| | | | cgtgcatcccgtgtctgtgtgcagggacattattggcttgagggagagagagaaaaaaatctccaggaaaggggaat |
| | | | gactttcaagttaaagctgtctgaacatttaaaaaatatatgaggttttttgcaattggcgattccttcatttgtattaaaa |
| | | | ttactgacaaagatgtgatgttccttaatcatagaatataattccttgttgtattttaaacacaaatgactaagtgcttag |
| | | | atgcattctaatgaaatagtaatttttcccccaaaccctacttttcatttcaacaaaacttccttgtgccaagtttaaaaga |
| | | | aaaacataaaactgtctttttgcattgtgggcagattgtgtatcctccaaaggttgtccagtgaaggcccagaggcaga |
| | | | gaagggacctaactatattcagaggctgctggttgccccacctcagcactactgcagaactctaaaatcagaggctgc |
| | | | tctgtttgtgtgagtcggtgcttgaattggtgcttcaggccatggggctgtgtgtgtgcaggcgcatatgggttgtatgct |
| | | | gtggcagggtaccgcttttcgggactcagaggtggtgttgcttgagctaagcctggaaagacaggtcagttttctcctt |
| | | | gcttatgaagggttcaaagtctgactccaccattcatctgctgtttgttcttggagtttcctcatgagcagagcatatttac |
| | | | ataatgcctacctcacacagtcctggtgttggttgaatgtagtcatgtatgtgagagcttcacaaaatgaaagattgcac |
| | | | tgaatattataattattgtgatttcaagtgcatttgttttactagcaggcagcctgtcttttcttgacaccttctcaagtgca |
| | | | gccagactgaatacttggcagctaggtgggtgacaacagaggattgaggatttttaaaaagataacactgtcctagat |
| | | | ccaacaccactgtgggcaaatgctgggggtctgtgaaacagttggtaccctgctgctgtctgatattggaaactatgag |
| | | | atctataaggtgatcctggtgacctttggcccactgtttttacgcaagcaatggatctacagccctgctttgatgatgga |
| | | | gataagcaattttgcaactacattacaaggatcagaagattgaatgttcagggttagtctcttgtggacaggtaaagat |
| | | | ggaagaaggagaaacccaggaaacatggcaagcctgcttctgggggttagaagctgagaagttgagtgtaggggg |
| | | | aggggcagtgtactaatagctgtcacacaaccttctctggaagtgctttcctggctgtatgaaatcgatggaataacgt |
| | | | gcttagcatccgttcatgaaaccaaatgatgtgatacgataaataggatgtgatatgataaatgataaatagtacagtg |
| | | | gaaaagcatcctactggaagaccatatcttactctgtgacatcttccaaggcagttcttggagttggcccagggagatc |
| | | | agagatgctatcttatggatccagactattatgtgtgaatagggaaacctcagtcctgccatgaacaagagtaagcac |
| | | | cacagacagcagaaaagggcaacttttggacagaaggccatgtttctcaaactttgtcttccagaacaaatctctaatc |
| | | | cattcaggtgggttaatatctaacccaatcatgcccagcgaaaaggtgaatgaccctcctcgtttgttcaagagcagtg |
| | | | tccttggttttcatgtccctgccaggactgggacactatatattaggaacactatataatcagaacctggagaggcctcc |
| | | | aggttcacacaactggaacccatctccaggaacaaacagctggaacccatctcccgttgaagggaaactgccagattt |
| | | | ttgtaagattcttcctcctgggtaaactattgttcaatttgttttatatattatttctagccatccatccattttaatgaaacc |
| | | | attttcctccctttaaagcctccaggctcaagctcatgaattaaaaagaaaatggatcagattctaaagtttaaaatgaa |
| | | | aacaggtttcttttgggatggcctatgtgtggataaagagaaattagtgagactgggaggaagtggaagtttaggagg |
| | | | gatttctaaattgtgataaaatccccaaaggcctgggggagccatttgctgaaggccatcgggagtcctcccccgtcgt |
| | | | ccacccctgttacaggcattacacgtggcttaacaaggaaagaaacagtatgtttttagcattgtttccttctaaccttcc |
| | | | ctgctaatttctcttcttagaaaagttttggatgctctttcaaacaaagtggaacacatcccaagatcaaaagaaggctc |
| | | | actggcaggttgaagggaagaaggaaggaaaagcaataaagggatgaatatgccttgggtgttaggctcttactttg |
| | | | ggggattttgccctaggatgtataaaagtgtgaagccacattgatgaaaatgataactatagttactcatcataaactg |
| | | | tcaagttaactccctggtgtgtaaaatttgatgaatcttttgaatactgaacattgcctgtggggagagggagagaata |
| | | | attcttgtgaatactaacagattcatttgatacagaagataaagttactctaaagaacagttggggccaggtgtggtgg |
| | | | ctcacacctgtaatccccgcactttgggaagccgaggtagccggatcaactgaggtcaggagttcgagaccagcctg |
| | | | gccgacatggcgaaaccccgtctctaccaaatacacaaaattagccaagcgtggtggtggccgcctgtaatcccagct |
| | | | acttgggaggctgaggcaggagaattgcctgaacctgggaggtggaggttgcagtgagccaagatagcaccactgc |
| | | | gatctagcctgggcgatagaacaagactctgtctcaaaaaaaaaaaaaaaacaggccgggcgcggtggctcacgcc |
| | | | tgtaatcccagcactttgggaggccgaggcgggtggatcatgaggtcaggagatcgagaccatcctggctaacaagg |
| | | | tgaaaccccgtctctactaaaaatacaaaaaattagccgggcgcggtggcgggcgcctgtagtcccagctactcggg |
| | | | aggctgaggcaggagaatggcgtgaacccgggaggcggagcttgcagtgagccgagattgcgccactgcagtccgc |
| | | | agtccggcctgggcgacagagcgagactccgtctcaaaaaaaaaaaaaaaaaaaaaaaaaaagaaaagaaaaga |
| | | | aaaaaaggacagagaacagctgggactaattccactgacaaaaagctgacagacagttacgtgtttgggtacagaa |
| | | | ggatatatctataggacttttgccaccagatgtgaggctttgcacccttggcttaggcagattcacttttacaccacaaa |
| | | | cgatctgtctcttactggttttctcccaattccattttaccttgctttttgaggaatttagagttgaatatattgagaaagttt |
| | | | tgagtgagggagcagatgttagatattggtggttttggtgtacgcttgctgagatttggttcctggatgtcttagggaatc |
| | | | aagattacaggcccccaactaaacttatacaaaatttgtctctgtaactgagggtcactggacctttgctattgaaacta |
| | | | gcaaagcctctattatgtttaatgagaaccatgaaacttcagatttttagagctagcaactcttttattttcttgatgaaca |
| | | | aacctaggcctggagagaaaaatgaagtcaattacttaaaaaatattaagggttataataaggcaagcatgttgattc |
| | | | aaggtgaataatatgagttctgtggaatatacaccagtaagaaacactaacaagtaaatacttcattgttcatccacga |
| | | | tcacgtgggtaatggcatgtttggaaatggcacccgtgaaacctgacaacaggtcggtctttattttgtgatatccttat |
| | | | gggtagattctgaactttgtggcttttactttggcacttatatctaatgaggtgtccttggtcttaatatctaatgcagagc |
| | | | gagtttggacagagattctaaacaggttcaccagctcatactcccttctgtgaacagcaatatccccacaagttataca |
| | | | gcattggcacctcctgcaagtacggtatttcctagagaagacattagtaacattcctatccaccactgccacctaacga |
| | | | ctcttttaggtactagcagctctggttccgtttcctgatttctgatttctgggattctggcaccaggcagaagaatgttgac |
| | | | tgtgtaaggcagacctttgacattagaagtcagtatttgttcattacagagcaagagacttaattgattacaaatcaata |
| | | | aaactttaataagatcttccaaaaaactagaaacaggaaacacacccaggagactgaaaagcatgatgtaaatgtga |
| | | | agaacgtggcaagtgggtggcttggcagcccagcagctatactggagcagctgcatgagtgaatacttgttactccttt |
| | | | agggctggctctccattctacaatcatgtacctctttcttgataaactcccaagaacacactttcagcatggaatttcaga |
| | | | cttttagagatagcaactctctcaagtatggacaaatgtcggtgcggacaaatttgatgcactgctgaagaataccaaa |
| | | | taatatgctaaggaattttttcattccacaataatggagtaaatagcagctggaaatgtttgcattaagttcatagattat |
| | | | aatttgtaatggaatcaacaccaaatgcaaattagaaagagagcccactttgctcacccagtcacgtcttcccatgtaa |
| | | | ccatagaacattggggtcctgtgtctttctagatccacagtcttgctctcagaacaggctagccacaccacaggcctag |
| | | | tgccaggacccatggcctttttttaagctcagactcccttctgtgaacagcaatatccccacaacttgtacaacattggt |
| | | | gcttcctgcaagggctacagaactatttgatacgaaaatgttcattgacttacacacaagagaagcacaaaataaaaa |
| | | | attaataattaatttaatgtctttgaaaatgtaccatttatttttacatttggggtcataagaattgtattacacttaagaat |
| | | | gcaatacaatttgaagatcagatttttctccctttgtgagaatttctcagtatgtgtgatgactaccaagaaatcatagcc |
| | | | agtcataaattcagtgagttactcataaacgaacaagaaccacctacttcttggggaggtaggtctgcttcccttcaact |
| | | | caggatacaactgctttcaactgctttcttcacattagctgactaattagctagaagcctgtcgtaaacaattttatggtt |
| | | | gactccttccctgggctcagggttccctagaacagaggtccccaaatcccggtctgtggcctgtccgcctaagctctgc |
| | | | ctcctgccagatcagcaggcagcattagattctcataggagctggacgcctattgtgaactgcgcatgtgcgggatcc |
| | | | agattgtgcactctttatgagaatctaactaatgcttgatgatctatctgaaccagaacaatttcatcctgaaaccatccc |
| | | | ccaccaatccatagaaatactgtcttccacaaaaatgatccctggtgccaaaaatgttagagaccactcccctaaaact |
| | | | ctcttcttagctctcacctcctgtattactatctcatctcagtacattgaagcccccatcttttccccatggatgcctcatttc |
| | | | ctattagggaggcatttttttattttttgtttttatttttttccgagacggagtctcgctctgtcgccaaggctggagtgcag |
| | | | tggcgcgatctcggctcactgcaagctccgcctcccgggttcacgccattctcctgcctcagcctcccgagtagctggg |
| | | | actacaggcgcccgccactacgcccggctaattttttgtatttttagtagagacggggtttcaccgtggtagccaggatg |
| | | | gtctcgatctcctgacctcgtgatccgcccgccttggcctcccaaagtgctgggattacaggcgtgagccaccgcgccc |
| | | | ggccgtcatttggtatgtcttaatgtgcctcaggacctagcacagtccctggtacccagtagagacctatgtaatgttca |
| | | | ttattcaattaataaatacatgaattaaagagtgagagtggattttgtaatgttacgactgatagagaaatactcagtga |
| | | | ttctaagggatggggaagaacggttggagctagaggttgtgctcaggaaactattaaatagacgttccgcaggaagg |
| | | | gattgacgaagtgtgaggttaatgaggaagggaaaatagaatataaaatttggtggtggaaaagatctgattcatgat |
| | | | gccgtgtcagagagcaaagctcctgtccttttggcctaatttggtgatgctgttcttgggtctaccacacctccttttgccc |
| | | | tccgcaggagcctgtgttggaagagatggtgatgggcctgggcgttttgttgttggtcttcgtgctgggtctgggtctga |
| | | | ccccaccgaccctggctcaggataactccaggtacacacacttcctgacccagcactatgatgccaaaccacagggcc |
| | | | gggatgacagatactgtgaaagcatcatgaggagacggggcctgacctcaccctgcaaagacatcaacacatttatt |
| | | | catggcaacaagcgcagcatcaaggccatctgtgaaaacaagaatggaaaccctcacagagaaaacctaagaataa |
| | | | gcaagtcttctttccaggtcaccacttgcaagctacatggaggttccccctggcctccatgccagtaccgagccacagc |
| | | | ggggttcagaaacgttgttgttgcttgtgaaaatggcttacctgtccacttggatcagtcaattttccgtcgtccgtaacc |
| | | | agcgggcccctggtcaagtgctggctctgctgtccttgccttccatttcccctctgcacccagaacagtggtggcaacat |
| | | | tcattgccaagggcccaaagaaagagctacctggaccttttgttttctgtttgacaacatgtttaataaataaaaatgtc |
| | | | ttgatatcagtaagaatcagagtcttctcactgattctgggcatattgatctttcccccattttctctacttggctgctccct |
| | | | gagaggactgcataggatagaaatgcctttttcttttcttttcgttttttttttttttttttttgagatggagtctcactctgtc |
| | | | gcccaggcttaagtgcaatggcacaatctcggctcactgcaacctctctctcctgggttcaagtgattctcctgcctcag |
| | | | cctcccaaatagctgagattacaggcatgcaccaccacacctggctaatttttgtgtttttagtagagacagggtttcac |
| | | | cgttttggccaggttggtcttgaactcctgacctcgggagatccgcccaccttggcctctcaaagtgctgggattacagg |
| | | | catgagccactgagccgggccactttttccttatcagtcagtttttacaagtcattagggaggtagactttacctctctgt |
| | | | gaaggaaagtatggtatgttgatctacagagagagatggaaaaattccagggctcgtagctactaagcagaatttcca |
| | | | agataggcaaattgttttttctgtcaaataataagctaatattacttctacaaatatgagaccttggagagaagtttcca |
| | | | aggaccaagtaccaacataccaacagattattatagtttctctcactcttacacacacacacacacatatacacatatg |
| | | | taatccagcatgaataccaaaattcattcagggtagccaccttttgtcttaagcgagagataattttgatgtttgaatgg |
| | | | aatgctcccaggatattctcttgtcatggttattttatataaaattcaaaaaccaattacattatttcctctgtaatctttta |
| | | | ctttatcaactaatgtctggcaagtgtgatgttttggggaagttatagaagattccggccaggcgctgtggctcacgcct |
| | | | gtaatccagcactttgggaagctgaggcggacagatcacgaggtcaagagatcaagaccatcctggacaacatggtg |
| | | | aaaccttgtctctactaaaaatgtgaaaattagctgggcgtggtggcacacacctatagtcccagctactcgggaggct |
| | | | gaggcaggagaatcgcttgaacctaggaggcggaggttgcactgagccgagatcacgccactgcactccagcctgg |
| | | | gcgacagagcgagactccatctcaaaaaaaaaaaaaaaagaaagatcccagtttatcccagtttatcccttattcttcc |
| | | | tcaattctcaagatttgtttttaagttaacataacttaggttaacacactctttgtaaaatacactgttcaatctacagact |
| | | | cagtggttagcttcctgttaactaatttctgttgacaggtacttggatattttatttagaaagtggttgccaataaattagt |
| | | | tataagtcgccagtttcactgccttgtgaacacataattattgtggtctcagtattccctatggtggcttctcctgctcctg |
| | | | gtattgccctgaaatgggccaaaagccgtggctccccaatactcaggttatagaacattgtccaggtaccacctagga |
| | | | gagcccagcctcactgaaagtattcaaatttaggaatgggtttgagaagtaggtagctggtatgtgcttagcacaaga |
| | | | atctctcttccttgggttagtctgtttcaaaactgaaaacactgtcattccttaagaaaataggaaaaagtattccaaac |
| | | | ctctgtcactagaaaatttgccatattaccaaatctcaaaaacctctcaggaaatgagaaagtcccagtttctggtaaa |
| | | | ctatttgggcccttttctcaagttctccaaccagtgctatttccttgaggtgaggcaaagttactcaagatcatcgctgcc |
| | | | actcaaggccttgatagggcaagtgaaaggcatggaccattattatattgatcacagcataagctgtgaaaacccaca |
| | | | tcttctccaaacatctgcttggagcattatcatcgcatagtttgctctggtgttcagggaaatcgctgtttcataggaaat |
| | | | cacatggcagtgggatgggagtgtttcctgacctgccgatggtactggcacctgagcaagcattcctagtcctttttggt |
| | | | ctgggcctcttgttctatcacaaccacaagctgtttaaaataaaacgtcaagtcacaggcaggtcattttatcctgcgtg |
| | | | aatcaattgaagaattgaagtcctggtcagctgtgattgttggctttgcaaggaaaaagaaggaacactccaaaatgc |
| | | | taaatgctttgaagaatagtgacatgcaaggaagaaaattccatgcaatttggagaagaaacatttttctaggaatcta |
| | | | gtagctgagatattttatagtttaaaaacagacttccaatatcttgagaatacagttagctctacgaaataaagaggag |
| | | | tcaataatatatgcaactctgtagtcattcattattttatccagattgacagaagcagaggaaaccaagaaaaatggaa |
| | | | gtggtaaaaaaaaaaaaatgcagtaattgccaatatttactgagcatttaccctgtgctaggccttgttataggcgctta |
| | | | gattgattttcttttcataacatccctaagaggtaggtattacatgctcattttgcttatgaggaaatctgctgagagaga |
| | | | ttaagtaattttctcatggtcacagcctcagtcttttagaccacggtgtcaaaagaagttcttactgtgagtcagggtggt |
| | | | gcagtggagaaagcgtttggggaaaatcctgacccaaaagtgtgcgttgggatgatgtggagatcattcaattttctct |
| | | | tgtagctacagctttgagatgcacccattggtatccacactggcctgctgtcctcaaatatttctctatgtcttgtttacttg |
| | | | agtagtgcagaaaagctgacgtcctgggtctgctctcgcatgcataggtgaggttttctctgtagctagtcatcttgcta |
| | | | aaccatatttttggtcacaaagaatcaggatgagagaaagtagataaaaccatggaaactcgaggtgccaccggaag |
| | | | agcaaaccaaagtatattgatctgctgaagctataatccaagtactaagtataccttcagaagttgtatgcattgttctt |
| | | | ggggcaaaaaggaaatcaaacatctgcccttcctcccacaacctctttacttccctctcttcctaacactgtatctcacat |
| | | | ctacagctgcaggcacacagatatgacatacagcttgatatctttatgtgcaaaggcaaaagatggctatggatatag |
| | | | gaggtggcatttaaaagaacaatatccatgtttgatactgctttcattatcaataatgaaaaggtagcctttcagcagaa |
| | | | caaagtaaggtacaggtcgtttggaagccatttcagtcattcacacagtgtgatccaccattacgaagcagaatttgaa |
| | | | atcaaaagacaatagtacccatatacatcaaactaggagttcaactggggtcagtccttggactgcacatacagagtt |
| | | | gggctgcctgactctcaaaacattgcccttgctaatttgcgttgctcacttcctattgaatcagggatgatttttctttgct |
| | | | gtagtgaatcagccctgctacactgcaagaatcaagccgattcacaattcacatcaattccaggagtgacggccagat |
| | | | ggcagcatctggctagtcagttggtgagtgagtccacctccagtcttgattaaactctgctgctgggaagtagcaattct |
| | | | gagcataaatgtctttgatatcctgaatattttgggtgtgggatgagagaggcagtagatagtggaaagagcacaggg |
| | | | tgtagactaagataggcctggattcctttctttaatctgtcaatgaaagcaatgaatgttctgaaactacatggcaatgc |
| | | | tttttttgttgccactctggcttttttttttaatgctataaaatatatgcctatttttcactgccaagaggtgacaacacaatg |
| | | | atgctgcaaccagagaagacagacagctaccaacaactattgacaagacagaaggaacatcaagaatagatgcaa |
| | | | gacaaagcaatgtatctccatggcctagattttttaggttatgcatagaaacagcaaggagaagaagacacaatccaa |
| | | | gttttcttttgagaatattgtgctgtttaaatttattttttaaacatttcttagcttttcttattagtcaatttcataagcctggg |
| | | | aaacatctttactctatccatttaattgcatttgttcattagcttactcatttcacttattagttcattcaaacagtagctatt |
| | | | atcaactgtcttctaagaactgtaactgggtattctggttttgcttttttttaaaaaatagaattaaaaggatttccaaaag |
| | | | gacctgattttacattagaattagaataatacattttaaattaatgtataacatatattattctagaattagaatagatttt |
| | | | aaatcacattagaataatagccacatgacaaaaaagaaaagaaaaataataaaattaatgacaaagaaaaacaata |
| | | | gtcatgtgagaggaaaggcaaagcctaaatattcctctgtcctcttcctccacctagattcccagatgtaaggtattaaa |
| | | | agtagtttgtaaactttctgtgacgtatgctgagtctatccaaactgtcgggaattggagcctcccgggaaccaaacac |
| | | | tccccaaagatcatacacctacggagttcccccaagagacacacaactgcagtttcatgtaggttcaccttttctctcttt |
| | | | ttatactctgtccacacaaacctggcagccacgcagtcagtccctgccctgtgtcattgtactggccacagcaacttctc |
| | | | tctgtaaatgagaaaggtcttgctttcagaaaagatcatgtttgcaaactactcactcttgaggaagaagggtgggaa |
| | | | aagtagcagagatgagtggggagatggtgcatataagaaggaggaaagaggagactatggagtcaggatgccggc |
| | | | ttgctattctcaacacctttttccagtgcccagttcttaccttatttctcctgccccttgctttcttttctaggcacctctaaga |
| | | | tactgatggctctgcagaggacccattcattgcttctgcttttgctgctgaccctgctggggctggggctggtccagccct |
| | | | cctatggccaggatggcatgtaccagcgattcctgcggcaacacgtgcaccctgaggagacaggtggcagtgatcgc |
| | | | tactgcaacttgatgatgcaaagacggaagatgactttgtatcactgcaagcgcttcaacaccttcatccatgaagata |
| | | | tctggaacattcgtagtatctgcagcaccaccaatatccaatgcaagaacggcaagatgaactgccatgagggtgtag |
| | | | tgaaggtcacagattgcagggacacaggaagttccagggcacccaactgcagatatcgggccatagcgagcactag |
| | | | acgtgttgtcattgcctgtgagggtaacccacaggtgcctgtgcactttgacggttagatgccaccatgtagggattatc |
| | | | gcgagtggttgaccttacacttactccttaaatagcagtgagtaatgcatttgagctgtcccaggctctgtctcctcagct |
| | | | catttcctactctttttctctatataactcattctattaaatacattgcaccaaagagatatggagacataaacctgtaatg |
| | | | aatgaggctgggcttttctgtaataagcttccttttataatactggtcagcttagctctctcagatcctatcctgtggaatt |
| | | | tagttattatgtgtatttatgtagtatttcaaacatttcaaaatgctttcatctatgtttatcacattttaataccacagcact |
| | | | tataatgatgtcactacatatagaagctcaaagttaagggatttgctgaagactgtaaagttaatggaagaattgagac |
| | | | aaaaatccagtgtagctggccacttatccagggctttttctacttcatcacaaggaatgttttgaaagtgtctgcttttttt |
| | | | atccttaaaattcacctgtcagggaggcattaaaaatttggaaatgtatgccagcaaaatgtgagctctgtattttttgg |
| | | | cattcttatgtttgggtttaataagattaagaaaatgatactgggaattttctttttcctgaaactttgaatcaccctagta |
| | | | agtcaaagtactaaaaaatgtactagatcattaagacttatgtgctcttactgattgaaagattttttatgttttccttgta |
| | | | ataaaggacctaaaccgaaggtacctgagaagactgtgctatggtattattttttttctctgacttttaaattcttgtttag |
| | | | tttataaacatgcatgcaccttaataacctcataaactctggtcaaagactaatgcctatcagatccatgaccacaaca |
| | | | cagaagatttgtctcatttactccagagagaatgattcctctcaaagacaattctcacagctcttccttctcccttagaat |
| | | | atttagaagcaaattagggagctgtcaggtctctgagcccaagcctgcacgtatacatccagatggcctgaagcaact |
| | | | gaagaaccacaaaagaagtgaaaataaccaattcctgccttaactgatgacattccaccactgtgatttgttcctgccc |
| | | | c |
| | TARDBP | 8 | atgtctgaatatattcgggtaaccgaagatgagaacgatgagcccattgaaataccatcggaagacgatgggacggt |
| | (CR533534.1) | | gctgctctccacggttacagcccagtttccaggggcgtgtgggcttcgctacaggaatccagtgtctcagtgtatgaga |
| | | | ggtgtccggctggtagaaggaattctgcatgccccagatgctggctggggaaatctggtgtatgttgtcaactatccaa |
| | | | aagataacaaaagaaaaatggatgagacagatgcttcatcagcagtgaaagtgaaaagagcagtccagaaaacat |
| | | | ccgatttaatagtgttgggtctcccatggaaaacaaccgaacaggacctgaaagagtattttagtacctttggagaagt |
| | | | tcttatggtgcaggtcaagaaagatcttaagactggtcattcaaaggggtttggctttgttcgttttacggaatatgaaa |
| | | | cacaagtgaaagtaatgtcacagcgacatatgatagatggacgatggtgtgactgcaaacttcctaattctaagcaaa |
| | | | gccaagatgagcctttgagaagcagaaaagtgtttgtggggcgctgtacagaggacatgactgaggatgagctgcgg |
| | | | gagttcttctctcagtacggggatgtgatggatgtcttcatccccaagccattcagggcctttgcctttgttacatttgca |
| | | | gatgatcagattgcgcagtctctttgtggagaggacttgatcattaaaggaatcagcgttcatatatccaatgccgaac |
| | | | ctaagcacaatagcaatagacagttagaaagaagtggaagatttggtggtaatccaggtggctttgggaatcagggt |
| | | | ggatttggtaatagcagagggggtggagctggtttgggaaacaatcaaggtagtaatatgggtggtgggatgaacttt |
| | | | ggtgcgttcagcattaatccagccatgatggctgccgcccaggcagcactacagagcagttggggtatgatgggcatg |
| | | | ttagccagccagcagaaccagtcaggcccatcgggtaataaccaaaaccaaggcaacatgcagagggagccaaac |
| | | | caggccttcggttctggaaataactcttatagtggctctaattctggtgcagcaattggttggggatcagcatccaatgc |
| | | | agggtcgggcagtggttttaatggaggctttggctcaagcatggattctaagtcttctggctggggaatgtagacagtg |
| | | | gggttgtggttggttgg |
| | FIG 4 | 9 | acgtcctccagccccgctcccgacgtgaggggcggggcttgcctggaggcggggcgcagggatccggaaacacctg |
| | (NM_014845. | | atcatctataggtttagtgcctaatgggtgttgttcctggctggacttgatgtccagggcctgaggggttttctcgccgag |
| | 5) | | tctcctggggcggtccggaggctcgtgccctgttgtggggcccccatttgccgccgccatgcccacggccgccgccccc |
| | | | atcatcagctcggtccagaagctggttctgtatgagactagagctagatactttctagttgggagcaataatgcagaaa |
| | | | cgaaatatcgtgtcttgaagattgatagaacagaaccaaaagatttggtcataattgatgacaggcatgtctatactca |
| | | | acaagaagtaagggaacttcttggccgcttggatcttggaaatagaacaaagatgggacagaaaggatcctcgggct |
| | | | tatttcgagcggtttcagcttttggtgttgtgggttttgtcaggttcttagaaggctattatattgtgttaataactaaaag |
| | | | gaggaagatggcggatattggaggtcatgcaatctataaggtcgaagatacaaatatgatctatatacccaatgattc |
| | | | tgtacgggttactcatcctgatgaagctaggtatctacgaatatttcaaaatgtggacctatctagcaatttttactttagt |
| | | | tacagctatgatttgtcccactcacttcaatataatctcactgtcttgcgaatgcccctggagatgttaaagtcagaaatg |
| | | | acccagaatcgccaagagagctttgacatctttgaagatgaaggattaattacacaaggtggaagcggggtatttggg |
| | | | atctgtagtgagccttatatgaaatatgtatggaatggtgaacttctggatataattaaaagtactgtgcatcgtgactg |
| | | | gcttttgtatattattcatgggttctgtgggcagtcaaagctgttgatctatggacgaccagtgtatgtcactctaatagct |
| | | | agaagatccagtaaatttgctggcacccgttttcttaaaagaggtgcaaactgtgagggtgatgttgcaaatgaagtg |
| | | | gagactgaacaaatactctgcgatgcttctgtgatgtctttcactgcaggaagttattcttcatatgtacaagttagagg |
| | | | atctgtgcccttatactggtctcaggacatttcaactatgatgcctaaaccacctattacattggatcaggcagatccatt |
| | | | tgcacatgtggctgcccttcactttgaccagatgttccagaggtttggctctcccatcatcatcttgaatttagtgaagga |
| | | | acgagagaaaagaaagcatgaaagaattctgagtgaagaacttgttgctgctgtgacctatctcaaccaatttttgcct |
| | | | cctgagcacactattgtttatattccctgggacatggccaagtataccaaaagcaagctgtgtaatgttcttgatcgact |
| | | | aaatgtgattgcagaaagtgtggtgaagaaaacaggtttctttgtaaaccgccctgattcttactgtagcattttgcggc |
| | | | cagatgaaaagtggaatgaactaggaggatgtgtgattcccactggtcgcctgcagactggcatccttcgaaccaact |
| | | | gtgtggactgtttagatcgcaccaacacagcacagtttatggtgggaaaatgtgctctggcctatcagctgtattcactg |
| | | | ggactgattgacaaacctaatctacagtttgatacagatgcagttaggttatttgaggaactctatgaagatcatggtg |
| | | | ataccctatcccttcagtatggtggttctcaacttgttcatcgtgtgaaaacctacagaaagatagcaccatggacccag |
| | | | cactccaaagacatcatgcaaaccctgtctagatattacagcaatgctttttcagatgccgatagacaagattccatta |
| | | | atctcttcctgggagttttccatcccactgaagggaaacctcatctctgggagctcccaacagatttttatttgcatcaca |
| | | | aaaataccatgagacttttgccaacaagaagaagttatacttactggtggacaccagaggtgataaagcatttaccatt |
| | | | gccctatgatgaagttatctgtgctgtgaacttaaagaagttgatagtgaagaaattccacaaatatgaagaagagatt |
| | | | gatatccacaatgagttctttcggccatatgagttgagcagctttgatgataccttttgcttggctatgacaagctcagca |
| | | | cgtgactttatgcctaagaccgttggaattgatccaagtccatttactgtgcgtaaaccagatgaaactggaaaatcag |
| | | | tattgggaaacaaaagcaatagagaagaagctgtattacagcggaaaacggcagccagcgccccgccgccccccag |
| | | | cgaggaggctgtgtccagcagctctgaggatgactctgggactgatcgggaagaagagggctctgtgtctcagcgctc |
| | | | cactcccgtgaagatgactgatgcaggagacagtgccaaagtgaccgagaatgtggtccaacccatgaaggagctat |
| | | | atggaattaacctctcagatggcctctcagaagaagatttctccatttattcaagatttgttcagctggggcagagtcaa |
| | | | cataaacaagacaagaatagccagcagccctgttctaggtgctcagatggagttataaaactaacacccatctcggct |
| | | | ttctcgcaagataacatctatgaagttcagcccccaagagtagacagaaaatctacagagatcttccaagcccacatc |
| | | | caggccagccaaggtatcatgcagcccctaggaaaagaggactcctccatgtaccgagagtacatcaggaaccgcta |
| | | | cctgtgaaaagagcgcaggtccacctggtggacacgtctgattagcttagaacctgtcttgtctcatcttcaaaaggta |
| | | | acttattaaaagtcctttgcgtctgaagcctttctccttttctgtcacttgcaaattccaaattatagctaataaagatgac |
| | | | tagataatttgcaaaaaaaaaaaaaaaaaa |
| | OPTN | 10 | gttcgttgcaacaaattgatgagcaatgcttttttataatgccaactttgtacaaaaaagttggcatgtcccatcaacctc |
| | (KJ892931.1) | | tcagctgcctcactgaaaaggaggacagccccagtgaaagcacaggaaatggacccccccacctggcccacccaaa |
| | | | cctggacacgtttaccccggaggagctgctgcagcagatgaaagagctcctgaccgagaaccaccagctgaaagaa |
| | | | gccatgaagctaaataatcaagccatgaaagggagatttgaggagctttcggcctggacagagaaacagaaggaag |
| | | | aacgccagttttttgagatacagagcaaagaagcaaaagagcgtctaatggccttgagtcatgagaatgagaaattg |
| | | | aaggaagagcttggaaaactaaaagggaaatcagaaaggtcatctgaggaccccactgatgactccaggcttccca |
| | | | gggccgaagcggagcaggaaaaggaccagctcaggacccaggtggtgaggctacaagcagagaaggcagacctg |
| | | | ttgggcatcgtgtctgaactgcagctcaagctgaactccagcggctcctcagaagattcctttgttgaaattaggatggc |
| | | | tgaaggagaagcagaagggtcagtaaaagaaatcaagcatagtcctgggcccacgagaacagtctccactggcacg |
| | | | gcattgtctaaatataggagcagatctgcagatggggccaagaattacttcgaacatgaggagttaactgtgagccag |
| | | | ctcctgctgtgcctaagggaagggaatcagaaggtggagagacttgaagttgcactcaaggaggccaaagaaagag |
| | | | tttcagattttgaaaagaaaacaagtaatcgttctgagattgaaacccagacagaggggagcacagagaaagagaat |
| | | | gatgaagagaaaggcccggagactgttggaagcgaagtggaagcactgaacctccaggtgacatctctgtttaagga |
| | | | gcttcaagaggctcatacaaaactcagcgaagctgagctaatgaagaagagacttcaagaaaagtgtcaggcccttg |
| | | | aaaggaaaaattctgcaattccatcagagttgaatgaaaagcaagagcttgtttatactaacaaaaagttagagctac |
| | | | aagtggaaagcatgctatcagaaatcaaaatggaacaggctaaaacagaggatgaaaagtccaaattaactgtgct |
| | | | acagatgacacacaacaagcttcttcaagaacataataatgcattgaaaacaattgaggaactaacaagaaaagagt |
| | | | cagaaaaagtggacagggcagtgctgaaggaactgagtgaaaaactggaactggcagagaaggctctggcttccaa |
| | | | acagctgcaaatggatgaaatgaagcaaaccattgccaagcaggaagaggacctggaaaccatgaccatcctcagg |
| | | | gctcagatggaagtttactgttctgattttcatgctgaaagagcagcgagagagaaaattcatgaggaaaaggagca |
| | | | actggcattgcagctggcagttctgctgaaagagaatgatgctttcgaagacggaggcaggcagtccttgatggagat |
| | | | gcagagtcgtcatggggcgagaacaagtgactctgaccagcaggcttaccttgttcaaagaggagctgaggacagg |
| | | | gactggcggcaacagcggaatattccgattcattcctgccccaagtgtggagaggttctgcctgacatagacacgttac |
| | | | agattcacgtgatggattgcatcatttacccaactttcttgtacaaagttggcattataagaaagcattgcttatcaattt |
| | | | gttgcaacgaac |
| | VCP | 11 | agggctcttgtgatcttgctcaagtcagctctcagccttagtctccttattagtaactcaaatatggtccttgccctgagtg |
| | (NG_007887. | | aggaggcacgctggaatagatcatcactgccccaggtttttcttccttttttttaattcttgcctcatagctatctatctctt |
| | 1) | | agaattgggggaaactacaggcattatgcatagcttcttgctttgtaagggtagctgaggcctgtgtcctcaaggatca |
| | | | agaggacccttgagctcaaggtctagccaggatagatggagataggagaagacgaggaggttgtaggcttggagctt |
| | | | ctggttgtccttctttccccagagtggagcctctaaggatctgctgttacttctgaaaacttggagtcccccagctgagg |
| | | | aattagatgctccattgaccctgcaggatgcccagggattgaaggatgtcctcctgacagcatttgcctaccgccaagg |
| | | | tcagctagcaacctgactcatctcttctacctttaagtggtcctccagactcatgccctctctcttgtcccattcagttccat |
| | | | gggcttcttagacccttgttccccatcatgacccctgctctatagccctctccaagctcacaccctgactgtcctcaggtc |
| | | | tccaggagctgatcacagggaacccagacaaggcactaagcagccttcatgaagcggcctcaggcctgtgtccacgg |
| | | | cctgtgttggtccaggtgtacacagcactggggtcctgtcaccgtaagatggtaaagacagtcctagggtggattggg |
| | | | gaaaccacaagcattatgtatagctttttgctttgtaagggtagctgaggcccgtgtcctcctatccctttagggaaatcc |
| | | | acagagagcactgttgtacttggttgcagccctgaaagagggatcagcctggggtcctccacttctggaggcctctag |
| | | | gctctatcagcaactgggggacacaacagcagagctggagagtctggagctgctagttgaggtagggaactgccag |
| | | | gtgaagatgtggggtggaggattcctgaggtgcttgtgatggggtgacttgactagtcaagatccaagtataaggaag |
| | | | tatggctcccaaagattatagatacaatttttttctttctcttttaggccttgaatgtcccatgcagttccaaagccccgca |
| | | | gtttctcattgaggtagaattactactgccaccacctgacctagcctcaccccttcattgtggcactcagagccagacca |
| | | | agcacatactagcaagcaggtgcctacagacggggaggtgaggttccccttgctcacctgagcttctcttcccacttct |
| | | | gatgcctcccctggggtgtgactctgcttttgttcttgtgacttgggggatgaatgaggcagtggcaggacataggtcg |
| | | | ccactcctcagcctcttaccttgcttctagacctgaactgaggactcttctctaagacattaaccttctctatctttgacac |
| | | | tctgaagtctcttccccagtccattctgtaactggggtagatccatactgagccaaaattggagccctaagaccttccct |
| | | | ccctttctcctcctcagggcccatgaacatccatctgttagctaagagagaacattctcaggacccttgcaggttccctg |
| | | | ctagggcctagggtgaggcttatgggcttttactcctcagggcaggagacgctgcagagcattacttggacctgctggc |
| | | | cctgttgctggatagctcggagccaagggtgggtgtgtcttcaagcttctctgcaatggggtagacgggttggtgtccc |
| | | | ctttgaaatacagcaagactgcaattttttgttttttcctctattctgtctgtccagtctgggggcccaggactctgcatgg |
| | | | taccagagcctggtgaagaggttggggatggtggctcatgcctttcaagacactgcttcttcttcccactccagttctcc |
| | | | ccacccccctcccctccagggccctgtatgcctgaggtgtttttggaggcagcggtagcactgatccaggcaggcaga |
| | | | gcccaagatgccttgactctatgtgaggagttgctcagccgcacatcatctctgctacccaagatgtcccggctgtggg |
| | | | aagatgccagaaaaggaaccaaggaactgccatactgcccactctgggtctctgccacccacctgcttcagggccag |
| | | | gcctgggttcaactgggtgcccaaaaagtggcaattagtgaatttagcaggtgagcccggggtcctagagggggtgt |
| | | | ggagggatgattttctgattgggacctgagttggtgagctccatgttcacctacttaccctagagtttctgaaattgattc |
| | | | ctcttcacccctgttctgtgtcctacttcaggtgcctcgagctgctcttccgggccacacctgaggaaaaagaacaaggt |
| | | | gatctaaactttcagttttcctcttacctcctggaagtggtagtggtggaagtgggttccctttaatgtgtcccagcccac |
| | | | caggagacttaagagaaagggactgtggacacaagaagaaatagaggaggatttggggttttggtgactgtgaggc |
| | | | tgggaagacacttcttgactttgggagtggtccccaggggcagctttcaactgtgagcagggatgtaagtcagatgcg |
| | | | gcactgcagcagcttcgggcagccgccctaattagtcgtggactggaatgggtagccagcggccaggataccaaagc |
| | | | cttacaggacttcctcctcagtgtgcagatgtgcccaggtatgtatacttgcatgacgtcggctatgcatagatgtgcaa |
| | | | ggacagagaggcccatgggaaatacttgggcgtaagtgtaagagtggtgttctcagacaggaggggtgattggggtt |
| | | | gtgctatatatacctagggatatatccaggtgttcaaatatacacagtgtgtatgcagggctcccaagagttccctggtt |
| | | | tccccgatatcctcagacacaaagaccatgactctgctgttgtgggttgcacaggagtagggggcagatgatggacac |
| | | | tgggatgggaatggttgtgaatgatctgcccccgcttccatatgtgagtgtaggcaagatacttcctctctctaggcctc |
| | | | agtttccctttttataaaacaatgcctaagactatgtcaagttctaagactctgtactctgggctggggctacaggtaatc |
| | | | gagacacttactttcacctgcttcagactctgaagaggctagatcggagggatgaggccactgcactctggtggaggc |
| | | | tggaggcccaaactaaggggtcacatgaagatgctctgtggtgaggctcaggcctgctggcttggggctgaggggca |
| | | | gttggtgtcctagagtccttggacctattgtgggtggtgaacactgaaagcgggggtatggctgcccagctttctgcca |
| | | | atctttcacccttaggcatcatccttctttctctccaggtctctccccctgtacctagaaagctatttgagctggatccgtc |
| | | | cctctgatcgtgacgccttccttgaagaatttcggacatctctgccaaagtcttgtgacctgtagctgccacgttttgaag |
| | | | agcttgagctgggtccccagtgggctgtctctctgtggggagggctttctgcttcaccatcattaggaatgtgaccattc |
| | | | ctatataattcctggactggtgagattggtggtaggcctgtgaaatttgccctagttactaccattctcgttttggaggaa |
| | | | acaatctctgccaccaccaagtcattgactttgctcgaggcaccttttttcctgtttctccttttctgttgtcgagtaaaatt |
| | | | tcatatttatctcgtctgcctactacatatacttgcgtgggaaggcttgtgaccgtctagttgagtgctagttgtctatgat |
| | | | gccctttcctgacatcgtctaccttatgaacttgaagtctgggtatgaggaaagggctacaggaatcctcactttggag |
| | | | gcagtggctttagtccttcacattgcctgaatgcagagccccaaagcccaggacagtgacaatgagtggagttgggtc |
| | | | tgaagggtggtctagataggcctctcaggacaagggcaggtacctgttacttagagacaggcaggtttctctcagagt |
| | | | aactcttcactcccttcctaattccaaattctagaaattcgactcgtcttagtcaaatgtgtctgtgtgggtacagggtga |
| | | | aaggtgaattgagtcagcatatcaccaacagctgcctgttgactactaacagggacccctgttatttggggcccaagg |
| | | | attttcaagtcccagcatatgcagttttcttgctaatttgggttctactgaattggctgtgtgttctgtggttgccctttgact |
| | | | tctctggatttgagtgccttattataaggtattaatattaatgacgggaggaagaaaggcacaagcgctgagaaactg |
| | | | caaaggctgtgggattggggttaccagagggtgcagtgacaatggggtaggagtggggtgctagtcagaaagttcag |
| | | | agtaggggtggttggaggcagcgttgagaaggagcaagaagtgtcagggtggtgatggcgtcttagaagatctccag |
| | | | gttttgggagaagatgggtgtatgggttaccacaataggactagactaatacaagggttcagtctattgtgccacgttc |
| | | | ttccaggaggataaactccggagccaatgaactggagccaagtgggagagtccgcctctaagggattaaagggtctc |
| | | | ctttcactaatcgatctcgccctattcctttcgttgattggctgagaattccaatccgtcgaggaagcgtagcgttgcggc |
| | | | caattgacgtggcgttactaggcgtgtcgcatcactgaggcgggagccaggccgcaagcgaatttcctgattggctgt |
| | | | gatctgcgggttgctggggagaggcgcggagaggcgggcgagagtccgcagggcaggcgctgattggctgaggtg |
| | | | ggagcagcttcccttccgatgattcggctcttctcggctcagtctcagcgaagcgtctgcgaccgtcgtttgagtcgtcg |
| | | | ctgccgctgccgctgccactgccactgccacctcgcggatcaggagccagcgttgttcgcccgacgcctcgctgccggt |
| | | | gggaggaagcgagagggaagccgcttgcgggtttgtcgccgctgctcgcccaccgcctggaagagccgagccccgg |
| | | | cccagtcggtcgcttgccaccgctcgtagccgttacccgcgggccgccacagccgccggccgggagaggcgcgcgcc |
| | | | atggcttctggagccgagtgagtgtgcgcgcgccgttcgcttgctgcggcggcgcgcaccgggaccagccgcgcagg |
| | | | gtaggcccggcggggcctggccgtgggcgcgtcagagaggtggagaccaggaaagagggaagggaagctagggg |
| | | | gcgagtgaggggccgtggacccggcaggcccggctggggccagctgcgcacccgcgcgccccctaggcggggcttg |
| | | | cgttgggtccgggtcggagcctgggccggatcgtgctgtgtcatgcaggccccggcccgcccgattggctcccttaga |
| | | | acggacgtctgggcctggccaggtcttccggcccactccgccggcgcggcgccccggggctttggggcgccagtctgc |
| | | | cgtccggcctaccacccgccgaaagcctttggtccccggagagagcaggccccgcgagcccgaggccccagccggg |
| | | | cccggtgggcgtggactttgcgccatgtgaaggcctcaggagctctgccaccgaggcggagcccggggtcggggaa |
| | | | ggcccgccgagctcagggaagctcacagccgcctttttggagccgggtcggcggggccggaggtgggcatcctctgt |
| | | | gtgtgacctgagtcgtgaaggactgttagggagagggcgagccctacctttcgcttctggtcttcttactttaggcctgtt |
| | | | gttcatactagaaagtttgagccagttttccttaatgttatgactcgtgggtggggagaaaggaatttttctttaacatttt |
| | | | aggttcttgctaaagttggaatctcagttcgtgttttgtgcgcctttccagatttccaggtttcgtggtgtgcagtttagag |
| | | | ttgcactaactcttcaaaaacacaaacgtgcgcgcactcctttccccaaccagctatattgagaaattacccgggctct |
| | | | ggggctttctgaccccacctccatccttaggaaagcgttagataaaacttggctacctcagcccattcaatttagaatag |
| | | | agagcttttagaggcaaaaaaaaaaaaaaaaaaaaaaaaaaaaacacagccagcccaaggaaactctatgcaaat |
| | | | taccttccttccttgaagagattagttttttgaggtgtgttcctttttctcaagtttctgaattcggataaagtattactcagt |
| | | | tgcttatttaaaagagtcacttattctgaaagtattcttttggtttatatgcagtcagcctcttattttcaaaagtaaaaatc |
| | | | taaaattcttggaacctggcatcatcagctgtttctaacctcttggtcaccttgatgactcttagctgaaacccttccaag |
| | | | tcccgtggagtcccctgtgcagttctggaaagggattgacttatacagaagacaatggaggattgccccaaagagtgt |
| | | | ccagaactgctgttggcccatggtcaggccaaattcagtcttttagagattaccacatctgatctcagtaggtagatgg |
| | | | ggaattagaggctttaccttaagaaaagggcataccactcatctccagagccagagtccgggatggtaggcagaatg |
| | | | gaggttttcctaggatacagctttgagattaaaatacaaactattgaccgggcacagtggctcaggcctgtaatcccag |
| | | | cattttgggaggccgaggcaggcagatcacttgaggtcaggagtttgagaccagcccgggcaacatggagaaaccc |
| | | | catctctattaaaatgcaaaaagtagccaggcatggtgatggtgcacacctgtagtcccagccactctggaggctgag |
| | | | acaggagaatcacttgaacccagggaggcagagattgcagtgagggagatcgcgccactgcactccagcctgggca |
| | | | acagagggagactccgtttcaaaaaataaaaaactatggtgactagaggcatctggcgttttatttttctccagtccca |
| | | | gttctataagtcaagcaagaagatgggcagtccctggaagagtattttgatgggacaggagtgggagagctcgtggg |
| | | | tttacattgctctcaactgtcacattgagcatgcttggcctctagtgtgttgataagcattggaagagtctgcctactcag |
| | | | cagcattgtgcctggagtggcagacttttggaatgggggaagcaaatttgagcagaggaaactgtcgttagaaacta |
| | | | gtttagaggcagtggttaaaaatgcagcctatgtgtgagggttagcagaaggcctaccattttgttagatgaatggggt |
| | | | ttgtttttctcttgggtgtatcaggacccaaagatgtaagaaccccatggcttcctagctgagcacagcatttttctttgtc |
| | | | tcttgcaaattgtgaggatatttccaatgggaatactattttgtcttgttttgttgacttcagtatccccagcccttagaac |
| | | | aatgcctggtacataatagagactcatgaatttgttgaatgaagaaattcgttttaaaaatttattttctttgcttccttcat |
| | | | tgtctggccttcctactttggttaatgcttatgttttcctgagccttactaacacgaggccgctcttaaaaaagagagagc |
| | | | gctgggtgctgtggctcacgcctgtaatcccagtactttgggaggccgaggtgggcagatcacctgaggtcaggagtt |
| | | | cgagaccagcctgaccaccatggtgaaaccccatctctagtaaaaatgcaaaattagccaggtgtggtggcgcatgc |
| | | | ctgtaatcccagctactcgggaggctgaggcaggagaatcacttgaacccgggaggtggaggttgcagttagctgag |
| | | | atcgcagcattgcattccagcctggacaacaaaagcgaaactccatctcaaaaaaaaaaaaaaaaaaaaaaaaag |
| | | | ggagagggagcttgctgagtctagtaagtgacagctggaaacgggctaggtaataagttggtgtcactgtctggtgaa |
| | | | tgatcctagcttctaggaaataacactgagtgtagacccagtcgactttgatttgggtgagagggatttggatttgcccc |
| | | | atgtctcagcatttcttggttttgattttttgagccaactttatggaattgtgtacttttgcagatattactgtgaagttcctt |
| | | | ttgaccttgagcctctttctggcggtttgatgtctgttagtgttttttccaaatatgatggtctttatgcttggcattcctttg |
| | | | gtactatggaatgccctggcatcagtaggtttagcctataaggagggtagcaccaatgattctgcttcgttgtccccag |
| | | | gcttgcttggtagaacttaaggcctccctgagcctcaagggaggttgtctggcttacttaattccctggaaagttagctt |
| | | | atgctttactctatactgttaatcatggtgaccattaataccatgtgccaggtattatgttaagcattttacattcattatct |
| | | | cccttaagacaataagcctctgagataagtattatattccttcttttatagaggaggacatcaaagttcagaggttaggt |
| | | | aacttgcccaagatcacaattaagcagtggcataattggaattcagacccaatgggtctgactatagagttcctgctctt |
| | | | aaccactcttctgtaagtctaagactatttttatttctctaacaactattcagcctccatttctattatgtcttcttcttaggc |
| | | | cattattttctgaccctggggcaggatcactcacttggggccttatagctgggacactgacgctcaaaataccaggagc |
| | | | tgctggaatgggtattgtaatatgtatggtagatactgctcctcgtgaccttggctgcctttccttcatctgagctttctgg |
| | | | tctagggacagcttcatctattcactgtttctttcctaagtatgagttttagagactggcgaggcgcttggggcaggagt |
| | | | atctactgactccatttcctccttctagttcaaaaggtgatgacctatcaacagccattctcaaacagaagaaccgtccc |
| | | | aatcggttaattgttgatgaagccatcaatgaggacaacagtgtggtgtccttgtcccaggtaagctgtggccacagac |
| | | | tagtctttccttactgcacttacttgagggattttcccaggtttcttttctcatttttcttgcagtgactgcagatagagtgg |
| | | | ggtttactgggaatcccaatctccagggctgctgcttactccccgtcagcccaatgacccaaaggccttaactttctttc |
| | | | ctcagcccaagatggatgaattgcagttgttccgaggtgacacagtgttgctgaaaggaaagaagagacgagaagct |
| | | | gtttgcatcgtcctttctgatgatacttgttctgatgagaagattcggatgaatagagttgttcggaataaccttcgtgta |
| | | | cgcctaggggatgtcatcaggtgtgtgtggggtttttggcttcacagggatgggaggccagagatagcctgcattaca |
| | | | ggcaggacccatgtattacaggcaggaccaagttcttggcacctgtcgatgcaggaagcctcctggtcatgggaaga |
| | | | cttatgcttcagggttgtctttaggttttggttctgcctccctgggacttcaaaatccatttctgcaggtcccttgagacaa |
| | | | attggcgttcctgtaactttcttgatggctttattttttcctactagaggtgtaatttatcatacttatatcctggcttaggat |
| | | | accactcgagggtgtgtatgtatatgccaaataatctcctcactactctcactagtatgtctaattgatggcttgtgttgg |
| | | | ggttggaatgaggtgggggtatgggcatggaaggtgagctgctagcaggtcttttaagcccctaagttaacccgggag |
| | | | agaggaatagttggagccagacctgggatagctctcaatgtgagtgattttgcttgttcttgcataattttaggcaagag |
| | | | gttaccccatcttggagtcatctctagccactcctacccaaccaaccatcacctggccaggatcatctcaggcttttgatt |
| | | | cttttgaatggagtctaagtttcatgtagctttcttcttgggagtgcttagtcacttcctcaaggtgttctgaccacctggct |
| | | | gagataattttgtttttttcacttctgtttcactgaccctgacaattgttagcttaagaccttcccttgtaatattgggtcacc |
| | | | agtattagctagaaggggatcatccttggatatctccctgaagaccctgcatgtctttgtggggtttctaaatgtgtggct |
| | | | cttgattttggctcactgattaggagtgagtggggctgttccttcgccctcacttccaccctgttctccttcctctcttcgcc |
| | | | taaagccatcctgccttttctttttcacttactatcagctatctgtgccaggcccttttggacacccagtgcttgggcccga |
| | | | agtgtggttggtaatatggagtctgcttgtcatcctcagcatccagccatgccctgatgtgaagtacggcaaacgtatcc |
| | | | atgtgctgcccattgatgacacagtggaaggcattactggtaatctcttcgaggtataccttaagccgtacttcctggaa |
| | | | gcgtatcgacccatccggaaaggtgagagctaattctgagcttaaggattattgactgtagggaataaaccttggaac |
| | | | atctttatctcattttctttttctttttttttttttaaatcttttatgcttttcccctgtatttatttattcattttttaagagatggg |
| | | | gtcagctgggcaccgtggctcacacctataatcccagcaatttgggaggctgaggcgggtggatcacttgaggccag |
| | | | gagtttgagaccagcctggccaacatggcgaaaccccatcgtgggcacctgtaatcccagctacctgggaggctgag |
| | | | gcatgagaattgcttgaacccaagaggtggaggttgcagtgagccaagattgggccacagtactgcagcctgggtga |
| | | | cagagcaagactctgtctcaaaaaaaaaaaaaaagagacagggtctcactatgatgcccaggctggtctcaaactgc |
| | | | tgggctcaagtgatccatctgccatggcctcccaaagtgctgggattacaggcatgagccatcaagcctagtctcatttt |
| | | | cttttcttttttttttgagacagagtgtcgcgctgtcccccaggctggagcgcagtggtgcaatctcggctcactgcaacc |
| | | | tccgcctcctgggttcaagcaattctcctgcctcagcctcccaagtagctgggattacaggcgtctgccaccacgcccg |
| | | | gctaatttttgtgtttttagtagagacggggtttcaccatgttggtcaggctcgtctcgaactcttgacctcaggtgagcc |
| | | | actgtgcccggccgctagactcattttcatatatttgtatacacacacatgcaaaccctgcacacatattcatatgtctta |
| | | | ccctctttttttcctccatccttcctttgctccatctctccccttctctgttccaggagagtaagctatctttatggatctctga |
| | | | aggagaaagtggtccattttggctgggtcagggtccagagtgcacagttctaccattggtggttgtagtgaaaacttgg |
| | | | gctacctatatggcagaagtcagaacttgatgggcttctgacatgtcaggttttgttcactgacctcttgtcagagggac |
| | | | tcttcacagtttacctttctcatcttgcctgctgcttattaagacaggtggggtggagttggggagaggtagggcaatat |
| | | | ctaatgaagggcactatctaatgagcttggcattttgaccccagggtctgatgagttctcactttgtcttgtagttgacac |
| | | | ctctaactgtgcttgtactgtttgctctcgcaggagacatttttcttgtccgtggtgggatgcgtgctgtggagttcaaagt |
| | | | ggtggaaacagatcctagcccttattgcattgttgctccagacacagtgatccactgcgaaggggagcctatcaaacg |
| | | | agaggtgagttttctccctgattccagtatccgattttatgattactcagtgtggcatcatgtggtaactgtcaggactgg |
| | | | gtgctcggccggctgcggtggctgacacctgtaatcccagtactttgggagactgagatgggcagatcacttgaggtc |
| | | | aggtgttcaagaccagcctgggcaacatggtgaaatcccatctctactaaaaatacaaaaattagccaggcatggtgg |
| | | | tacacatctgtaatcccagctactcaggaggctgaggcaggagaatcggttgaacccaggagtcggaggttgcagtg |
| | | | agctgagattgtgccactgcactccagcctgggtgacagagtgagactctgtctcaaaaaagaaaaagactgggtgtt |
| | | | ctttggagaactaaccatctttcagggatgagaaacctgccagctattcatttctgggcctaattgtttcttggatttacct |
| | | | aatgccaggaatttcaaaaaactagactgaacccaaaatatataagtgattgaaatcatttttgaagtaaagctgatg |
| | | | gtggcttcaggcctctgcccattcccagggtttccagcttcagattttagagaccccttctcagtaagactacgagtaat |
| | | | gtgagaggcaaggactgtgctagaaatctttgccttgggatttttgtagttgttctttgaggccggatccctttagagga |
| | | | gaatcttttttaaatttaatttaatttttaatgagatggagtcttgctgtattgcccaggaactcctggactcaagcattcct |
| | | | cccacctctgcctcccaaagtgctgggattacagatgtgagccaccatgccgggttgagaatcttcttatacggtaggtt |
| | | | tttgcacactaggtagtggaatgatttagagaaactcagcttttgctgatataatattcttgccttctcctttctttatctcc |
| | | | tccatattcaggatgaggaagagtccttgaatgaagtagggtatgatgacattggtggctgcaggaagcagctagctc |
| | | | agataaaggagatggtggaactgcccctgagacatcctgccctctttaaggcaattggtgtgaaggtgagcatcctgg |
| | | | gctctggaatcaagtctaaagtggtgccaatgtctaatcctgtcccaatgtctaatcctgggactgttttcatgcatggct |
| | | | ttcattattgccttggattagaggggcaataacgtatcctttagtttacctaaggctctaaattcattagagctgatggtct |
| | | | aaaaccagagtaggctaatcaaattgtctgttgtgtgcgtgtgcgcacaaaacacacacacatatatatatgggtttttc |
| | | | tttacaactcttagaatataaaagccattcttgtatcaatggaccctgtaaaaacaaatctcaccatagtttgccagcct |
| | | | gtctagagcaatgtcacccagtagaagtaaggaagttaaggaaattttcagagtgttaaagggttctgagtctaaaac |
| | | | atttgagaactattggtctagagtgtagcttctcaatcttttcctagtgggaaagtgtttccatggaacacactgaagatg |
| | | | aagttactcattttcctagtgggtggcacacaaataatttcattttctatgtggacagtttacatgttctgcttgtggatga |
| | | | ggccatagaaagggtagtgttgaagaagaaaaatgatgattgtaaggaacagcattccagtgtgataaattctggag |
| | | | ggcatgattactggagtgagtgatcctctggcaatgaagaaaatagaccctgctctcttaaatggcttagctagtctttg |
| | | | gcccttggtctgtctaaaattgagcccttagtgtaatggcctcttgcctttccctagtcatgtatcttcaaacgcatttgga |
| | | | ctacagtttctctgcccttagtctcctatgcaagttgcaatcataaatgttgcccactttctagcagtattttccctgctagt |
| | | | aatagaaatgagtgtggcctaaagtaattgtcttcttagcatttactgcggagggcttattcttaatattgtcagggttga |
| | | | agcctgattctcaccctctctggagcgctagtcaagccattttagggtttgggagaaggtgggaacctaatcacactct |
| | | | gcattggtccacagcctcctagaggaatcctgctttacggacctcctggaacaggaaagaccctgattgctcgagctgt |
| | | | agcaaatgagactggagccttcttcttcttgatcaatggtgagatatttggttcatcttatgtctagctagacccaattttg |
| | | | aactgggcttatgagctggagcacttatgaacacatcctttttgcacccatgccctccttcatgtttatagcatatttctta |
| | | | tgctggggtatgttacagacagaagagcaataaagggaagatattttacattggtgctccctgtcctgccccctttgag |
| | | | aaagattgtggacagactgcagagcgggagcaagctagaatgagaaatcaaagggtgaatggttagtgatttgaga |
| | | | gggtttggggcaaatgaactttgatcactggctcttggagaatgctgtttagtggtgtgccatctggtgtgccatctctct |
| | | | tgctctagccagaggtcctagagcatttgctgtcacctttacagttcaactgtgagaagagtatagtgagtccctgggct |
| | | | tctctccagccttgcctggtggctgtcctgggataatggctggtagaggatgtgagaagtaggcagaggttaccacctt |
| | | | ctcacccaggacctgtctctgggccaaacaagcaagataactgatttttgggaggaattgggaaagactatcattttgt |
| | | | tattgtctccattctgtatcctttcaggtcctgagatcatgagcaaattggctggtgagtctgagagcaaccttcgtaaag |
| | | | cctttgaggaggctgagaagaatgctcctgccatcatcttcattgatgagctagatgccatcgctcccaaaagagaga |
| | | | aagtaggagcttacctgaggggatagaggggggttgaaaggccctgacttcacttctgaccagacatcctgttctggc |
| | | | agactcatggcgaggtggagcggcgcattgtatcacagttgttgaccctcatggatggcctaaagcagagggcacat |
| | | | gtgattgttatggcagcaaccaacagacccaacagcattgacccagctctacggcgatttggtaaggactccagatac |
| | | | ttttgaccccgtccttgcttaggtcctacttctctccttcatctaagtcacctaatcctcttgaagcccttcacagtgattgg |
| | | | gtccaggggtctttttcctttaccctacgtcctgtctagagtgaccaaccaccctggttttcctgagactgaaaggtttcc |
| | | | cagagcttgagacttttttagtgctggcattaggacaatcctgtgctggctgagatggttggtcaccctaggcctgtctct |
| | | | tacctctggactagagatgagccctgtttatgtttgtgtactgtcccacaggtcgctttgacagggaggtagatattgga |
| | | | attcctgatgctacaggacgcttagagattcttcagatccataccaagaacatgaagctggcagatgatgtggacctg |
| | | | gaacaggtgaagtgatgatgatggctgaccaggcgttacagtgtctctaggcagttgctgggaactggctagagacat |
| | | | aaggttaagatgtgaggagatgggttttgatttctggacaggggaaaggaagtaatctgagattgaatccaggaaatg |
| | | | ggagttggcatttttcatagttgacgctgcatttagagtaaatcagaattgttggagcagccttatttctaggtcccaagt |
| | | | ccagaattaagtacttaaaacccagcccataaaggtattgatagtatatattcaaggaaatgagaggacccagggat |
| | | | agcagtcaggggaaggattctattgtctctgagcctcctgcagcagctgggtctttgaggcagcatagtaagtagatct |
| | | | ttctctgcaggtagccaatgagactcacgggcatgtgggtgctgacttagcagccctgtgctcagaggctgctctgcaa |
| | | | gccatccgcaagaagatggatctcattgacctagaggatgagaccattgatgccgaggtcatgaactctctagcagtt |
| | | | actatggatgacttccgggtaaggaccacacccgtgcctcaggtacacacatacgtgctttgacccctcccttgataag |
| | | | tctcatccccagttttccctccttttctagtgggccttgagccagagtaacccatcagcactgcgggaaaccgtggtaga |
| | | | ggtgccacaggtaacctgggaagacatcgggggcctagaggatgtcaaacgtgagctacaggagctggtccaggta |
| | | | gggcaacttggtccagggtgagtcactgtctcagtacattgtaattgatctgggtgatctcagggtgtcaacacatttgc |
| | | | tgcaagagttgtgagagcacgacttaggaacctactgttcttaggtttgaggcactaaggagtcttcttctagagaacc |
| | | | tggatctgataccattgggtacaccatgaaataatggaggggatgcttctgtttagttaggtttctttcaaaatgtggag |
| | | | gtagccttgaaccctctttccttttcctcctagtatcctgtggagcacccagacaaattcctgaagtttggcatgacacct |
| | | | tccaagggagttctgttctatggacctcctggctgtgggaaaactttgttggccaaagccattgctaatgaatgccagg |
| | | | ccaacttcatctccatcaagggtcctgagctgctcaccatgtggtttggggagtctgaggccaatgtcagagaaatcttt |
| | | | gacaaggtgagctacaataggctgaactatgtattgatttgcctgagggcaaggaatagaggctgtttttctttaagag |
| | | | ggttgaaatattcttagtgctggctgctcaactgcacagtaagtcacttgattttctttctgaggtctgagagacctagtg |
| | | | ttattttttttttctctctctctctcttgagacagggtctggctctgttgcccaggttggagggcagtggtacagtcatggct |
| | | | cactgtaaccttgaaacctgggcttaagcaattctcctacttcagcctcctgagtagctgggactataggcatgcgtcac |
| | | | cacatctggctaattttttattttttgtagagacaaagtctcagtatgttgcccatgctggtttcggatttctggcctcaagt |
| | | | gatcctcccaccttggcctcccaaagtgctgggaatacaggtgtgagccaccacgtttgcctagagacatctagttttgt |
| | | | tagtgcttgaatcaatccattcctcctacaggcccgccaagctgccccctgtgtgctattctttgatgagctggattcgat |
| | | | tgccaaggctcgtggaggtaacattggagatggtggtggggctgctgaccgagtcatcaaccagatcctgacagaaa |
| | | | tggatggcatgtccacaaaaaaaaatgtgttcatcattggcgctaccaaccggcctgacatcattgatcctgccatcct |
| | | | cagacctggccgtcttgatcagctcatctacatcccacttcctgatgagaagtcccgtgttgccatcctcaaggctaacc |
| | | | tgcgcaagtccccagttgccaaggcaggtgcaagatcatgggctgtgggagacttgcatgagtcctcaggctggtacg |
| | | | gagtgctctttagtttctggacaagattccactggggttagggttggtctaaagggaaggtagaatttttgaggatatca |
| | | | agataatctagaatcaggaataaaatggggtggccaaagaaggggcaaactgtagttgggagtgctcgggtagccc |
| | | | aaagatctgcgtatctcgagaggagaggctaaatgctaaggtacctctgctgctgcttttaggatgtggacttggagtt |
| | | | cctggctaaaatgactaatggcttctctggagctgacctgacagagatttgccagcgtgcttgcaagctggccatccgt |
| | | | gaatccatcgagagtgagattaggcgagaacgagagaggcagacaaacccatcagccatggtgagtctgcatccttt |
| | | | ccccagatgtgccaatcatggagagccaggcagcagccaccaccatgccctggagttgagagtagaagctgttggaa |
| | | | agatcatctaactgagaagaattttaatagggcatcaaagataaagaatgctgaggtgaatccattcaatttggaata |
| | | | aggccgagaagagatggtcaggctccattctcagtctgaaccaagctccatgagggaaatcaaagtatgagagtgca |
| | | | gcaaacacagcaaggttttttttgttttttgttttttgttttttttttgagacgaagtctcactctgttgcccagactggagtg |
| | | | cagtggcacgatcttggctcactgcaacttctgcctcccaggttcaagcgattctcctgcctcagcctcccgagtagctg |
| | | | ggactacaggcacatgccaccatgtccggctagttttttgtattttttttttagtagaaacgtggtttcaccacgttagcca |
| | | | ggatggtctcgatctcctgaccttgtgatgtgcccacctcggcctcccaaagtgctgggattacaggcgtgaaccacag |
| | | | agcaaggttttgagctgagatgagactcatatacttatccctgatggttggggaagggatagggtccacagacctccc |
| | | | aaaatgaaaaggcaaattcatgtgtttgtaagttccataaaggtaagatctctgtcatctcacttgttttccactatgtctt |
| | | | gtgttacccttaattaattcattaagttccaaacatgggacttaatgagcaaataaatggctttctttcccttttgaagggt |
| | | | ctgtgacatcccttctctctcccataaaagcttaacaactactgatgaactaatcctaggaggtagtcacataagtcaca |
| | | | gaaattggcctctcaatggaagagataggttttgagctgggctgtgaagagagtagaatttgaataaagggaataag |
| | | | cagcccaaataatgtgctctagtagtaggattgcaatcattgggaaaccctgggtagatttaagagtatatatgtcact |
| | | | ggaagtgagaccgctaggtaggatgtaatccaaatgtggtaagcactgaaagccattggcatttccttttaaagtatta |
| | | | aggtttattaaggtatgatataaatacaataaaattcactctttctatataccatttccatgctttatgacaagtgtgtgta |
| | | | agttctataactactaccacagttgagacttaaaatttctactatctcaaaaagtttccttagccacttcagtcaacatct |
| | | | cccctccttaagccccatcactgatgtgatttctgtccctacagttttcccttttccagagtgcattgacaagtttttaagc |
| | | | agagcagtgactcaattttaggaagcatggcctagcatcttacctcaggttggattggaagggcaaggagaccaataa |
| | | | actgcagtaatgggaggcctgggatgaaatccaggctgggctttaactagccctagtgatctgtgtttaccaactatag |
| | | | gaggtagaagaggatgatccagtgcctgagatccgtcgagatcactttgaagaagccatgcgctttgcgcgccgttct |
| | | | gtcagtgacaatgacattcggaagtatgagatgtttgcccagacccttcagcagagtcggggctttggcagcttcaggt |
| | | | aagttggttgggagcattagacagtgcttaagttactttggggacctacaccaaaagggatgggagtcctaaggaagc |
| | | | tagaggggtagttgtggaaatcttacacaggccctgtcctaaccctcttttttggctttgctcttgtacacagattcccttc |
| | | | agggaaccagggtggagctggccccagtcagggcagtggaggcggcacaggtggcagtgtatacacagaagacaa |
| | | | tgatgatgacctgtatggctaagtggtggtggccagcgtgcagtgagctggcctgcctggaccttgttccctgggggtg |
| | | | ggggcgcttgcccaggagagggaccaggggtgcgcccacagcctgctccattctccagtctgaacagttcagctaca |
| | | | gtctgactctggacagggggtttctgttgcaaaaatacaaaacaaaagcgataaaataaaagcgattttcatttggta |
| | | | ggcggagagtgaattaccaacagggaattgggccttgggcctatgccatttctgttgtagtttggggcagtgcagggg |
| | | | acctgtgtggggtgtgaaccaaggcactactgccacctgccacagtaaagcatctgcacttgactcaatgctgcccga |
| | | | gccctcccttccccctatccaacctgggtaggtgggtaggggccacagttgctggatgtttatatagagagtaggttgat |
| | | | ttattttacatgcttttgagttaatgttggaaaactaatcacaagcagtttctaaaccaaaaaatgacatgttgtaaaag |
| | | | gacaataaacgttgggtcaaaatggagcctgagtcctgggccctgtgcctgcttcttttcctgggaacagccttgggct |
| | | | acccaccactcccaaggcattcttccaaatgtgaaatcctggaagtaagattgcaccttcttcctctcctgatcaacatc |
| | | | ggtatgatgtctcctgttgcctcaccctttgtctgcagtatcactggataggactggtggaaagggagcagcctgacag |
| | | | agctccaaatgtggagaatatggcatccctccacctatatttgatgtggacggtaaggctaggcctgcaggatccctta |
| | | | tcctgaccaaagactgtgttggggtgccatttgaaaatcgcagggttgcaaaagaatacaatcttacttgcaggtggat |
| | | | attctctatactctcttttaatgcatctaaaaatcccaaacatcccctggttggtgatcacttacagttgtgtccacctttat |
| | | | tttatgtactttgattaaaaaaaaaaaactttttgttaatataaaattttagtattgaattttttttttttccaaacagaaaat |
| | | | agactatcctcttcaacagtaatcacttagtgcttctagggtcagtacagtgatgccttacccagagagagagtagtgc |
| | | | agagaaaataaattactaaattaaatatatgttgattggctttgggacataatctcaaaagacagtcctgaacaccgta |
| | | | attttgaataaaatactgtaatctccaaagatcaaaatccctaaagtctaaaattctgaaaatcacaatcccaaaaggt |
| | | | caaaatcccaaaatacaattctggaagaaatactaaacattcttcgaaaatttacttacatttttaaaagcgtatttattt |
| | | | gagaaacaacacaacagaacgtttcataggccactacacgataaaatagggaatagtaacatttttgcaagataaac |
| | | | actcaggtataccaatgacagttgcacggatataacggtgatgagcagatgaaacattcataaagaaataggtcaaa |
| | | | aagtgaaatgtataaatgctttgtcactatgcttggtaattgtgggcacctagctttatataactggtcatctgtaatact |
| | | | gtgacagaaaacctaagtcttgatgagatggttcaaaaactgttgcgttaccactgcattgtctcccaaagagacgag |
| | | | gtcttgagaaattttatccttcacaaatgcacgtgtacaaaaaacagatgtctcttcgtttattgaggaactttcaacatt |
| | | | tttatgcacacatataatgcttacacacagagtcaacattgcggtcatggagtcaaatttttaatgtccaaggcaccag |
| | | | aagaaaaatctgtcctaggctgggcatggtggctcatgcctgtaatcccagcactttgggatgctgaggcgggcagat |
| | | | cacctgaggtcgggagctcaagaccagcctgaccaaaatggagaaaccccatctctactacaaatacaaaattagcc |
| | | | aggcgtggtggcgcttgcctgtactcccagctacttgggaggctgaggcagagaatagcttgaacccgggaggcgga |
| | | | ggttgtcgtgagctgagatggagccattgcactccagcctgggcaacaagagcaaaactcaatcttaaaaaagaaga |
| | | | aaaatctatcctacctcttagagaccaatttgccttctgtatttgttctctctgggcccccgcctgttggatggtacccacc |
| | | | aacattgaaggcagatcttccccactcagtccactcagacttacaaactaatccccggaaacaacctcacagacacac |
| | | | ccagataatgctttaccaggtttctaaatactccttaatccagtaaaattgacacctaaaattaagtccacaattctgctc |
| | | | attggcaacttggcacccttatgtgtctaaaccatacttaagacaataacaaggtaatacttctgtataacatgatgcaa |
| | | | ctgtcttgtttacaacgaaaaacatactaatcctttccccagaatttgaaattttattttgtccaggttgtgattttaaggat |
| | | | tttgatcttttgggacttcaacatttggggttatggtgtttgggattgtgtccttcaggattatgatcaacactggtattagt |
| | | | catcctagactttatgctaggcaagaccaggctccgttttggtctacatagataatcttcaaggatgcatgatacttaca |
| | | | ctggttgactaaactggagcagctctggaactcatgctgtgaaatgggtatttttgcacttttggtatttgttatttcagga |
| | | | ggtttttagggaacaggtggtggttggttacgtgaataagttctttagtggtgatttctaagattttggtgcacctgtcatc |
| | | | tgagcggtgtacaccgtacccaatgtgtagtcttttatccctcaccgcccccccaccttttccctgagtccccagagtccg |
| | | | ttgtatcattcttacgcctttgcattctcatagcttagctcccatg |
| | UBQLN2 | 12 | tgccagagttgctgggagtgcgcgcggtcggatcacaaggcggcggcggaggaggcccagagaccggagcgcgga |
| | (NM_013444. | | gacctcagccagcggcctacgcccaggcctttctccaccggaggaccagggaaccgcagtcttcatcacagaggtac |
| | 3) | | cgtgctccgcgctccccgcctgacccggcccagcccgctgcggcggtgcctccttccttcctccttccctcgcgctctctc |
| | | | tttcgcccgcccgcgccttccctgcccgcctgcgtcaccgcggccgccatggctgagaatggcgagagcagcggcccc |
| | | | ccgcgcccctcccgcggccctgctgcggcccaaggctcggctgctgccccggctgagcctaaaatcatcaaagtcacg |
| | | | gtgaagactcccaaagagaaagaggagttcgcggtgcccgagaacagctcggttcagcagtttaaggaagcgatttc |
| | | | gaaacgcttcaaatcccaaaccgatcagctagtgctgatttttgccggaaaaatcttaaaagatcaagataccttgatc |
| | | | cagcatggcatccatgatgggctgactgttcaccttgtcatcaaaagccagaaccgacctcagggccagtccacgcag |
| | | | cctagcaatgccgcgggaactaacactacctcggcgtcgactcccaggagtaactccacacctatttccacaaatagc |
| | | | aacccgtttgggttggggagcctgggaggacttgcaggccttagcagcctgggcttgagctcgaccaacttctctgag |
| | | | ctccagagccagatgcagcagcagcttatggccagccctgagatgatgatccaaataatggaaaatccctttgttcag |
| | | | agcatgctttcgaatcccgatctgatgaggcagctcattatggctaatccacagatgcagcaattgattcagagaaacc |
| | | | cagaaatcagtcacctgctcaacaacccagacataatgaggcagacactcgaaattgccaggaatccagccatgatg |
| | | | caagagatgatgagaaatcaagacctggctcttagcaatctagaaagcatcccaggtggctataatgctttacggcgc |
| | | | atgtacactgacattcaagagccgatgctgaatgccgcacaagagcagtttgggggtaatccatttgcctccgtgggg |
| | | | agtagttcctcctctggggaaggtacgcagccttcccgcacagaaaatcgcgatccactacccaatccatgggcacca |
| | | | ccgccagctacccagagttctgcaactaccagcacgaccacaagcactggtagtgggtctggcaatagttccagcaat |
| | | | gctactgggaacaccgttgctgccgctaattatgtcgccagcatctttagtaccccaggcatgcagagcctgctgcaac |
| | | | agataactgaaaacccccagctgattcagaatatgctgtcggcgccctacatgagaagcatgatgcagtcgctgagcc |
| | | | agaatccagatttggctgcacagatgatgctgaatagcccgctgtttactgcaaatcctcagctgcaggagcagatgc |
| | | | ggccacagctcccagccttcctgcagcagatgcagaatccagacacactatcagccatgtcaaacccaagagcaatg |
| | | | caggctttaatgcagatccagcaggggctacagacattagccactgaagcacctggcctgattccgagcttcactcca |
| | | | ggtgtgggggtgggggtgctgggaaccgctataggccctgtaggcccagtcacccccataggccccataggccctat |
| | | | agtcccttttacccccataggccccattgggcccataggacccactggccctgcagccccccctggctccaccggctct |
| | | | ggtggccccacggggcctactgtgtccagcgctgcacctagtgaaaccacgagtcctacatcagaatctggacccaac |
| | | | cagcagttcattcagcaaatggtgcaggccctggctggagcaaatgctccacagctgccgaatccagaagtcagattt |
| | | | cagcaacaactggaacagctcaacgcaatggggttcttaaaccgtgaagcaaacttgcaggccctaatagcaacagg |
| | | | aggcgacatcaatgcagccattgaaaggctgctgggctcccagccatcgtaatcacatttctgtacctggaaaaaaaa |
| | | | tgtatcttatttttgataatggctcttaaatctttaaacacacacacaaaatcgttctttactttcattttgattcttttaaatc |
| | | | tgtctagttgtaagtctaatatgatgcattttaagatggagtccctccctcctacttccctcactccctttctcctttgcttat |
| | | | ttttcctaccttcccttcctcttgtctccccactccctccctctttgtttccttccttccttatttcctttagtttccttccttagcc |
| | | | gttttgagtggtgggaatcaatgctgtttcactcaaaagtgttgcatgcaaacacttctctttattctgcatttattgtgatt |
| | | | tttggaaacaggtatcaaccttcacagttgggtgaacaagtgttgtcctacagatgtccaatttatttgcatttttaaaca |
| | | | ttagcctatgatagtaatttaatgtagaatgaagatattaaaaacagaagcaaattatttgaagctctctaatttgtggt |
| | | | acgatattgcttattgtgactttggcatgtatttttgctagcaaaatgctgtaagatttataccattgatcttttttgctatat |
| | | | ttgtatacagtacagtaagcacaattggcactgtacatctaaaaatattacagtagaatctgagtgtaatatgtgtaacc |
| | | | aaaatgagaaagaatacaagaaatgtttctggagctagttatgtctcacaattttgtagaatcttacagcatctttgata |
| | | | aacttctcagtgaaaatgttggctaggcaagttcagttaaaatatagtagaaatgtttatcctggtatctctaagtatac |
| | | | atttaattgtacagaaaatttacagtgtaacattgtgtcaacatttgcagattgactgtatatgaccttaatctttgtgcag |
| | | | cctgaaggatcagtgtagtaatgccaggaaagtgctttttacctaagacttccttctcagcttctcccataaagagaccc |
| | | | taatatgcattttgatttgtaattggaaatgtaactttcactgaaagtgtcatgtgatgtttgcattacttttaactgctatg |
| | | | tataaaggaaagtgtgtcttttgacttcatcagttatttctcttgtgcacagagaaaaatgcattaaaaatgactaaaaa |
| | | | aaataaaaaattaaaaaatggataaatcttttctttttgccttttggccctaggatcgtgtttaggaggattatcccaccc |
| | | | cgaga |
| | C9ORF72 | 13 | ggtggcgagtggatatctccggagcatttagataatgtgacagttggaatgcagtgatgtcgactctttgcccaccgcc |
| | (BC068445.1) | | atctccagctgttgccaagacagagattgctttaagtggcaaatcacctttattagcagctacttttgcttactgggaca |
| | | | atattcttggtcctagagtaaggcacatttgggctccaaagacagaacaggtacttctcagtgatggagaaataacttt |
| | | | tcttgccaaccacactctaaatggagaaatccttcgaaatgcagagagtggtgctatagatgtaaagttttttgtcttgt |
| | | | ctgaaaagggagtgattattgtttcattaatctttgatggaaactggaatggggatcgcagcacatatggactatcaatt |
| | | | atacttccacagacagaacttagtttctacctcccacttcatagagtgtgtgttgatagattaacacatataatccggaa |
| | | | aggaagaatatggatgcataaggaaagacaagaaaatgtccagaagattatcttagaaggcacagagagaatgga |
| | | | agatcagggtcagagtattattccaatgcttactggagaagtgattcctgtaatggaactgctttcatctatgaaatcac |
| | | | acagtgttcctgaagaaatagatatagctgatacagtactcaatgatgatgatattggtgacagctgtcatgaaggctt |
| | | | tcttctcaatgccatcagctcacacttgcaaacctgtggctgttccgttgtagtaggtagcagtgcagagaaagtaaat |
| | | | aagatagtcagaacattatgcctttttctgactccagcagagagaaaatgctccaggttatgtgaagcagaatcatcat |
| | | | ttaaatatgagtcagggctctttgtacaaggcctgctaaaggattcaactggaagctttgtgctgcctttccggcaagtc |
| | | | atgtatgctccatatcccaccacacacatagatgtggatgtcaatactgtgaagcagatgccaccctgtcatgaacata |
| | | | tttataatcagcgtagatacatgagatccgagctgacagccttctggagagccacttcagaagaagacatggctcagg |
| | | | atacgatcatctacactgacgaaagctttactcctgatttgaatatttttcaagatgtcttacacagagacactctagtga |
| | | | aagccttcctggatcaggtctttcagctgaaacctggcttatctctcagaagtactttccttgcacagtttctacttgtcct |
| | | | tcacagaaaagccttgacactaataaaatatatagaagacgatacgcagaagggaaaaaagccctttaaatctcttc |
| | | | ggaacctgaagatagaccttgatttaacagcagagggcgatcttaacataataatggctctggctgagaaaattaaac |
| | | | caggcctacactcttttatctttggaagacctttctacactagtgtgcaagaacgagatgttctaatgactttttaaatgt |
| | | | gtaacttaataagcctattccatcacaatcatgatcgctggtaaagtagctcagtggtgtggggaaacgttcccctgga |
| | | | tcatactccagaattctgctctcagcaattgcagttaagtaagttacactacagttctcacaagagcctgtgaggggat |
| | | | gtcaggtgcatcattacattgggtgtctcttttcctagatttatgcttttgggatacagacctatgtttacaatataataaa |
| | | | tattattgctatcttttaaagatataataataggatgtaaacttgaccacaactactgtttttttgaaatacatgattcatg |
| | | | gtttacatgtgtcaaggtgaaatctgagttggcttttacagatagttgactttctatcttttggcattctttggtgtgtagaa |
| | | | ttactgtaatacttctgcaatcaactgaaaactagagcctttaaatgatttcaattccacagaaagaaagtgagcttga |
| | | | acataggatgagctttagaaagaaaattgatcaagcagatgtttaattggaattgattattagatcctactttgtggattt |
| | | | agtccctgggattcagtctgtagaaatgtctaatagttctctatagtccttgttcctggtgaaccacagttagggtgttttg |
| | | | tttattttattgttcttgctattgttgatattctatgtagttgagctctgtaaaaggaaattgtattttatgttttagtaattgtt |
| | | | gccaactttttaaattaattttcattatttttgagccaaattgaaatgtgcacctcctgtgccttttttctccttagaaaatct |
| | | | aattacttggaacaagttcagatttcactggtcagtcattttcatcttgttttcttcttgctaagtcttaccatgtacctgctt |
| | | | tggcaatcattgcaactctgagattataaaatgccttagagaatatactaactaataagatctttttttcagaaacagaa |
| | | | aatagttccttgagtacttccttcttgcatttctgcctatgtttttgaagttgttgctgtttgcctgcaataggctataagga |
| | | | atagcaggagaaattttactgaagtgctgttttcctaggtgctactttggcagagctaagttatcttttgttttcttaatgc |
| | | | gtttggaccattttgctggctataaaataactgattaatataattctaacacaatgttgacattgtagttacacaaacaca |
| | | | aataaatattttatttaaaattcaaaaaaaaaaaaaaaaaaaaaaaaaaaaaaaaaaaaaaaaacaaaaaaaaaa |
| | | | aaaaaa |
| | DCTN1 | 14 | MAQSKRHVYSRTPSGSRMSAEASARPLRVGSRVEVIGKGHRGTVAYVGATLFATGKWV |
| | (NG_008735. | | GVILDEAKGKNDGTVQGRKYFTCDEGHGIFVRQSQIQVFEDGADTTSPETPDSSASKVL |
| | 2) | | KREGTDTTAKTSKLRGLKPKKAPTARKTTTRRPKPTRPASTGVAGASSSLGPSGSASAG |
| | | | ELSSSEPSTPAQTPLAAPIIPTPVLTSPGAVPPLPSPSKEEEGLRAQVRDLEEKLETLRLK |
| | | | RAEDKAKLKELEKHKIQLEQVQEWKSKMQEQQADLQRRLKEARKEAKEALEAKERY |
| | | | MEEMADTADAIEMATLDKEMAEERAESLQQEVEALKERVDELTTDLEILKAEIEEKGS |
| | | | DGAASSYQLKQLEEQNARLKDALVRMRDLSSSEKQEHVKLQKLMEKKNQELEVVRQQ |
| | | | RERLQEELSQAESTIDELKEQVDAALGAEEMVEMLTDRNLNLEEKVRELRETVGDLEA |
| | | | MNEMNDELQENARETELELREQLDMAGARVREAQKRVEAAQETVADYQQTIKKYRQ |
| | | | LTAHLQDVNRELTNQQEASVERQQQPPPETFDFKIKFAETKAHAKAIEMELRQMEVA |
| | | | QANRHMSLLTAFMPDSFLRPGGDHDCVLVLLLMPRLICKAELIRKQAQEKFELSENCSE |
| | | | RPGLRGAAGEQLSFAAGLVYSLSLLQATLHRYEHALSQCSVDVYKKVGSLYPEMSAHER |
| | | | SLDFLIELLHKDQLDETVNVEPLTKAIKYYQHLYSIHLAEQPEDCTMQLADHIKFTQSAL |
| | | | DCMSVEVGRLRAFLQGGQEATDIALLLRDLETSCSDIRQFCKKIRRRMPGTDAPGIPAA |
| | | | LAFGPQVSDTLLDCRKHLTWVVAVLQEVAAAAAQUAPLAENEGLLVAALEELAFKAS |
| | | | EQIYGTPSSSPYECLRQSCNILISTMNKLATAMQEGEYDAERPPSKPPPVELRAAALRAE |
| | | | ITDAEGLGLKLEDRETVIKELKKSLKIKGEELSEANVRLSLLEKKLDSAAKDADERIEKV |
| | | | QTRLEETQALLRKKEKEFEETMDALQADIDQLEAEKAELKQRLNSQSKRTIEGLRGPPP |
| | | | SGIATLVSGIAGEEQQRGAIPGQAPGSVPGPGLVKDSPLLLQQISAMRLHISQLQHENSIL |
| | | | KGAQMKASLASLPPLHVAKLSHEGPGSELPAGALYRKTSQLLETLNQLSTHTHVVDITR |
| | | | TSPAAKSPSAQLMEQVAQLKSLSDTVEKLKDEVLKETVSQRPGATVPTDFATFPSSAFL |
| | | | RAKEEQQDDTVYMGKVTFSCAAGFGQRHRLVLTQEQLHQLHSRLIS |
| | HTT gene | 15 | MATLEKLMKAFESLKSFQQQQQQQQQQQQQQQQQQQQQQQPPPPPPPPPPPQLPQPP |
| | mutation | | PQAQPLLPQPQPPPPPPPPPPGPAVAEEPLHRPKKELSATKKDRVNHCLTICENIVAQS |
| | (CAG | | VRNSPEFQKLLGIAMELFLLCSDDAESDVRMVADECLNKVIKALMDSNLPRLQLELYKE |
| | segment) | | IKKNGAPRSLRAALWRFAELAHLVRPQKCRPYLVNLLPCLTRTSKRPEESVQETLAAAV |
| | (NM_002111. | | PKIMASFGNFANDNEIKVLLKAFIANLKSSSPTIRRTAAGSAVSICQHSRRTQYFYSWLL |
| | 8) | | NVLLGLLVPVEDEHSTLLILGVLLTLRYLVPLLQQQVKDTSLKGSFGVTRKEMEVSPSAE |
| | | | QLVQVYELTLHHTQHQDHNVVTGALELLQQLFRTPPPELLQTLTAVGGIGQLTAAKEE |
| | | | SGGRSRSGSIVELIAGGGSSCSPVLSRKQKGKVLLGEEEALEDDSESRSDVSSSALTASVK |
| | | | DEISGELAASSGVSTPGSAGHDIITEQPRSQHTLQADSVDLASCDLTSSATDGDEEDILSH |
| | | | SSSQVSAVPSDPAMDLNDGTQASSPISDSSQTTTEGPDSAVTPSDSSEIVLDGTDNQYLG |
| | | | LQIGQPQDEDEEATGILPDEASEAFRNSSMALQQAHLLKNMSHCRQPSDSSVDKFVLR |
| | | | DEATEPGDQENKPCRIKGDIGQSTDDDSAPLVHCVRLLSASFLLTGGKNVLVPDRDVRV |
| | | | SVKALALSCVGAAVALHPESFFSKLYKVPLDTTEYPEEQYVSDILNYIDHGDPQVRGATA |
| | | | ILCGTLICSILSRSRFHVGDWMGTIRTLTGNTFSLADCIPLLRKTLKDESSVTCKLACTAV |
| | | | RNCVMSLCSSSYSELGLQUIDVLTLRNSSYWLVRTELLETLAEIDFRLVSFLEAKAENLH |
| | | | RGAHHYTGLLKLQERVLNNVVIHLLGDEDPRVRHVAAASLIRLVPKLFYKCDQGQADP |
| | | | VVAVARDQSSVYLKLLMHETQPPSHFSVSTITRIYRGYNLLPSITDVTMENNLSRVIAAV |
| | | | SHELITSTTRALTFGCCEALCLLSTAFPVCIWSLGWHCGVPPLSASDESRKSCTVGMAT |
| | | | MILTLLSSAWFPLDLSAHQDALILAGNLLAASAPKSLRSSWASEEEANPAATKQEEVW |
| | | | PALGDRALVPMVEQLFSHLLKVINICAHVLDDVAPGPAIKAALPSLTNPPSLSPIRRKGK |
| | | | EKEPGEQASVPLSPKKGSEASAASRQSDTSGPVTTSKSSSLGSFYHLPSYLKLHDVLKAT |
| | | | HANYKVTLDLQNSTEKFGGFLRSALDVLSQILELATLQDIGKCVEEILGYLKSCFSREPM |
| | | | MATVCVQQLLKTLFGTNLASQFDGLSSNPSKSQGRAQRLGSSSVRPGLYHYCFMAPYT |
| | | | HFTQALADASLRNMVQAEQENDTSGWFDVLQKVSTQLKTNLTSVTKNRADKNAIHN |
| | | | HIRLFEPLVIKALKQYTTTTCVQLQKQVLDLLAQLVQLRVNYCLLDSDQVFIGFVLKQFE |
| | | | YIEVGQFRESEAIIPNIFFFLVLLSYERYHSKQIIGIPKIIQLCDGIMASGRKAVTHAIPALQ |
| | | | PIVHDLFVLRGTNKADAGKELETQKEVVVSMLLRLIQYHQVLEMFILVLQQCHKENED |
| | | | KWKRLSRQIADIILPMLAKQQMHIDSHEALGVLNTLFEILAPSSLRPVDMLLRSMFVTP |
| | | | NTMASVSTVQLWISGILAILRVLISQSTEDIVLSRIQELSFSPYLISCTVINRLRDGDSTSTL |
| | | | EEHSEGKQIKNLPEETFSRFLLQLVGILLEDIVTKQLKVEMSEQQHTFYCQELGTLLMCL |
| | | | IHIFKSGMFRRITAAATRLFRSDGCGGSFYTLDSLNLRARSMITTHPALVLLWCQILLLV |
| | | | NHTDYRWWAEVQQTPKRHSLSSTKLLSPQMSGEEEDSDLAAKLGMCNREIVRRGALIL |
| | | | FCDYVCQNLHDSEHLTWLIVNHIQDLISLSHEPPVQDFISAVHRNSAASGLFIQAIQSRC |
| | | | ENLSTPTMLKKTLQCLEGIHLSQSGAVLTLYVDRLLCTPFRVLARMVDILACRRVEMLL |
| | | | AANLQSSMAQLPMEELNRIQEYLQSSGLAQRHQRLYSLLDRFRLSTMQDSLSPSPPVSS |
| | | | HPLDGDGHVSLETVSPDKDWYVHLVKSQCWTRSDSALLEGAELVNRIPAEDMNAFM |
| | | | MNSEFNLSLLAPCLSLGMSEISGGQKSALFEAAREVTLARVSGTVQQLPAVHHVFQPEL |
| | | | PAEPAAYWSKLNDLFGDAALYQSLPTLARALAQYLVVVSKLPSHLHLPPEKEKDIVKFV |
| | | | VATLEALSWHLIHEQIPLSLDLQAGLDCCCLALQLPGLWSVVSSTEFVTHACSLIYCVHF |
| | | | ILEAVAVQPGEQLLSPERRTNTPKAISEEEEEVDPNTQNPKYITAACEMVAEMVESLQS |
| | | | VLALGHKRNSGVPAFLTPLLRNIIISLARLPLVNSYTRVPPLVVVKLGWSPKPGGDFGTA |
| | | | FPEIPVEFLQEKEVFKEFIYRINTLGWTSRTQFEETWATLLGVLVTQPLVMEQEESPPE |
| | | | EDTERTQINVLAVQAITSLVLSAMTVPVAGNPAVSCLEQQPRNKPLKALDTRFGRKLSII |
| | | | RGIVEQEIQAMVSKRENIATHHLYQAWDPVPSLSPATTGALISHEKLLLQINPERELGS |
| | | | MSYKLGQVSIHSVWLGNSITPLREEEWDEEEEEEADAPAPSSPPTSPVNSRKHRAGVDI |
| | | | HSCSQFLLELYSRWILPSSSARRTPAILISEVVRSLLVVSDLFTERNQFELMYVTLTELRR |
| | | | VHPSEDEILAQYLVPATCKAAAVLGMDKAVAEPVSRLLESTLRSSHLPSRVGALHGVLY |
| | | | VLECDLLDDTAKQLIPVISDYLLSNLKGIAHCVNIHSQQHVLVMCATAFYLIENYPLDVG |
| | | | PEFSASIIQMCGVMLSGSEESTPSIIYHCALRGLERLLLSEQLSRLDAESLVKLSVDRVNV |
| | | | HSPHRAMAALGLMLTCMYTGKEKVSPGRTSDPNPAAPDSESVIVAMERVSVLFDRIRK |
| | | | GFPCEARVVARILPQFLDDFFPPQDIMNKVIGEFLSNQQPYPQFMATVVYKVFQTLHST |
| | | | GQSSMVRDWVMLSLSNFTQRAPVAMATWSLSCFFVSASTSPWVAAILPHVISRMGKLE |
| | | | QVDVNLFCLVATDFYRHQIEEELDRRAFQSVLEVVAAPGSPYHRLLTCLRNVHKVTTC |
| | LRRK2 | 16 | MASGSCQGCEEDEETLKKLIVRLNNVQEGKQIETLVQILEDLLVFTYSEHASKLFQGKNI |
| | (NM_198578. | | HVPLLIVLDSYMRVASVQQVGWSLLCKLIEVCPGTMQSLMGPQDVGNDWEVLGVHQLI |
| | 3) | | LKMLTVHNASVNLSVIGLKTLDLLLTSGKITLLILDEESDIFMLIFDAMHSFPANDEVQK |
| | | | LGCKALHVLFERVSEEQLTEFVENKDYMILLSALTNFKDEEEIVLHVLHCLHSLAIPCNN |
| | | | VEVLMSGNVRCYNIVVEAMKAFPMSERIQEVSCCLLHRLTLGNFFNILVLNEVHEFVVK |
| | | | AVQQYPENAALQISALSCLALLTETIFLNQDLEEKNENQENDDEGEEDKLFWLEACYK |
| | | | ALTWHRKNKHVQEAACWALNNLLMYQNSLHEKIGDEDGHFPAHREVMLSMLMHSSS |
| | | | KEVFQASANALSTLLEQNVNFRKILLSKGIHLNVLELMQKHIHSPEVAESGCKMLNHLF |
| | | | EGSNTSLDIMAAVVPKILTVMKRHETSLPVQLEALRAILHFIVPGMPEESREDTEFHHK |
| | | | LNMVKKQCFKNDIHKLVLAALNRFIGNPGIQKCGLKVISSIVHFPDALEMLSLEGAMDS |
| | | | VLHTLQMYPDDQEIQCLGLSLIGYLITKKNVFIGTGHLLAKILVSSLYRFKDVAEIQTKGF |
| | | | QTILAILKLSASFSKLLVHHSFDLVIFHQMSSNIMEQKDQQFLNLCCKCFAKVAMDDYL |
| | | | KNVMLERACDQNNSIMVECLLLLGADANQAKEGSSLICQVCEKESSPKLVELLLNSGSR |
| | | | EQDVRKALTISIGKGDSQIISLLLRRLALDVANNSICLGGFCIGKVEPSWLGPLFPDKTSN |
| | | | LRKQTNIASTLARMVIRYQMKSAVEEGTASGSDGNFSEDVLSKFDEWTFIPDSSMDSVF |
| | | | AQSDDLDSEGSEGSFLVKKKSNSISVGEFYRDAVLQRCSPNLQRHSNSLGPIFDHEDLLK |
| | | | RKRKILSSDDSLRSSKLQSHMRHSDSISSLASEREYITSLDLSANELRDIDALSQKCCISVH |
| | | | LEHLEKLELHQNALTSFPQQLCETLKSLTHLDLHSNKFTSFPSYLLKMSCIANLDVSRN |
| | | | DIGPSVVLDPTVKCPTLKQFNLSYNQLSFVPENLTDVVEKLEQLILEGNKISGICSPLRLK |
| | | | ELKILNLSKNHISSLSENFLEACPKVESFSARMNFLAAMPFLPPSMTILKLSQNKFSCIPE |
| | | | AILNLPHLRSLDMSSNDIQYLPGPAHWKSLNLRELLFSHNQISILDLSEKAYLWSRVEKL |
| | | | HLSHNKLKEIPPEIGCLENLTSLDVSYNLELRSFPNEMGKLSKIWDLPLDELHLNFDFK |
| | | | HIGCKAKDIIRFLQQRLKKAVPYNRMKLMIVGNTGSGKTTLLQQLMKTKKSDLGMQSA |
| | | | TVGIDVKDWPIQIRDKRKRDLVLNVWDFAGREEFYSTHPHFMTQRALYLAVYDLSKG |
| | | | QAEVDAMKPWLFNIKARASSSPVILVGTHLDVSDEKQRKACMSKITKELLNKRGFPAIR |
| | | | DYHFVNATEESDALAKLRKTIINESLNFKIRDQLVVGQLIPDCYVELEKIILSERKNVPIE |
| | | | FPVIDRKRLLQLVRENQLQLDENELPHAVHFLNESGVLLHFQDPALQLSDLYFVEPKW |
| | | | LCKIMAQILTVKVEGCPKHPKGIISRRDVEKFLSKKRKFPKNYMSQYFKLLEKFQIALPIG |
| | | | EEYLLVPSSLSDHRPVIELPHCENSEIIIRLYEMPYFPMGFWSRLINRLLEISPYMLSGRE |
| | | | RALRPNRMYWRQGIYLNWSPEAYCLVGSEVLDNHPESFLKITVPSCRKGCILLGQVVD |
| | | | HIDSLMEEWFPGLLEIDICGEGETLLKKWALYSFNDGEEHQKILLDDLMKKAEEGDLLV |
| | | | NPDQPRLTIPISQIAPDLILADLPRNIMLNNDELEFEQAPEFLLGDGSFGSVYRAAYEGE |
| | | | EVAVKIFNKHTSLRLLRQELVVLCHLHHPSLISLLAAGIRPRMLVMELASKGSLDRLLQQ |
| | | | DKASLTRTLQHRIALHVADGLRYLHSAMHYRDLKPHNVLLFTLYPNAAIIAKIADYGIA |
| | | | QYCCRMGIKTSEGTPGFRAPEVARGNVIYNQQADVYSFGLLLYDILTTGGRIVEGLKFPN |
| | | | EFDELEIQGKLPDPVKEYGCAPWPMVEKLIKQCLKENPQERPTSAQVFDILNSAELVCL |
| | | | TRRILLPKNVIVECMVATHHNSRNASIWLGCGHTDRGQLSFLDLNTEGYTSEEVADSRI |
| | | | LCLALVHLPVEKESWIVSGTQSGTLLVINTEDGKKRHTLEKMTDSVTCLYCNSFSKQSK |
| | | | QKNFLLVGTADGKLAIFEDKTVKLKGAAPLKILNIGNVSTPLMCLSESTNSTERNVMWG |
| | | | GCGTKIFSFSNDFTIQKLIETRTSQLFSYAAFSDSNIITVVVDTALYIAKQNSPVVEVWDK |
| | | | KTEKLCGLIDCVHFLREVMVKENKESKHKMSYSGRVKTLCLQKNTALWIGTGGGHILL |
| | | | LDLSTRRLIRVIYNFCNSVRVMMTAQLGSLKNVMLVLGYNRKNTEGTQKQKEIQSCLTV |
| | | | WDINLPHEVQNLEKHIEVRKELAEKMRRTSVE |
| | PARK7 | 17 | tgagtctgcgcagtgtggggctgagggaggccggacggcgcgcgtgcgtgctggcgtgcgttcattttcagcctggtg |
| | (NM_007262. | | tggggtgagtggtacccaacgggccggggcgccgcgtccgcaggaagaggcgcggggtgcaggcttgtaaacatat |
| | 4) | | aacataaaaatggcttccaaaagagctctggtcatcctggctaaaggagcagaggaaatggagacggtcatccctgt |
| | | | agatgtcatgaggcgagctgggattaaggtcaccgttgcaggcctggctggaaaagacccagtacagtgtagccgtg |
| | | | atgtggtcatttgtcctgatgccagccttgaagatgcaaaaaaagagggaccatatgatgtggtggttctaccaggag |
| | | | gtaatctgggcgcacagaatttatctgagtctgctgctgtgaaggagatactgaaggagcaggaaaaccggaagggc |
| | | | ctgatagccgccatctgtgcaggtcctactgctctgttggctcatgaaataggttttggaagtaaagttacaacacaccc |
| | | | tcttgctaaagacaaaatgatgaatggaggtcattacacctactctgagaatcgtgtggaaaaagacggcctgattctt |
| | | | acaagccgggggcctgggaccagcttcgagtttgcgcttgcaattgttgaagccctgaatggcaaggaggtggcggct |
| | | | caagtgaaggctccacttgttcttaaagactagagcagcgaactgcgacgatcacttagagaaacaggccgttagga |
| | | | atccattctcactgtgttcgctctaaacaaaacagtggtaggttaatgtgttcagaagtcgctgtccttactacttttgcg |
| | | | gaagtatggaagtcacaactacacagagatttctcagcctacaaattgtgtctatacatttctaagccttgtttgcagaa |
| | | | taaacagggcatttagcaaactaaaaaaaaaaaaaaaaaaa |
| | PINK1 | 18 | cgcagaggcaccgccccaagtttgttgtgaccggcgggggacgccggtggtggcggcagcggcggctgcgggggca |
| | (NM_032409. | | ccgggccgcggcgccaccatggcggtgcgacaggcgctgggccgcggcctgcagctgggtcgagcgctgctgctgc |
| | 2) | | gcttcacgggcaagcccggccgggcctacggcttggggcggccgggcccggcggcgggctgtgtccgcggggagcg |
| | | | tccaggctgggccgcaggaccgggcgcggagcctcgcagggtcgggctcgggctccctaaccgtctccgcttcttccg |
| | | | ccagtcggtggccgggctggcggcgcggttgcagcggcagttcgtggtgcgggcctggggctgcgcgggcccttgcg |
| | | | gccgggcagtctttctggccttcgggctagggctgggcctcatcgaggaaaaacaggcggagagccggcgggcggtc |
| | | | tcggcctgtcaggagatccaggcaatttttacccagaaaagcaagccggggcctgacccgttggacacgagacgctt |
| | | | gcagggctttcggctggaggagtatctgatagggcagtccattggtaagggctgcagtgctgctgtgtatgaagccac |
| | | | catgcctacattgccccagaacctggaggtgacaaagagcaccgggttgcttccagggagaggcccaggtaccagtg |
| | | | caccaggagaagggcaggagcgagctccgggggcccctgccttccccttggccatcaagatgatgtggaacatctcg |
| | | | gcaggttcctccagcgaagccatcttgaacacaatgagccaggagctggtcccagcgagccgagtggccttggctgg |
| | | | ggagtatggagcagtcacttacagaaaatccaagagaggtcccaagcaactagcccctcaccccaacatcatccggg |
| | | | ttctccgcgccttcacctcttccgtgccgctgctgccaggggccctggtcgactaccctgatgtgctgccctcacgcctcc |
| | | | accctgaaggcctgggccatggccggacgctgttcctcgttatgaagaactatccctgtaccctgcgccagtacctttgt |
| | | | gtgaacacacccagcccccgcctcgccgccatgatgctgctgcagctgctggaaggcgtggaccatctggttcaacag |
| | | | ggcatcgcgcacagagacctgaaatccgacaacatccttgtggagctggacccagacggctgcccctggctggtgat |
| | | | cgcagattttggctgctgcctggctgatgagagcatcggcctgcagttgcccttcagcagctggtacgtggatcggggc |
| | | | ggaaacggctgtctgatggccccagaggtgtccacggcccgtcctggccccagggcagtgattgactacagcaaggc |
| | | | tgatgcctgggcagtgggagccatcgcctatgaaatcttcgggcttgtcaatcccttctacggccagggcaaggccca |
| | | | ccttgaaagccgcagctaccaagaggctcagctacctgcactgcccgagtcagtgcctccagacgtgagacagttggt |
| | | | gagggcactgctccagcgagaggccagcaagagaccatctgcccgagtagccgcaaatgtgcttcatctaagcctct |
| | | | ggggtgaacatattctagccctgaagaatctgaagttagacaagatggttggctggctcctccaacaatcggccgcca |
| | | | ctttgttggccaacaggctcacagagaagtgttgtgtggaaacaaaaatgaagatgctctttctggctaacctggagtg |
| | | | tgaaacgctctgccaggcagccctcctcctctgctcatggagggcagccctgtgatgtccctgcatggagctggtgaat |
| | | | tactaaaagaacatggcatcctctgtgtcgtgatggtctgtgaatggtgagggtgggagtcaggagacaagacagcg |
| | | | cagagagggctggttagccggaaaaggcctcgggcttggcaaatggaagaacttgagtgagagttcagtctgcagtc |
| | | | ctctgctcacagacatctgaaaagtgaatggccaagctggtctagtagatgaggctggactgaggaggggtaggcct |
| | | | gcatccacagagaggatccaggccaaggcactggctgtcagtggcagagtttggctgtgacctttgcccctaacacga |
| | | | ggaactcgtttgaagggggcagcgtagcatgtctgatttgccacctggatgaaggcagacatcaacatgggtcagca |
| | | | cgttcagttacgggagtgggaaattacatgaggcctgggcctctgcgttcccaagctgtgcgttctggaccagctactg |
| | | | aattattaatctcacttagcgaaagtgacggatgagcagtaagtaagtaagtgtggggatttaaacttgagggtttccc |
| | | | tcctgactagcctctcttacaggaattgtgaaatattaaatgcaaatttacaactgcagatgacgtatgtgccttgaact |
| | | | gaatatttggctttaagaatgattcttatactctgaaggtgagaatattttgtgggcaggtatcaacattggggaagaga |
| | | | tttcatgtctaactaactaactttatacatgatttttaggaagctattgcctaaatcagcgtcaacatgcagtaaaggttg |
| | | | tcttcaactgaaaaaaaaaaaaaaaaaaaaa |
| | PRKN | 19 | attcctagggccgggcgcgggggcggggaggcctggaggatttaacccaggagagccgctggtgggaggcgcggct |
| | (NM_004562. | | ggcgccgctgcgcgcatgggcctgttcctggcccgcagccgccacctacccagtgaccatgatagtgtttgtcaggttc |
| | 2) | | aactccagccatggtttcccagtggaggtcgattctgacaccagcatcttccagctcaaggaggtggttgctaagcgac |
| | | | agggggttccggctgaccagttgcgtgtgattttcgcagggaaggagctgaggaatgactggactgtgcagaattgtg |
| | | | acctggatcagcagagcattgttcacattgtgcagagaccgtggagaaaaggtcaagaaatgaatgcaactggaggc |
| | | | gacgaccccagaaacgcggcgggaggctgtgagcgggagccccagagcttgactcgggtggacctcagcagctcag |
| | | | tcctcccaggagactctgtggggctggctgtcattctgcacactgacagcaggaaggactcaccaccagctggaagtc |
| | | | cagcaggtagatcaatctacaacagcttttatgtgtattgcaaaggcccctgtcaaagagtgcagccgggaaaactca |
| | | | gggtacagtgcagcacctgcaggcaggcaacgctcaccttgacccagggtccatcttgctgggatgatgttttaattcc |
| | | | aaaccggatgagtggtgaatgccaatccccacactgccctgggactagtgcagaatttttctttaaatgtggagcacac |
| | | | cccacctctgacaaggaaacatcagtagctttgcacctgatcgcaacaaatagtcggaacatcacttgcattacgtgc |
| | | | acagacgtcaggagccccgtcctggttttccagtgcaactcccgccacgtgatttgcttagactgtttccacttatactgt |
| | | | gtgacaagactcaatgatcggcagtttgttcacgaccctcaacttggctactccctgccttgtgtggctggctgtcccaa |
| | | | ctccttgattaaagagctccatcacttcaggattctgggagaagagcagtacaaccggtaccagcagtatggtgcaga |
| | | | ggagtgtgtcctgcagatggggggcgtgttatgcccccgccctggctgtggagcggggctgctgccggagcctgacca |
| | | | gaggaaagtcacctgcgaagggggcaatggcctgggctgtgggtttgccttctgccgggaatgtaaagaagcgtacc |
| | | | atgaaggggagtgcagtgccgtatttgaagcctcaggaacaactactcaggcctacagagtcgatgaaagagccgcc |
| | | | gagcaggctcgttgggaagcagcctccaaagaaaccatcaagaaaaccaccaagccctgtccccgctgccatgtacc |
| | | | agtggaaaaaaatggaggctgcatgcacatgaagtgtccgcagccccagtgcaggctcgagtggtgctggaactgtg |
| | | | gctgcgagtggaaccgcgtctgcatgggggaccactggttcgacgtgtagccagggcggccgggcgccccatcgcca |
| | | | catcctgggggagcatacccagtgtctaccttcattttctaattctcttttcaaacacacacacacacgcgcgcgcgcgc |
| | | | acacacactcttcaagtttttttcaaagtccaactacagccaaattgcagaagaaactcctggatccctttcactatgtc |
| | | | catgaaaaacagcagagtaaaattacagaagaagctcctgaatccctttcagtttgtccacacaagacagcagagcc |
| | | | atctgcgacaccaccaacaggcgttctcagcctccggatgacacaaataccagagcacagattcaagtgcaatccat |
| | | | gtatctgtatgggtcattctcacctgaattcgagacaggcagaatcagtagctggagagagagttctcacatttaatat |
| | | | cctgccttttaccttcagtaaacaccatgaagatgccattgacaaggtgtttctctgtaaaatgaactgcagtgggttctc |
| | | | caaactagattcatggctttaacagtaatgttcttatttaaattttcagaaagcatctattcccaaagaaccccaggcaa |
| | | | tagtcaaaaacatttgtttatccttaagaattccatctatataaatcgcattaatgaaataccaactatgcgtaaatcaac |
| | | | ttgtcacaaagtgagaaattatgaaagttaatttgaatgttgaatgtttgaattacagggaagaaatcaagttaatgta |
| | | | ctttcattccctttcatgatttgcaactttagaaagaaattgtttttctgaaagtatcaccaaaaaatctatagtttgattct |
| | | | gagtattcattttgcaacttggagattttgctaatacatttggctccactgtaaatttaatagataaagtgcctataaagg |
| | | | aaacacgtttagaaatgatttcaaaatgatattcaatcttaacaaaagtgaacattattaaatcagaatctttaaagag |
| | | | gagcctttccagaactaccaaaatgaagacacgcccgactctctccatcagaagggtttatacccctttggcacaccct |
| | | | ctctgtccaatctgcaagtcccagggagctctgcataccaggggttccccaggagagaccttctcttaggacagtaaac |
| | | | tcactagaatattccttatgttgacatggattggatttcagttcaatcaaactttcagcttttttttcagccattcacaacac |
| | | | aatcaaaagattaacaacactgcatgcggcaaaccgcatgctcttacccacactacgcagaagagaaagtacaacca |
| | | | ctatcttttgttctacctgtattgtctgacttctcaggaagatcgtgaacataactgagggcatgagtctcactagcacat |
| | | | ggaggcccttttggatttagagactgtaaattattaaatcggcaacagggcttctctttttagatgtagcactgaaatcct |
| | | | tgctggagggaagagaggggatgaactcaagttttccacatcctgggacacctgtccctcttttcctaactgcctaaga |
| | | | taacccatttcttccaaccatctgaggacagtcccgtcgtctcagaggccctgcaccggggagagactgggctctgca |
| | | | gcagccacatcagcattcacagcttcatgtggcttcactgtctgaaaatctaccgactccaacatggccccacggtgac |
| | | | aacagacctgtgacaggaagcccaaagctcacatagaaatggtggacagatcaaagtctctatagtaagggaaaaa |
| | | | aagagaggtggcaggcatgagccccctgcacccagtggctcgtgtccatactgagtccagaccctgatcaaggcctg |
| | | | acttagtgtcactggcagtcccactaaattacacttccttacactggcccgatgcgacaaatcaggtggctcccttctgt |
| | | | cacgtggagcacacagtgttttccatcatccatagctttcttcctgatggtgtttgcattattgcgccttcccaatctgcat |
| | | | gctgcgttgggcttgcggtgcctgaacaaggtttgctcccatgagctcaggcaccctaggatcccctgttagactattag |
| | | | gctgtccagcatggtctcctttcccttcttggtggtggtcttttccctttccagaatagaacagtgattcttaaaataagtt |
| | | | agagcaggccgggcgcggtggctcatgcctgtaatcccagcactttgggaggccgaggtgggtggatcacgaggtca |
| | | | ggagttcaagaccagcctggccaagatgatgaaaccccgtctctattaaaaatacaaaaattagctgggcgtggtggc |
| | | | aggcacctgtaatcccagcttcctgggaggctgaggcaggagaatcacttgaacccggggggcagaggttgcagtga |
| | | | gccgagatcacgccactgaactccagcctgggcaacagagtgagactctgtctcaaaaaaaaaaaaaaaacaaaaa |
| | | | caaaaaagcaagatcatccactacacatgaacatgaatcacagtattatttgcaca |
| | SNCA | 20 | aggagaaggagaaggaggaggactaggaggaggaggacggcgacgaccagaaggggcccaagagagggggcg |
| | (NM_000345. | | agcgaccgagcgccgcgacgcggaagtgaggtgcgtgcgggctgcagcgcagaccccggcccggcccctccgaga |
| | 3) | | gcgtcctgggcgctccctcacgccttgccttcaagccttctgcctttccaccctcgtgagcggagaactgggagtggcc |
| | | | attcgacgacagtgtggtgtaaaggaattcattagccatggatgtattcatgaaaggactttcaaaggccaaggaggg |
| | | | agttgtggctgctgctgagaaaaccaaacagggtgtggcagaagcagcaggaaagacaaaagagggtgttctctat |
| | | | gtaggctccaaaaccaaggagggagtggtgcatggtgtggcaacagtggctgagaagaccaaagagcaagtgaca |
| | | | aatgttggaggagcagtggtgacgggtgtgacagcagtagcccagaagacagtggagggagcagggagcattgca |
| | | | gcagccactggctttgtcaaaaaggaccagttgggcaagaatgaagaaggagccccacaggaaggaattctggaag |
| | | | atatgcctgtggatcctgacaatgaggcttatgaaatgccttctgaggaagggtatcaagactacgaacctgaagcct |
| | | | aagaaatatctttgctcccagtttcttgagatctgctgacagatgttccatcctgtacaagtgctcagttccaatgtgccc |
| | | | agtcatgacatttctcaaagtttttacagtgtatctcgaagtcttccatcagcagtgattgaagtatctgtacctgccccc |
| | | | actcagcatttcggtgcttccctttcactgaagtgaatacatggtagcagggtctttgtgtgctgtggattttgtggcttca |
| | | | atctacgatgttaaaacaaattaaaaacacctaagtgactaccacttatttctaaatcctcactatttttttgttgctgttg |
| | | | ttcagaagttgttagtgatttgctatcatatattataagatttttaggtgtcttttaatgatactgtctaagaataatgacgt |
| | | | attgtgaaatttgttaatatatataatacttaaaaatatgtgagcatgaaactatgcacctataaatactaaatatgaaa |
| | | | ttttaccattttgcgatgtgttttattcacttgtgtttgtatataaatggtgagaattaaaataaaacgttatctcattgcaa |
| | | | aaatattttatttttatcccatctcactttaataataaaaatcatgcttataagcaacatgaattaagaactgacacaaag |
| | | | gacaaaaatataaagttattaatagccatttgaagaaggaggaattttagaagaggtagagaaaatggaacattaac |
| | | | cctacactcggaattccctgaagcaacactgccagaagtgtgttttggtatgcactggttccttaagtggctgtgattaa |
| | | | ttattgaaagtggggtgttgaagaccccaactactattgtagagtggtctatttctcccttcaatcctgtcaatgtttgctt |
| | | | tacgtattttggggaactgttgtttgatgtgtatgtgtttataattgttatacatttttaattgagccttttattaacatatatt |
| | | | gttatttttgtctcgaaataattttttagttaaaatctattttgtctgatattggtgtgaatgctgtacctttctgacaataaa |
| | | | taatattcgaccatgaataaaaaaaaaaaaaaagtgggttcccgggaactaagcagtgtagaagatgattttgactac |
| | | | accctccttagagagccataagacacattagcacatattagcacattcaaggctctgagagaatgtggttaactttgttt |
| | | | aactcagcattcctcactttttttttttaatcatcagaaattctctctctctctctctctttttctctcgctctctttttttttttttt |
| | | | tttacaggaaatgcctttaaacatcgttggaactaccagagtcaccttaaaggagatcaattctctagactgataaaaa |
| | | | tttcatggcctcctttaaatgttgccaaatatatgaattctaggatttttccttaggaaaggtttttctctttcagggaagat |
| | | | ctattaactccccatgggtgctgaaaataaacttgatggtgaaaaactctgtataaattaatttaaaaattatttggtttc |
| | | | tctttttaattattctggggcatagtcatttctaaaagtcactagtagaaagtataatttcaagacagaatattctagaca |
| | | | tgctagcagtttatatgtattcatgagtaatgtgatatatattgggcgctggtgaggaaggaaggaggaatgagtgact |
| | | | ataaggatggttaccatagaaacttccttttttacctaattgaagagagactactacagagtgctaagctgcatgtgtca |
| | | | tcttacactagagagaaatggtaagtttcttgttttatttaagttatgtttaagcaaggaaaggatttgttattgaacagt |
| | | | atatttcaggaaggttagaaagtggcggttaggatatattttaaatctacctaaagcagcatattttaaaaatttaaaag |
| | | | tattggtattaaattaagaaatagaggacagaactagactgatagcagtgacctagaacaatttgagattaggaaagt |
| | | | tgtgaccatgaatttaaggatttatgtggatacaaattctcctttaaagtgtttcttcccttaatatttatctgacggtaatt |
| | | | tttgagcagtgaattactttatatatcttaatagtttatttgggaccaaacacttaaacaaaaagttctttaagtcatataa |
| | | | gccttttcaggaagcttgtctcatattcactcccgagacattcacctgccaagtggcctgaggatcaatccagtcctagg |
| | | | tttattttgcagacttacattctcccaagttattcagcctcatatgactccacggtcggctttaccaaaacagttcagagt |
| | | | gcactttggcacacaattgggaacagaacaatctaatgtgtggtttggtattccaagtggggtctttttcagaatctctg |
| | | | cactagtgtgagatgcaaacatgtttcctcatctttctggcttatccagtatgtagctatttgtgacataataaatatatac |
| | | | atatatgaaaata |
| | HLA-DRB1 | 21 | tgagtgtcatttcttcaacgggacggagcgggtgcggttcctggacagatacttctatcaccaagaggagtacgtgcg |
| | (AB698844.1) | | cttcgacagcgacgtgggggagtaccgggcggtgacggagctggggcggcctagcgccgagtactggaacagccag |
| | | | aaggacctcctggagcagaggcgggccgcggtggacacctactgcagacacaactacggggttgtggagagcttca |
| | | | cagtgcagcggcgaggtgagcgcggcgcggggcggggcctgagtccctgtgagcggagaatctgagtgtgtgtgtgt |
| | | | gtgtgtgtgtgtgtgtgtgtgtgtgtgtgtgagagagagagagagagagagagagagagagagagagagagagag |
| | | | agcgccatctgtgagcatttagaatcctctctatcctgagcaaggagttctgcgggcacaggtgtgtgtgtagagtgtg |
| | | | gatttgtccgtgtctgtgaggctgttgtgggaggggaggcaggagggggctgcttcttattcttggagacttctgtgggg |
| | | | aggtgacaagggaggtgggtgctgggggctggagagagaggcgaccttgattgtctcgggtccttagagatgcaag |
| | | | gaagggaaatgtatggggtgtgtggttggggtgaaggtttaggggaggagagctgaggggtaaggaaggtttggga |
| | | | taatgtgaagaggccagtttcagactgtccctggcacacacccttcatgtaatctctgaaataaaagtgtgtgctgtttg |
| | | | tttgtaaaagcattagattaacttctaggggaattgagtagacctctgaggcacctctgaagcttctttaggtataaattt |
| | | | cttgctagttttttgttttcttagtgttatatttttacatagttgaaatgactgtgaaactaactttttgaattaaagtttgaa |
| | | | aacactgttactattttattataatgctaataatttcatagttactttttaaatatataatagttgtgacacaaattacctca |
| | | | ctttctttgtttttttttcttacactttaagttttagggtacatgtgcacaacgtgcaggtttgttacatatgtatacatgtgc |
| | | | catgttggtgtgctgcacccattaactcgtcatttaacattaggtatatctcctaatgctatccctccccaccccccaccc |
| | | | cacaacaggccccagtgtgtgatgttccccttcctgtgtccatgtgttctcactgttcaattcccacctatgagtgagaac |
| | | | atgcggtgttcggttttttgtccttgccatagtttgctgagaatgatggtttccagcttcatccatgtccctacaaaggaca |
| | | | tgaactcattcttttttgtggctgcatagtattccatagtgtatatgtgccacattttcttaatccagtctatcattgttggac |
| | | | atttgggttggttccaagtctttgctattgtgaatagtgccgcaataaacatacatgtgcatatgtctttatagcagcatg |
| | | | atttataatccttgggttatatacccagtaatgggatggctgggtcaaatggtatttctagttctagatccctgaggaatc |
| | | | gccacactgacttccacaatggttgaactagtttagagtcccaccaacagggtaaaagtgttcctatttctccacatcct |
| | | | ctccagcacctgttgcttcctgactttttaatgatcgccattctaactggtgtgagatggtatctcattgtggttttgatttg |
| | | | caattctctgatggccagtgatgatgagcattttttcatgtgtcttttggctgcataaatgtcttcttttgagaagtgtctgt |
| | | | tcatgtcctttgcccactttttgatggggtattttgttttttcttgtaaatttgtttgagttcattgcagattctggatattagc |
| | | | cctttgtcatatgagtagattgcaaaaattttctcccattctgtaggttgcctcttcactctgatggtagtttcttttgctgtg |
| | | | cagaagctctttagtttaattagatcccatttgtccattttggcttttgttgccattgcttttggtgttttagacatgaagttc |
| | | | ttgcccatgcctatgtcctgaatggtattgcctaggttttcttctagggtttttatggtttcaggtctaacatttaagtcttta |
| | | | atccatcttgaattaatttttgtataagcaaattacgtcactttccccattgatgacctttattatgacattcaccaatagtt |
| | | | gaaaatgtatgtttctggttaatttttgatttatattttttgatttgtaattattttgaattattttgacctatttattggccagt |
| | | | tgtaattactgctctgctctacgaattacctgttgtatttggtaggtaatggacaatgatctattgtctcttatctttagggc |
| | | | ttagtatttttctcagtgactttgtgggtttgttgtactgtaagattattaacactttattgatatttgattcagtattttctcc |
| | | | agtttgtggtatgtatattttgaaaattcttttccatgttaagaatttgaacatttttatttaataaaatatattgcaaaatgt |
| | | | taattaatgattcacaaactagctcaagtctaccattttgtggtattgatgtctccaggtttctccttccttcttaaaaaaa |
| | | | aatgtatttattgagagtatgctagtgtcagggatttccctaggcataagcactccaagtaatgagtcccagacactgc |
| | | | cttgatccaaatgtcattctggaaagaaaaatcattttacagtgataagcctaataatagttatacttgttttgcctggga |
| | | | gatgcattgatcagctaaatgtaaatataagaactttcaaaactaaaatgacgttccttaatctttctctctgctttagga |
| | | | atcatgctttcttaggaacttaaagatttggagaatcatttctgtctgtcccaccttcccaggagcataaccatttctgtg |
| | | | gtgttctaaggtgtgagtgcatggcagtagtattcctaaaaatccatattcagtttcctcatgtgccctactccgtcccttt |
| | | | ctctatccacattgctttaaatcatatttttctctcaaggtgtacaaggatgataaataggtgccaagtggagaacccaa |
| | | | gtgtgacgagccctctcacagtagaatggagtgagaagctttctgacctcataaattgaaggctatcgtaattcattctt |
| | | | ttatatattttacttgcattaatcctcatataacctcaagaggtaaattaatataattatcctccattattggagagaaagt |
| | | | tgagacacaaaagaatcaaaaactcttccaggatcaaccagtaaaaggcagaccttggatttgaaccaggcaacctg |
| | | | gctcagaagtcagttttaattaccacactctgtactttcaaagatttgtaaacgctttgacaatgcatgtcaatttcaagc |
| | | | tatgaagagccaaacataatttttcacaatatctctcaaatctaatgggtccccactataaagattaaattccaggctga |
| | | | tgacactgtgaggccacatggccagctgtgctggaggcctgctcaaggccagagcctaggtttacagagaagcagac |
| | | | aaaaagctaaacaaggagacttactctgtctgcatgacttattccctctaccttgttttctcctagtctatcctgaggtga |
| | | | ctgtgtatcctgcaaagacccagcccctgcagcaccacaacctcctggtctgctctgtgaatggtttctatccaggcagc |
| | | | attgaagtcaggtggttccggaacggccaggaagagaagactggggtggtgtccacaggcctgatccagaatggag |
| | | | actggaccttccagaccctggtgatgctggaaacagttcctcggagtggagaggtttacacctgccaagtggagcacc |
| | | | caagcctgacgagccctctcacagtggaatggagtgagcagctttctgacttcataaatttctcacccaccaagacgcg |
| | | | aactttactaatccctgagtatcaggcttctcctatcccacatcctattttcatttgctccacgttctcatctccatcagcac |
| | | | aggtcactggggggtagccctgtaatactttctagaaacacctgtaccccctggggaagcagtcatgcctgccaggca |
| | | | ggagaggctgtccctcttttgaacctccccatgatgtcacaagtcggggtcacctgctgtctgtgggctccaggccctgc |
| | | | ctctgggtctgagactgagtttctggtactgttgctctgagtcgtttgttgtaatctgagaagaggagaagtatagggac |
| | | | cttcctgacatgaggggagtccaatctcagctccgccttttattagatctgtcactctaggcaactacttaacctcattgg |
| | | | gtctcaggctttctgttcatcagatgttgaagtcctgtcttacatcaaggctgtaatatttgaatgagtttgatgactgaac |
| | | | cttgtaactgttcagtgtgatttgaaaacctttctcaagaaatggtcagttattttagttcttgcagagcagccttctttctc |
| | | | attttcaaagctctgaatctcaaggtgtcaattaaagaggttccatttgggataaaaatcactaaacctggcttcctctct |
| | | | caggagcacggtctgaatctgcacagagcaagatgctgagtggagtcgggggctttgtgctgggcctgctcttccttg |
| | | | gggccgggctgttcatctacttcaggaatcagaaaggtgaggagcctttggtagctggctgtctccatacgcttttctgg |
| | | | aggaggaactatggctttgctgaagttggttctcagcatatgaatggccctggataaagcctctctactcccaaatgac |
| | | | ctccaatgttctgcaaatccagaaatcatcagtgcatggttgctatgtcaaagcataatagcttgtggcctacagagat |
| | | | aacagaaagattaacaggtataggtgctttggttgagatcgtggagcaaattaaggaagagcaactaaagctaatac |
| | | | aattacactggatcctgtgacagacacttcacacttcatgggtcacatggtctgtttctgctcctctctgccctggctggt |
| | | | gtgggttgtggtgtcagagaactctcaggtgggagatctggagctgggacattgtgttggaggacagatttgcttccat |
| | | | atcctttaagtgtatatcttctctttttcctaggacactctggacttcagccaacaggtaataccttttcatcctctttaaga |
| | | | aacagatttggaggccaggcgcagtggctcacgcctgtaatcccagcactttgggaggccgaggcgggcgaatcatg |
| | | | aggtcaggagttcgagaccagcctgaccaacgtggtgaaaccccgtctctactaaaaatacaaaaaaaaatcagtcg |
| | | | ggcgtggtggtgtgcgcctgtaatcccagctactcaggaggccaaggcaggagaatcgctggaacccgggaggcag |
| | | | aggttgcagtgagccgagattgggccactgcactccagcctaggtgacagagtgagaccccatctcaaaaaaacaaa |
| | | | aaaagaaagaaagaaacagatttcctttccctagaatgatggtagaggtaataaggcatgagacagaagtaatagc |
| | | | aaagacattggatccaaatttctgatcaggcaatttacaccagaactcctcctctccacttagaaaaggcctgtgctctg |
| | | | caggagtattgactcatggagacttcagaacttgtttttcttcttcctgcagtgctctcatctgagtccttgaaagagggc |
| | | | aaaataaactgttagtagagccaggtctgaaaacaacactttcttgcgtctctgcaggattcctgagctgaagtgaaga |
| | | | tgaccacattcaaggaagaaccttctgccccagctttgcaggatgaaacacttccccgcttggctctcattcttccacaa |
| | | | gagagacctttctccggacctggttgctactggttcagcagctctgcagaaaatgtcctcccttgtggctgcctcagctc |
| | | | gtacctttggcctgaagtcccagcattaatggcagcccctcatcttccaagttttgtgctcccctttacctaa |
| | IL7R | 22 | atacctaggcactaatttagttccatatgtactatgtgtacctgaaaagttgtgtggcaatcaaattttcacaaatagaat |
| | (AH007043.2) | | cctgttttaaatacactaagaaagtacctactttatcctttaaacaagaggtcagcagactttttctacaaagggtcaga |
| | | | tagtaaagattttacaccttttgtacaatacaatctctatctcatctacttagctctgccattgttgcataaaagcagctgt |
| | | | agatgatacacaaatgggtgaggctgtattccaaatgaaacgttatttgcaaaaacaggtggtagattaaatttggtcc |
| | | | caaggcttacttgggaaaaaaaaagatcttttgaaaaagaaaaaataaatgaataatttttttaaaaaattgttcccta |
| | | | ggtcatagtttgccagcccctgccctaaacaaataattcttgaatgcctactgtggtgtgtaagatatgagtaaatacca |
| | | | gggatacacagagaacaaaagagaaaaactgctattcttgtgaaacttggaagttggaggtaagctatttaaaataa |
| | | | acccacaataaagtacttcacatagtgcagactgtttctttaaatcaaaactcactccaaacaaccaattgattcacttt |
| | | | gtaagtttgaatttttgtcttcagattcttttaaagtgggcccttagtcaggagcggtggctcatgcctgtagtcctagca |
| | | | ctttgggaggctgaggcaggcagatcacttgaggtcaggagttcgagacaagcctggccaacatggcgaaaccccgt |
| | | | ctccactgaaaacacaaaaattaggctggcatagtggcatttgcctgtagtcctagctactcaggaggctgaggcagg |
| | | | agaattgcttgaacctgggaggtgaaaattgcagtgagccgagatcatgctattgtactccagcctgggcaacaaagc |
| | | | aagactcgtctcaaaaaaataaaaattaaaaaaataaagtagcctctagcctaagatagcttgagcctaggtgtgaat |
| | | | ctactgccttactctgatgtaagcacagtaagtgtgggggctgcagggaatatccaggaggaacaataatttcagagg |
| | | | ctctgtctcttcatgtccttgacctctgcttacagcagcaatacttttactcagacttcctgtttctggaacttgccttcttttt |
| | | | tgctgtgtttatacttcccttgtctgtggttagataagtataaagccctagatctaagcttctctgtcttcctccctccctcc |
| | | | cttcctcttactctcattcatttcatacacactggctcacacatctactctctctctctatctctctcagaatgacaattcta |
| | | | ggtacaacttttggcatggttttttctttacttcaagtcgtttctggagaaagtggctatgctcaaaatggtgagtcatttc |
| | | | taacttttcttatggattttggattatctgtagcatggtttcaggttattcagttccctaagagacctgagtcaggcactgg |
| | | | gtttgagtgcnnnnnnnnnnnnnnnnnnnnnnnnnnnnnnnnnnnnnnnnnnnnnnnnnnnnnnn |
| | | | nnnnnnnnnnnnnnnnnnnnnnnnnnnnnnnnnnnnnnnnnnnnngagcgtttttgttttttgttttttg |
| | | | ttggttggttggttttgaagcatttttcttgtctttgcccttcccattttcttccttgaatactacataatccattactatttcat |
| | | | gtctgccacagagtctgctattttattaaggtcatgccatatttcaaaaggatgcatttatttgtttcattaacagctgcat |
| | | | gtttgttcctccccaggagacttggaagatgcagaactggatgactactcattctcatgctatagccagttggaagtga |
| | | | atggatcgcagcactcactgacctgtgcttttgaggacccagatgtcaacaccaccaatctggaatttgaaatatggtg |
| | | | agggatggtggttttaatggttgcttagacatcctctgtctctcttttcatatgctgtttttaatagccacaaaagaaaga |
| | | | atatgtggcctaattaacaaatgttaacatctaaggaattcccaaaggcctcctgnnnnnnnnnnnnnnnnnnn |
| | | | nnnnnnnnnnnnnnnnnnnnnnnnnnnnnnnnnnnnnnnnnnnnnnnnnnnnnnnnnnnnnn |
| | | | nnnnnnnnnnnnnnnnnnncctacctgaatcaagacatacccccttttattcctacagtggggccctcgtggag |
| | | | gtaaagtgcctgaatttcaggaaactacaagagatatatttcatcgagacaaagaaattcttactgattggaaagagc |
| | | | aatatatgtgtgaaggttggagaaaagagtctaacctgcaaaaaaatagacctaaccactataggtaagaagttgtat |
| | | | ataaaagtatggttgtcacttttgggctacctgaaaacactgtgtctggacattctgtaggttaaaagtagacaaatagt |
| | | | ggaaagaactggcaatagataatagataattccctactgtaantttttataataaatgaaaagcttgaaatttatacttt |
| | | | cctgcagttgaaagaattctgaggatcttcaaacccaggtgtgaaagatagtgtttgtgcaacctacannnnnnnnn |
| | | | nnnnnnnnnnnnnnnnnnnnnnnnnnnnnnnnnnnnnnnnnnnnnnnnnnnnnnnnnnnnnn |
| | | | nnnnnnnnnnnnnnnnnnnnnnnnnnnnntcaaagtgacttgcagaggagatgaattttaaatactataatt |
| | | | atttccttggctgccctttagacagaatttatttctttttcttttccagttaaacctgaggctccttttgacctgagtgtcatc |
| | | | tatcgggaaggagccaatgactttgtggtgacatttaatacatcacacttgcaaaagaagtatgtaaaagttttaatgc |
| | | | acgatgtagcttaccgccaggaaaaggatgaaaacaaatggacggtatgtacttcaactacattaataaaataaaaa |
| | | | cttatgaatgttttctattttgttggcctagtagtgcatttcccctnnnnnnnnnnnnnnnnnnnnnnnnnnnnn |
| | | | nnnnnnnnnnnnnnnnnnnnnnnnnnnnnnnnnnnnnnnnnnnnnnnnnnnnnnnnnnnnnn |
| | | | nnnnnnnnngagctccttattctaacaaatacgagacaacttcagagaatgcttatgggactaaaggaatcccaat |
| | | | tgaaatgatttgggagatttaggcaacacctcttttcccatcctaagaatgtaactgcactctactctctagcatgtgaat |
| | | | ttatccagcacaaagctgacactcctgcagagaaagctccaaccggcagcaatgtatgagattaaagttcgatccatc |
| | | | cctgatcactattttaaaggcttctggagtgaatggagtccaagttattacttcagaactccagagatcaataatagctc |
| | | | aggtaaggaatggtggtagagtttttgttccctcagagtgctttgcatgtcaaagtgtgggagcaagtgagaggaaga |
| | | | ttgttgaaactaacctgcannnnnnnnnnnnnnnnnnnnnnnnnnnnnnnnnnnnnnnnnnnnnnnn |
| | | | nnnnnnnnnnnnnnnnnnnnnnnnnnnnnnnnnnnnnnnnnnnnnnnnnnnnatacttcaagtgg |
| | | | cagatgctctgggcctggtcacccaagtcaatgccttttaaaccaaatccctccataaagctgtcaaatatgtctcttaa |
| | | | ctgaaaagcaactttcaggaaataataagtgggcccacattactaagtaaatgcaaatcaccctgagaccctaccccc |
| | | | actgcatggctactgaatgctcaccacaatctattcttgctttccaggggagatggatcctatcttactaatcatcagcat |
| | | | tttgagttttttctctgtcgctctgttggtcatcttggcctgtgtgttatggaaaaaaaggtgaccttcttcaacgtaataa |
| | | | agagggtgattgtgtgggtcnnnnnnnnnnnnnnnnnnnnnnnnnnnnnnnnnnnnnnnnnnnnnn |
| | | | nnnnnnnnnnnnnnnnnnnnnnnnnnnnnnnnnnnnnnnnnnnnnnnnnnnnnnttgtcatgtctt |
| | | | gttcacagaatggattgatatctgtggtctctggtccaacccctccttgaattgatagggccccgaggcccagagaaag |
| | | | ccagtctcttgaccatggtcacccacctaattgtgttagagccaagactagaaatctgttcttctgattccaagctcaga |
| | | | ataagtgggaagactcagtgtgcctgtgccctctgccattcacttcatctttcaatgttctctgatttcaggattaagccta |
| | | | tcgtatggcccagtctccccgatcataagaagactctggaacatctttgtaagaaaccaagaaaagtgagtgtttttgg |
| | | | tgcttaaaaagtgttgtgttggcaacatcccagtggccaagaatgatattccaggacaaggaacagttgaacctcacc |
| | | | ttttggtatttgattcatcctgtaactagggtccctcctaagaccctagctgcagtagggaactgaaataagatacacat |
| | | | ctcagaacttctgggctccctggggctggagggcacagccagtggtcacttcaagtcttgaagtgtctcagaagctcca |
| | | | gaagcaaagagtccattgaggaacatgctggcaattctgtgacattccctgtcagaaaactctatagacctactcctga |
| | | | actgaacatttgatggtgtgtctctctggtgccatcttaataccctttctcctttttctgtgcagaatttaaatgtgagtttc |
| | | | aatcctgaaagtttcctggactgccagattcatagggtggatgacattcaagctagagatgaagtggaaggttttctgc |
| | | | aagatacgtttcctcagcaactagaagaatctgagaagcagaggcttggaggggatgtgcagagccccaactgccca |
| | | | tctgaggatgtagtcatcactccagaaagctttggaagagattcatccctcacatgcctggctgggaatgtcagtgcat |
| | | | gtgacgcccctattctctcctcttccaggtccctagactgcagggagagtggcaagaatgggcctcatgtgtaccagga |
| | | | cctcctgcttagccttgggactacaaacagcacgctgccccctccattttctctccaatctggaatcctgacattgaaccc |
| | | | agttgctcagggtcagcccattcttacttccctgggatcaaatcaagaagaagcatatgtcaccatgtccagcttctacc |
| | | | aaaaccagtgaagtgtaagaaacccagactgaacttaccgtgagcgacaaagatgatttaaaagggaagtctagag |
| | | | ttcctagtctccctcacagcacagagaagacaaaattagcaaaaccccactacacagtctgcaagattctgaaacatt |
| | | | gctttgaccactcttcctgagttcagtggcactcaacatgagtcaagagcatcctgcttctaccatgtggatttggtcac |
| | | | aaggtttaaggtgacccaatgattcagctatttaaaaaaaaaagaggaaagaatgaaagagtaaaggaaatgattg |
| | | | aggagtgaggaaggcaggaagagagcatgagaggaaataaataaaggaaaataaaaatgatagttgccattatta |
| | | | ggatttaatatatatccagtgctttgcaagtgctctgcgcaccttgtctcactccatcctgacaataatcctgggaggtgt |
| | | | gtgcaattactacgactactctcttttttatagatcgagctc |
| | MECP2 | 23 | ccggcgtcggcggcgcgcgcgctccctcctctcggagagagggctgtggtaaaagccgtccggaaaatggccgccgc |
| | (NM_004992. | | cgccgccgccgcgccgagcggaggaggaggaggaggcgaggaggagagactgctccataaaaatacagactcac |
| | 3) | | cagttcctgctttgatgtgacatgtgactccccagaatacaccttgcttctgtagaccagctccaacaggattccatggt |
| | | | agctgggatgttagggctcagggaagaaaagtcagaagaccaggacctccagggcctcaaggacaaacccctcaag |
| | | | tttaaaaaggtgaagaaagataagaaagaagagaaagagggcaagcatgagcccgtgcagccatcagcccaccac |
| | | | tctgctgagcccgcagaggcaggcaaagcagagacatcagaagggtcaggctccgccccggctgtgccggaagctt |
| | | | ctgcctcccccaaacagcggcgctccatcatccgtgaccggggacccatgtatgatgaccccaccctgcctgaaggct |
| | | | ggacacggaagcttaagcaaaggaaatctggccgctctgctgggaagtatgatgtgtatttgatcaatccccagggaa |
| | | | aagcctttcgctctaaagtggagttgattgcgtacttcgaaaaggtaggcgacacatccctggaccctaatgattttga |
| | | | cttcacggtaactgggagagggagcccctcccggcgagagcagaaaccacctaagaagcccaaatctcccaaagct |
| | | | ccaggaactggcagaggccggggacgccccaaagggagcggcaccacgagacccaaggcggccacgtcagaggg |
| | | | tgtgcaggtgaaaagggtcctggagaaaagtcctgggaagctccttgtcaagatgccttttcaaacttcgccaggggg |
| | | | caaggctgaggggggtggggccaccacatccacccaggtcatggtgatcaaacgccccggcaggaagcgaaaagc |
| | | | tgaggccgaccctcaggccattcccaagaaacggggccgaaagccggggagtgtggtggcagccgctgccgccgag |
| | | | gccaaaaagaaagccgtgaaggagtcttctatccgatctgtgcaggagaccgtactccccatcaagaagcgcaagac |
| | | | ccgggagacggtcagcatcgaggtcaaggaagtggtgaagcccctgctggtgtccaccctcggtgagaagagcggg |
| | | | aaaggactgaagacctgtaagagccctgggcggaaaagcaaggagagcagccccaaggggcgcagcagcagcgc |
| | | | ctcctcaccccccaagaaggagcaccaccaccatcaccaccactcagagtccccaaaggcccccgtgccactgctcc |
| | | | cacccctgcccccacctccacctgagcccgagagctccgaggaccccaccagcccccctgagccccaggacttgagc |
| | | | agcagcgtctgcaaagaggagaagatgcccagaggaggctcactggagagcgacggctgccccaaggagccagct |
| | | | aagactcagcccgcggttgccaccgccgccacggccgcagaaaagtacaaacaccgaggggagggagagcgcaa |
| | | | agacattgtttcatcctccatgccaaggccaaacagagaggagcctgtggacagccggacgcccgtgaccgagaga |
| | | | gttagctgactttacacggagcggattgcaaagcaaaccaacaagaataaaggcagctgttgtctcttctccttatggg |
| | | | tagggctctgacaaagcttcccgattaactgaaataaaaaatatttttttttctttcagtaaacttagagtttcgtggcttc |
| | | | agggtgggagtagttggagcattggggatgtttttcttaccgacaagcacagtcaggttgaagacctaaccagggcca |
| | | | gaagtagctttgcacttttctaaactaggctccttcaacaaggcttgctgcagatactactgaccagacaagctgttgac |
| | | | caggcacctcccctcccgcccaaacctttcccccatgtggtcgttagagacagagcgacagagcagttgagaggaca |
| | | | ctcccgttttcggtgccatcagtgccccgtctacagctcccccagctccccccacctcccccactcccaaccacgttggg |
| | | | acagggaggtgtgaggcaggagagacagttggattctttagagaagatggatatgaccagtggctatggcctgtgcg |
| | | | atcccacccgtggtggctcaagtctggccccacaccagccccaatccaaaactggcaaggacgcttcacaggacagg |
| | | | aaagtggcacctgtctgctccagctctggcatggctaggaggggggagtcccttgaactactgggtgtagactggcct |
| | | | gaaccacaggagaggatggcccagggtgaggtggcatggtccattctcaagggacgtcctccaacgggtggcgcta |
| | | | gaggccatggaggcagtaggacaaggtgcaggcaggctggcctggggtcaggccgggcagagcacagcggggtg |
| | | | agagggattcctaatcactcagagcagtctgtgacttagtggacaggggagggggcaaagggggaggagaagaaa |
| | | | atgttcttccagttactttccaattctcctttagggacagcttagaattatttgcactattgagtcttcatgttcccacttca |
| | | | aaacaaacagatgctctgagagcaaactggcttgaattggtgacatttagtccctcaagccaccagatgtgacagtgt |
| | | | tgagaactacctggatttgtatatatacctgcgcttgttttaaagtgggctcagcacatagggttcccacgaagctccga |
| | | | aactctaagtgtttgctgcaattttataaggacttcctgattggtttctcttctccccttccatttctgccttttgttcatttca |
| | | | tcctttcacttctttcccttcctccgtcctcctccttcctagttcatcccttctcttccaggcagccgcggtgcccaaccaca |
| | | | cttgtcggctccagtccccagaactctgcctgccctttgtcctcctgctgccagtaccagccccaccctgttttgagccct |
| | | | gaggaggccttgggctctgctgagtccgacctggcctgtctgtgaagagcaagagagcagcaaggtcttgctctccta |
| | | | ggtagccccctcttccctggtaagaaaaagcaaaaggcatttcccaccctgaacaacgagccttttcacccttctactct |
| | | | agagaagtggactggaggagctgggcccgatttggtagttgaggaaagcacagaggcctcctgtggcctgccagtca |
| | | | tcgagtggcccaacaggggctccatgccagccgaccttgacctcactcagaagtccagagtctagcgtagtgcagca |
| | | | gggcagtagcggtaccaatgcagaactcccaagacccgagctgggaccagtacctgggtccccagcccttcctctgct |
| | | | cccccttttccctcggagttcttcttgaatggcaatgttttgcttttgctcgatgcagacagggggccagaacaccacac |
| | | | atttcactgtctgtctggtccatagctgtggtgtaggggcttagaggcatgggcttgctgtgggtttttaattgatcagttt |
| | | | tcatgtgggatcccatctttttaacctctgttcaggaagtccttatctagctgcatatcttcatcatattggtatatccttttc |
| | | | tgtgtttacagagatgtctcttatatctaaatctgtccaactgagaagtaccttatcaaagtagcaaatgagacagcagt |
| | | | cttatgcttccagaaacacccacaggcatgtcccatgtgagctgctgccatgaactgtcaagtgtgtgttgtcttgtgtat |
| | | | ttcagttattgtccctggcttccttactatggtgtaatcatgaaggagtgaaacatcatagaaactgtctagcacttcctt |
| | | | gccagtctttagtgatcaggaaccatagttgacagttccaatcagtagcttaagaaaaaaccgtgtttgtctcttctgga |
| | | | atggttagaagtgagggagtttgccccgttctgtttgtagagtctcatagttggactttctagcatatatgtgtccatttcc |
| | | | ttatgctgtaaaagcaagtcctgcaaccaaactcccatcagcccaatccctgatccctgatcccttccacctgctctgct |
| | | | gatgacccccccagcttcacttctgactcttccccaggaagggaaggggggtcagaagagagggtgagtcctccaga |
| | | | actcttcctccaaggacagaaggctcctgcccccatagtggcctcgaactcctggcactaccaaaggacacttatccac |
| | | | gagagcgcagcatccgaccaggttgtcactgagaagatgtttattttggtcagttgggtttttatgtattatacttagtca |
| | | | aatgtaatgtggcttctggaatcattgtccagagctgcttccccgtcacctgggcgtcatctggtcctggtaagaggagt |
| | | | gcgtggcccaccaggcccccctgtcacccatgacagttcattcagggccgatggggcagtcgtggttgggaacacag |
| | | | catttcaagcgtcactttatttcattcgggccccacctgcagctccctcaaagaggcagttgcccagcctctttcccttcc |
| | | | agtttattccagagctgccagtggggcctgaggctccttagggttttctctctatttccccctttcttcctcattccctcgtc |
| | | | tttcccaaaggcatcacgagtcagtcgcctttcagcaggcagccttggcggtttatcgccctggcaggcaggggccctg |
| | | | cagctctcatgctgcccctgccttggggtcaggttgacaggaggttggagggaaagccttaagctgcaggattctcac |
| | | | cagctgtgtccggcccagttttggggtgtgacctcaatttcaattttgtctgtacttgaacattatgaagatgggggcctc |
| | | | tttcagtgaatttgtgaacagcagaattgaccgacagctttccagtacccatggggctaggtcattaaggccacatcca |
| | | | cagtctcccccacccttgttccagttgttagttactacctcctctcctgacaatactgtatgtcgtcgagctccccccaggt |
| | | | ctacccctcccggccctgcctgctggtgggcttgtcatagccagtgggattgccggtcttgacagctcagtgagctgga |
| | | | gatacttggtcacagccaggcgctagcacagctcccttctgttgatgctgtattcccatatcaaaagacacaggggaca |
| | | | cccagaaacgccacatcccccaatccatcagtgccaaactagccaacggccccagcttctcagctcgctggatggcg |
| | | | gaagctgctactcgtgagcgccagtgcgggtgcagacaatcttctgttgggtggcatcattccaggcccgaagcatga |
| | | | acagtgcacctgggacagggagcagccccaaattgtcacctgcttctctgcccagcttttcattgctgtgacagtgatg |
| | | | gcgaaagagggtaataaccagacacaaactgccaagttgggtggagaaaggagtttctttagctgacagaatctctg |
| | | | aattttaaatcacttagtaagcggctcaagcccaggagggagcagagggatacgagcggagtcccctgcgcgggac |
| | | | catctggaattggtttagcccaagtggagcctgacagccagaactctgtgtcccccgtctaaccacagctccttttccag |
| | | | agcattccagtcaggctctctgggctgactgggccaggggaggttacaggtaccagttctttaagaagatctttgggca |
| | | | tatacatttttagcctgtgtcattgccccaaatggattcctgtttcaagttcacacctgcagattctaggacctgtgtccta |
| | | | gacttcagggagtcagctgtttctagagttcctaccatggagtgggtctggaggacctgcccggtgggggggcagagc |
| | | | cctgctccctccgggtcttcctactcttctctctgctctgacgggatttgttgattctctccattttggtgtctttctcttttag |
| | | | atattgtatcaatctttagaaaaggcatagtctacttgttataaatcgttaggatactgcctcccccagggtctaaaatta |
| | | | catattagaggggaaaagctgaacactgaagtcagttctcaacaatttagaaggaaaacctagaaaacatttggcag |
| | | | aaaattacatttcgatgtttttgaatgaatacgagcaagcttttacaacagtgctgatctaaaaatacttagcacttggc |
| | | | ctgagatgcctggtgagcattacaggcaaggggaatctggaggtagccgacctgaggacatggcttctgaacctgtct |
| | | | tttgggagtggtatggaaggtggagcgttcaccagtgacctggaaggcccagcaccaccctccttcccactcttctcat |
| | | | cttgacagagcctgccccagcgctgacgtgtcaggaaaacacccagggaactaggaaggcacttctgcctgaggggc |
| | | | agcctgccttgcccactcctgctctgctcgcctcggatcagctgagccttctgagctggcctctcactgcctccccaagg |
| | | | ccccctgcctgccctgtcaggaggcagaaggaagcaggtgtgagggcagtgcaaggagggagcacaacccccagct |
| | | | cccgctccgggctccgacttgtgcacaggcagagcccagaccctggaggaaatcctacctttgaattcaagaacattt |
| | | | ggggaatttggaaatctctttgcccccaaacccccattctgtcctacctttaatcaggtcctgctcagcagtgagagcag |
| | | | atgaggtgaaaaggccaagaggtttggctcctgcccactgatagcccctctccccgcagtgtttgtgtgtcaagtggca |
| | | | aagctgttcttcctggtgaccctgattatatccagtaacacatagactgtgcgcataggcctgctttgtctcctctatcctg |
| | | | ggcttttgttttgctttttagttttgcttttagtttttctgtcccttttatttaacgcaccgactagacacacaaagcagttga |
| | | | atttttatatatatatctgtatattgcacaattataaactcattttgcttgtggctccacacacacaaaaaaagacctgtta |
| | | | aaattatacctgttgcttaattacaatatttctgataaccatagcataggacaagggaaaataaaaaaagaaaaaaaa |
| | | | gaaaaaaaaacgacaaatctgtctgctggtcacttcttctgtccaagcagattcgtggtcttttcctcgcttctttcaagg |
| | | | gctttcctgtgccaggtgaaggaggctccaggcagcacccaggttttgcactcttgtttctcccgtgcttgtgaaagagg |
| | | | tcccaaggttctgggtgcaggagcgctcccttgacctgctgaagtccggaacgtagtcggcacagcctggtcgccttcc |
| | | | acctctgggagctggagtccactggggtggcctgactcccccagtccccttcccgtgacctggtcagggtgagcccatg |
| | | | tggagtcagcctcgcaggcctccctgccagtagggtccgagtgtgtttcatccttcccactctgtcgagcctgggggctg |
| | | | gagcggagacgggaggcctggcctgtctcggaacctgtgagctgcaccaggtagaacgccagggaccccagaatca |
| | | | tgtgcgtcagtccaaggggtcccctccaggagtagtgaagactccagaaatgtccctttcttctcccccatcctacgagt |
| | | | aattgcatttgcttttgtaattcttaatgagcaatatctgctagagagtttagctgtaacagttctttttgatcatcttttttt |
| | | | aataattagaaacaccaaaaaaatccagaaacttgttcttccaaagcagagagcattataatcaccagggccaaaag |
| | | | cttccctccctgctgtcattgcttcttctgaggcctgaatccaaaagaaaaacagccataggccctttcagtggccgggc |
| | | | tacccgtgagcccttcggaggaccagggctggggcagcctctgggcccacatccggggccagctccggcgtgtgttc |
| | | | agtgttagcagtgggtcatgatgctctttcccacccagcctgggataggggcagaggaggcgaggaggccgttgccg |
| | | | ctgatgtttggccgtgaacaggtgggtgtctgcgtgcgtccacgtgcgtgttttctgactgacatgaaatcgacgcccg |
| | | | agttagcctcacccggtgacctctagccctgcccggatggagcggggcccacccggttcagtgtttctggggagctgg |
| | | | acagtggagtgcaaaaggcttgcagaacttgaagcctgctccttcccttgctaccacggcctcctttccgtttgatttgtc |
| | | | actgcttcaatcaataacagccgctccagagtcagtagtcaatgaatatatgaccaaatatcaccaggactgttactca |
| | | | atgtgtgccgagcccttgcccatgctgggctcccgtgtatctggacactgtaacgtgtgctgtgtttgctccccttcccctt |
| | | | ccttctttgccctttacttgtctttctggggtttttctgtttgggtttggtttggtttttatttctccttttgtgttccaaacatga |
| | | | ggttctctctactggtcctcttaactgtggtgttgaggcttatatttgtgtaatttttggtgggtgaaaggaattttgctaag |
| | | | taaatctcttctgtgtttgaactgaagtctgtattgtaactatgtttaaagtaattgttccagagacaaatatttctagaca |
| | | | ctttttctttacaaacaaaagcattcggagggagggggatggtgactgagatgagaggggagagctgaacagatgac |
| | | | ccctgcccagatcagccagaagccacccaaagcagtggagcccaggagtcccactccaagccagcaagccgaatag |
| | | | ctgatgtgttgccactttccaagtcactgcaaaaccaggttttgttccgcccagtggattcttgttttgcttcccctccccc |
| | | | cgagattattaccaccatcccgtgcttttaaggaaaggcaagattgatgtttccttgaggggagccaggaggggatgt |
| | | | gtgtgtgcagagctgaagagctggggagaatggggctgggcccacccaagcaggaggctgggacgctctgctgtgg |
| | | | gcacaggtcaggctaatgttggcagatgcagctcttcctggacaggccaggtggtgggcattctctctccaaggtgtgc |
| | | | cccgtgggcattactgtttaagacacttccgtcacatcccaccccatcctccagggctcaacactgtgacatctctattcc |
| | | | ccaccctccccttcccagggcaataaaatgaccatggagggggcttgcactctcttggctgtcacccgatcgccagca |
| | | | aaacttagatgtgagaaaaccccttcccattccatggcgaaaacatctccttagaaaagccattaccctcattaggcat |
| | | | ggttttgggctcccaaaacacctgacagcccctccctcctctgagaggcggagagtgctgactgtagtgaccattgcat |
| | | | gccgggtgcagcatctggaagagctaggcagggtgtctgccccctcctgagttgaagtcatgctcccctgtgccagcc |
| | | | cagaggccgagagctatggacagcattgccagtaacacaggccaccctgtgcagaagggagctggctccagcctgg |
| | | | aaacctgtctgaggttgggagaggtgcacttggggcacagggagaggccgggacacacttagctggagatgtctcta |
| | | | aaagccctgtatcgtattcaccttcagtttttgtgttttgggacaattactttagaaaataagtaggtcgttttaaaaaca |
| | | | aaaattattgattgcttttttgtagtgttcagaaaaaaggttctttgtgtatagccaaatgactgaaagcactgatatattt |
| | | | aaaaacaaaaggcaatttattaaggaaatttgtaccatttcagtaaacctgtctgaatgtacctgtatacgtttcaaaaa |
| | | | caccccccccccactgaatccctgtaacctatttattatataaagagtttgccttataaattt |
| Prion | FXN | 24 | atgtggactctcgggcgccgcgcagtagccggcctcctggcgtcacccagcccagcccaggcccagaccctcacccg |
| disease | (KU178079.1) | | ggtcccgcggccggcagagttggccccactctgcggccgccgtggcctgcgcaccgacatcgatgcgacctgcacgc |
| | | | cccgccgcgcaagttcgaaccaacgtggcctcaaccagatttggaatgtcaaaaagcagagtgtctatttgatgaattt |
| | | | gaggaaatctggaactttgggccacccaggctctctagatgagaccacctatgaaagactagcagaggaaacgctgg |
| | | | actctttagcagagttttttgaagaccttgcagacaagccatacacgtttgaggactatgatgtctcctttgggagtggt |
| | | | gtcttaactgtcaaactgggtggagatctaggaacctatgtgatcaacaagcagacgccaaacaagcaaatctggcta |
| | | | tcttctccatccagtggacctaagcgttatgactggactgggaaaaactgggtgtactcccacgacggcgtgtccctcc |
| | | | atgagctgctggccgcagagctcactaaagccttaaaaaccaaactggacttgtcttccttggcctattccggaaaaga |
| | | | tgct |
| | PRNP | 25 | atggcgaaccttggctgctggatgctggttctctttgtggccacatggagtgacctgggcctctgcaagaagcgcccga |
| | (AF085477.2) | | agcctggaggatggaacactgggggcagccgatacccggggcagggcagccctggaggcaaccgctacccacctc |
| | | | agggcggtggtggctgggggcagcctcatggtggtggctgggggcagcctcatggtggtggctgggggcagccccat |
| | | | ggtggtggctgggggcagcctcatggtggtggctgggggcagcctcatggtggtggctgggggcagcctcatggtgg |
| | | | tggctgggggcagccccatggtggtggctggggacagcctcatggtggtggctggggtcaaggaggtggcacccaca |
| | | | gtcagtggaacaagccgagtaagccaaaaaccaacatgaagcacatggctggtgctgcagcagctggggcagtggt |
| | | | ggggggccttggcggctacatgctgggaagtgccatgagcaggcccatcatacatttcggcagtgactatgaggacc |
| | | | gttactatcgtgaaaacatgcaccgttaccccaaccaagtgtactacaggcccatggatgagtacagcaaccagaac |
| | | | aactttgtgcacgactgcgtcaatatcacaatcaagcagcacacggtcaccacaaccaccaagggggagaacttcac |
| | | | cgagaccgacgttaagatgatggagcgcgtggttgagcagatgtgtatcacccagtacgagagggaatctcaggcct |
| | | | attaccagagaggatcgagcatggtcctcttctcctctccacctgtgatcctcctgatctctttcctcatcttcctgatagt |
| | | | gggatga |
| | SMN1 | 26 | ccacaaatgtgggagggcgataaccactcgtagaaagcgtgagaagttactacaagcggtcctcccggccaccgtac |
| | (NM_001297 | | tgttccgctcccagaagccccgggcggcggaagtcgtcactcttaagaagggacggggccccacgctgcgcacccgc |
| | 715.1) | | gggtttgctatggcgatgagcagcggcggcagtggtggcggcgtcccggagcaggaggattccgtgctgttccggcg |
| | | | cggcacaggccagagcgatgattctgacatttgggatgatacagcactgataaaagcatatgataaagctgtggcttc |
| | | | atttaagcatgctctaaagaatggtgacatttgtgaaacttcgggtaaaccaaaaaccacacctaaaagaaaacctgc |
| | | | taagaagaataaaagccaaaagaagaatactgcagcttccttacaacagtggaaagttggggacaaatgttctgcca |
| | | | tttggtcagaagacggttgcatttacccagctaccattgcttcaattgattttaagagagaaacctgtgttgtggtttaca |
| | | | ctggatatggaaatagagaggagcaaaatctgtccgatctactttccccaatctgtgaagtagctaataatatagaaca |
| | | | aaatgctcaagagaatgaaaatgaaagccaagtttcaacagatgaaagtgagaactccaggtctcctggaaataaat |
| | | | cagataacatcaagcccaaatctgctccatggaactcttttctccctccaccaccccccatgccagggccaagactggg |
| | | | accaggaaagccaggtctaaaattcaatggcccaccaccgccaccgccaccaccaccaccccacttactatcatgctg |
| | | | gctgcctccatttccttctggaccaccaataattcccccaccacctcccatatgtccagattctcttgatgatgctgatgct |
| | | | ttgggaagtatgttaatttcatggtacatgagtggctatcatactggctattatatggaaatgctggcatagagcagcac |
| | | | taaatgacaccactaaagaaacgatcagacagatctggaatgtgaagcgttatagaagataactggcctcatttcttc |
| | | | aaaatatcaagtgttgggaaagaaaaaaggaagtggaatgggtaactcttcttgattaaaagttatgtaataaccaaa |
| | | | tgcaatgtgaaatattttactggactctattttgaaaaaccatctgtaaaagactggggtgggggtgggaggccagcac |
| | | | ggtggtgaggcagttgagaaaatttgaatgtggattagattttgaatgatattggataattattggtaattttatgagctg |
| | | | tgagaagggtgttgtagtttataaaagactgtcttaatttgcatacttaagcatttaggaatgaagtgttagagtgtctta |
| | | | aaatgtttcaaatggtttaacaaaatgtatgtgaggcgtatgtggcaaaatgttacagaatctaactggtggacatggc |
| | | | tgttcattgtactgtttttttctatcttctatatgtttaaaagtatataataaaaatatttaatttttttttaaa |
| | CLN3 | 27 | cccctagacaagccggagctgggaccggcaatcgggcgttgatccttgtcacctgtcgcagaccctcatccctcccgt |
| | (U32680.1) | | gggagccccctttggacactctatgaccctggaccctcgggggacctgaacttgatgcgatgggaggctgtgcaggct |
| | | | cgcggcggcgcttttcggattccgagggggaggagaccgtcccggagccccggctccctctgttggaccatcagggc |
| | | | gcgcattggaagaacgcggtgggcttctggctgctgggcctttgcaacaacttctcttatgtggtgatgctgagtgccg |
| | | | cccacgacatccttagccacaagaggacatcgggaaaccagagccatgtggacccaggcccaacgccgatccccca |
| | | | caacagctcatcacgatttgactgcaactctgtctctacggctgctgtgctcctggcggacatcctccccacactcgtca |
| | | | tcaaattgttggctcctcttggccttcacctgctgccctacagcccccgggttctcgtcagtgggatttgtgctgctggaa |
| | | | gcttcgtcctggttgccttttctcattctgtggggaccagcctgtgtggtgtggtcttcgctagcatctcatcaggccttgg |
| | | | ggaggtcaccttcctctccctcactgccttctaccccagggccgtgatctcctggtggtcctcagggactgggggagct |
| | | | gggctgctgggggccctgtcctacctgggcctcacccaggccggcctctcccctcagcagaccctgctgtccatgctgg |
| | | | gtatccctgccctgctgctggccagctatttcttgttgctcacatctcctgaggcccaggaccctggaggggaagaaga |
| | | | agcagagagcgcagcccggcagcccctcataagaaccgaggccccggagtcgaagccaggctccagctccagcctc |
| | | | tcccttcgggaaaggtggacagtattcaagggtctgctgtggtacattgttcccttggtcgtagtttactttgccgagtat |
| | | | ttcattaaccagggactttttgaactcctctttttctggaacacttccctgagtcacgctcagcaataccgctggtaccag |
| | | | atgctgtaccaggctggcgtctttgcctcccgctcttctctccgctgctgtcgcatccgtttcacctgggccctggccctgc |
| | | | tgcagtgcctcaacctggtgttcctgctggcagacgtgtggttcggctttctgccaagcatctacctcgtcttcctgatca |
| | | | ttctgtatgaggggctcctgggaggcgcagcctacgtgaacaccttccacaacatcgccctggagaccagtgatgagc |
| | | | accgggagtttgcaatggcggccacctgcatctctgacacactggggatctccctgtcggggctcctggctttgcctctg |
| | | | catgacttcctctgccagctctcctgatactcgggatcctcaggacgcaggtcacattcacctgtgggcagagggacag |
| | | | gtcagacacccaggcccaccccagagaccctccatgaactgtgctcccagccttcccggcaggtctgggagtaggga |
| | | | agggctgaagccttgtttccttgcaggggggccagccattgtctcccacttggggagtttcttcctggcatcatgccttct |
| | | | gaataaatgccgattttgtccatgg |
| | Tou | 28 | caggtgaactttgaaccaggatggctgagccccgccaggagttcgaagtgatggaagatcacgctgggacgtacggg |
| | (BC114948.1) | | ttgggggacaggaaagatcaggggggctacaccatgcaccaagaccaagagggtgacacggacgctggcctgaaa |
| | | | gctgaagaagcaggcattggagacacccccagcctggaagacgaagctgctggtcacgtgacccaagctcgcatgg |
| | | | tcagtaaaagcaaagacgggactggaagcgatgacaaaaaagccaagggggctgatggtaaaacgaagatcgcca |
| | | | caccgcggggagcagcccctccaggccagaagggccaggccaacgccaccaggattccagcaaaaaccccgcccg |
| | | | ctccaaagacaccacccagctctggtgaacctccaaaatcaggggatcgcagcggctacagcagccccggctcccca |
| | | | ggcactcccggcagccgctcccgcaccccgtcccttccaaccccacccacccgggagcccaagaaggtggcagtggt |
| | | | ccgtactccacccaagtcgccgtcttccgccaagagccgcctgcagacagcccccgtgcccatgccagacctgaaga |
| | | | atgtcaagtccaagatcggctccactgagaacctgaagcaccagccgggaggcgggaaggtgcagataattaataa |
| | | | gaagctggatcttagcaacgtccagtccaagtgtggctcaaaggataatatcaaacacgtcccgggaggcggcagtg |
| | | | tgcaaatagtctacaaaccagttgacctgagcaaggtgacctccaagtgtggctcattaggcaacatccatcataaac |
| | | | caggaggtggccaggtggaagtaaaatctgagaagcttgacttcaaggacagagtccagtcgaagattgggtccctg |
| | | | gacaatatcacccacgtccctggcggaggaaataaaaagattgaaacccacaagctgaccttccgcgagaacgcca |
| | | | aagccaagacagaccacggggcggagatcgtgtacaagtcgccagtggtgtctggggacacgtctccacggcatctc |
| | | | agcaatgtctcctccaccggcagcatcgacatggtagactcgccccagctcgccacgctagctgacgaggtgtctgcc |
| | | | tccctggccaagcagggtttgtgatcaggcccctggggcggtcaataattgtggagaggagagaatgagagagtgtg |
| | | | gaaaaaaaaagaataatgacccggcccccgccctctgcccccagctgctcctcgcagttcggttaattggttaatcact |
| | | | taacctgcttttgtcactcggctttggctcgggacttcaaaatcagtgatgggagtaagagcaaatttcatctttccaaat |
| | | | tgatgggtgggctagtaataaaatatttaaaaaaaaacattcaaaaacatggccacatccaacatttcctcaggcaatt |
| | | | ccttttgattcttttttcttccccctccatgtagaagagggagaaggagaggctctgaaagctgcttctgggggatttcaa |
| | | | gggactgggggtgccaaccacctctggccctgttgtgggggtgtcacagaggcagtggcagcaacaaaggatttgaa |
| | | | acttggtgtgttcgtggagccacaggcagacgatgtcaaccttgtgtgagtgtgacgggggttggggtggggcggga |
| | | | ggccacgggggaggccgaggcaggggctgggcagaggggagaggaagcacaagaagtgggagtgggagaggaa |
| | | | gccacgtgctggagagtagacatccccctccttgccgctgggagagccaaggcctatgccacctgcagcgtctgagcg |
| | | | gccgcctgtccttggtggccgggggtgggggcctgctgtgggtcagtgtgccaccctctgcagggcagcctgtgggag |
| | | | aagggacagcgggtaaaaagagaaggcaagctggcaggagggtggcacttcgtggatgacctccttagaaaagact |
| | | | gaccttgatgtcttgagagcgctggcctcttcctccctccctgcagggtagggggcctgagttgaggggcttccctctgc |
| | | | tccacagaaaccctgttttattgagttctgaaggttggaactgctgccatgattttggccactttgcagacctgggacttt |
| | | | agggctaaccagttctctttgtaaggacttgtgcctcttgggagacgtccacccgtttccaagcctgggccactggcatc |
| | | | tctggagtgtgtgggggtctgggaggcaggtcccgagccccctgtccttcccacggccactgcagtcacccctgtctgc |
| | | | gccgctgtgctgttgtctgccgt |

In some embodiments, at least one cell in the disclosed systems comprises any one or more of the mutations at the loci identified in TABLE B. If TABLE B discloses a mRNA sequence the one or more mutations of the cell may be in the complementary endogenous DNA sequence disclosed at GenBank sequence above. In some embodiments, the cell comprises a mutation of one or more of the above-identified sequences of Table B, or a sequence that comprises at least about 70%, 75%, 80%, 85%, 90%, 91%, 92%, 93%, 94%, 95%, 96%, 97%, 98%, or 99% identity to any of the sequences disclosed in Table B, or, if the sequence is a mRNA sequence, the cell may comprise a mutation of one or more of the complementary DNA sequence of those sequences identified in Table B, or a mutation that is or is complementary to a sequence that comprises at least about 70%, 75%, 80%, 85%, 90%, 91%, 92%, 93%, 94%, 95%, 96%, 97%, 98%, or 99% sequence identity to any of the sequences disclosed in Table B.

In some embodiments, the spheroids disclosed herein comprise 2, 3, 4 or 5 or more mutations in the genes identified in Table B. If a spheroid comprising the particular mutation identified in Table B is used within the system disclosed herein, that corresponding system may be used as an in vitro model for the above-identified corresponding disease state.

In some embodiments, any of the compositions, systems, or methods as described in PCT/US2015/050061 may be used in embodiments of the present disclosure.

In some embodiments, the methods relate to a method of manufacturing a system. culture plate or device for culturing cells, the method comprising obtaining a stem cell, such as a induce pluripotent stem cell, exposing the cell to one or plurality of cellular growth factors, differentiating the stem cells into a neuronal cell, and seeding the cell into a solid substrate comprising a first and/or second cavity or well. In some embodiments, the first and/or second cavity is a U bottom well, a curved-bottom well or flat-bottom well. In some embodiments, the method comprise seeding about 10, 15, 20, 25, 30, 35, 40, 45, 50, 55, 60, 65, 70, 75, 80, 90, 100, 125, 150, 175, 200, 225 or 250 thousand cells. In some embodiments, the step of seeding the cells comprises seeding one or a plurality f cells in a series of cavities or wells separated within a solid substrate and each cavity or well comprising cell culture medium. In some embodiments, the step of seeding the cavities or wells comprises seeding the cells in a pattern positioned within the solid substrate such that each well comprises a spheroid of cells and each spheroid is grown in a suspension or hanging drop format. In some embodiments, the method of manufacturing a system, culture plate or device for culturing cells comprises allowing the cells to culture undisturbed for sufficient time for the cells to spontaneously form one or a plurality of spheroids.

The disclosure also relates to a method of testing the toxicity of an agent by exposing an agent to one or a plurality of spheroids on or within a cavity or well within a solid substrate. In some embodiments, the methods further comprises allowing the agent to be exposed to the one or plurality of spheroids for a time sufficient for the agent to become absorbed by one or a plurality of cells of the one or plurality of spheroids and then measuring the viability of the cells through a recording, observation of morphological changes or a combination of both.

The disclosure also relates to the method of forming a spheroid of cells derived from stem cells or cells from the nervous system of a subject. In some embodiments, the method of forming a spheroid comprises (i) differentiating cells from a stem cell to a cell or plurality of cell types that are one or a combination of a neuronal cell, astrocyte, Schwann cell, or any other cell disclosed herein, and then (ii) mixing the one or plurality of cells for a time period sufficient to form a spheroid. In some embodiments, the method does not comprise a step of differentiating any cells after a spheroid is formed. In some embodiments, the methods are free of exposing the spheroid or any cell to one or a plurality of DRGs.

The following examples are meant to be non-limiting examples of how to make and use the embodiments disclosed in this application. Any publications, patents or patent applications disclosed in the examples or the body of the specification are incorporated by reference in their entireties.

### EXAMPLES

### Example 1: Human Motor-Nerve-On-A-Chip for Preclinical Neurotoxicity Testing

The objective was to develop an organotypic, micro-physiological model to mimic the morphology of peripheral nerves and support clinically analogous physiological measurements. The nerve-on-a-chip design was fabricated using microengineered hydrogel scaffolding (FIG. 1A-FIG. 1B).

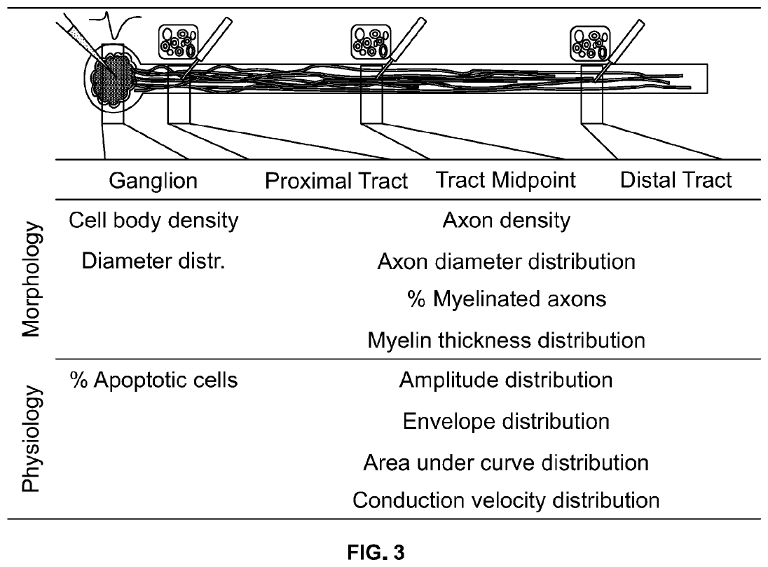

The goal of the design was to direct and confine 3D axon growth and cellular positioning to mimic the nerve fiber tract (FIG. 2). The robust nerve growth, fasciculation, and glial interactions facilitated morphological and physiological outputs as a high-content screening assay for neurotoxicity and pharmacological manipulation (FIG. 3).

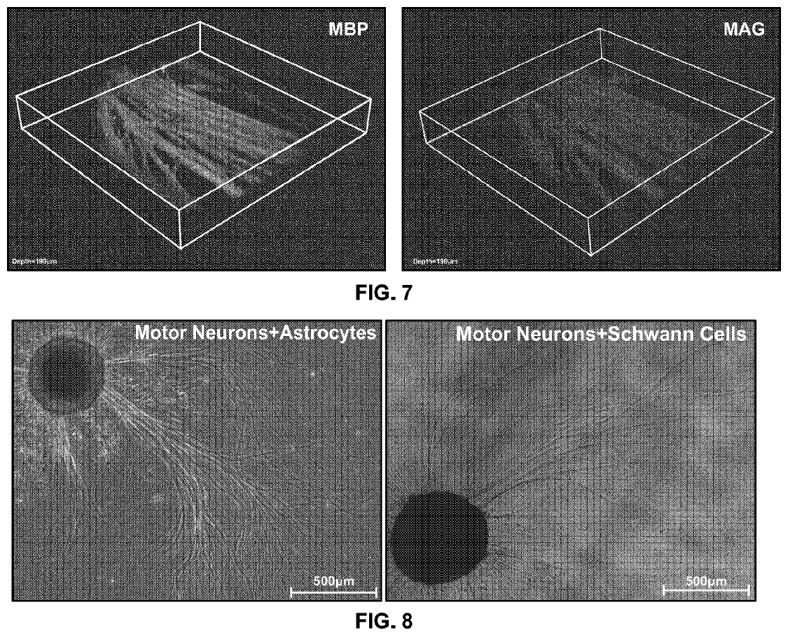

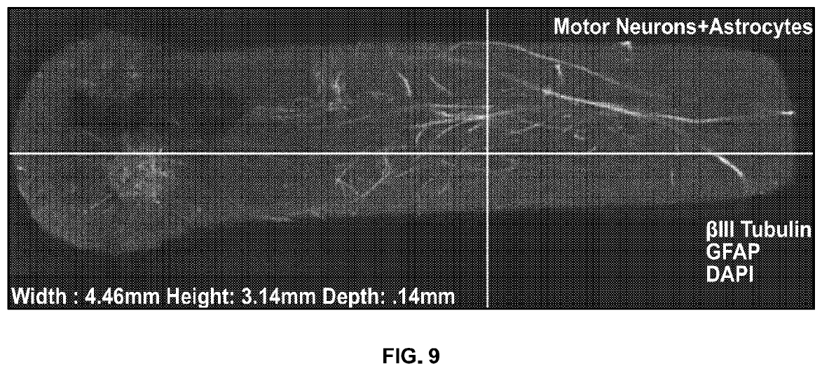

Results showed an architecture that resembled native peripheral nerve anatomy. This allowed for tests of nerve density, fiber type, and myelination, as well as studies of axon growth, cell migration, and glial differentiation (FIG. 4-FIG. 7).

### Example 2: Rat Spinal Cord Nanoshuttle Spheroid Protocol

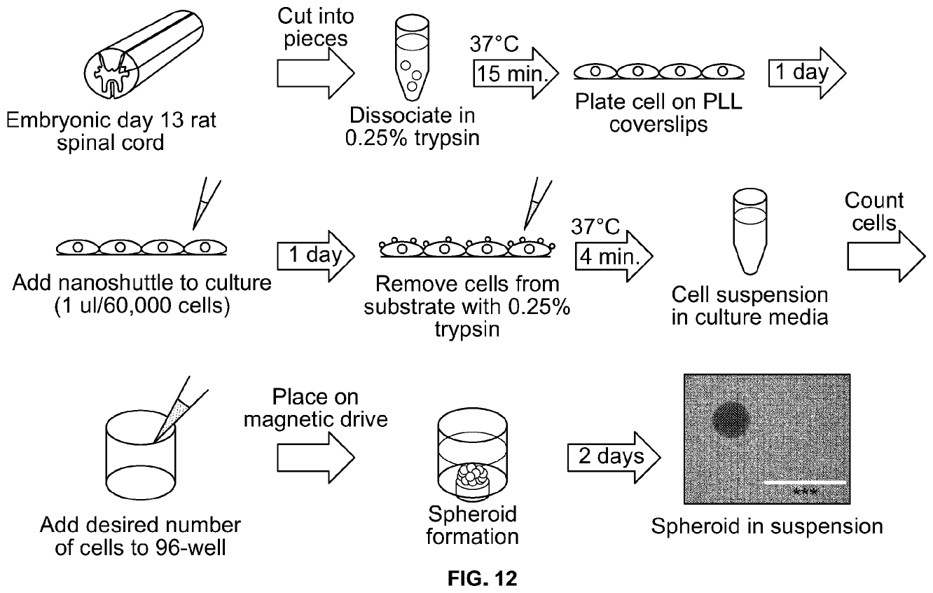

### Day 1 (FIG. 12):

1. Isolate 6 spinal cords from E15 rat pups ensuring that all dorsal root ganglia are removed.
2. Using spring scissors, cut all 6 spinal cords into small chunks.
3. Using a 1000 μL pipet, transfer the chunked spinal cord and media to a 1.5 mL microcentrifuge tube.
4. Pellet the spinal cord chunks by centrifuging for 2 minutes, 30 seconds at 700 rcf.
5. Remove the supernatant from the microcentrifuge tube, taking care not to disrupt the pellet.
6. Add 1 mL of 0.25% trypsin-EDTA to the microcentrifuge tube, ensuring that the pellet is broken apart and suspended in the trypsin.
7. Incubate for 15 minutes at 37° C.
8. Quench the trypsin-EDTA by adding the trypsin+cell suspension to a 15 mL tube containing 1.5 mL of the trypsin inhibitor solution.
9. Pellet the spinal cord chunks by centrifuging for 5 minutes at 700 rcf.
10. Remove the supernatant from the 15 mL conical tube, taking care not to disrupt the pellet.
11. Add 2 mL of spinal cord plating media to the 15 mL conical tube containing the pellet.
12. Triturate 15× (set pipet to 1 mL volume) with 100 μL pipet to break chunks apart.
13. Add additional 3 mL of spinal cord plating media to the dissociated spinal cord solution.
14. Pass all 5 mL of dissociated spinal cord solution through a 40 μm cell strainer, collecting the strained media in a petri dish.
15. Add 300 μL of dissociated spinal cord solution on top of each 12-well PLL coverslip. The media should remained bubbled on top of the coverslip and not run over the edges of the coverslip to flood the well. To ensure complete coverage of the coverslip, gently tilt the 12-well plate to allow the dissociated spinal cord solution to spread over the entire coverslip. Repeat, tilting in additional directions to get complete coverage as necessary.
16. Incubate the plated cells for 2 hours at 37° C.
17. After 2 hours, gently remove the plating media and replace with 700 μL of N2:NG media. Add the N2:NG media to the side of the well to prevent fluid flow from removing the plated cells from the substrate.

### Day 2:

1. Sacrifice one well of plated cells for counting by adding 0.5 mL of 0.25% trypsin-EDTA to a well.
2. Incubate for 4 minutes at 37° C.
3. Transfer trypsin and cells lifted from the substrate to a 1.5 mL microcentrifuge tube containing 0.5 mL of neural basal media to dilute trypsin.
4. Triturate 10× to break apart cell-cell adhesions giving a cell solution.
5. Count the number of cells present in the well using trypan blue and a hemocytometer.
6. Add nanoshuttle to the remaining wells at a ratio of 1 μL per every 60,000 cells. Try to disperse the nanoshuttle solution over the entire area of the coverslip.
7. Return to incubator for further culture.

### Day 3:

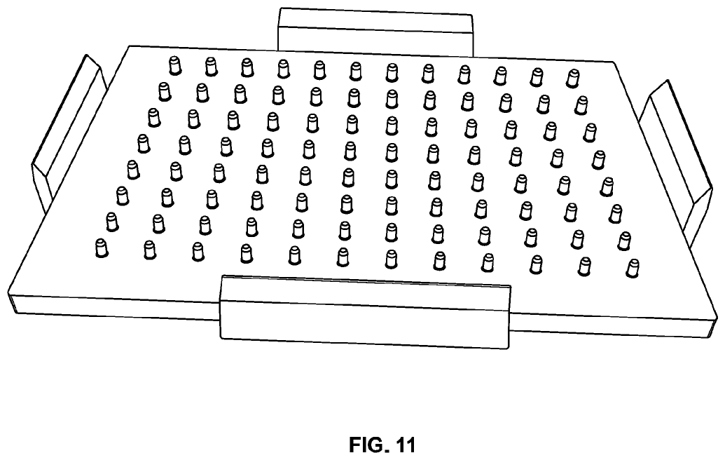

1. Add 0.5 mL of 0.25% trypsin-EDTA to each well.
2. Incubate for 4 minutes at 37° C.
3. Transfer trypsin and cells lifted from the substrate to a 15 mL conical tube containing a volume of N2:NG media equal to the amount of trypsin diluting.
4. Centrifuge the cell solution for 5 minutes at 700 rcf.
5. Remove supernatant and replace with 2 mL of N2:NG media.
6. Using a 1 mL syringe and a 20 gauge needle, break apart the cell pellet by triturating 5×.
7. Add an additional 5.2 mL of N2:NG media to the cell suspension (for a total of 7.2 mL).
8. Take a 10 μL sample of this cell suspension and using trypan blue and a hemocytometer, calculate the number of cells per mL.
9. Calculate the volume of cell suspension that needs to be added to each 96-well.
   Ex: 500,000; 400,000; or 300,000 cells per well
10. Ensure that a non-adherent 96-well plate is sitting on top of a magnetic drive (FIG. 11) then add the appropriate volume of cell solution to each of the non-adherent 96-wells. Add additional N2:NG media to the well as necessary so that each of the 96-wells has a volume of 150 μL.
11. Return the 96-well plate to the incubator and allow spheroids to form undisrupted for 2 days.

### Day 5:

1. Make PEG constructs. See other protocols if guidance is needed.
2. Wash 3× with 2% anti/anti wash solution.
3. Store in wash solution overnight at 37° C.

### Day 6:

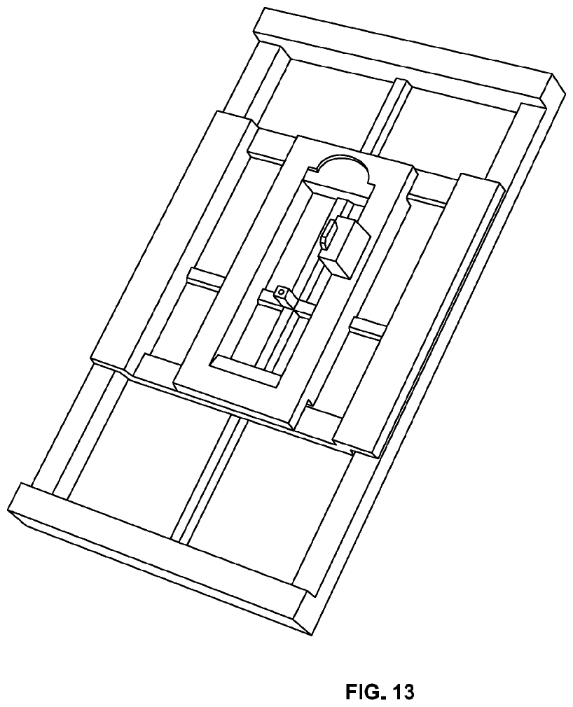

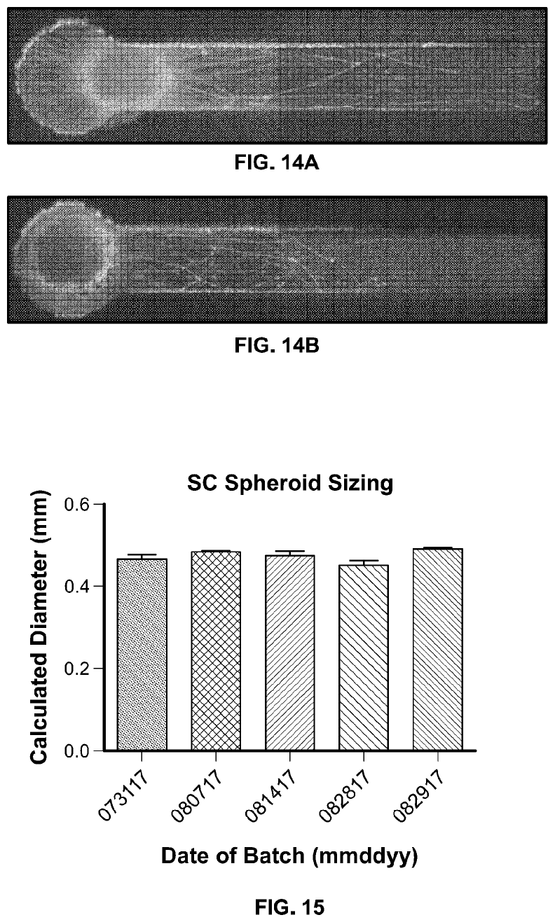

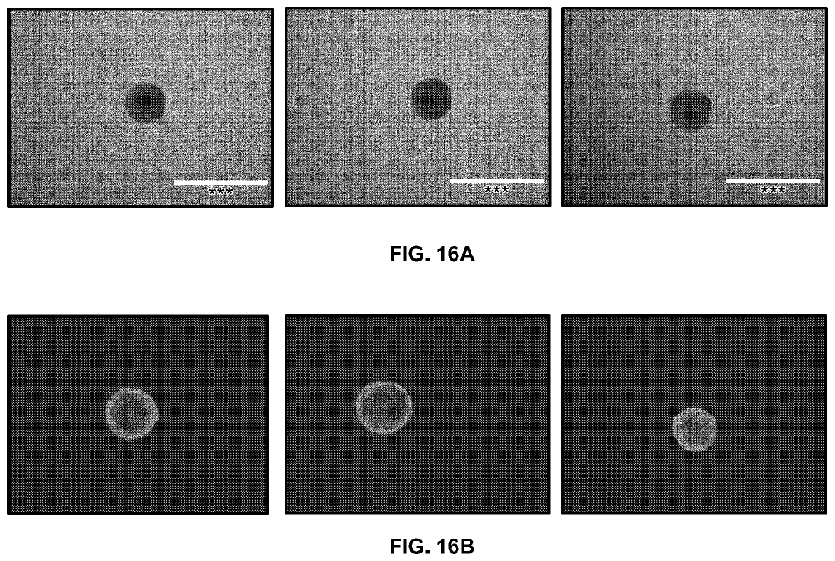

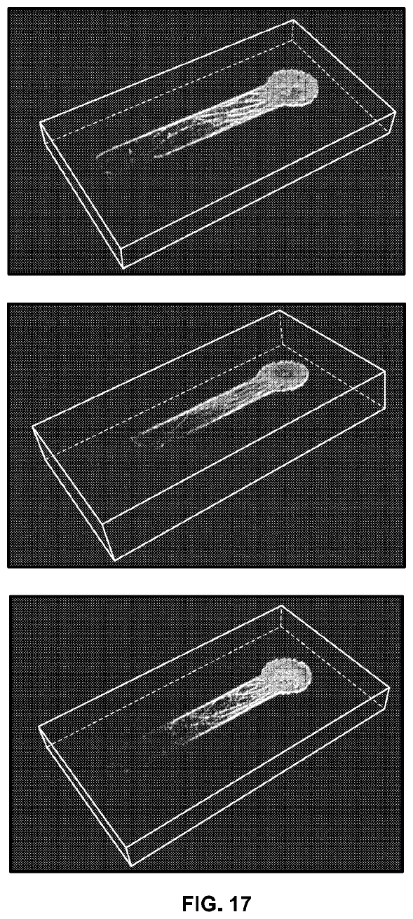

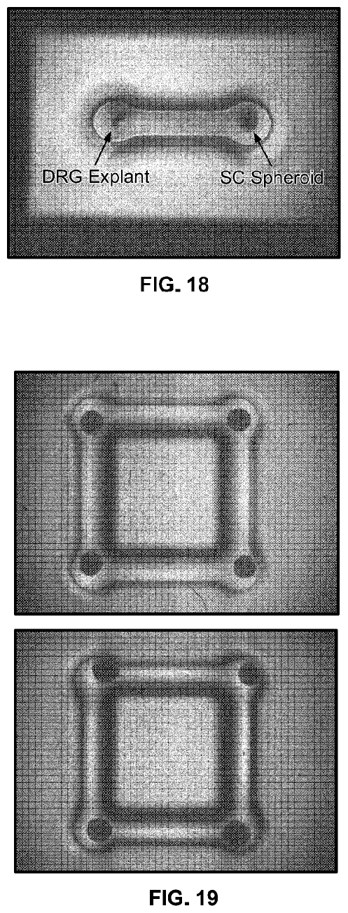

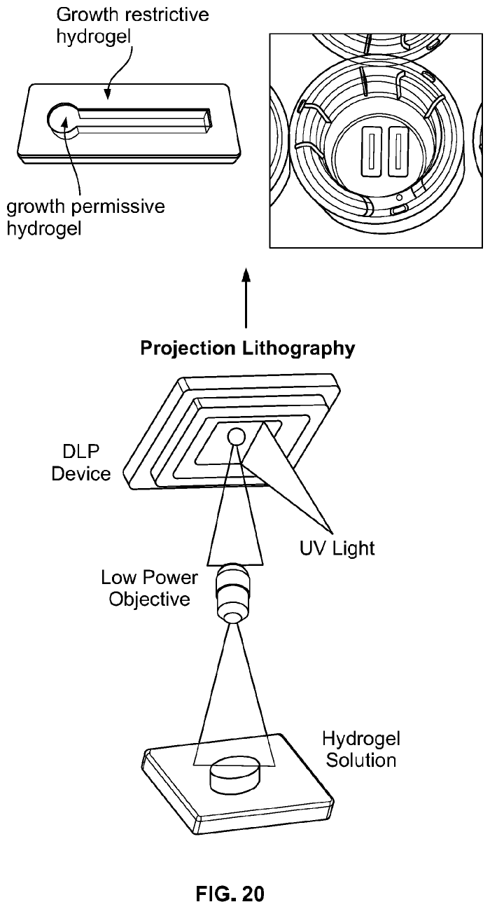

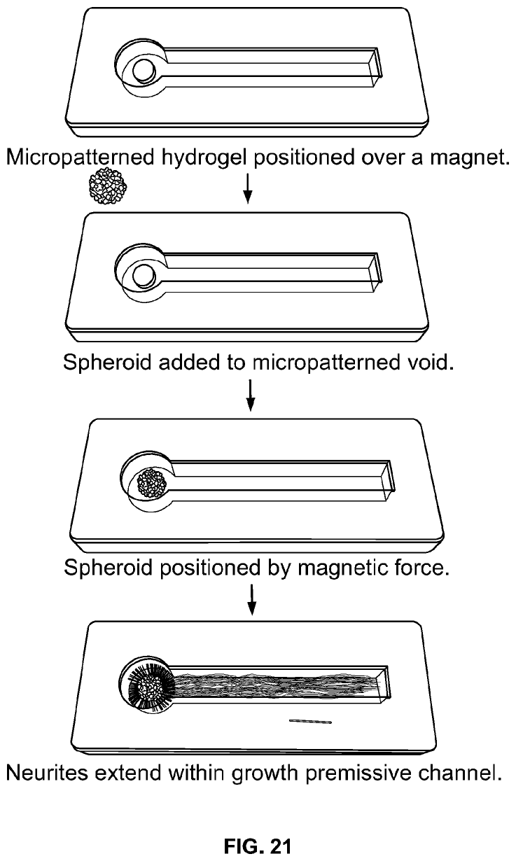

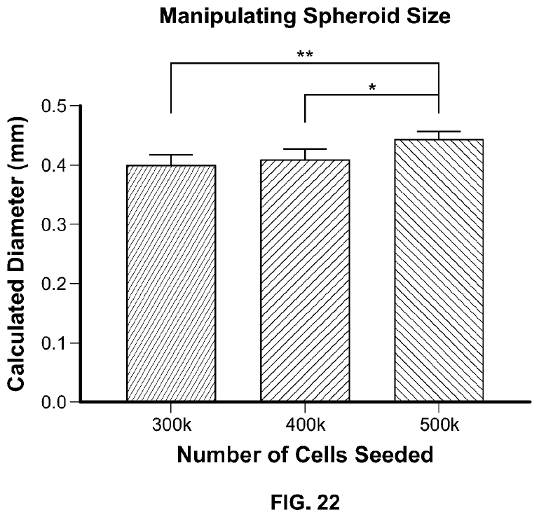

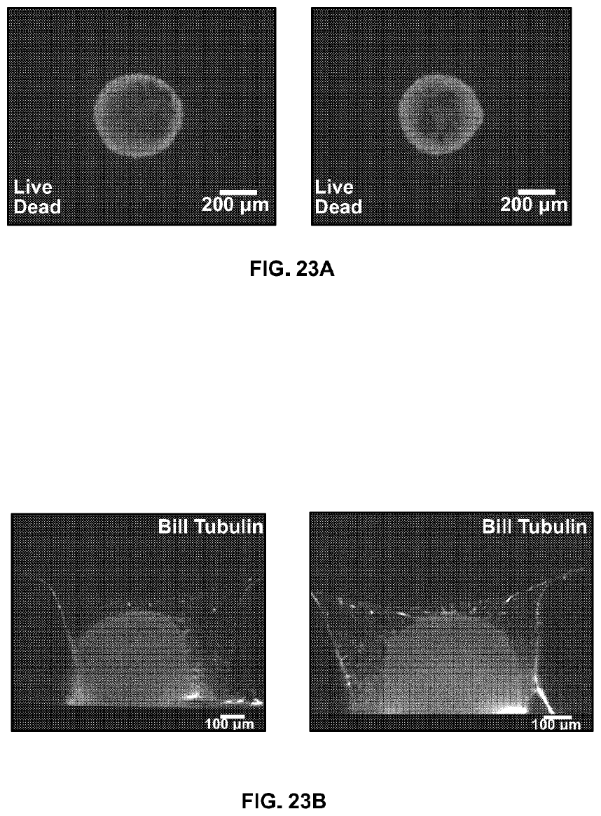

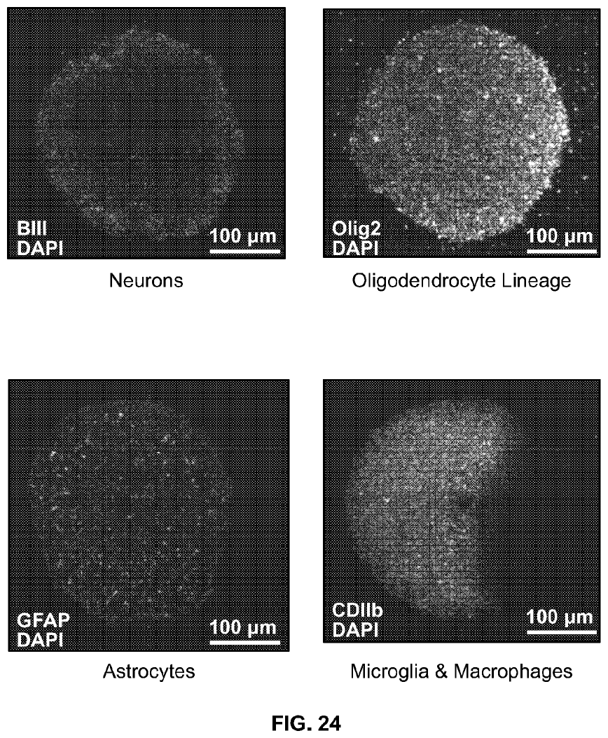

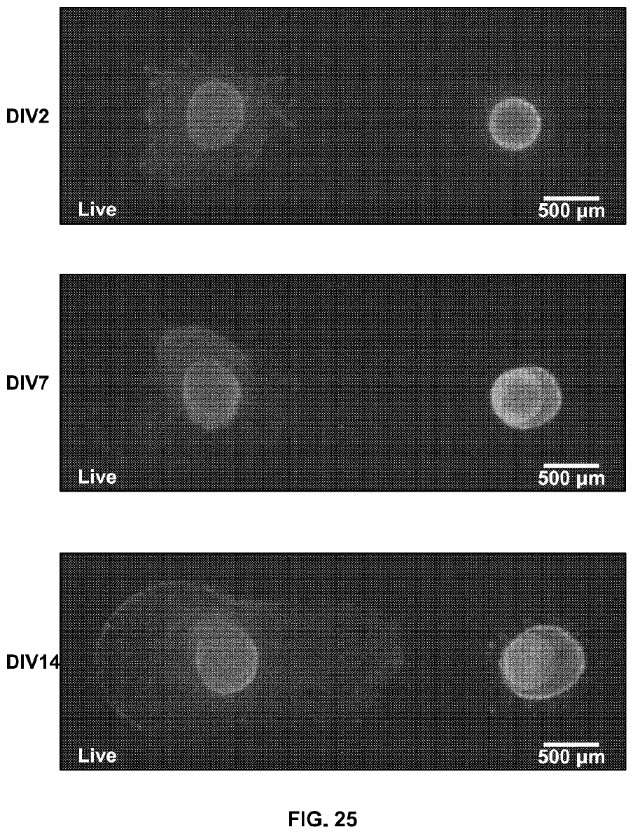

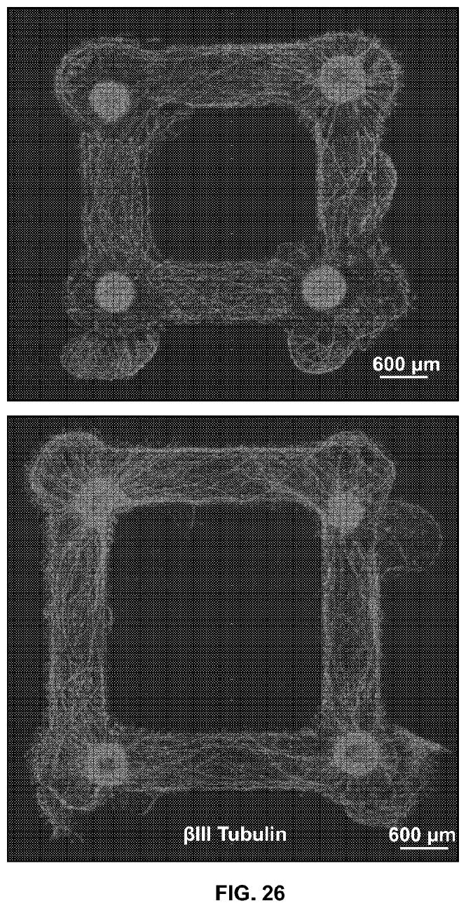

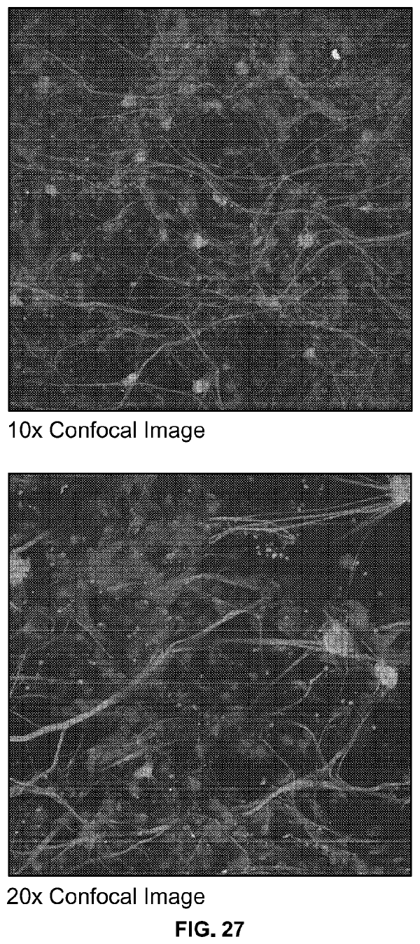

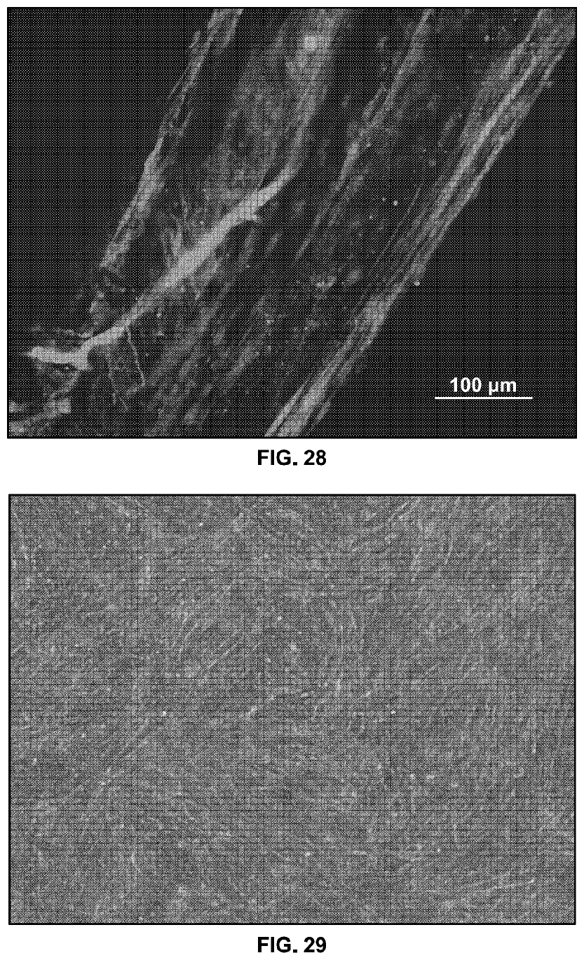

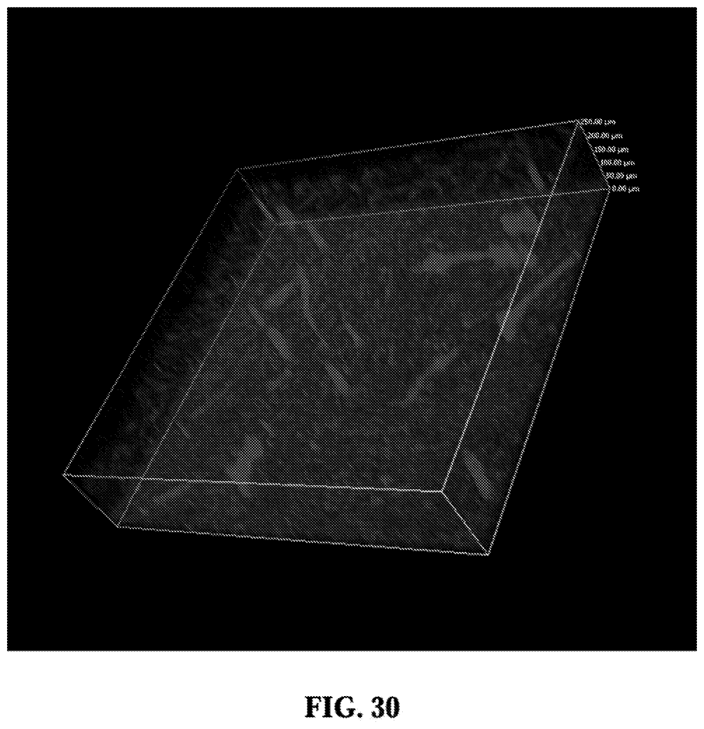

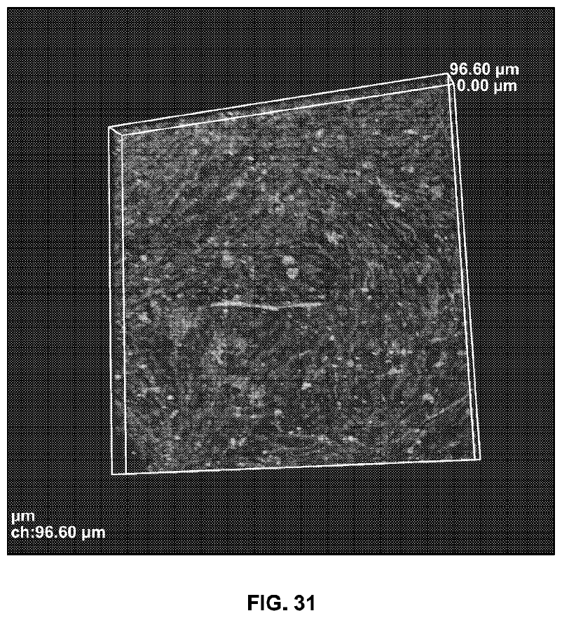

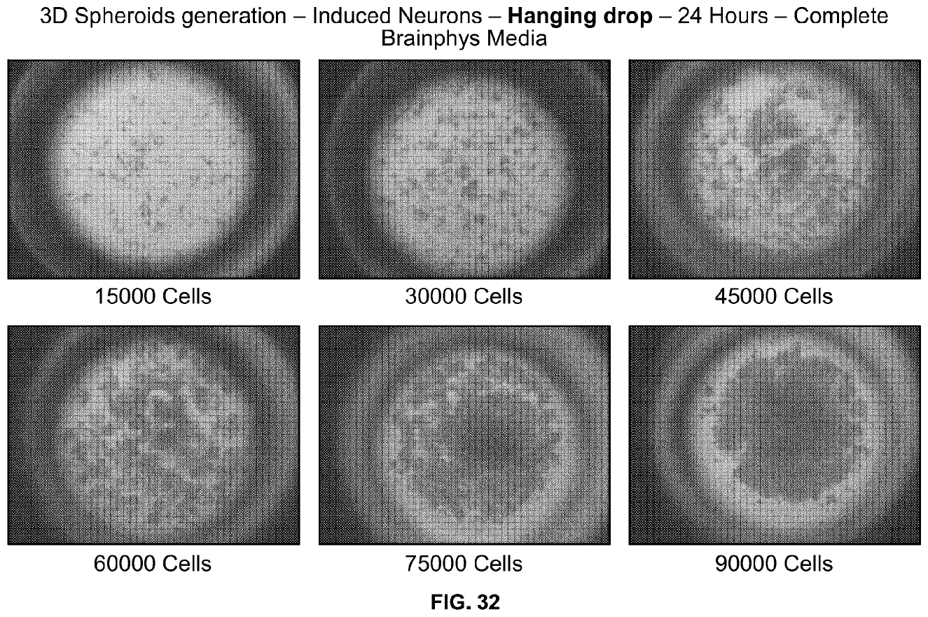

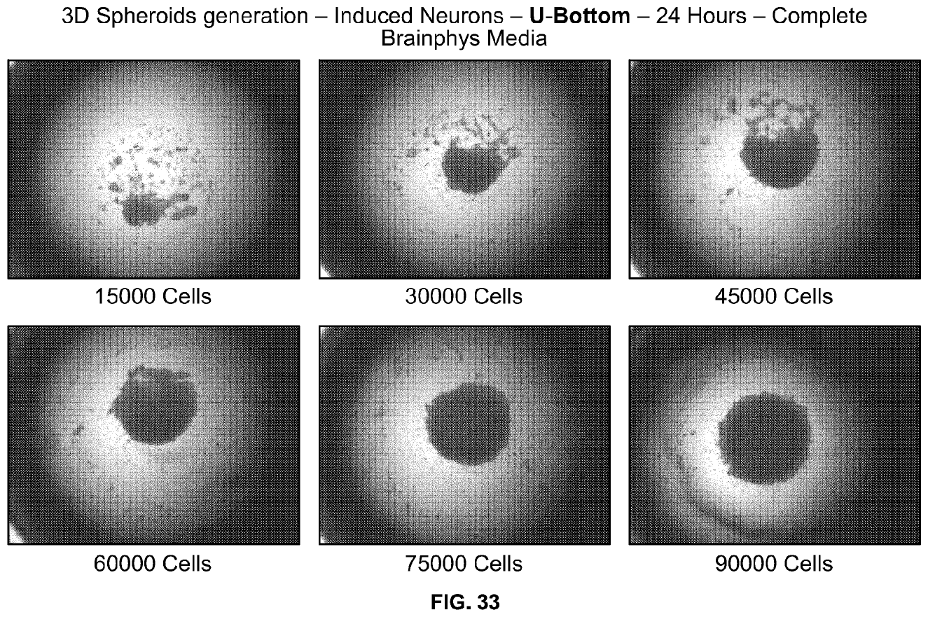

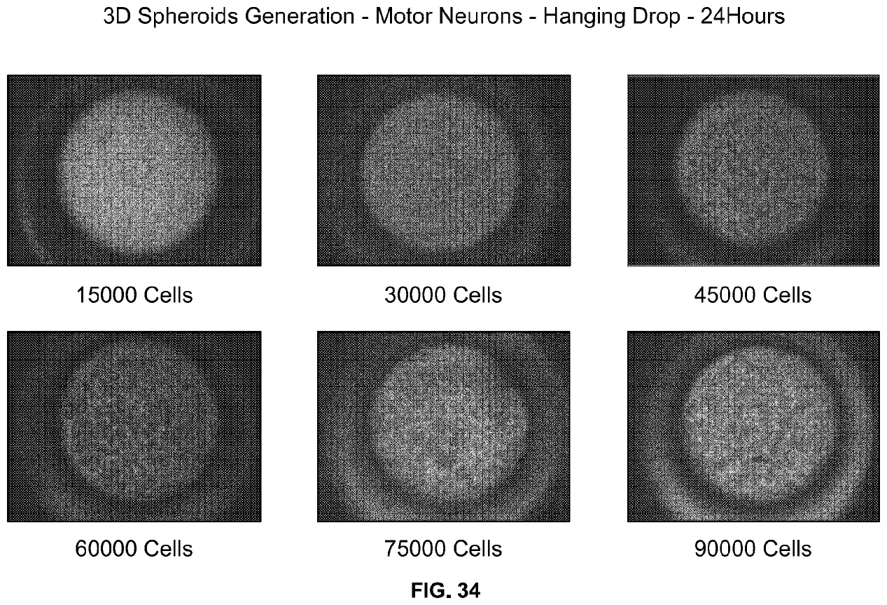

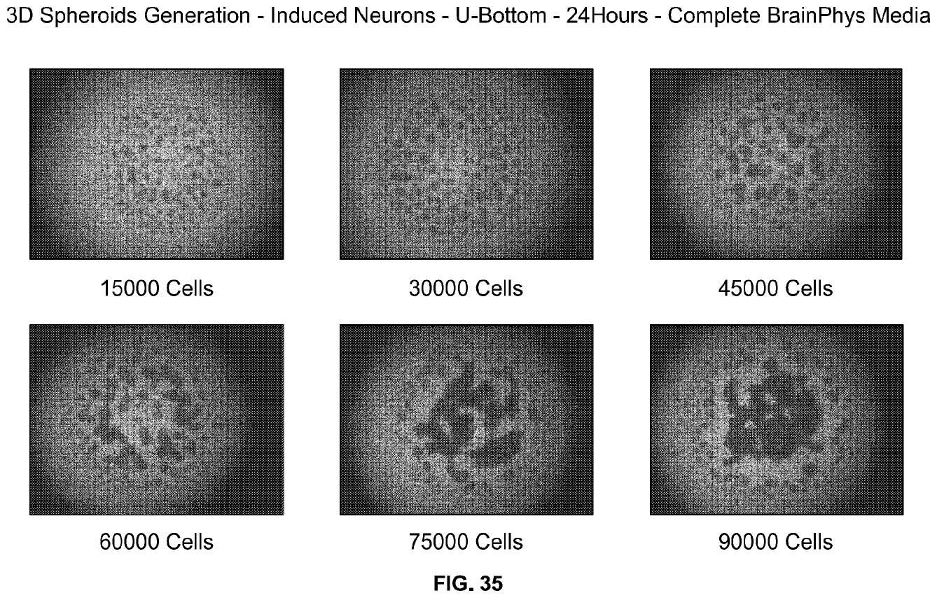

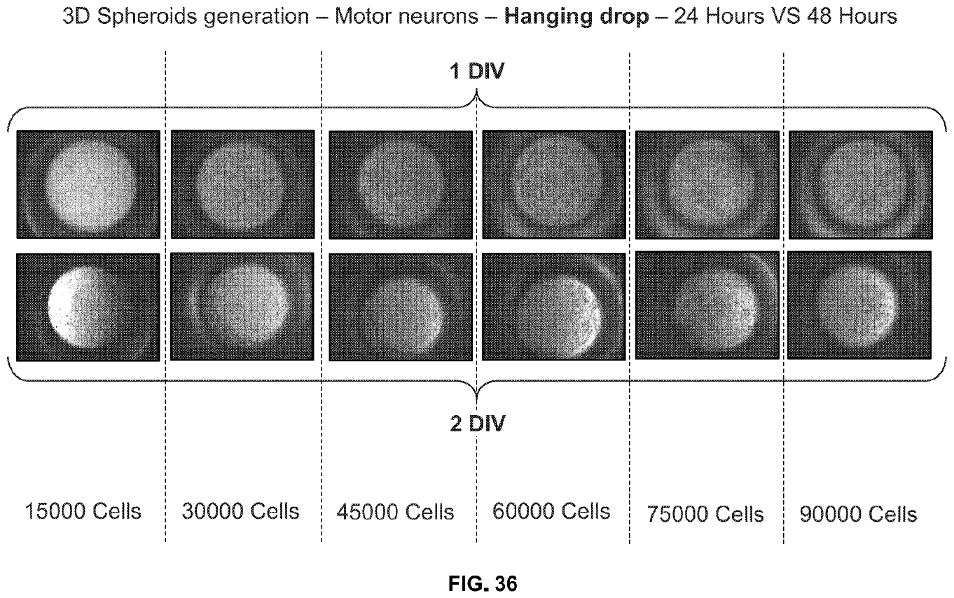

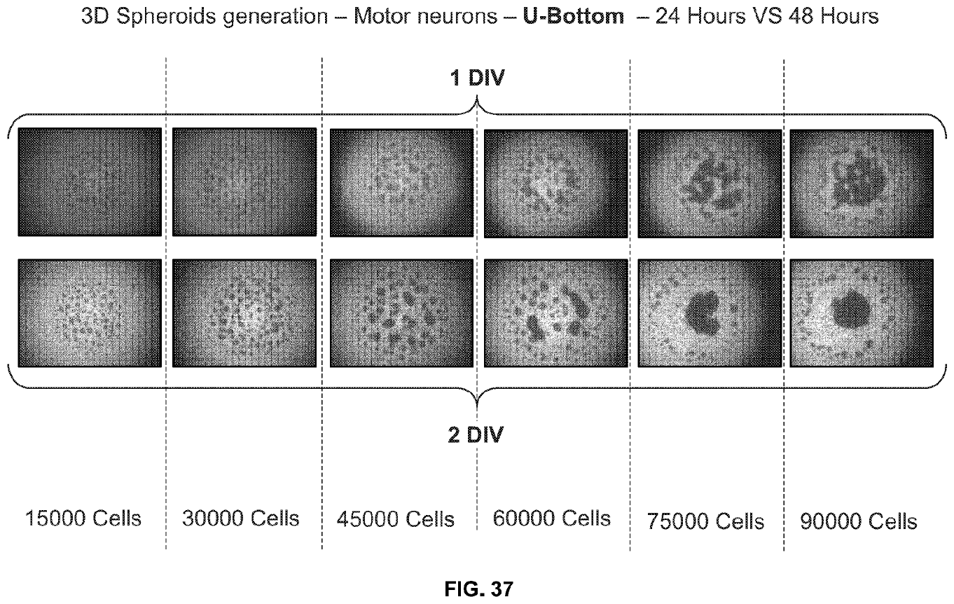

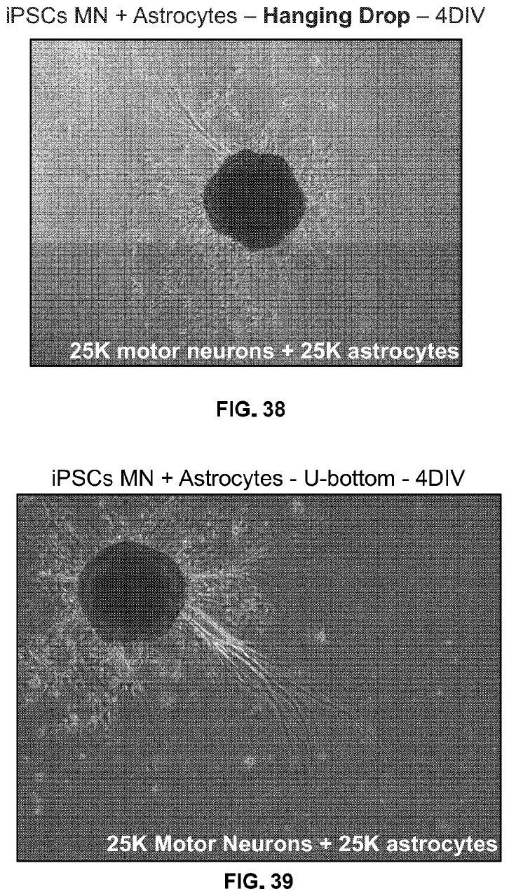

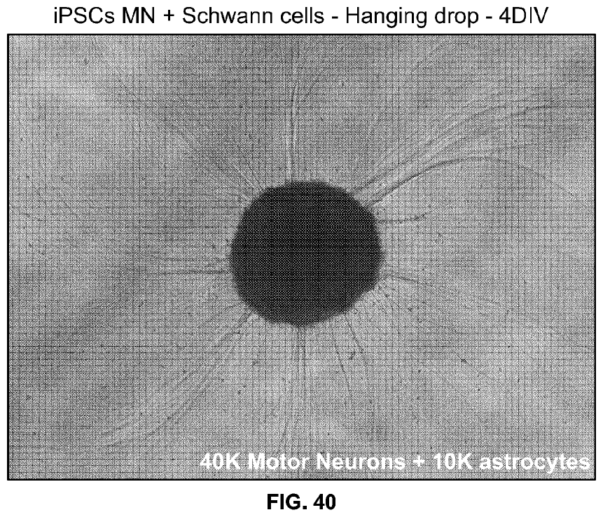

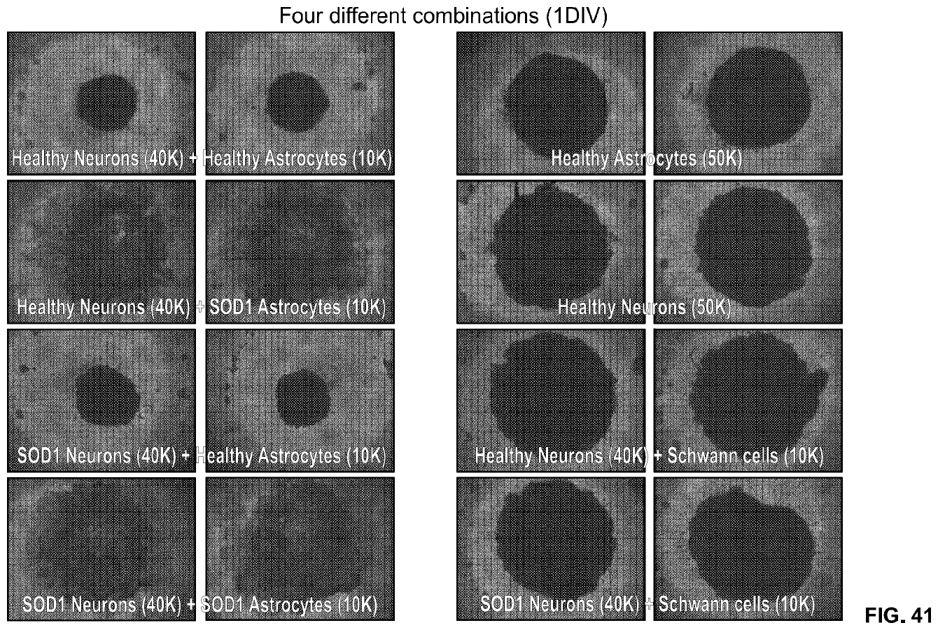

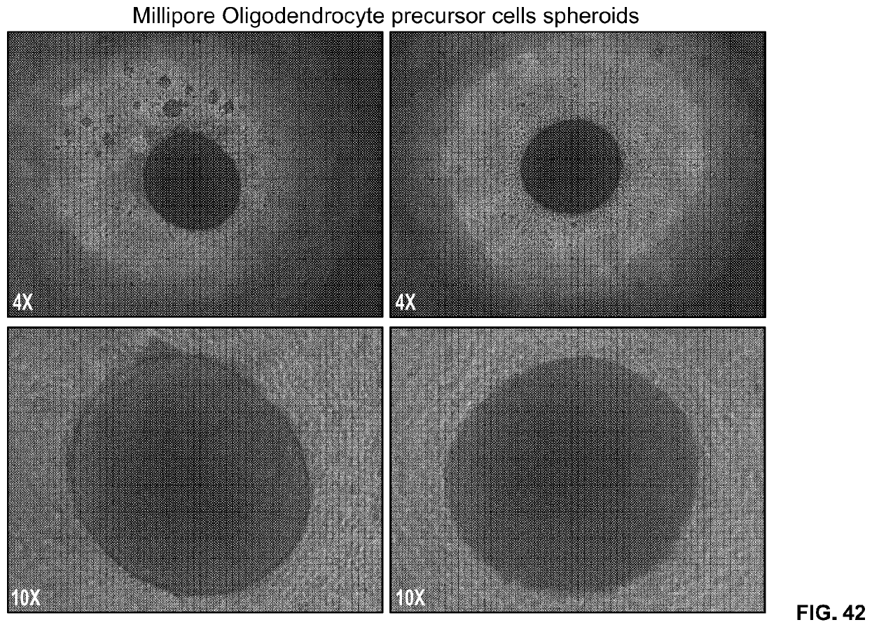

1. Remove 96-well plate from on top of the magnetic drive.
2. Using a 1000 μL pipet (set to 100 μL), gently pipet the fluid in each 96-well to suspend the spheroid.
3. Use a 10 μL pipet (set to 10 μL) to remove the spheroid from the 96-well.
4. Add the spheroid to 2 mL of media in a petri dish on ice.
5. Transfer the spheroids a second time to an empty petri dish on ice. Use 10 μL pipet set to 4 μL. This helps to remove any cell debris transferred from the 96-well.
6. Create a 1:20 dilution of matrigel for cell encapsulation (must account for media added by spheroid transfer) by mixing matrigel with N2:NG media making sure to keep all solutions on ice (below 10° C.) to prevent gelation.
   Ex: For 200 μL of 1:20 matrigel with 4 spheroids, 10 μL of matrigel, 178 μL of N2:NG media, and 12 μL of media is transferred with spheroid. Therefore in this step, 10 μL of matrigel is added to 178 μL of N2:NG media.
7. Add the mixed matrigel and N2:NG media to the spheroids in the empty petri dish.
8. Set a rain-xed glass slide into the magnetic placement device (FIG. 13).
9. Place a Transwell insert containing the PEG constructs on top of the glass slide.
10. Manipulate the magnetic device to align a magnet under the void where you want the spheroid.
11. Using a 10 μL pipet set, transfer the spheroid and 1:20 matrigel solution to the void. Release the spheroid on top of the magnet (FIG. 14B).
12. Repeat steps 10-11 for all constructs in the Transwell insert.
13. Allow the matrigel to gel for 30 minutes at 37° C.
14. Add N2:NG media below the insert.
15. Culture as desired.

### Example 3

### Spheroids for Directional Growth of Neurites

Round-Bottom/U-Bottom Plate:

A non-treated Spheroid microplate, 96 well, with a clear “U” round bottom (Corning REF:4415) was used for either one differentiated cell type, or a combination of differentiated cell types. Resuspended cells are re-counted using a hemocytometer. The density needed for each spheroid, from five thousand, up to one-hundred thousand cells, is added to each well using a micro pipettor accordingly. The Spheroid microplate is then centrifuged at centrifugation speed corresponding to the cell type in suspension for 5 mins, placed in a 37° C. incubator, for 24 hours or more until spheroid formation.

Hanging Drop Plate:

Perfecta3D hanging drop plate from 3D biomatrix was used for spheroid fabrication. A known amount of differentiated induced pluripotent cells derived neurons and glial cells, anywhere from 5,000 to 100,000 were suspended in a small amount of media. Ratios of differentiated cells were altered to enable spheroid formation as well as growth properties after spheroid formation. Cells were suspended in 40 ul volume and was pipetted into the access holes on the top of the plate. Cells were then left for self-assembly in a conventional 5% CO₂ incubator for at least 24 hours to enable spheroid formation.

The Tables below shows different methods of spheroid fabrication:

TABLE 1
Spheroid production

| Method of | | | |
|---|---|---|---|
| fabrication | Cell type | Cell numbers | Hours |
| Hanging drop | CDI Induced neurons | ≥60,000 cells to | 24 hours |
| | | create a sheet like | |
| | | structure | |
| | CDI iCell Motor | ≥75,000 cells | ≥24 hours |
| | neurons | | |
| U-bottom | CDI Induced neurons | ≥30,000 cells to | 24 hours |
| | | create a spheroid | |
| | | like structure | |
| | CDI iCell Motor | ≥50,000 cells | ≥24 hours |
| | neurons | | |
| | Millipore | ≥50,000 cells | ≥24 hours |
| | oligodendrocyte | | |
| | precursor cells | | |
| | (SCR600) | | |
| | Peri.4U sensory | ≥12,500 | For 0-25,000 |
| | neurons | | (>48 hours) |
| | | | For >25,000 |
| | | | (>24 hours) |
| | Axol Sensory | ≥25,000 cells | >24 hours |
| | neurons | | |

TABLE 2
Cell ratios

| Method of | | | |
|---|---|---|---|
| fabrication | Cell type | Cell numbers | Hours |
| U-bottom | CDI ICell Motor | 25000:25000 | ≥24 |
| | Neurons:CDI ICell | Results in shorter | |
| | astrocytes | but thicker neurites | |
| | CDI ICell Motor | 40000:10000 | ≥24 |
| | Neurons:CDI ICell | Results in longer and | |
| | astrocytes | thicker neurites | |
| | CDI ICell Motor | Ratio greater than 10 | ≥24 |
| | neurons:ScienCell | to 1 works. Results | |
| | human SCs | in thinner but much | |
| | | longer neurites. | |
| | | Different ratios of | |
| | | MN (>25,000)/SCs | |
| | | (>25,000) | |
| | | (Ratios tried - 1:1, | |
| | | 3:2, 3:1) (Other | |
| | | ratios are applicable | |
| | | as well) | |
| | CDI MyCell SOD1 | Worked well for | ≥24 |
| | (G93A) motor | ratio 4:1 | |
| | neurons:CDI ICell | | |
| | astrocytes | | |
| | CDI MyCell SOD1 | Worked well for | ≥24 |
| | (G93A) motor | ratio 4:1 | |
| | neurons:ScienCell | | |
| | human SCs | | |

| | | |
|---|---|---|
| 1. | CDI Motor neurons | ~80-90% Neurons |
| | | ~10-20% undifferentiated or |
| | | progenitor cells |
| 2. | Peri.4U neurons | ~90-95% Neurons |
| | | ~5-10% undifferentiated or |
| | | progenitor cells |
| 3. | CDI Motor neurons/ | All kinds of ratios work for |
| | Primary human Schwann cells | fabricating spheroid. |
| | | 25,000:75,000 Schwann |
| | | cells:Neurons worked well for |
| | | myelination. |
| 4. | Human Oligodendrocyte | 70% express the appropriate |
| | precursor cells (Millipore | early-to-intermediate |
| | SCR600) | oligodendrocyte markers. |
| | | Remaining progenitor cells or |
| | | undifferentiated embryonic |
| | | stem cells. |

| | | |
|---|---|---|
| 1. | Matrigel | Elastic modulus ≤ 1000 Pa |
| 2. | Collagen I | Elastic modulus ≤ 10 kPa |
| 3. | Gelatin methacrylate | Elastic modulus ≤ 30 kPa |

### Transferring Spheroids to Nerve-On-a-Chip Construct

To move the spheroid for 3-D construct placement, constructs are dried by removing 500 ul of a total of 1500 ul PBS used within a 6 well, tissue culture treated plate (Transwell, 24 mm diameter, 0.4 um pore size, REF: 3450-Clear) for each well, where the top of the membrane is partially dried for spheroid placement. Matrigel at 8% is then added to inner portion of 3-D construct, then placed in a 37 C incubator for 30 mins.

The spheroids are then taken out, using a p1000 pipettor, and placed in droplets onto a 35 mm tissue culture treated dish (Cell Treat CAT:229635). Spheroids were then placed into the 3D construct “bulb portion” using sterilized Dumont #4 Forceps (Length 11 cm, standard 0.13×0.08 mm Dumostar 11294-00). 1500 ul of Media was then placed under the membrane of the 6 well plate, and placed in a 37 C incubator.

### Example 4

### 3D Muscle Cell Encapsulation Portion of the Neuromuscular Junction (NMJ)

Undifferentiated primary human myoblasts are seeded onto uncoated tissue culture vessels at a vendor specified density, fed serum-containing Growth Media on days 1, 2, 4, and so on to trigger cell division until 60% confluence of is reached. When 60% confluence is reached, cells are passaged with trypsin until Passage 6. Upon reaching P6 and 60% confluence, the primary human muscle myoblast cells are removed from culture vessel with trypsin, centrifuged and re-suspended in media for counting purposes, spun down a second time and re-suspended in DMEM/F12 to a concentration of 8 million cells/mL.

A solution of 5% GelMA, 0.05% LAP solution with added Laminin and n-vinylpyrrolidone is made and mixed with cell suspension such that the cells are at a concentration of 2 million cells/mL. The myoblast/GelMA/LAP solution is pipetted into a specific chamber of a previously fabricated cell-impenetrable polyethylene glycol (PEG) constructs where it is separate from any motor neuron containing chambers. Polymerization of the cell-laden GelMA/LAP containing 2 million cells/mL is achieved by exposing solution in chamber to UV Light.

An alternate method entails directly re-suspending the cells in the GelMA/LAP solution to a concentration of 2 million cells/mL. (Suspension in media and second centrifuge step is omitted).

Differentiation of the myoblasts in three dimensions is achieved by media changes. Days 1-3 entail feeding the construct with the same Growth Media. The media change on Day 4 changes media to a Differentiation Media made of DMEM/F12 and Horse Serum.

Constructs differentiate over the course of up to three weeks as a result of media and paracrine signaling achieved by the method of high-density encapsulation.

Differentiation is confirmed by using histological techniques that include fluorescently marking muscle cells with antibodies for proteins expressed exclusively by multinucleated myotubes including anti-desmin and anti-alpha heavy chain myosin and DAPI or seeing if single cell bodies contain more than two nuclei.

### Example 5: Spheroid Study

In this study, we describe an in vitro, microengineered, biomimetic, all-human peripheral nerve (Human-Nerve-on-a-Chip [HNoaC]) comprised of induced pluripotent stem cell (iPSC)-derived neurons (hNs) and primary human Schwann cells (hSCs) that can provide data suitable for integrated nerve conduction velocity (NCV) and histopathological assessments. This all-human system is an significant extension of our in vitro “Nerve-on-a-chip” (NoaC) platform previously developed using embryonic rat dorsal root ganglion (DRG) neurons and rat SCs ₅. To our knowledge, this combination of hNs and hSCs has not previously been achieved for any other stem cell-based in vitro neural system. This model mimicked robust axonal outgrowth (˜5 mm), showed first evidence ever of human Schwann cell myelination of human iPSC-derived neurons and first ever proof of nerve conduction velocity testing in an all human in vitro system, like in vivo models. Therefore, the innovative HNoaC model of human peripheral nerve has the potential to accelerate the field of human disease modeling, drug discovery, and toxicity screening.

Schwann Cell Culture

A T-75 culture flask (353136; Corning, Corning, N.Y.) was prepared by coating with a sterile-filtered, 0.1% poly-L-ornithine (PLO; Sigma-Aldrich, St. Louis, Mo.) solution in sterile water (Sigma-Aldrich, St. Louis, Mo.). The flask was then washed four times with sterile water. 7.5 mL of a 10 μg/mL Laminin (Sigma-Aldrich, St. Louis, Mo.) in phosphate-buffered saline (PBS; Caisson Labs, Smithfield, Utah) was added to the flask, which was held at 4° C. overnight. The Laminin solution was aspirated, and 15 mL of culture medium was directly placed into the T-75 culture flask, which was then equilibrated in a 37° C. incubator before cell plating. Human Schwann Cells (hSC) medium was purchased from ScienCell (Carlsbad, Calif.). The human Schwann cell line (cat. No. 1700; ScienCell) was received in a cryovial with reportedly more than 5×10₅ cells/mL. The vial was removed from cryopreservation and thawed in a 37° C. water bath. The contents of the vial were dispensed evenly onto the PLO/Laminin-coated T-75 Flask. The culture was left undisturbed at 37° C. in a 5% CO₂ atmosphere for at least 16 hrs to promote attachment and proliferation. Culture medium was changed every 24 hours. Upon reaching 80% confluency, the hSCs were passaged by using 3 mL of Accutase® (Sigma-Aldrich), which was added to the flask for 3 mins at 37° C. Once cells detached completely, 8 mL of hSC medium was placed in the flask. The 11 mL solution of detached hSCs was moved to a 15 mL conical tube and spun at 200×g (Eppendorf 5810 R centrifuge, 18 cm radius; Eppendorf, Hamburg, Germany) for 5 minutes at room temperature (RT, approximately 22° C.). The supernatant was aspirated, and the pellet was resuspended in 1 mL of hSC culture medium. The cells were counted using a conventional hemocytometer (Hausser Scientific, Horsham, Pa.).

Motor Neuron Culture

iCell® Motor Neurons (hNs) medium was prepared using 100 mL of iCell® Neurons Base Medium (FUJIFILM Cellular Dynamics, Inc, Madison, Wis.) supplemented with 2 mL of iCell® Neural Supplement A (FUJIFILM Cellular Dynamics, Inc) and 1 mL of iCell® Nervous System Supplement (FUJIFILM Cellular Dynamics, Inc). To prepare for thawing motor neurons, hNs medium was warmed to RT and 1 mL of hNs medium was added to a sterile 50 mL conical tube. One vial of iCell® Human Motor Neurons (hNs; FUJIFILM Cellular Dynamics, Inc) was thawed in a 37° C. water bath for approximately 2 mins and 30 seconds. The vial contents were transferred to the 50 mL conical tube containing 1 mL of hNs medium, drop-wise with a swirling motion, to mix the cell solution completely and minimize osmotic shock on thawed cells. The cell vial was then rinsed with 1 mL of hNs medium and transferred to the 50 mL tube. The volume of the solution was then brought to 10 mL by slowly adding hNs medium to the 50 mL centrifuge tube dropwise (2-3 drops/sec) while swirling. The cell solution was then transferred to a 15 mL conical tube and centrifuged at 200×g for 5 mins at RT. Supernatant was aspirated, and cells were resuspended in 1 mL of hNs medium by flicking the tube and then pipetting up and down 2-3 times. A 10 μL sample of cell solution then was taken to perform a cell count using a hemocytometer.

Spheroid Fabrication

A non-treated, clear, “U” round bottom, 96 well, spheroid microplate (4515; Corning) was used for both monocultures of human Neurons (hNs) and human Schwann cells (hSCs) as well as co-cultures of hNs/hSCs. The concentration in cells/μL of media was calculated for both hSCs and hNs to permit calculation of the volumes needed to produce spheroids of the following sizes and compositions: hNs mono-cultures—100000, 75000, 50000, or 25000; hSCs mono-cultures—75000, 50000, or 25000; and co-cultures, 75000 hNs with either 75000, 50000 or 25000 hSCs. The volume calculated was added to microwell plates, then the volume of each well was brought to 200 μL by adding media warmed to 37° C. The spheroid microplate was then centrifuged at 200×g for 5 mins and placed in a 37° C. incubator in a 5% CO₂ atmosphere until spheroid formation was observed (typically around 48 h). hNs medium was replenished every other day, at a half changing of 95 μL, and replacing with 100 μL of fresh, warmed (to 37° C.) hNs medium.

Fabrication of 3D Dual-Hydrogel Nerve Growth Constructs

A dual-hydrogel scaffold was created on the membranes of Transwell® inserts (0.4 μm/PES; Corning) using a micro-photolithography technique similar to methods previously described ₆. All solutions were created with sterile-filtered PBS unless otherwise noted. The outer cell-restrictive (i.e., growth-resistant) photo-translinkable hydrogel was created using a solution of polyethylene glycol dimethacrylate 1000 (PEGDMA; Polysciences, Warrington, Pa.) and lithium phenyl-2,4,6-trimethylbenzoylphosphinate (LAP; Sigma Aldrich). First, 10% w/v PEGDMA and 1.1 mM LAP solutions were created and mixed in a 1:1 solution. The resulting solution was sterile-filtered and added to Transwell® inserts placed in a volume of 0.6 mL while positioned under the lens of a Digital Micromirror Device (DMD, PRO4500 Wintech Production Ready Optical Engine; Wintech Digital Systems Technology Corp, Carlsbad, Calif.) on Rain-X (ITW Global Brands, Glenview, Ill.)-treated glass slides (FIG. 1). The mask and polymerization parameters were selected using commercial software (DLP Lightcrafter 4500 Control Software, Texas Instruments, Dallas, Tex.), and irradiation of the photo-translinkable solution occurred for 28-32 seconds using the ultraviolet light of 385 nm wavelength. After treatment, excess PEGDMA/LAP solution was removed from the insert and from within the void created by the photomask. The constructs were then washed using 2% Antibiotic/Antimycotic wash buffer (Thermo Fischer Scientific, Walton, Mass.) three times on the top and bottom of the insert for 10 minutes each. Wash buffer was removed from the insert and the inner keyhole-shaped channels. The void was carefully filled with 8% Growth Factor-Reduced Matrigel® Matrix (Corning) to create a cell-permeable scaffold and allowed to polymerize in a 37° C. incubator.

Transferring Spheroids to Hydrogel Construct

Two types of media were created using hNs medium (described above) to induce myelination in 3D constructs. A Pre-myelination medium was created using hNs medium, 10% of HyClone Characterized Fetal Bovine Serum (FBS; LaCell LLC, New Orleans, La.), and 1% Antibiotic-Antimycotic buffer. A Myelination medium was created with hNs medium, 10% FBS, 10 ng/mL of recombinant rat beta-Nerve Growth Factor (NGF; R&D Systems, Minneapolis, Minn.), and 50 μg/mL of L-ascorbic acid (Sigma-Aldrich). After formation, spheroids were transferred from the microplate using a pipette and placed onto a 35 mm tissue culture-treated dish (Cell Treat, Pepperell, Mass.) in a droplet of hNs medium. Spheroids were then placed into the 3D construct “bulb portion” within the Matrigel, using sterilized Dumont #5 fine-tipped forceps (11295-10; Dumont, Montignez, Switzerland). 1.5 mL of Pre-myelination medium was finally placed under the Transwell® membrane of the 6-well plates, and the loaded hydrogel constructs were placed in a 37° C. incubator in a 5% C0₂ atmosphere for culture. Half-changes of the medium were performed every other day. The constructs were kept in Pre-myelination medium for 1 week before being switched to Myelination medium for 3 weeks.

Immunocytochemistry.

All wells in the 6-well culture plate were fixed with 4% paraformaldehyde (PFA; Electron Microscopy Sciences, Hatfield, Pa.), pH 7.4, for 30 min at RT and then washed with PBS 4 times for 15 minutes each. Fixed samples were then placed in a 1× blocking solution containing PBS; 5% normal goat serum (Jackson ImmunoResearch, West Grove, Pa.); 0.2% Triton-X-100 (Sigma-Aldrich); and 0.4% bovine serum albumin (Sigma-Aldrich) for one hour at RT, followed by labeling with the following primary antibodies overnight in blocking solution at 4° C.: rabbit-α-s100 (ab868, 1:400; Abcam, Cambridge, Mass.); or mouse-α-β111 Tubulin (ab78078, 1:500; Abcam). Rabbit-α-myelin basic protein (MBP, ab133620, 1:500; Abcam) was also used in a separate trial under the same incubation conditions. The following day, wells were washed with PBS 4 times for 8 min each at RT. The plate was then labeled with secondary antibodies, Alexa 488 goat anti-rabbit IgG (1:300, Abcam) or Alexa 568 goat anti-mouse IgG (1:300, Abcam), and DAPI (1:200, Sigma-Aldrich). Secondary antibodies and DAPI was dissolved in 1× blocker solution for 90 minutes in the dark at RT. The plate was washed 5 times, for 8 mins each, with PBS in the dark at RT. The plate was then parafilmed, foiled, and kept at 4° C. until microscopy was performed using a Nikon A1 confocal microscope (Nikon, Tokyo, Japan).

Plastic Resin Embedding.

All materials used for embedding were purchased from Electron Microscopy Sciences unless otherwise noted and were handled under a chemical flow hood and used with recommended personal protective equipment. Hydrogel constructs were removed from culture and washed three times on both sides of the transmembrane well with PBS at RT prior to fixation. The hydrogel constructs were then soaked in a solution of 4% PFA/0.5% glutaraldehyde for 30 minutes at RT. Secondary fixation and staining of cellular lipids was achieved by post-fixation with 1% osmium tetroxide in PBS, pH 7.4, for 2 hours under dark conditions at RT. The constructs were then washed with PBS 3 times for 15 minutes prior to counterstaining with 2% aqueous uranyl acetate for 30 minutes under dark conditions at RT. Dehydration was done with graded ethanol washes at RT, beginning with a 10-minute wash with 50% ethanol/PBS, a 10-minute wash with 70% ethanol/PBS and an overnight wash with 90% ethanol/PBS. The following day, the constructs were washed twice with 100% ethanol for 30 minutes at RT. With a scalpel, hydrogel constructs were dissected individually from the transmembrane wells, without removal of the PEGDMA, under a dissecting microscope. Constructs were placed in Flat Embedding Molds (EMS 70902, Electron Microscopy Sciences). Remaining ethanol was given time to evaporate from the fixed hydrogels before replacement with infiltration medium consisting of a 1:1 mixture of Spurr's resin (Low Viscosity Embedding Media Spurr's Kit; Electron Microscopy Sciences) and propylene oxide. Infiltration medium was left for 75 minutes before it was replaced by 100% Spurr's Resin, which was cured overnight in a 70° C. oven and for 48 more hours at RT before ultramicrotome sectioning.

### Sectioning and Transmission Electron Microscopy (TEM)

Sectioning and TEM evaluation were performed at the Shared Instrumentation Facility (SIF) at Louisiana State University (Baton Rouge, La.). Ultrathin sections were cut to a thickness of 80-100 nm at four locations within the HNoaC specimen: within the bulb of the tissue, where the bulb met the channel and the proximal channel (i.e., near the bulb), and distal channel. Sections were placed on Formvar carbon-coated copper grids, 200 mesh, and impregnated with metal by floating on droplets of 2% uranyl acetate for 20 mins at RT. They were then rinsed with deionized water droplets 3 times, for 1 min. To visualize, a JEOL 1400 TEM (Peabody, Mass.) was used with an accelerating voltage of 120 kV at varying magnifications.

Histomorphometric Analysis

Metrics acquired from TEM images of HNoaC cross sections included axon diameter and G-ratio (i.e., the ratio of the axon diameter to the diameter of the whole fiber [axon+myelin sheath]). Axon diameter and G-ratio were elucidated by two different, independent, blinded researchers by measuring both unmyelinated axons and axons encircled by 3 or more layers of dark myelin wrapping. G-ratio and axon diameter were measured using the scale, threshold and measure functions in Fiji ₇. The G-ratio metrics were calculated by randomly sampling 10 images to find axons with 3 or more myelin laminae, while the unmyelinated fibers were measured by randomly sampling 10 images of axons from the distal channel. Axon diameter was measured by using a thresholding function to find the total area of the axon. The diameter was then calculated from area by assuming the axon was circular. G-ratio calculations were based on a simple linear estimation of inner axon diameter while the outer diameter of the whole fiber (consisting of the axon and surrounding dark-stained myelin lamellae) was calculated by taking an average of the smallest and largest diameter of a given nerve fiber. This averaging technique to obtain the outer diameter was needed because the proximity of the myelin layers was inconsistent along the entire circumference of the myelin sheath. G-ratio was calculated by taking the inner diameter over the average outer diameter. The large nucleated bodies of Schwann cells were excluded when measuring the outer extent of the myelin sheath.

Electrophysiology.

After a month in co-culture, the Transwell® insert with reconstituted nerve was placed on a stage for electrophysiological testing. Two tubes, one for dispensing and the other for aspiration, were placed along the edges of the Transwell® insert for perfusing oxygenated artificial cerebrospinal fluid (ACSF) 5 over tissue samples. For recording compound action potentials (CAP), a pulled glass micropipette electrode (1-4 MΩ) was inserted into the bulb of the channel near the clustered cell bodies, and the axons growing in the channel were stimulated with a concentric bipolar platinum-iridium electrode positioned 1-3 mm distal to the bulb. A platinum recording electrode was placed in the ACSF-filled glass micropipette and was connected to an amplifier set at 100× gain and 0.1 Hz high pass to 3 kHz low pass filtering. Stimulation pulse height and width were kept at 10 volts and 200 μsec, respectively. Samples were stimulated at a maximum repeat rate of 1 Hz, and at least 50 stimulations were applied per sample. Using an analog-to-digital converter (PowerLab; AD Instruments, Colorado Springs, Colo.), CAP waveforms were visualized and further stored using LabChart software (AD instruments). After CAP recording, using a stereomicroscope and camera, a snapshot of the stimulating and recording electrodes were taken to determine the distance between them for nerve conduction velocity (NCV) calculation. Latency was measured by subtracting the location of stimulus artifact by CAP peak location. NCV for myelinated hMN/hSC co-cultures and unmyelinated hMN monocultures was evaluated by dividing the distance between stimulating and recording electrodes by latency.

Statistics

One-way analysis of variance (ANOVA) with Tukey post-hoc test was conducted using GraphPad prism software (GraphPad Software, Inc., La Jolla, Calif., USA) to evaluate size differences between different kind of spheroids. For analysis of electrophysiology, mean and standard deviation were calculated and an unpaired t test was performed (GraphPad Software.) A p-value≤0.05 was used to assign significant differences between the means.

Results

Schwann Cells Enhanced Assembly of Neurons into Spheroids.

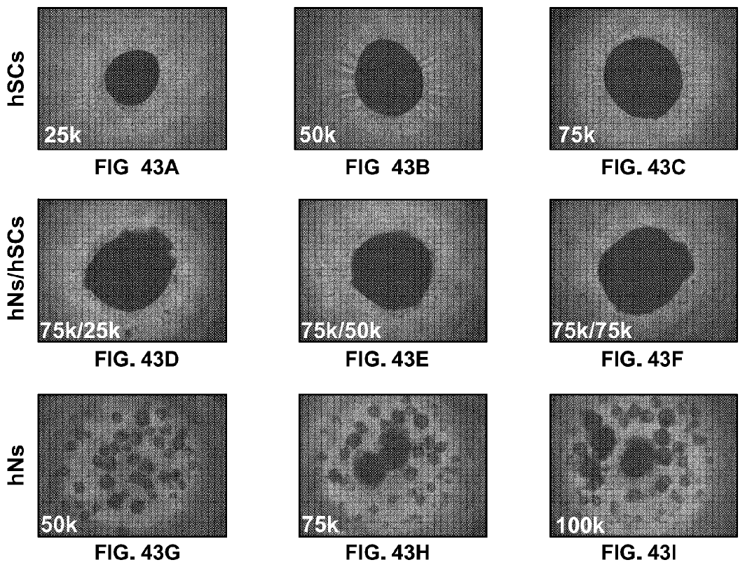

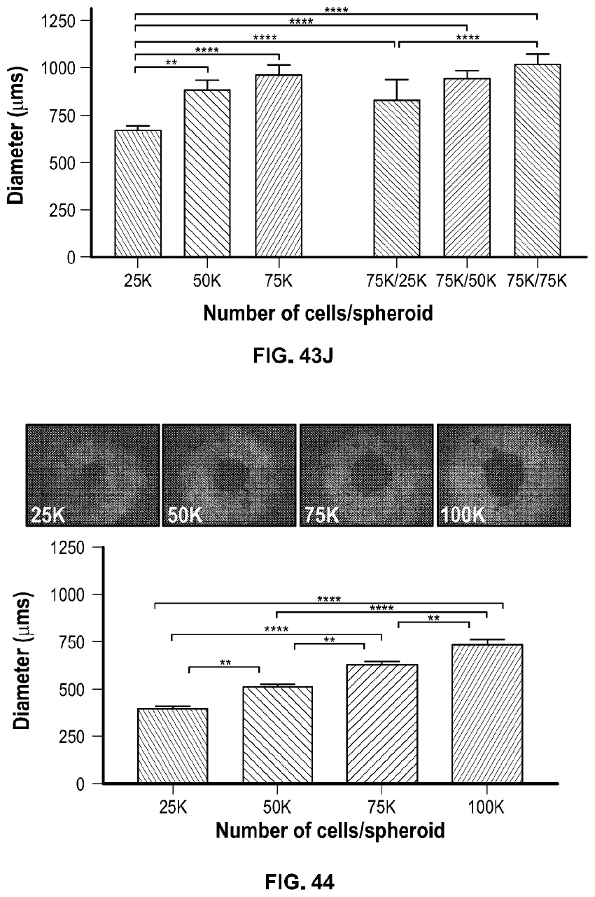

In order to create a spheroid which fits appropriately within the dimensions of the Nerve-on-a-chip (NoaC) system (i.e., less than 1,000 μM in diameter, and maintain a high number of cells), we fabricated spheroids with different cellular densities. We also compared the sizes of different spheroids to understand the interactions between the hNs and hSCs After placing the desired number of cells in low attachment, round-bottom plates, we monitored the formation of spheroids every day. Mono-cultures of hSCs formed spheroids within approximately 2 days and were found to be regular in shape with sharp edges (FIG. 43a-43c). In contrast, mono-cultures of hNs did not self-assemble into spheroids in 2 days and instead formed many smaller spherical structures (FIG. 43g-43i). In co-cultures, hSCs facilitated incorporation of hNs into spheroids (˜2 days) when compared to spheroids formed from hNs alone (˜3-9 days, FIG. 44). The edges of the co-culture spheroids (FIG. 43d-43f) were less sharply defined compared to spheroids containing the hSCs alone possibly due to the non-homogeneous nature of the coculture spheroid. Interestingly, the size of co-culture spheroids comprised of 75,000 hNs and 75,000 hSCs (1025±52 μm) was found to be very similar to the size of 75,000 hSCs alone (967±51 μm), suggesting that co-culture spheroids were more tightly packed and thereby confirming that the two cell types had affinity for each other.

By measuring the diameters of each of the different spheroid types (FIG. 43 and FIG. 44), we determined that having 75,000 neurons is the optimal number of hNs for constituting the hNoaC system as the spheroid size was found to be about 833±108, 948±39 and 1025±52 μm when we created co-culture spheroids with 25,000, 50,000 and 75,000 hSCs respectively. Neuron-only cultures revealed that spheroids increased in size predictably as the number of cells increased. All four hNs conditions (FIG. 44) saw a successive, significant increase in the spheroid size revealing that packing density across the four spheroids (25K, 50K, 75K and 100K) does not vary substantially and that the total number of cells contributes to spheroid size more than the interaction between the various cell types in the spheroid.

Co-Culture Spheroids Showed Robust Neurite Outgrowth in the NoaC System

While the outer portion of the dual hydrogel system was constructed with growth-resistant 10% PEGDMA, the inner part of the channel was filled with fully concentrated (8-12 mg/mL) Matrigel as a growth-promoting substrate. After gel formation, spheroids were gently transferred on top of the bulb part of the channel and left to grow in medium containing 10% FBS but lacking NGF in order to enhance the proliferation and migration of hSCs while delaying neurite extension from hNs. After a week, the incubation solution was switched to medium supplemented with NGF and L-ascorbic acid to facilitate neurite growth and myelination by the hSCs in contact with the growing axons.

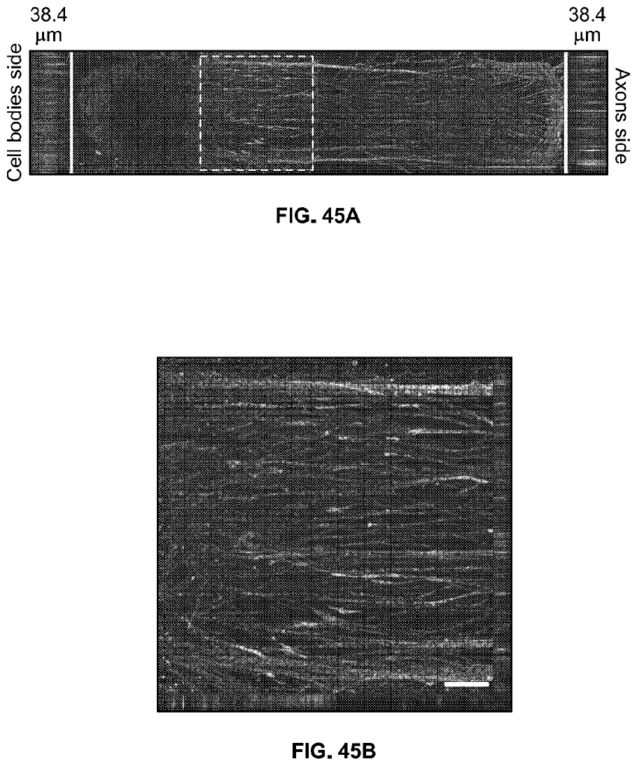

Confocal imaging revealed the 3D nature of the reconstituted in vitro nerves and showed that both cell bodies and axons were present throughout the depth of the channel (FIG. 45). Neurites grew an average of ˜1 mm every week. Addition of FBS was a key factor in optimizing myelination as the basal media with ascorbic acid but without FBS did not support hSC migration and myelination (data not shown). Immunostaining after four weeks with S100 revealed that hSCs cells migrated about 1-1.5 mm outside the spheroid and elongated along the growing axons (FIG. 45A-45C), while the axons reached to the very end of the Matrigel-filled channel (˜5 mm). Interestingly, for many co-culture samples, spheroids influenced the growth of axons such that they appeared to turn back after growing for some distance, possibly because of the chemotactic effect created by growth factors released from hSCs in the spheroid. The effect on axons was more prominent as the number of hSCs in the spheroid increased.

Myelination and Nerve Fiber Structure of In Vitro Human Nerves

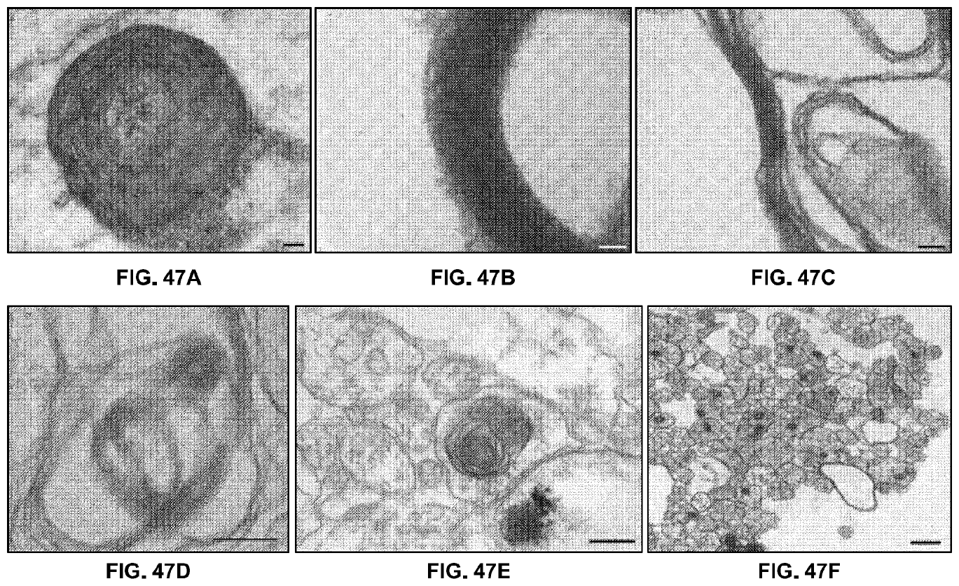

Finally, along with immunostaining and confocal microscopy, we also performed plastic resin embedding and sectioning to evaluate the level and quality of myelination in the system with TEM. Evidence of effective myelination in the system included but was not limited to non-compacted myelin (FIG. 47A), compact myelin (FIG. 47B), and myelin in the process of compacting (FIG. 47C). For the axons, where we saw evidence of myelination, the G-ratio of myelinated nerve fibers was 0.57±0.16. Axonal diameters of the myelinated and unmyelinated axons were 0.55±0.33 and 0.40±0.15 μm, respectively. We also saw evidence of laminar myelin formation without axons (FIG. 5D), the presence of intracytoplasmic lamellar bodies (FIG. 47E), and naked (unmyelinated) axons (FIG. 47F). The appearance of the intracytoplasmic lamellar bodies, which consisted of membrane whorls with relatively regular spacing, was interpreted to represent autophagosome production consistent with recycling of senescent organelles. The distribution of lamellar bodies was sparse and apoptotic nuclei were not observed, indicating that affected cells were not engaged in programmed cell death.

In Vitro Human Nerves Exhibit Effective, Composition-Dependent Electrical Conductivity

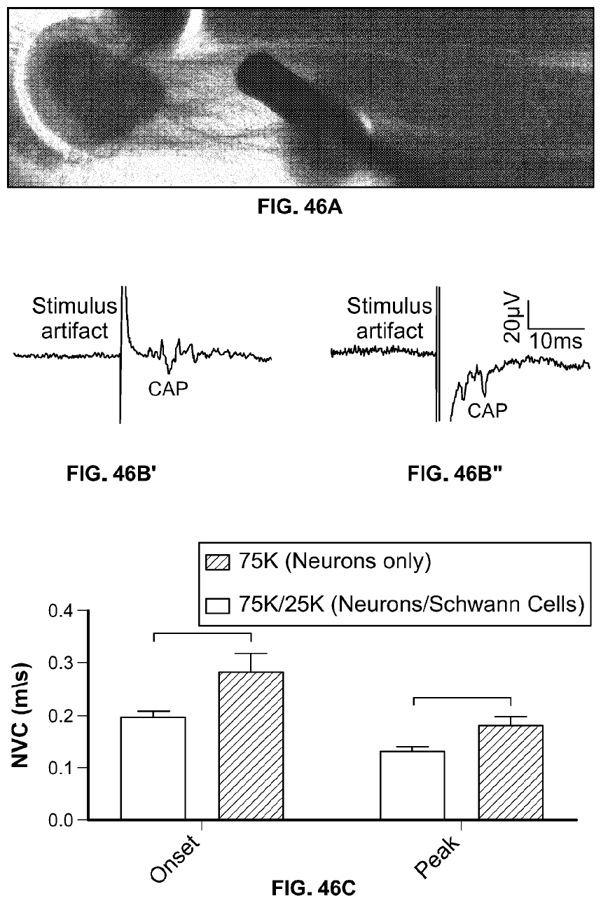

To determine whether we can measure nerve conduction velocity (NCV) of iPSC-derived human neurons (hNs) with or without human Schwann cells (hSCs), we used a technique similar to brain slice electrophysiology. We stimulated the axons inside the channel and recorded the compound action potential (CAP) from the cell bodies (FIG. 46A). Axons were stimulated about ˜1-3 mm away from the cell bodies, and the distance the impulse traveled was calculated between the stimulating and recording electrodes. We evaluated two types of NCV, onset NCV and peak NCV, in order to determine the difference between the fastest signal and the peak signal (FIG. 46B′-46B″). To our surprise, we found the onset and peak NCV with the hN/hSC co-culture samples was slower compared to hN mono-culture samples. Onset NCVs for 75K hNs mono-cultures and 75K/25K hNs/hSCs co-cultures were determined to be 0.28±0.07 and 0.20±0.02 m/s, respectively, while the peak NCVs were found to be 0.18±0.04 and 0.13±0.02 m/s, respectively (FIG. 45C). Onset and peak NCVs were determined with difficulty from samples where the number of SCs were higher (co-cultures of hNs/SCs at 75K/75K and 75K/50K). Qualitative inspection of the samples revealed slightly less dense neurite outgrowth with co-culture samples, which can reduce the NCV.

In this study, we present the first biomimetic, all-human in vitro model of peripheral nerve, assembled as a Nerve-on-a-Chip (NoaC) platform. This microengineered dual hydrogel system retains the neuronal cell bodies in a defined location (i.e, the “ganglion”) and confines dense 3D axonal outgrowth within a narrow channel that extends linearly (i.e., the “nerve”) away from the clustered cell bodies. The system supports current “gold standard” functional (e.g., electrophysiological testing) and structural (e.g., qualitative and quantitative microscopic analyses) endpoints required for assessing neuropathological conditions associated with peripheral neuropathies, which represent a growing medical concern. Innovative aspects of this study include reproducible fabrication of neuron-Schwann cell co-culture spheroids, robust viability (˜4 weeks) and extensive neurite outgrowth (˜5 mm) in vitro, effective myelination of human iPSC-derived neurons (hNs) by primary human Schwann cells (hSCs), and the ability to measure nerve conduction velocity (NCV) in an in vitro setting amenable to human disease modeling, drug discovery, and toxicity screening.

Challenges in Producing In Vitro Nerve Systems.

In vitro myelination using primary hSCs has long been a challenge, due in part to complications associated with extracting hSCs from adult nerves _{8,9}, contamination by fibroblasts ₈₋₁₀ and the transformation of SCs to a proliferative/non-myelinating phenotype in vitro _{11,12}. Co-culture conditions are well established for myelination of rat dorsal root ganglion (DRG) sensory neurons by embryonic, neonatal, and adult rodent SCs ₁₃₋₁₅. However, similar co-culture conditions fail to recapitulate myelination using human SCs cultured with rat DRG neurons ₁₁. Rigorous purification of primary human SCs or differentiation of human stem cells or human fibroblasts to SC-like cells results in limited levels of myelination of rat sensory neurons, but the extent seen in the mixed-species cultures is significantly less compared to that achieved using embryonic rat SCs _{11,16}, possibly due to species differences or density of SCs compared to the number of axons. Recently, Clark and coworkers ₁₇ successfully demonstrated myelination of human stem cell-derived sensory neurons by rat SCs. Still, an in vitro system exhibiting myelination of human iPSC-derived neurons by human Schwann Cells has remained elusive.

In the last few years, many studies focused on creating neuronal-glial organoids to create brain-like tissue in vitro₁₈₋₂₂. Interestingly, all these strategies focused on differentiating the aggregates of neural progenitor cells into more defined neural structures. In contrast, we reverse-engineered the process by bringing together two differentiated cell types to evaluate their interactions with each other and the potential of self-assembly. To mimic the growth of embryonic dorsal root ganglia (DRGs) in vitro, we produced neuron-Schwann cell spheroids using ultra-low attachment, 96-well plates to facilitate crosstalk between axons and SCs, which is important for the differentiation of SCs toward a myelinating phenotype _{23,24}; thus, by bringing axons and SCs close together in a 3D spheroid, we enhanced the chances of cross communication and successful myelination. Following addition of an anti-oxidant, ascorbic acid, we observed the first-ever evidence of myelination in vitro of stem cell-derived human neurons by primary human Schwann cells. Both hNs and hSCs had different rates of self-assembly and spheroid fabrication individually, but when put together hSCs enhanced the quality and improved the speed of spheroid self-assembly as compared to the neuron-only condition. Based on spheroid diameter, coculture spheroids were found to be more compact as compared to either hNs or hSCs spheroids showing the enhanced interaction between the two cells types.

Migration of Schwann Cells Out of the Spheroids.

Schwann cell migration is a critical phenomenon during development and peripheral nerve regeneration following injuries ₂₅. Cues that direct the fate of neural crest cells to Schwann cell precursors and ultimately to Schwann cells are largely unknown; however, it has been known for decades that both precursor cells and Schwann cells rely on growing axons for differentiation, proliferation and functional maturation ₂₆. Here, for the first time, we were able to observe this migration in vitro for tissues of human origin by creating this mini-ganglion comprised of hNs and hSCs. Axons extended outwards along with migrating hSCs which aligned themselves with the growing axons in this process. It was interesting to observe that hSCs only migrated to about ˜1 mm outside the spheroid as compared to total axonal growth of about 5 mm. This could possibly be due to a lower number of hSCs relative to neurons added during these experiments as compared to typical 2D co-culture experiments, where the usual convention is to add >100,000 Schwann cells in a smaller 2D area _{17,27}. This modest extension of hSC compared to axon outgrowth could also be a result of only one week of pre-myelination period and addition of NGF to the media after the first week of growth. NGF has been shown to enhance neuron-Schwann cell interaction and also myelination 28, and thus could be a factor which reduced the migration of SCs. Based on the migration of human SCs outside the spheroid, this HNoaC model can also be used for studying the migration potential of SCs in the presence of therapeutic molecules and thus create possible therapies for patients with peripheral nerve injuries.

hSCs are known to behave differently in vitro as compared to rat Schwann cells in terms of their reactivity to mitogens and growth factors as well as their failure to recapitulate myelination ₂₉. Our 3D spheroid model of human nerve exhibited typical features of nerve trunks observed in nerves acquired during autopsy or biopsy procedures. Axons had a complete complement of organelles including cytoskeletal filaments and mitochondria, and often but not always were associated with sheaths of myelin characterized by closely approximated myelin laminae. The apposition of myelin layers varied among nerve fibers, and in some cases laminar myelin formed in the absence of axons; both these findings are rarely encountered in differentiated nerves harvested in vivo, indicating that some differences in differentiation state do occur (as expected) in culture. That said, sufficient numbers of myelinated axons were observed in the mini-nerve portion of the co-culture system to render it a suitable surrogate for mixed somatic nerves (i.e., those containing densely myelinated, thinly myelinated, and unmyelinated axons).

Evaluation of Nerve Conduction Velocity (NCV)

Different neuropathies are known for showing different kinds of neurophysiological characteristics ₃₀. In vitro microengineered nerve thus should be capable of defining electrophysiological changes as a way of conducting investigative and mechanistic toxicological studies. In this study, which is the first to use human iPSC-derived neurons to study nerve conduction, we were able to see differences in the nerve conduction velocity (NCV) between the myelinated and unmyelinated human axons which shows that this system is sensitive enough to evaluate nerve function. To our surprise, myelinated hNs/hSCs co-culture samples showed a slower NCV as compared to unmyelinated hNs-only mono-culture samples. A qualitative inspection of the culture revealed that the number of hSCs in the spheroid may have reduced the outgrowth and axonal density in the channel of HNoaC, which could easily result in reduction of NCV. Also, because many axons appeared to be turning back in high SCs (50K and 75K) density co-cultures, we were not able to determine the most optimal length between the point of stimulation and point of recording which can impact the NCV calculations. Furthermore, the presence of non-neuronal cell bodies in co-culture spheroids decreases the probability of recording from the appropriately stimulated cell bodies. Importantly, NCV from hNs was found to be considerably lower as compared to the NCV values obtained in human patients₃₁₋₃₃. This is not particularly surprising, considering an in vitro system, at room temperature, comprised of iPSC-derived neurons that are less mature as compared to mature, myelinated axons of nerves assessed in vivo.

The simple design of this fully Human NoaC system opens new avenues in translational research. The platform can be used not only for screening drug candidates on the basis of clinically relevant electrophysiological and histopathological metrics but can also be used for investigating basic mechanisms driving nerve diseases, including but not limited to toxic, demyelinating, and other neurodegenerative conditions. For a given manipulation or treatment, comparison of data acquired from our conceptually identical Rat NoaC₆ and Human NoaC will help close the gap between nonclinical testing and our ability to anticipate responses and potential safety risks in humans.

### REFERENCES

1 Huh, D., Hamilton, G. A. & Ingber, D. E. From 3D cell culture to organs-on-chips. Trends in Cell Biology 21, 745-754, doi:https://doi.org/10.1016/j.tcb.2011.09.005 (2011).
2 Huh, D., Torisawa, Y.-s., Hamilton, G. A., Kim, H. J. & Ingber, D. E. Microengineered physiological biomimicry: Organs-on-Chips. Lab on a chip 12, 2156-2164, doi:10.1039/C2LC40089H (2012).
3 Pankevich, Diana E., Altevogt, Bruce M., Dunlop, J., Gage, Fred H. & Hyman, Steve E. Improving and Accelerating Drug Development for Nervous System Disorders. Neuron 84, 546-553, doi:https://doi.org/10.1016/j.neuron.2014.10.007 (2014).
4 Bespalov, A. et al. Failed trials for central nervous system disorders do not necessarily invalidate preclinical models and drug targets. Nature Reviews Drug Discovery 15, 516, doi:10.1038/nrd.2016.88 https://www.nature.com/articles/nrd.2016.88#supplementary-information (2016).
5 Huval, R. M. et al. Microengineered peripheral nerve-on-a-chip for preclinical physiological testing. Lab on a chip 15, 2221-2232, doi:10.1039/c4lc01513d (2015).
6 Parastoo, K., Ashwin, S., Lauren, A. P., Daniel, W. S. & Michael, J. M. Methods for fabrication and evaluation of a 3D microengineered model of myelinated peripheral nerve. Journal of Neural Engineering 15, 064001 (2018).
7 Schindelin, J. et al. Fiji: an open-source platform for biological-image analysis. Nature methods 9, 676-682, doi:10.1038/nmeth.2019 (2012).
8 Morrissey, T. K., Kleitman, N. & Bunge, R. P. Isolation and functional characterization of Schwann cells derived from adult peripheral nerve. J Neurosci 11, 2433-2442 (1991).
9 Scarpini, E., Kreider, B. Q., Lisak, R. P. & Pleasure, D. E. Establishment of Schwann cell cultures from adult rat peripheral nerves. Experimental Neurology 102, 167-176, doi:https://doi.org/10.1016/0014-4886(88)90090-8 (1988).
10 Scarpini, E. et al. Cultures of human Schwann cells isolated from fetal nerves. Brain Research 440, 261-266, doi:https://doi.org/10.1016/0006-8993(88)90994-8 (1988).
11 Morrissey, T. K., Kleitman, N. & Bunge, R. P. Human Schwann cells in vitro. II. Myelination of sensory axons following extensive purification and heregulin-induced expansion. J Neurobiol 28, 190-201, doi:10.1002/neu.480280206 (1995).
12 Porter, S., Glaser, L. & Bunge, R. P. Release of autocrine growth factor by primary and immortalized Schwann cells. Proceedings of the National Academy of Sciences 84, 7768 (1987).
13 Podratz, J. L., Rodriguez, E. H. & Windebank, A. J. Antioxidants are necessary for myelination of dorsal root ganglion neurons, in vitro. Glia 45, 54-58, doi:doi:10.1002/glia.10302 (2004).
14 Windebank, A. J., Wood, P., Bunge, R. P. & Dyck, P. J. Myelination determines the caliber of dorsal root ganglion neurons in culture. J Neurosci 5, 1563-1569 (1985).
15 Päiväläinen, S. et al. Myelination in mouse dorsal root ganglion/Schwann cell cocultures. Molecular and Cellular Neuroscience 37, 568-578, doi:https://doi.org/10.1016/j.mcn.2007.12.005 (2008).
16 Lehmann, H. C. et al. Human Schwann cells retain essential phenotype characteristics after immortalization. Stem Cells Dev 21, 423-431, doi:10.1089/scd.2010.0513 (2012).
17 Clark, A. J. et al. Co-cultures with stem cell-derived human sensory neurons reveal regulators of peripheral myelination. Brain 140, 898-913, doi:10.1093/brain/awx012 (2017).
18 Koito, H. & Li, J. Preparation of Rat Brain Aggregate Cultures for Neuron and Glia Development Studies. Journal of Visualized Experiments: JoVE, 1304, doi:10.3791/1304 (2009).
19 Reynolds, B. A. & Weiss, S. Generation of neurons and astrocytes from isolated cells of the adult mammalian central nervous system. Science (New York, N.Y.) 255, 1707-1710 (1992).
20 Paca, A. M. et al. Functional cortical neurons and astrocytes from human pluripotent stem cells in 3D culture. Nature methods 12, 671-678, doi:10.1038/nmeth.3415 (2015).
21 Bae, B. I. & Walsh, C. A. Neuroscience. What are mini-brains? Science (New York, N.Y.) 342, 200-201, doi:10.1126/science.1245812 (2013).
22 Lancaster, M. A. et al. Cerebral organoids model human brain development and microcephaly. Nature 501, 373-379, doi:10.1038/nature12517 (2013).
23 Sulaiman, O. A. R. & Gordon, T. Effects of short- and long-term Schwann cell denervation on peripheral nerve regeneration, myelination, and size. Glia 32, 234-246, doi:doi:10.1002/1098-1136(200012)32:3<234::AID-GLIA40>3.0.CO;2-3 (2000).
24 Salzer, J. L. Schwann Cell Myelination. Cold Spring Harbor Perspectives in Biology 7, doi:10.1101/cshperspect.a020529 (2015).
25 Anton, E. S., Hadjiargyrou, M., Patterson, P. H. & Matthew, W. D. CD9 plays a role in Schwann cell migration in vitro. The Journal of Neuroscience 15, 584 (1995).
26 Bhattacharyya, A., Brackenbury, R. & Ratner, N. Axons arrest the migration of Schwann cell precursors. Development (Cambridge, England) 120, 1411-1420 (1994).
27 Zanazzi, G. et al. Glial Growth Factor/Neuregulin Inhibits Schwann Cell Myelination and Induces Demyelination. The Journal of Cell Biology 152, 1289 (2001).
28 Chan, J. R. et al. NGF Controls Axonal Receptivity to Myelination by Schwann Cells or Oligodendrocytes. Neuron 43, 183-191, doi:https://doi.org/10.1016/j.neuron.2004.06.024 (2004).
29 Monje, P. V., Sant, D. & Wang, G. Phenotypic and Functional Characteristics of Human Schwann Cells as Revealed by Cell-Based Assays and RNA-SEQ. Molecular Neurobiology 55, 6637-6660, doi:10.1007/s12035-017-0837-3 (2018).
30 Fuller, G. How to get the most out of nerve conduction studies and electromyography. Journal of Neurology, Neurosurgery &amp;amp; Psychiatry 76, ii41 (2005).
31 Buchthal, F. & Rosenfalck, A. Evoked action potentials and conduction velocity in human sensory nerves. Brain Research 3, v-122, doi:https://doi.org/10.1016/0006-8993(66)90056-4 (1966).
32 Palve, S. S. & Palve, S. B. Impact of Aging on Nerve Conduction Velocities and Late Responses in Healthy Individuals. Journal of Neurosciences in Rural Practice 9, 112-116, doi:10.4103/jnrp.jnrp_323_17 (2018).
33 Mallik, A. & Weir, A. I. Nerve conduction studies: essentials and pitfalls in practice. Journal of Neurology, Neurosurgery &amp;amp; Psychiatry 76, ii23 (2005).

### Example 6: Sensory Synapse Model

Co-culture of rat dorsal root ganglion (DRG) neurons with cells of the dorsal horn (DH) of the rat spinal cord has been shown previously (Ohshiro et al., 2007; Vikman et al., 2001). When cultured together, the DRG neurons synapse onto the dorsal horn cells. The development of this rat DRG to DH synapse model in a 3D format will be the first step towards the development of the human spinal cord DH afferent sensory synapse model.

The key to this experiment is for the DRG neurons to extend axons through GelMA and synapse on the DH neurons. Previous experiments in the lab have shown that spinal cord spheroid growth can be controlled by the stiffness of gels and do not grow well in GelMa. Using this characteristic, we aim to create a mono-directional neuronal circuit, where DH axons do not grow into the GelMA, but do grow throughout the Matrigel, where the compound action potentials (CAPs) can be recorded.

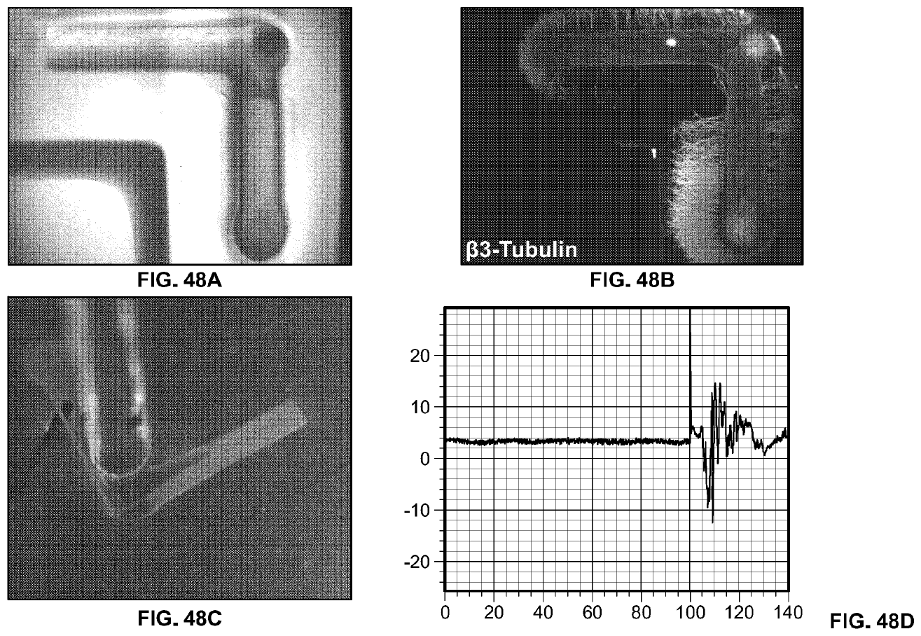

The dorsal horns and DRGs were isolated from embryonic day 15 rat spinal cords and dissociated. Cells were then cultured separately, in spheroid cultures in a 96 well U-bottom plate. Two days after plating, spheroids formed and were then placed into dual hydrogel constructs to allow for neuronal growth in 3D (FIG. 48A). DRGs were placed in the bottom key-hole of the construct in GelMA, while the DH spheroids were placed in the middle key-hole into Matrigel. Staining this culture with β-tubulin confirmed axonal growth from both spheroids in culture at 28 DIV (FIG. 48B). CAP recordings were taken at the DH spheroid, while the stimulating electrode was placed on the DRG axon. This distance was measured to be 3.1 mm between the electrodes for this culture (FIG. 48C). Example of trace recordings of CAPs showed a response at the DH spheroid when the DRG axons were stimulated (FIG. 48D). This suggests that DRGs are synapsing on and activating DH neurons, which the CAP responses were recorded from. Future experiments will be focused on confirming the formation of these synapses and converting this model into a human format.
Ohshiro H, Ogawa S, and Shinjo K. Visualizing sensory transmission between dorsal root ganglion and dorsal horn neurons in co-culture with calcium imaging. J Neurosci Methods, 2007, 165: 49-54.
Vikman K, Backstrom E, Kristensson K, and Hill R. A two-compartment in vitro model for studies of modulation of nociceptive transmission. J Neurosci Methods 2001; 105:175-184.

1. A composition comprising a spheroid of cells comprising one or a combination of cells and/or tissues chosen from: a neuronal cell, nervous system ganglia, a stem cell, an immune cell, dorsal root ganglia, and trigeminal ganglia.

2. (canceled)

3. The composition of claim 1, wherein the spheroid further comprises one or a plurality of cells chosen from: a glial cell, an embryonic cell, a mesenchymal stem cell, a cell derived from an induced pluripotent stem cell, a sympathetic neuron, a parasympathetic neuron, a spinal motor neurons, a central nervous system neuron, a peripheral nervous system neuron, an enteric nervous system neurons, a motor neuron, a sensory neuron, a cholinergic neuron, a GABAergic neuron, a glutamatergic neuron, a dopaminergic neuron, a serotonergic neuron, an interneuron, an adrenergic neuron, a trigeminal ganglion, an astrocyte, an oligodendrocyte, a Schwann cell, a microglial cell, an ependymal cell, a radial glial cell, a satellite cell, an enteric glial cell, a pituicyte, an embryonic stem cell, an induced pluripotent stem cell, a T cell, a B cell, a macrophage, and combinations thereof.

4-5. (canceled)

6. The composition of claim 1, wherein the neuronal cell is derived from a stem cell chosen from: an embryonic stem cell, a mesenchymal stem cell, and an induced pluripotent stem cell and/or any one or plurality of cells are differentiated from induced pluripotent stem cells.

7. The composition of claim 1, wherein the spheroid;
(a) has a diameter from about 200 microns to about 700 microns; and/or
(b) comprises no less than about 30.000 cells.

8. The composition of claim 1, wherein the spheroid comprises;
(a) Lone or a plurality of neuronal cells and one or a plurality of Schwann cells at a ratio of cell types equal to about 4 neuronal cells for every 1 Schwann cell;
(b) one or a plurality of neuronal cells and one or a plurality of astrocytes at a ratio of about 4 neuronal cells for every 1 astrocyte;
(c) one or a plurality of neuronal cells and one or a plurality of astrocytes at a ratio of about 1 neuronal cell for every 1 astrocyte;
(d) one or a plurality of neuronal cells and one or a plurality of Schwann cells at a ratio of about 10 neuronal cells for every 1 Schwann cell; and/or
(e) one or a plurality of neuronal cells and one or a plurality of glial cells at a ratio equal to about 4 neuronal cells for every 1 glial cell.

9-13. (canceled)

14. The composition of claim 1, wherein the spheroid is free of induced pluripotent stem cells, undifferentiated stem cells and/or immune cells.

15-18. (canceled)

19. A system comprising:
(i) a cell culture vessel comprising a hydrogel;
(ii) one or a plurality of spheroids comprising one or plurality of neuronal cells and/or isolated tissue explants;
(iii) an amplifier comprising a generator for electrical current;
(iv) a voltmeter and/or ammeter; and
(v) at least a first stimulating electrode and at least a first recording electrode;
wherein the amplifier, voltmeter and/or ammeter, and electrodes are electrically connected to the each other via a circuit in which electrical current is fed to the at least one stimulating electrode from the amplifier and electrical current is received at the recording electrode and fed to the voltmeter and/or ammeter; wherein the stimulating electrode is positioned at or proximate to one or a plurality of soma of the neuronal cells and/or isolated tissue explants and the recording electrode is positioned at a predetermined distance distal to the soma, such that an electrical field is established across the cell culture vessel.

20-21. (canceled)

22. The system of claim 19, wherein the culture vessel comprises 96, 192, 384 or more interior chambers, wherein the 96, 192, 384 or more interior chambers.

23. (canceled)

24. The system of claim 19, further comprising a solid substrate onto which the hydrogel matrix is crosslinked, said solid substrate comprising at least one plastic surface with pores from about 1 micron to about 5 microns in diameter, and optionally:
(a) wherein the solid substrate comprises a contiguous exterior surface and an interior surface, such solid substrate comprising at least one portion in a cylindrical or substantially cylindrical shape and at least one hollow interior defined at its edge by at least one portion of the interior surface, said interior surface comprising one or a plurality of pores from about 0.1 microns to about 1.0 microns in diameter wherein the hollow interior of the solid substrate is accessible from a point exterior to the solid substrate through at least one opening; wherein the hollow interior portion comprises a first portion proximate to the opening and at least a second portion distal to the opening; wherein the one or plurality of neuronal cells and/or the one or plurality of tissue explants are positioned at or proximate to the first portion of the hollow interior and are in physical contact with the hydrogel matrix, and wherein the second portion of the at least one hollow interior is in fluid communication with the first portion such that axons are capable of growth from the one or plurality of neuronal cells and/or the one or plurality of tissue explants into the second interior portion of the hollow interior; and/or
(b) wherein at least one portion of the solid substrate is cylindrical or substantially cylindrical such that at least one portion of the interior surface of the solid substrate defines a cylindrical or substantially cylindrical hollow interior chamber in which the spheroids are positioned.

25. (canceled)

26. The system of claim 19, wherein the hydrogel comprises:
(a) at least a first cell-impenetrable polymer and a first cell-penetrable polymer, wherein the at least one cell-impenetrable polymer optionally comprises no greater than about 15% PEG and the at least one cell-penetrable polymer optionally comprises from about 0.05% to about 1.00% of one or a combination of self-assembling peptides chosen from: RAD 16-I, RAD 16-II, EAK 16-I, EAK 16-II, and dEAK 16;

27-29. (canceled)

30. The system of claim 19 further comprising;
(a) a cell medium comprising nerve growth factor (NGF) at a concentration from about 5 to about 20 picograms per milliliter and/or ascorbic acid in a concentration ranging from about 0.001% weight by volume to about 0.01% weight by volume; and/or
(b) one or a plurality of stem cells, pluripotent cells, myoblasts and osteoblasts.

31. The system of claim 19, wherein the one or plurality of spheroids;
(a) comprises at least one or a combination of cells chosen from: a glial cell, an embryonic cell, a mesenchymal stem cell, a cell derived from an induced pluripotent stem cell, a sympathetic neuron, a parasympathetic neuron, a spinal motor neurons, a central nervous system neuron, a peripheral nervous system neuron, an enteric nervous system neuron, a motor neuron, a sensory neuron, a cholinergic neuron, a GABAergic neuron, a glutamatergic neuron, a dopaminergic neuron, a serotonergic neuron, an interneuron, an adrenergic neuron, a trigeminal ganglion neuron, an astrocyte, an oligodendrocyte, a Schwann cell, a microglial cell, an ependymal cell, a radial glial cell, a satellite cell, an enteric glial cell, and a pituicyte;
(b) are in culture for no less than about 3, 30, 90, or 365 days; and/or
(c) comprises one or a plurality of neuronal cells with axonal growth from about 100 microns to about 500 microns in width and from about 0.11 to about 10.000 microns in length.

32-39. (canceled)

40. The system of claim 26, wherein the three-dimensional axon is at least about 10 microns in height at its lowest point or is at least three cellular monolayers in height, and wherein the cavities are wells with a U-shaped or rounded wells positioned in a horizontal or substantially horizontal plane of the solid substrate with each channel comprising one or a plurality of axons connecting the one or plurality of spheroids.

41. The system of claim 40, wherein the system comprises:
(a) a first spheroid comprising:
(i) one or a plurality of neuronal cells; and/or
(ii) one or a plurality of Schwann cells or oligodendrocytes; and
a second spheroid comprising:
(i) one or a plurality of peripheral neurons;
wherein each spheroid is positioned in the cavity; or
(b) a first, second and third cavity each configured to hold a spheroid and at least 50 microliters of cell culture medium, wherein the cavities are aligned such that the first cavity is positioned proximal to the second cavity and distal to the third cavity.

42-44. (canceled)

45. A method of manufacturing a three-dimensional culture of one or a plurality of spheroids in a culture vessel comprising a solid substrate, said method comprising:
(a) contacting one or a plurality of neuronal cells with the solid substrate, said substrate comprising at least one exterior surface, at least one interior surface and at least one interior chamber defined by the at least one interior surface and accessible from a point exterior to the solid substrate through at least one opening;
(b) positioning one or a plurality of spheroids comprising neuronal cells and/or tissue explants selected from one or a combination of: an isolated dorsal root ganglion, a spinal cord explant, a retinal explant, and a cortical explant to the at least one interior chamber; and
(c) applying a cell medium into the culture vessel with a volume of cell medium sufficient to cover the at least one spheroid; wherein at least one portion of the interior surface comprises a first cell-impenetrable polymer and a first cell-penetrable polymer.

46. The method of claim 45 further comprising:
(d) allowing the spheroids to grow neurites and/or axons after step (c) for a period of from about 12 hours to about 1 year.

47. The method of claim 45, wherein the spheroids are formed as a suspension of neuronal cells selected from one or a combination of: motor neurons, sensory neurons, sympathetic neurons, parasympathetic neurons, cortical neurons, spinal cord neurons, peripheral neurons, optionally derived from a stem cell.

48-49. (canceled)

50. The method of claim 45, further comprising the step of:
positioning at least one stimulating electrode at or proximate to soma of the one or plurality of neuronal cells or tissue explants and positioning at least one recording electrode at or proximate to an axon at a point most distal from the soma, such that, upon introducing a current in the stimulating electrode, the recording electrode is capable of receiving a signal corresponding to one or a plurality of electrophysiological metrics capable of being measured at the recording electrode, wherein the one or plurality of electrophysiological metrics are one or a combination of: electrical conduction velocity, action potential, amplitude of the wave associated with passage of an electrical impulse along a membrane of one or a plurality of neuronal cells, a width of an electrical impulses along a membrane of one or a plurality of neuronal cells, latency of the electrical impulse along a membrane of one or a plurality of neuronal cells, and envelope of the electrical impulse along a membrane of one or a plurality of neuronal cells.

51. (canceled)

52. A method of evaluating the toxicity and/or neuroprotective effects of an agent comprising:
(a) culturing one or more spheroids in the composition of claim 1;
(b) exposing at least one agent to the one or more spheroids; and
(c) measuring and/or observing one or more morphometric changes and/or one or more electrophysiological metrics of the one or more spheroids;
and optionally, the method further comprises correlating one or more morphometric changes and/or one or more electrophysiological metrics of the one or more spheroids with the toxicity of the agent, such that, if the morphometric changes and/or electrophysiological metrics are indicative of decreased cell viability, the agent is characterized as toxic and, if the morphometric changes and/or electrophysiological metrics are indicative of unchanged or increased cell viability, the agent is characterized as non-toxic and/or neuroprotective.

53. A method of measuring myelination or demyelination of one or more axons of one or a plurality of spheroids, said method comprising:
(a) culturing one or more spheroids in the composition of claim 1 in the presence or absence of an agent for a time and under conditions sufficient to grow at least one axon; and
(b) detecting the amount of myelination on one or a plurality of axons from the one or more spheroids; wherein detecting optionally comprises the steps of:
(i) measuring and/or observing one or more morphometric changes and/or one or more electrophysiological metrics of the one or more spheroids in the presence or absence of an agent; and
(ii) correlating one or more morphometric changes and/or one or more electrophysiological metrics of the one or more spheroids in the presence or absence of an agent with a quantitative or qualitative change of myelination of the spheroids.

54. A method of detecting and/or quantifying neuronal cell growth and/or axon degeneration comprising:
(a) quantifying one or a plurality of spheroids and/or number or density of axons grown from spheroids;
(b) culturing the one or more spheroids in the composition of claim 1; and
(c) calculating the number of cells within the spheroid and/or number or density of axons grown from spheroids in the composition after culturing the spheroids for a time period sufficient to allow growth of the one or plurality axons or of growth of cells in the spheroid;
wherein step (b) optionally comprises contacting the one or more spheroids with one or more agents; wherein step (c) optionally comprises detecting an internal and/or external recording of such one or more spheroids after culturing one or more spheroids and correlating the recording with a measurement of the same recording corresponding to a known or control number of cells; or
wherein step (c) optionally comprises the additional steps of: (i) measuring an intracellular and/or extracellular recording and/or a morphometric change before and after the step of contacting the one or more spheroids to one or more agents; and (ii) correlating the difference in the recordings and/or morphometric changes before contacting the one or more spheroids to the one or more agents to the recordings and/or morphometric changes after contacting the one or more spheroids to the one or more agents to a change in cell number and/or number or density of axons.

55. A method of measuring or quantifying a neuromodulatory effect of an agent comprising:
(a) culturing one or a plurality of spheroids in a composition of claim 1 in the presence and absence of the agent;
(b) applying a voltage potential across the one or a plurality of spheroids in the presence and absence of the agent;
(c) measuring one or a plurality of electrophysiological metrics from the one or plurality of spheroids in the presence and absence of the agent; and
(d) correlating the difference in one or a plurality of electrophysiological metrics through the one or plurality of spheroids to the neuromodulatory effect of the agent, such that a change in electrophysiological metrics in the presence of the agent as compared to the electrophysiological metrics measured in the absence of the agent is indicative of a neuromodulatory effect, and no change of electrophysiological metrics in the presence of the agent as compared to the electrophysiological metrics measured in the absence of the agent is indicative of the agent not conferring a neuromodulatory effect;
or
(a) culturing one or a plurality of spheroids in a composition of claim 1 in the presence and absence of the agent;
(b) measuring and/or observing one or more morphometric changes of the one or plurality of spheroids in the presence and absence of the agent; and
(c) correlating the one or more morphometric changes with the neuromodulatory effect of the agent, such that a change in morphometrics in the presence of the agent as compared to the morphometrics measured and/or observed in the absence of the agent is indicative of a neuromodulatory effect, and no change of morphometrics in the presence of the agent as compared to the morphometrics measured and/or observed in the absence of the agent is indicative of the agent not conferring a neuromodulatory effect.

56. A method of making the system of claim 19, the method comprising:
(a) culturing neuronal cells in a cell culture medium for a time period sufficient for the cells to form a spheroid;
(b) positioning the spheroid within the hydrogel, wherein the spheroid is optionally positioned within a cavity of the hydrogel using a magnetic force, a sonic force, mechanical force, or fluidic force; and
(c) exposing the spheroid to a cell culture medium for a time period sufficient to grow a neurite or axon.

57-61. (canceled)

62. The system of claim 19 further comprising: on or a combination of
(i) a first region and a second region, the first region is formed in the shape of a cylinder or rectangular prism oriented with its longitudinal axis passing through the top and bottom of the cell culture vessel and each of either the cylinder or rectangular prism comprising a space defined by an inner surface of the cylinder or rectangular prism, said space and accessible by one or more openings through the top of the cell culture vessel; wherein the second region comprises a space formed in the shape of its interior walls with an opening on its side adjacent to and in fluid communication with the first region;
(ii) at least 1% polyethylene glycol (PEG); and/or
(iii) a series of two or more cavities in fluid communication with each other by a series of channels, at least one cavity comprising a spheroid and at least a second cavity comprising a second spheroid, suspension of cells, or DRG; wherein the spheroid and the second spheroid, suspension of cells, or DRG is connected by a three-dimensional axon.


SEQUENCE LISTING (28 sequences)

SEQ ID NO: 1 - DNA, 220 bp, Homo sapiens
gtttggggccagagtgggcgagacgcggaggtctggcctataaagtagtcgcggagacggggtgctggtttgcgtcgtagtctcctgcagcgtctggggtttccgttgcagtcctcggaaccaggacctcggcgtggcctagcgagttatggcgacgaaggccgtgtgcgtgctgaagggcgacggcccagtgcagggcatcatcaatttcgagcagaag

SEQ ID NO: 2 - DNA, 6394 bp, Homo sapiens
ggacccactgggttgccaagctcgcgccggatgcggagcgcggtgctgccggtggagcttcaggtcttgatagactttctgtaaagaaggaatgatttggtgatggagtgttcccactgaccgatggactcaaagaagagaagctcaacagaggcagaaggatccaaggaaagaggcctggtccatatctggcaggcaggatcctttcccataacaccagagagattgccaggctggggaggaaagactgttttgcaggcagccctcggagtgaaacatggagttcttctgactgaagatggtgaggtctacagctttgggactcttctctggagaagtggaccagtggagatttgtccaagtagccccattctagaaaatgccctggttgggcaatatgttattactgtggcaacaggaagcttccatagtggagcagtgacagacaatggtgtcgcgtacatgtggggagagaattctgctggccagtgtgcagtagccaaccagcagtatgtgccggaaccaaatcctgtcagcattgctgattctgaggccagccctttgttagcagtcaggattttacagttggcgtgtggcgaggagcacactctggcattgtcaataagcagagagatttgggcatggggtaccggttgtcagttgggtctcattaccactgccttcccagtgacaaagccgcaaaaggtagaacatcttgctgggcgagtggtgcttcaagttgcctgtggtgctttccacagcttagcccttgtacaatgcctcccttcccaggatctgaagccagtcccagaacgatgcaaccagtgcagccagctcttgattactatgactgacaaagaagaccatgtgattatatcagacagtcattgttgcccattaggtgtgacactgacagaatctcaggcagaaaaccatgccagcactgctctcagcccctccactgaaacccttgacaggcaggaagaagtatttgagaacactcttgtagcaaatgatcagtctgttgctactgaactgaatgcagtaagtgctcagatcacaagcagcgatgccatgtcctctcaacaaaatgtcatgggaacaactgaaatttcctctgccagaaacataccatcataccctgacacccaagcagtcaatgaatacctacggaaactgtcagatcattcagtaagagaggactcagagcatggtgaaaagccagtgccatctcagcctcttttagaagaagcaattcctaatctccacagcccgcctaccacaagcacctcagccctaaacagcctggtggtctcttgtgcatctgctgttggtgtgagagtggctgctacttatgaagctggtgccttgtcactgaagaaagttatgaacttttatagtacaaccccttgtgaaactggagctcaggcaggcagtagtgccattggccccgaaggtttgaaagatagcagggaagaacaggttaaacaggaatcaatgcaaggaaagaaaagttcaagtcttgtggatatcagagaagaagaaacagagggaggcagtcgaagactctccctccctggattgttgtcacaagtttcccccaggctcttaagaaaggctgcacgggtgaaaacgaggacagtggttctgacccccacatacagtggagaagcagatgcgctcctgccttctctgagaacagaagtgtggacctgggggaaagggaaggaagggcagctggggcacggcgatgttctgcctaggcttcaaccgttgtgtgtaaaatgtctggatggcaaagaagtaatccatctggaggcaggtggttaccattctcttgcacttactgcgaaatcccaggtttactcatggggtagcaatacctttggtcaacttgggcattccgattttccaacaacagttcctcgtcttgcaaagataagcagtgaaaatggagtctggagcatagctgcaggcagggattattccctgtttttagtggatacagaagacttccagcctgggttatattacagtggccgacaggaccctacagaaggtgacaaccttccagagaatcacagtggttctaagactccagtacttctctcctgtagtaagcttggatatataagcagagtgacagcaggaaaagatagctatttagccttggtggataaaaacattatggggtatattgccagtctccacgagttagctactacagaaagacgattctattcaaaactaagtgatatcaaatctcagattctcaggcctcttctcagtttagaaaatttgggcactacaactacagtccagctgttgcaggaggtggctagccgattcagcaagctgtgttacctcattggtcagcatggagcctcattgagcagcttccttcatggggtaaaggaagccaggagtttggtcatcctgaagcattcaagtctcttcttggatagttatacagagtattgcacatctattacaaatttcctggttatgggaggattccagcttcttgctaagcctgccattgatttcctaaataaaaaccaagagctgttgcaagatttgtcagaagtgaatgacgaaaacactcagttgatggaaatactgaatactttgtttttcttgccaatcagacgacttcataattacgcaaaagttttgctaaagcttgctacttgttttgaagtggcatctccagaatatcagaaactgcaggattccagttcttgttatgagtgtcttgctctccatctcggcaggaaaaggaaggaagcagaatacacactgggcttctggaagaccttccccggaaaaatgacggattccttgaggaagccagagcgtcgactgctgtgtgagagtagtaaccgagccctgtctctgcagcatgctgggaggttttccgtgaattggttcattctctttaatgatgccctggtccatgcccagttctccacgcaccatgttttccctctggccacgctgtgggcagagccactgtctgaagaagctggtggtgtgaatggcttaaagataactacacctgaggagcagttcactctcatttcatctacaccccaggaaaagacaaagtggctacgagctataagccaagccgtagatcaggctttgagagggatgtctgatctccccccttatggaagtggtagcagtgttcagagacaggaaccacccatttcacgcagtgccaaatatactttctacaaggatcctcgcctaaaggatgccacctatgatggacgctggctttcagggaagcctcatggcagaggggttttgaagtggcctgatggaaagatgtattctggcatgttcaggaatggcttggaagatgggtatggagaatacagaatcccaaacaaggcaatgaacaaagaagaccattatgtgggccattggaaagaaggaaaaatgtgcggtcaaggagtctacagctatgcttctggtgaagtatttgagggctgttttcaagataatatgcgtcatggtcatggtcttctacgaagtgggaaattgacgtcctcttctcctagtatgttcattggccagtgggtaatggataagaaagcaggatatggtgtctttgatgatatcactaggggggaaaagtatatgggaatgtggcaagatgatgtgtgtcaagggaatggtgtggtggttacccagtttggattatactacgagggcaactttcaccttaataaaatgatgggaaatggggttttgctttccgaagatgatactatctatgaaggagaattttcagatgactggactcttagtggaaagggaacactgactatgccaaatggagactacattgaaggttattttagtggagaatggggatctgggataaaaatcactggaacctacttcaaacctagtctatatgagagtgataaagacagacctaaagttttcaggaagctaggaaacctggcagtgccagctgatgagaagtggaaagcggtgtttgacgaatgttggcgccaactgggctgtgagggcccaggccaaggggaagtttggaaagcatgggacaatattgctgtggccttgaccaccagtcggcgccagcacagagacagtccagaaatactgagtcgttcacagactcagacactagagagtttggaattcattccacagcatgttggtgccttctctgtggagaaatatgatgacatcaggaaatatttaataaaggcctgtgacactcctctgcaccccctgggcaggcttgtggagacactggttgcagtgtatagaatgacatacgtgggcgtaggagccaaccgcaggttattgcaggaggctgtaaaggagattaagtcctatcttaagcgaattttccagctggtgaggttcttatttcctgagctgcctgaagaaggcagcacaattcctctctctgctcctctgccaaccgaaaggaagtctttttgcactgggaagtcagattcccgatctgaatcaccagagccaggttatgtagtaacgagttctggattattgcttcctgtgctgctacctcggctctacccaccgctgtttatgctttatgctttggataatgatcgcgaggaagacatttactgggaatgtgtccttcgactaaataagcagccagatattgctctcctgggctttcttggggtgcagaggaaattttggccagcaaccttgtcaatccttggagagagtaaaaaggttttgccaaccacgaaagatgcttgttttgcctcagcagtagaatgtctgcagcagatcagcacaacatttaccccatcagacaaacttaaggtcatccagcagacttttgaggagatctctcagagtgtcctggcgtcactccacgaagacttcttgtggtccatggatgacttgtttcctgttttcttatatgtggtgctacgggccaggattaggaatttaggctctgaggtacacctcattgaggatctaatggacccctatcttcagcatggggaacagggtataatgttcaccaccttgaaggcatgttactaccagattcagcgtgagaagcttaactaggctgcataacagcttgaaaactggattatctactacagagtgttataacaccatctggagtcttcctgtagtggcaaaaaagaacagtgttgaaattggaaaggactttgtgttatttaggttgttagaatgagccttaccaataataagagccctgagcccagaaaaaaggactgtatagtttaaagggaggattgaaagggaggtaaaaaatcagattagaccagttcttggcctatgataagttccaaaaataccatttatctactatttgaaaaaagaagaggatatcccttcctacagtaaagggtatgtcagctacatgaagttgtaagaaaagcttccagtagagcttcttatattaaagaagttgatggatatttttgaatttctggtttgcctgaatccacctgcagttaccccgatccgtttgcaagaaccagatcgtacttgaaactatagtggccacactctgccttcctgagtcccttccagtcatgtgtgcatcatgtctctttgccaagggaggggagaaaggaacttttaaactgcagttttaactttttctaagctgtttcttgatgggagaggttctgtgcaaaactaccacattctgtccccaaaatgtggaatgcatccaaataggagtcttctgcctcttaacttaaaagaacataggaattttgtttttggtttctttatcatgctacagagagtgaatacactggaattcagacaccgactctgagctgctaggaacctcatttgtccatgtgcaaacgctgtattccaaggcctgtgaatggcagcctgaggaagttttgcatgcaggctgtgttttcgagcaggactaacaactgggaaataagcaaaaaactgcatcgatccccagcctggtgttgttcttccctatacttcacactgaactcaggatgggaagaaaaaggaaacaagctttggctttttccatctcaaaagtattgtggcacctcaacatttcagtgttttgctttttaaaaaatgccctattgtaagttgttggtttatactgtataagtaacactagtagctgttttgaataacataggtgctcttcctcatctcatctcctacaccgtggtgagcatacagagtgtcctgatttgtgttaagtgactgagaagatgttaattacttttgaaaaaggatcatggtttttgctctactttataatcaagacaagtgtttattaaaatactgttttggaatgttggctgtaatgtaacagcaattttcataataaaaggcattcatcttt

SEQ ID NO: 3 - DNA, 11105 bp, Homo sapiens
acttccgctggtggcctagagcggggcccggtatggaggtgggctagaggccgacgccagccagagagcgaaatgttcttttggggccagagtctgggcatatatgaatgcaaatccgtgtttgttcacaactaagcccagctgagacgatcacttttctgtaggccatttgtccaggtacagaatgagcacatgttgttggtgtacgccaggtggtgcttccaccattgacttcctaaagcgctatgcttccaacactccgtccggtgaatttcaaacagccgacgaagacctctgctactgcttggagtgtgtggctgagtaccacaaagcaagagatgaattgccattcttgcatgaggttttatgggaattagaaaccttacgtctcataaatcactttgaaaaatccatgaaggcagaaattggagatgatgatgagttatatatagtagacaataatggagagatgccactgtttgacatcactgggcaagactttgaaaataagcttcgagttcctcttcttgaaatactgaaatatccttacttgcttctacatgaacgtgttaacgagttatgtgttgaagcactttgtcggatggaacaagccaattgctcctttcaggtgtttgataaacatccagggatctatttgtttttagtccatcccaatgaaatggttcggcgttgggctatcttgactgcaagaaacttggggaaagtggacagagatgattattatgacttacaagaagttttactttgcctttttaaagtcattgagttggggcttttagagagtccagacatttatacttcttctgtcctagagaagggtaaactgattcttctgccctcacacatgtatgatactaccaactacaaaagctattggttaggtatttgcatgttgctgaccattcttgaggaacaagccatggattccctgttgttgggctcagacaaacaaaatgattttatgcaatcgatacttcacactatggagagggaagcagatgatgatagtgtggatcctttctggccagcgttacactgttttatggtgattctggatcgccttggatctaaggtctggggtcaacttatggatcctattgtggcatttcaaaccattatcaacaacgcaagctacaatagagagatccgacatatacggaacagctctgtaaggaccaagttagaaccggagtcctatttggatgatatggtgacttgcagccagatcgtatacaattataatcctgaaaagaccaaaaaggattctggatggagaacagccatttgcccagattattgtcctaacatgtatgaagaaatggaaacattagccagtgtacttcagtcagatattggtcaagacatgcgtgttcataacagcacatttctatggttcatcccttttgtccagtccctcatggatcttaaggatttgggtgtggcttacatagcacaggttgttaatcatctgtactctgaagtcaaagaagtcctcaaccaaacagatgctgtgtgtgacaaagtcactgaattttttcttctaattttggtatcagtgattgaactgcatagaaataaaaaatgtttgcatttgctgtgggtaagttcccagcaatgggtggaagccgtcgtcaaatgtgccaagcttcctaccactgcgtttacacggagttctgagaaatcatctggaaattgctccaaaggaacagcaatgatatcttcactgtcattgcattccatgccatctaactctgtacaacttgcttatgtgcagctgattagaagtctccttaaagaaggttatcagcttgggcagcagtctctttgcaagcgattctgggataagctcaacttattccttagaggaaatttatctctaggttggcagttgactagtcaggaaacccatgagctacaaagttgcttaaagcaaattattagaaacataaaattcaaagcacctccatgtaacacttttgtggatctgacttctgcatgtaaaatctctcctgcatcttataataaagaagaaagtgaacaaatggggaagacgtctagaaaagatatgcattgtttggaagcttccagcccaacattttctaaagaaccaatgaaagtgcaagacagtgtattgatcaaagcagataacactatagaaggtgacaataatgagcaaaattatataaaggatgtgaaactagaggaccatctcttagctgggtcatgcttaaagcagagtagtaaaaacatttttactgaaagagctgaagatcaaattaaaataagtacaaggaagcagaagtctgtaaaagagatctcttcatatacaccaaaggactgtacttcaagaaatggtccagaaaggggatgtgacagaggaataatagtatcaacacgtttgttgactgattctagcactgatgctttggaaaaagtgtccacatcgaatgaagatttctctttaaaggatgatgctcttgctaaaacctcaaaacgaaaaactaaggtacagaaagatgaaatctgtgcaaagttatcacatgtaataaagaagcaacacaggaagagtactttggtcgataatactatcaatttagatgaaaatttgactgtatctaacattgagagtttctattcaaggaaagatacaggagttcagaaaggagatggtttcatacacaatctttctttagaccctagtggtgttctggatgataagaatggagaacaaaaatctcaaaacaatgtattgccaaaagagaaacaattaaagaatgaagaattagttattttctctttccatgaaaacaattgtaaaatacaggaatttcatgttgatggtaaagaattgatcccttttacagaaatgaccaatgcttcagagaagaaatcatctccctttaaagatcttatgactgtacctgaatcaagagatgaggagatgagtaatagtaccagtgtgatttattctaacttgacaagagaacaggcccctgacatcagtcctaaatctgacaccttaacggattctcagatagacagagaccttcacaaattatctttactagctcaagccagtgttattacgttcccatccgattcacctcagaactcatcgcagctgcaaaggaaagtaaaagaagataaaagatgtttcacagctaaccaaaataatgttggagatacctcccgtggacaggttattattatttcagattctgatgatgatgatgatgaaagaatcctgagtcttgagaaactcactaaacaggacaaaatatgccttgagagggaacatccagagcagcacgtttcaacagttaatagtaaggaggaaaagaatccagtaaaggaagaaaagacagagactctttttcagtttgaggaatctgattctcagtgttttgagtttgaaagttcatctgaagtgttttcagtttggcaagatcatccagacgataataattcagttcaagatggtgagaaaaaatgtttggctcctatagccaatactacaaatggtcagggttgtacagattatgtatctgaagttgttaaaaaaggagcagagggcattgaagaacacacaagaccacggagtatttctgttgaagaattttgtgaaattgaagtaaaaaagcctaagagaaaacgatctgaaaaaccaatggctgaagatcctgtgaggccttcatcttctgtcagaaatgagggccagtctgatactaataagagagatcttgtgggaaatgattttaaaagtattgatagaaggacttcaactcccaattcacgtattcagagagccactacggtttcacaaaagaagtcttcaaagctttgtacttgtacagaacccatcaggaaagttccagtttctaagacccctaagaaaactcattcagatgccaaaaaaggacagaatagaagttcaaattacctaagttgtagaacaactcctgctatagtgccgccaaagaaatttcgtcagtgtcctgagccaacttcaacagctgagaaacttggcctgaaaaagggtcctcgtaaggcatatgagttgtcccagcggtctttggattatgtagctcaattacgtgatcatggcaaaactgttggagtagttgatacccgaaaaaagactaaattaatttctcctcagaacctgtctgtcagaaataataagaaacttctgactagtcaagaacttcagatgcaaaggcagatcagacccaaatcacaaaaaaatagacgaagactttctgattgtgaaagtacagatgttaaaagagcagggtcacatacagcacagaattctgacatatttgtaccagaatctgataggtcagattataattgtacaggaggaactgaggtacttgccaacagtaacagaaaacagttaataaaatgcatgccttctgaaccagaaaccataaaagcaaaacatgggtctccagcaactgatgatgcttgccctttgaaccagtgtgattctgtagtgttaaatggaacagtaccaacaaatgaagtaattgtctccacttcagaagaccctctgggtggaggtgatccaacagcacgtcatatagagatggcagctttgaaagaaggagagcctgactccagcagtgatgcagaggaagataacttatttttaacccaaaatgatcctgaagatatggatttatgttcacaaatggagaatgacaattataaactcattgaactaattcatggaaaagatacagttgaggttgaagaagattctgtaagtcggcctcagttggaatctttgagtggcacaaagtgtaagtacaaagattgtcttgaaaccacaaaaaaccagggtgaatactgcccaaaacactctgaagtgaaagcagcagatgaagatgtatttcgtaaacctggcttgcctcctcctgcatctaaacctttgagacctaccactaagatttttagctcaaagagtacttcacgaattgctggtctttctaaatctttggaaacttcttcagcactttcaccgtctctaaaaaataagtcaaaggggatacagtcgattttgaaagtaccacagccagttcccctcatagctcagaagccagttggtgaaatgaagaattcgtgcaatgttcttcatcctcagtctccgaataattccaacaggcaaggttgcaaagttccatttggtgaaagcaaatattttccatcttcctctccagtaaacattcttttgtcatcacagtctgtctctgacaccttcgttaaagaggtcttaaaatggaaatatgaaatgtttttgaactttggtcagtgtgggccccctgcaagtctttgtcagtccatctcaagacctgtgcctgtcagatttcacaattatggagattattttaatgtttttttccctttgatggtattgaatacttttgaaacagtggcacaagaatggctcaactctccaaatagagagaatttctatcagttgcaagtacgaaaatttcctgccgattatataaaatactgggagtttgcagtttatctggaagaatgtgaactggctaaacagctttatccaaaggaaaacgatttggtgtttttagctcctgagagaataaatgaagagaagaaagatacagagagaaatgacatacaagatctccacgaatatcattctggttatgttcataaatttcgccgcacgtcagtcatgcgtaatgggaaaactgagtgttacctttccatccagactcaagagaactttccggccaatttaaacgaacttgtgaattgtattgtaatcagttctctggtaactacacaaaggaagttgaaagccatgtctctgttgggtagtcggaaccaactggctagagctgttctgaatccaaaccctatggacttctgtacaaaagatttactgactacaacatctgagagaattattgcgtacttaagagatttcaatgaagatcaaaagaaagcaatagaaactgcatatgctatggtgaaacactcaccatcagttgccaaaatctgcttgattcatggaccacctggaacaggaaaatcaaaaactattgttggcctcctctatcgtctactgacagagaaccagaggaaggggcattcagacgaaaactccaatgccaaaatcaaacaaaaccgtgtcctcgtgtgtgcaccttccaatgcagctgttgatgaactcatgaaaaaaattatccttgaattcaaagaaaaatgtaaagacaagaagaatcctttaggaaactgtggagatataaatttagtacgactgggtccagaaaagtctattaatagtgaggttctaaagttcagtttggacagccaagtaaaccacagaatgaaaaaagagttaccttctcatgttcaggcgatgcataaaagaaaggaatttctagattatcagctggatgagctttcccggcagcgagctctatgccgaggtggacgggaaatacagaggcaagaattagatgaaaacatttccaaagtttctaaggaaaggcaggaacttgcttctaaaattaaagaggttcaaggacgcccacagaaaacacagagtatcatcatcttagagtcccatatcatctgctgcacgttgagcacaagtggtggtttactacttgagtctgctttccgtgggcaagggggtgtccccttcagctgtgtcattgttgatgaggctggacagtcttgtgaaattgagactcttactccactcatccatcgctgcaataagctcatcctagtaggagatcctaagcagctccctccgacagtcatctctatgaaagcacaggagtatggctacgaccagtcaatgatggctcgcttctgcagactgctggaagagaatgtagaacacaacatgatcagcaggctgcccattctacagctcactgttcagtacaggatgcatccagacatatgcctcttcccttctaattatgtttataacagaaacttaaaaacaaatagacagacagaagccattcgatgttcatcagattggccatttcagccataccttgtgtttgatgttggagatggttcagaaagacgggataatgactcatatataaatgttcaagaaataaaactggtgatggaaataattaagcttattaaagacaaaagaaaggatgttagttttcgaaacattggcataataactcattacaaggcccagaagacgatgattcagaaggatttggacaaagagttcgatagaaaaggaccagcagaagtagacactgtggatgcattccagggtcggcagaaggattgtgttattgttacgtgtgtcagagcaaatagcatccaaggttcaattggattcctggcaagtttgcagagattgaatgtcaccatcacacgagccaagtacagcctcttcatcctcggacatttgaggaccctgatggaaaaccagcattggaatcagctgattcaggatgctcagaagcgtggtgccattattaagacctgtgacaaaaactatagacatgatgcagtgaagattctgaaactcaagcctgtgctgcagagaagtctcactcaccctcctaccatagccccagaggggtccagaccccagggtggtttgcccagcagcaagctagacagtggatttgccaagacatctgttgctgcttctctataccacacaccctctgactccaaggaaattactcttactgttacttcaaaggaccctgaaagacctcctgttcatgaccaacttcaggacccacgactgctgaagaggatgggcattgaggtcaaaggaggaatattcctttgggatccacaaccctcgagcccccagcatcctggagcaacacctcctacgggcgagccgggcttccctgtcgttcaccaggacctgagccatatacagcagcccgctgctgtagtggctgctctgagcagccacaaacctcccgtgcggggcgaacctccagctgccagtcccgaggcttccacgtgtcagagcaaatgtgatgacccggaagaggagctctgtcacaggagagaggccagggctttcagtgaaggggagcaggagaagtgtggttccgagacccatcacaccaggaggaactctaggtgggacaagaggacactggagcaggaggacagcagttccaagaaaagaaagcttttataggaaagcccagtgacatgggccagcagccacagcatattgtaaactgaagatgaccagctcgtgggaccatctagataagcttgttttttgtaaggagtttgtgtgctgttggaaaacatggaaaatgcatccttaacacctgagcctctggtcatcttcagtattttctgtcatttgcaaaagctttcagagggcattgtgtatccgtaataatgtccttgaagtcagagactggaaatgttgatctcttagtcttctatagacaaggaggtcagactggagtgaatgttgtaaaagttgaaatgtatatttgtatagcaaataacaaaatccttttaaaatttaatctagtagaccgcttttctttccccctgtttaaaatgttaatcagttttcaacagaacaaactttatattaccaaaaaaaaaaagatgggaggagggcgggtcctttcaaccattgttagagcaagacagataattatttaacagcctagcttggaataagctgagttagtgctggtgggtcaggtgtctctggctctattaaaaaaagcaaaaacccccccaaaaacctggagtctcctaggggacactttgggcagcacggttatgttaggtaaagtcttgctgacatggtgcatttttagatagagtgcattggcccagggtattatatttctgtgatgagttcatttacctgtttcagtatgcacatagttccctagctaaaattcctaatcttcttgagagagaagaatatggagtgaaagaataattcttgagctatcattcaaatgctcccagcatattgtgagccttgtgtgactggtgaggtacagacatttggttgccaaatgctagattagcgctgggtcagctgtggaagaatcgcctcagcttacagattgtcagacagatctaactagttttccagaaagcctgggaagctgtgtgttcaacatttcccaagggattctgatcaccaggcagcttgggaaccactggggcaggccaaatagaatattttgggcgggaaagaagcacccgatttaaaatgaagcgtaaccagaggagttcagaactgggaagagagtggtagacttcctgtgatcttcagaaatcatctacctggtaaaaatacatgctgtttagaatatctgataggtgtttccagctactattagaggtgatagtgcttttgtgggggaaaaaattggtcatggtgaatggagatcgaggaagctcgggacaagggaggggtgggctgcctgattttgtccagttttccaaatatccacgcagtgaactggagtatcctaaacatgagaatgtacagttgacagttgtaaaaactagggatctgtagtgaatgctgtgcagccccatatctcatttgggggtaggaaaatagctgaagattcatgtgcattatttgacatttcctttgtcatctgctttttaagcaaaaaagggttttgtgttagaaattctacttgagcagattataaagagctttaaaaaacaactttcggttgccaaaagtttgagcatttgatttcattacctgtgtctccctcactggtgtccagacggtcaactgaatactcctgaaacccagggagcaggtgacttcctggagtgctttgtccccagagtcagccactgcttcctctgtgggggtggagagtttgtctttggccatgcagtgtgcgacagttcaggacgggtagggatgggtcccattctgtctgggtcaagggctctatcagcttcttccatgtgcctttgggaagaaatctcgttactttaagtttgctttcctgttatcttgatgaagtgcccattttagcagacacttgtagtgctgaccacttagggaatgtacaaactcctaagcttctaaagggaggcatggcaaaaacgttggggtcaggatgtctctcacgctgctcatgttaatactattaacacatgatttgagaaataagttttctctaaaatgcatattttgccgccacacactgaacaatattatttccagtgaagtttgatgcctgttcttacgttgtgttcacctgttggttcaccactcagcagatctgattctgcaagaattaatggtagaactagatcatcctttctaacagacgagcctgtgtcctgtgacggcctttcacagcggaatgcagttgtacctcacattacttttgaaacttcactcgttccagttggtacaagtatttgccaaagccatttcctatgttcaccgtggcccctcctgatgtggctgtcagcgcagcgttgttgaacagggctattctttttacaaggtgtgaagtgtggctcttcgcttcgtctttgccatggcattaaaagaaagttccctgtcttctttcaatattagttatttcaaatgaatatgtgctacttaaaagcttgttttgtttctttgtatataatttgccttggatttattgtgcacagtttgttgagttgtatgtttttgtgaattatcaggagtaaatttgacaagtacatgtgaataacctcctgtaaatgaattttataacaaaaatgtactgaactattttttaaagttgtgcagattagcaattttttgctatagctttgacttttctatgctgtgaattaatagctgcgatttggcaaacagccctgttgtctttgttaaaccctaaattttaagaggaaatggcagaattaaaagcagaaacaagaagatggacatggattagaggttatgtattatgaagtaaactacaaggtactaacatcatttcgtctgccatttggtttgctttatgctgaaattacttggtggggatttgtgcaattcagatataaaaagtttcattatccaaaaa

SEQ ID NO: 4 - protein, 2443 aa, Homo sapiens
MAAEEGVASAASAGGSWGTAAMGRVLPMLLVPVPAEAMGQLGSRAQLRTQPEALGSLTAAGSLQVLSLTPGSRGGGRCCLEGPFWHFLWEDSRNSSTPTEKPKLLALGENYELLIYEFNLKDGRCDATILYSCSREALQKLIDDQDISISLLSLRILSFHNNTSLLFINKCVILHIIFPERDAAIRVLNCFTLPLPAQAVDMIIDTQLCRGILFVLSSLGWIYIFDVVDGTYVAHVDLALHKEDMCNEQQQEPAKISSFTSLKVSQDLDVAVIVSSSNSAVALNLNLYFRQHPGHLLCERILEDLPIQGPKGVDEDDPVNSAYNMKLAKFSFQIDRSWKAQLSSLNETIKNSKLEVSCCAPWFQDILHLESPESGNHSTSVQSWAFIPQDIMHGQYNVLQKDHAKTSDPGRSWKIMHISEQEEPIELKCVSVTGFTALFTWEVERMGYTITLWDLETQGMQCFSLGTKCIPVDSSGDQQLCFVLTENGLSLILFGLTQEEFLNRLMIHGSASTVDTLCHLNGWGRCSIPIHALEAGIENRQLDTVNFFLKSKENLFNPSSKSSVSDQFDHLSSHLYLRNVEELIPALDLLCSAIRESYSEPQSKHFSEQLLNLTLSFLNNQIKELFIHTEELDEHLQKGVNILTSYINELRTFMIKFPWKLTDAIDEYDVHENVPKVKESNIWKKLSFEEVIASAILNNKIPEAQTFFRIDSHSAQKLEELIGIGLNLVFDNLKKNNIKEASELLKNMGFDVKGQLLKICFYTTNKNIRDFLVEILKEKNYFSEKEKRTIDFVHQVEKLYLGHFQENMQIQSFPRYWIKEQDFFKHKSVLDSFLKYDCKDEFNKQDHRIVLNWALWWDQLTQESILLPRISPEEYKSYSPEALWRYLTARHDWLNIILWIGEFQTQHSYASLQQNKWPLLTVDVINQNTSCNNYMRNEILDKLARNGVFLASELEDFECFLLRLSRIGGVIQDTLPVQNYKTKEGWDFHSQFILYCLEHSLQHLLYVYLDCYKLSPENCPFLEKKELHEAHPWFEFLVQCRQVASNLTDPKLIFQASLANAQILIPTNQASVSSMLLEGHTLLALATTMYSPGGVSQVVQNEENENCLKKVDPQLLKMALTPYPKLKTALFPQCTPPSVLPSDITIYHLIQSLSPFDPSRLFGWQSANTLAIGDAWSHLPHFSSPDLVNKYAIVERLNFAYYLHNGRPSFAFGTFLVQELIKSKTPKQLIQQVGNEAYVIGLSSFHIPSIGAACVCFLELLGLDSLKLRVDMKVANIILSYKCRNEDAQYSFIRESVAEKLSKLADGEKTTTEELLVLLEEGTWNSIQQQEIKRLSSESSSQWALVVQFCRLHNMKLSISYLRECAKANDWLQFIIHSQLHNYHPAEVKSLIQYFSPVIQDHLRLAFENLPSVPTSKMDSDQVCNKCPQELQGSKQEMTDLFEILLQCSEEPDSWHWLLVEAVKQQAPILSVLASCLQGASAISCLCVWIITSVEDNVATEAMGHIQDSTEDHTWNLEDLSVIWRTLLTRQKSKTLIRGFQLFFKDSPLLLVMEMYELCMFFRNYKEAEAKLLEFQKSLETLNTAATKVHPVIPAMWLEDQVCFLLKLMLQQCKTQYELGKLLQLFVEREHLFSDGPDVKKLCILCQILKDTSIAINHTIITSYSIENLQHECRSILERLQTDGQFALARRVAELAELPVDNLVIKEITQEMQTLKHIEQWSLKQARIDFWKKCHENFKKNSISSKAASSFFSTQAHVACEHPTGWSSMEERHLLLTLAGHWLAQEDVVPLDKLEELEKQIWLCRITQHTLGRNQEETEPRFSRQISTSGELSFDSLASEFSFSKLAALNTSKYLELNSLPSKETCENRLDWKEQESLNFLIGRLLDDGCVHEASRVCRYFHFYNPDVALVLHCRALASGEASMEDLHPEIHALLQSAELLEEEAPDIPLRRVHSTSSLDSQKFVTVPSSNEVVTNLEVLTSKCLHGKNYCRQVLCLYDLAKELGCSYTDVAAQDGEAMLRKILASQQPDRCKRAQAFISTQGLKPDTVAELVAEEVTRELLTSSQGTGHKQMFNPTEESQTFLQLTTLCQDRTLVGMKLLDKISSVPHGELSCTTELLILAHHCFTLTCHMEGIIRVLQAAHMLTDNHLAPSEEYGLVVRLLTGIGRYNEMTYIFDLLHKKHYFEVLMRKKLDPSGTLKTALLDYIKRCRPGDSEKHNMIALCFSMCREIGENHEAAARIQLKLIESQPWEDSLKDGHQLKQLLLKALTLMLDAAESYAKDSCVRQAQHCQRLTKLITLQIHFLNTGQNTMLINLGRHKLMDCILALPRFYQASIVAEAYDFVPDWAEILYQQVILKGDFNYLEEFKQQRLLKSSIFEEISKKYKQHQPTDMVMENLKKLLTYCEDVYLYYKLAYEHKFYEIVNVLLKDPQTGCCLKDMLAG

SEQ ID NO: 5 - protein, 526 aa, Homo sapiens
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 6 - DNA, 2285 bp, Homo sapiens
gcgtgccgtcagctcgccgggcaccgcggcctcgccctcgccctccgcccctgcgcctgcaccgcgtagaccgacccccccctccagcgcgcccacccggtagaggacccccgcccgtgccccgaccggtccccgcctttttgtaaaacttaaagcgggcgcagcattaacgcttcccgccccggtgacctctcaggggtctccccgccaaaggtgctccgccgctaaggaacatggcgaaggtggagcaggtcctgagcctcgagccgcagcacgagctcaaattccgaggtcccttcaccgatgttgtcaccaccaacctaaagcttggcaacccgacagaccgaaatgtgtgttttaaggtgaagactacagcaccacgtaggtactgtgtgaggcccaacagcggaatcatcgatgcaggggcctcaattaatgtatctgtgatgttacagcctttcgattatgatcccaatgagaaaagtaaacacaagtttatggttcagtctatgtttgctccaactgacacttcagatatggaagcagtatggaaggaggcaaaaccggaagaccttatggattcaaaacttagatgtgtgtttgaattgccagcagagaatgataaaccacatgatgtagaaataaataaaattatatccacaactgcatcaaagacagaaacaccaatagtgtctaagtctctgagttcttctttggatgacaccgaagttaagaaggttatggaagaatgtaagaggctgcaaggtgaagttcagaggctacgggaggagaacaagcagttcaaggaagaagatggactgcggatgaggaagacagtgcagagcaacagccccatttcagcattagccccaactgggaaggaagaaggccttagcacccggctcttggctctggtggttttgttctttatcgttggtgtaattattgggaagattgccttgtagaggtagcatgcacaggatggtaaattggattggtggatccaccatatcatgggatttaaatttatcataaccatgtgtaaaaagaaattaatgtatgatgacatctcacaggtcttgcctttaaattacccctccctgcacacacatacacagatacacacacacaaatataatgtaacgatcttttagaaagttaaaaatgtatagtaactgattgagggggaaaaagaatgatctttattaatgacaagggaaaccatgagtaatgccacaatggcatattgtaaatgtcattttaaacattggtaggccttggtacatgatgctggattacctctcttaaaatgacacccttcctcgcctgttggtgctggcccttggggagctggagcccagcatgctggggagtgcggtcagctccacacagtagtccccacgtggcccactcccggcccaggctgctttccgtgtcttcagttctgtccaagccatcagctccttgggactgatgaacagagtcagaagcccaaaggaattgcactgtggcagcatcagacgtactcgtcataagtgagaggcgtgtgttgactgattgacccagcgctttggaaataaatggcagtgctttgttcacttaaagggaccaagctaaatttgtattggttcatgtagtgaagtcaaactgttattcagagatgtttaatgcatatttaacttatttaatgtatttcatctcatgttttcttattgtcacaagagtacagttaatgctgcgtgctgctgaactctgttgggtgaactggtattgctgctggagggctgtgggctcctctgtctctggagagtctggtcatgtggaggtggggtttattgggatgctggagaagagctgccaggaagtgttttttctgggtcagtaaataacaactgtcatagggagggaaattctcagtagtgacagtcaactctaggttaccttttttaatgaagagtagtcagtcttctagattgttcttataccacctctcaaccattactcacacttccagcgcccaggtccaagtctgagcctgacctccccttggggacctagcctggagtcaggacaaatggatcgggctgcagagggttagaagcgagggcaccagcagttgtgggtggggagcaagggaagagagaaactcttcagcgaatccttctagtactagttgagagtttgactgtgaattaattttatgccataaaagaccaacccagttctgtttgactatgtagcatcttgaaaagaaaaattataataaagccccaaaattaagaaaa

SEQ ID NO: 7 - DNA, 21795 bp, Homo sapiens
aaatatatatatatgtatatatatgtgtgtatatatatgtatatatgtgtgtatatatgtatatatatgtgtatatatgtgtgtatatatatgtatatatatgtgtatatatatgtatatgtatatggaagagaggttaaaataaatggaaaacaaggtaggaagctctaatagatgtttaatttcagttccaaaaagagagaaaaaaatagagtaaaaacaatataggaggctggctggtctgagtgcagtggtgtttacaactaattgatcacaagcagttacaggtctctttgttccttctccactcccactgcttcacttgactagcctaaaaaaaaaaattaaaaaaatatataaaaaaataggagaagataatggctactattagttaaaatgtatctcccccaaatttatatgttgaagccctaatccctagtatcccagaatgtaactgtatttggagatagggcgtttaaagagatgattaagttaaaatgaggccattaaggtgggccctaatccaacctggtgtccctgtaaggggagtaaatttagacacaaagagaggagctaagggtgtggacccacagaaggacagctatgtgaggcggcagcaagaggccagccatctctaagtgaaggagagaggcctcagaaggaaccaaacctgccaacaccttggtcttggatttttgacctccaggactgtgagaaaatgaatttcagttgtttaagtcacccagtctgtggtattttattatggaagccctagcaaactaacacaatgactgagagttttccagaactaaagaaagacagcagtatatggatttaagaaaacccatacccaaaaacttgacactgataccatatttggaaaaacatgggttttccttatggtatcctgtactcctgttttatgtttctaaatggaaagcttcctgggcgatgcatctgattggtggagcccaggtcacatgcccattctcagctgtaagctgggaaagcaagtgttctaggacagacttacttataatatgtgaaatatccacatgtaaggagaatttttaaaagatgttggacacacactaacataacatatgcctataacagggctattcaaaacttatgttctagtctcagctgttttctctgtattcttttccttcaagagtgcataactcgtagagtctctaactcaatggatgacttatgtaatttgattaattcatttatcaattcatgaaactctagacacgagactattctggctatcatgcaattcccatgaactgctccaccttcacttcatattcagcttacctaaatcctagcgtcacccctccgccctctccagatacgctgaagaagtaggcttccttttttctgcaggtgttatcttgcctcttcaagacacctttgagagtcccctctctttcctcttgatttcacttcatacctaggttcagcagaatcgccctgccctgtaacgctctcactcatcccttcctttccagtcccataacgattacctttttgcagctttatgtttgagcatcttcaccatggccagtcactggtgtttctgactctggttgctcgtcccttcaattaattccgcaaactcatcaatgtttggtgaatttctattttcttctactaatcttctcacctgctgccccagccagtgttcccttgaatgtctcagtctttctctcggactttgctcgtaattttattctaccagggaccaccctgctcccatttttgtctaagtcttaacctcccttcatagtgcagctcaggccaatgccctcttgaagacacccccggatggttcactttgtaaatgtgtggaccttggcatggccaacgccccttcacacctggcacagctacttttcattgttttccttcatgaaaactaaagttcttttgttccacaaaactgtgacagtgacttcaggataaagatcatatctggatccctaattcttagtgcagagcctagtacagaactggaatgcagtgtttgttgaatgaaggaatgaattaaactgcaaattaacgaatgcctgctctggcactgagactcgcagctgtgagaaaggtctctggaggcagacagttgctggtacgaagtcctgagtcaagcaggaaacaaaatgcggatgtcagcagtttcaagtgagacctgtggtgagctccttcctctgccggttttcccttcttaattttcagtctgaagtgagggagtggagaatctttctgtgttagactgtgtcgcgttgggcagatttctccaatcttaggatagtagtggtccctctcactggccttcactttgtaccatccccagcaagaacactgagtctgtgaaccgaattctagtagtcgggaaatggattgaaaggacatccaggttttacttagtttctatgctcagatgctggcagaagccctgcaaatcttttccttttctgtgaaaggaagaggcggtttctgcgtgaggccctatagctcaggggttagagcactggtcttgtaaaccaggggtcgcgagttcaaatctcgctggggccttgcgaaactactttcttgattcaggtgttttttgaaaaactccccttctagtctttctgaactaagtcacagaaaatgtggcaaattagagcttctttcctctgttcagagcttccaaacactagctcagcagaacgtttcctttaggttccagtagtaatgctgctttagctcctggaatgcggcggaatccattacacaggcggtggaaatcaccgtcagcctctgcccacgccctggcttgcggactggcggcgagagccctgctgactgccgggcgggaaatggggacagctaggcgagacaatagccctttaagctattctcaagccccgggtctgggcatatttggagtagagatcaaggttccacttctgaaacgggaagagccagcaatggccgctccggccaggcgtccagaagagagatgcacgttgtgaggcaaggtttcatgtgccagtggggttttgggaaattaagggcccgactttaggggcgagacttgggtcactaaatttgattttaacgcagagaaatgtggccttccttttgaatgagctgcaaatattatctccaaaagcgaattcacacagaaacattgggtttgcccagatgtcctctatttcaggggttcccaacccttgggccgcgcacctgtccatggcctgttagggacccggctgtacagcaggaggtgaactccgggctagctaccagtaccgcctgagctccgcctcctgtgagatcagtggtggcattagaatctcacaggagcgcgaaccctattgggaactgcgcatgccagggatctaaactgcactccttatgagaatctaagtgatgcctgatgatctgaggtggaacagtttcatcccgaaaccatcccccaactcttccccccgcccccgcggtccgtggaaaaactgtcttccacgaaaccggtccctagtgccaaaaatgttggggaccactgctccattccacaccctctccctcccgtgtctgtggacgtgtaagcggaagagccgagattgggagggaagcgcaggaagtgaggacatcagggagcccgggcctaagcctggaggcccgccctctccggcgttcacagttgcgcagtcagaattgtgtgcgtcccaccgcctcagcccccagttctccgacgcccaggtgccggtccccaccctacacacaaacacacgcatccacgctcgcgaggcccctgggtccgcgcccagtgaggccaacagccctcgggcgcccagaggatgagcagcctcggtatgcccgagtttcaggggcgcctgctctgcttgggagcccctcagtgaggcaggggaatccagatttagaaaggttgtcctcagaacctcggacgcaggactgtcttacaagctggctttgtgactctgcagtaattactttccgggtataaagagatttctttctttcccgtctctggttaaggatgtctcgagtagggatttttaggagagttgtttccgtgcttccttcgatagctcagctggtagagcggaggactgtagtacttaatgtgtggtcatccttaggtcgctggttcgattccggctcgaaggagacgctgctgttttggggtttacggttgctcttcgtttgtgcaaaaaatctctgtatcttgacgtgatcaaatctgtttctgtctctcacccttgtccgaaggaaacagcttagccgcgaggtgggctgcgttaatcgcgtcttgaaaagttggttacctttctgaaagatagtagacccgaatcgtcccaccgttgggggaaggcactgttgtcttaccgaagtgtgttatattttggcttctacgttaagtccaaaggccggcagatatatacgtatcgcgcgtctgtcctggggctcagtaccctacagccttgcaaccgagctcagctctggccctcgatctctatttcagccccctccaggttccctccaggccctagcgggcttaaatcgctccccgggctcagtgagacacaagcatgtgacctaaagtctaaaaagctgccttccttcaccccatcctgcatctggatcgaggagaataagccagagaataagccacacttgaaacttggtgaagatggaaggaataactaaataaggcctaagggaaatgtcacaacagaggaaaagaaacaaaacaaaaaaccttcagaacagcaggcggaaaagagaaattcagagtgataggaagcaccactcagctacagtggctcgttggtctaggggtatgattctcgctttgggtgcgagaggtcccgggttcaaatcccggacgagccctcgtggctactgtttttccacccccttttgtcaactactgaaaaaaagtctccgttaaatttgaaacacaaactgtctcgcgatgggctgtcctgccaaagaaagcagcagcgaataagtacgtggcagatggtgctgtcgaccagtgtcaagaccaagtttcatgggttcctggacgctcagccaggggtagtctctgaagggccgcctggcaattgggagcagaagccagtttcccgccatctgctctccgtggaggcagtgctctcgcggctgctgcccgttggggtccagctgaggaaggagcgcggatcccaggctcgttctttgcctgggctgcccctgcggcccctggggcgggtgctgctgcggcgccagctccaagggcggatccaggcgggaggggcctcctcggagaagcggggcgcggtcccaactacgcagaggctggcacgccgaccctccacacctcaccacgcccccatctccgtccgtgtacacacactcacacaaggacgccaaccccacctagatgcaaagcaggattcaaaagaacatctttgcgttttctaccggctccccatcatcgtactagggaggaagaagcgggtgagaaacaaaacttctttccattgtcctgcccgtttctgcggacttgttctgaggccgaggtaggttcacactcctgctcctcctcctcctctctggtagcttcataaggggcgtttaggccggggagtcctttcctagtagaggcagcttgggactccttggggaggagtttgcgggacggggctggagagggaggaatggggagggacgtgaaagtcccgtatttgtatgagttaacgactttacattcccactcaggtttactttgcgcagctgggcacagatgccagcttctattgattagtaggcttggagggggaccacagaatcgccgaacctaagggaagccctaggaggcctgcacttgcccacaactaagaaagctttgctagttattcaggctaatcaccagctttttgaagcaggccttagttttttgcaagcgtaactaacatttggggatcggtgttttctgtttactctcctctgcctcctcacccttaacccaccctcagccagtgttgtgcaattcttagagacacttgccaacagcctcttctctaagccttcctactggaaggttgggatcctccgttttctcgctctgagacagcacgtcaaatgctaatagcagcacatgagtaaagcgcaacatctttattaaaacaatttcagcaccactcagggggctagggaaatctcagatgagccaagaccctccctcgtgacagtaaaaatcttataatcaagtcataatgtaatcagtagaaacccaagggacatcattatctgtggcacaggaccaccttcccagaagcagcctcaggtattcaagacttacttaaccacagagccactaacaagttttgcaagaggataggtttattacatacgcttcaattcaagcaattcttttactgatgacaagcagagtaaaataatttttgaatttatggtaatagtcaatgtaggtttccttcgaacagcaactttcagtggcccctttaaagttagctctgattgtctagaaagtactagtatggaatataaaagttgtgtgggcccggcacggtggctcatgcctgtaatcccagcactttgggaggccgaggtgggtgcatcacaagatcaggagttcgagaccagcctggccaagatggtgaaacctcgtctctactaaaaatacaaaaattagccgggcacggtggtgagcacctgtaaccccagctactcgggaggctgagacaggagactctcttgaacctgggaggcggaggttacagtgaacagagattgcgtcactgcactctagcctgggtgacagagcaagactccgtcttaaaaaaaaaaaaaaaaaaaattgtgtggtgcattggaaagcttccaggccggcgaatgcaatgagttccttgggcaatacatgtctctttctctgttcctcagttggctcttctttaatatgaggggtggacagagtctcagaggcccctcccactcttgttctatgactccatggagtggtacgatgttagtaacaggacatgaggcactctagtaatgtggagatgaactatcgtttggcagagaggatggaaaaaatcattccaggaaaagaggaatggctttagctattgttgaaaacagataatcaggttataagcaggcaagagtaagaggtttgtcaagttattgttggaagcattggtcactgtgactcaaattaaagtagctggataaaaaagtggaccaatgaaggtcagtgaaacctttctttaggagtttttattgaatctgtatttaagcagattctctgtaattctctgtaaacagttattcccggaagaatgccatgaaatacttgggaaagagggaatgggaacaaagagtgagtgggcacaaagcccagagttggttgaaaagcaaatgtttaatttttgtctttgtttggaaatacaggcatgtcagcaggactttgtccccactgtccttcaatcagtagtgttgcctcttcctggagacgtggttcttcttgccacgtgtgcttttccttctttacctgttacttccgttattgaataagttcatgcttatagacctaaagtgttagaataataaagaaatatagaaattatttagtccattcttcccatcatctaattttataattagtcctaagacagaatgatacatatatactctgtatgaaggaggaaagtgctctaacactagggtccttggaaacaggacccgtgcatcccgtgtctgtgtgcagggacattattggcttgagggagagagagaaaaaaatctccaggaaaggggaatgactttcaagttaaagctgtctgaacatttaaaaaatatatgaggttttttgcaattggcgattccttcatttgtattaaaattactgacaaagatgtgatgttccttaatcatagaatataattccttgttgtattttaaacacaaatgactaagtgcttagatgcattctaatgaaatagtaatttttcccccaaaccctacttttcatttcaacaaaacttccttgtgccaagtttaaaagaaaaacataaaactgtctttttgcattgtgggcagattgtgtatcctccaaaggttgtccagtgaaggcccagaggcagagaagggacctaactatattcagaggctgctggttgccccacctcagcactactgcagaactctaaaatcagaggctgctctgtttgtgtgagtcggtgcttgaattggtgcttcaggccatggggctgtgtgtgtgcaggcgcatatgggttgtatgctgtggcagggtaccgcttttcgggactcagaggtggtgttgcttgagctaagcctggaaagacaggtcagttttctccttgcttatgaagggttcaaagtctgactccaccattcatctgctgtttgttcttggagtttcctcatgagcagagcatatttacataatgcctacctcacacagtcctggtgttggttgaatgtagtcatgtatgtgagagcttcacaaaatgaaagattgcactgaatattataattattgtgatttcaagtgcatttgttttactagcaggcagcctgtcttttcttgacaccttctcaagtgcagccagactgaatacttggcagctaggtgggtgacaacagaggattgaggatttttaaaaagataacactgtcctagatccaacaccactgtgggcaaatgctgggggtctgtgaaacagttggtaccctgctgctgtctgatattggaaactatgagatctataaggtgatcctggtgacctttggcccactgtttttacgcaagcaatggatctacagccctgctttgatgatggagataagcaattttgcaactacattacaaggatcagaagattgaatgttcagggttagtctcttgtggacaggtaaagatggaagaaggagaaacccaggaaacatggcaagcctgcttctgggggttagaagctgagaagttgagtgtagggggaggggcagtgtactaatagctgtcacacaaccttctctggaagtgctttcctggctgtatgaaatcgatggaataacgtgcttagcatccgttcatgaaaccaaatgatgtgatacgataaataggatgtgatatgataaatgataaatagtacagtggaaaagcatcctactggaagaccatatcttactctgtgacatcttccaaggcagttcttggagttggcccagggagatcagagatgctatcttatggatccagactattatgtgtgaatagggaaacctcagtcctgccatgaacaagagtaagcaccacagacagcagaaaagggcaacttttggacagaaggccatgtttctcaaactttgtcttccagaacaaatctctaatccattcaggtgggttaatatctaacccaatcatgcccagcgaaaaggtgaatgaccctcctcgtttgttcaagagcagtgtccttggttttcatgtccctgccaggactgggacactatatattaggaacactatataatcagaacctggagaggcctccaggttcacacaactggaacccatctccaggaacaaacagctggaacccatctcccgttgaagggaaactgccagatttttgtaagattcttcctcctgggtaaactattgttcaatttgttttatatattatttctagccatccatccattttaatgaaaccattttcctccctttaaagcctccaggctcaagctcatgaattaaaaagaaaatggatcagattctaaagtttaaaatgaaaacaggtttcttttgggatggcctatgtgtggataaagagaaattagtgagactgggaggaagtggaagtttaggagggatttctaaattgtgataaaatccccaaaggcctgggggagccatttgctgaaggccatcgggagtcctcccccgtcgtccacccctgttacaggcattacacgtggcttaacaaggaaagaaacagtatgtttttagcattgtttccttctaaccttccctgctaatttctcttcttagaaaagttttggatgctctttcaaacaaagtggaacacatcccaagatcaaaagaaggctcactggcaggttgaagggaagaaggaaggaaaagcaataaagggatgaatatgccttgggtgttaggctcttactttgggggattttgccctaggatgtataaaagtgtgaagccacattgatgaaaatgataactatagttactcatcataaactgtcaagttaactccctggtgtgtaaaatttgatgaatcttttgaatactgaacattgcctgtggggagagggagagaataattcttgtgaatactaacagattcatttgatacagaagataaagttactctaaagaacagttggggccaggtgtggtggctcacacctgtaatccccgcactttgggaagccgaggtagccggatcaactgaggtcaggagttcgagaccagcctggccgacatggcgaaaccccgtctctaccaaatacacaaaattagccaagcgtggtggtggccgcctgtaatcccagctacttgggaggctgaggcaggagaattgcctgaacctgggaggtggaggttgcagtgagccaagatagcaccactgcgatctagcctgggcgatagaacaagactctgtctcaaaaaaaaaaaaaaaacaggccgggcgcggtggctcacgcctgtaatcccagcactttgggaggccgaggcgggtggatcatgaggtcaggagatcgagaccatcctggctaacaaggtgaaaccccgtctctactaaaaatacaaaaaattagccgggcgcggtggcgggcgcctgtagtcccagctactcgggaggctgaggcaggagaatggcgtgaacccgggaggcggagcttgcagtgagccgagattgcgccactgcagtccgcagtccggcctgggcgacagagcgagactccgtctcaaaaaaaaaaaaaaaaaaaaaaaaaaagaaaagaaaagaaaaaaaggacagagaacagctgggactaattccactgacaaaaagctgacagacagttacgtgtttgggtacagaaggatatatctataggacttttgccaccagatgtgaggctttgcacccttggcttaggcagattcacttttacaccacaaacgatctgtctcttactggttttctcccaattccattttaccttgctttttgaggaatttagagttgaatatattgagaaagttttgagtgagggagcagatgttagatattggtggttttggtgtacgcttgctgagatttggttcctggatgtcttagggaatcaagattacaggcccccaactaaacttatacaaaatttgtctctgtaactgagggtcactggacctttgctattgaaactagcaaagcctctattatgtttaatgagaaccatgaaacttcagatttttagagctagcaactcttttattttcttgatgaacaaacctaggcctggagagaaaaatgaagtcaattacttaaaaaatattaagggttataataaggcaagcatgttgattcaaggtgaataatatgagttctgtggaatatacaccagtaagaaacactaacaagtaaatacttcattgttcatccacgatcacgtgggtaatggcatgtttggaaatggcacccgtgaaacctgacaacaggtcggtctttattttgtgatatccttatgggtagattctgaactttgtggcttttactttggcacttatatctaatgaggtgtccttggtcttaatatctaatgcagagcgagtttggacagagattctaaacaggttcaccagctcatactcccttctgtgaacagcaatatccccacaagttatacagcattggcacctcctgcaagtacggtatttcctagagaagacattagtaacattcctatccaccactgccacctaacgactcttttaggtactagcagctctggttccgtttcctgatttctgatttctgggattctggcaccaggcagaagaatgttgactgtgtaaggcagacctttgacattagaagtcagtatttgttcattacagagcaagagacttaattgattacaaatcaataaaactttaataagatcttccaaaaaactagaaacaggaaacacacccaggagactgaaaagcatgatgtaaatgtgaagaacgtggcaagtgggtggcttggcagcccagcagctatactggagcagctgcatgagtgaatacttgttactcctttagggctggctctccattctacaatcatgtacctctttcttgataaactcccaagaacacactttcagcatggaatttcagacttttagagatagcaactctctcaagtatggacaaatgtcggtgcggacaaatttgatgcactgctgaagaataccaaataatatgctaaggaattttttcattccacaataatggagtaaatagcagctggaaatgtttgcattaagttcatagattataatttgtaatggaatcaacaccaaatgcaaattagaaagagagcccactttgctcacccagtcacgtcttcccatgtaaccatagaacattggggtcctgtgtctttctagatccacagtcttgctctcagaacaggctagccacaccacaggcctagtgccaggacccatggcctttttttaagctcagactcccttctgtgaacagcaatatccccacaacttgtacaacattggtgcttcctgcaagggctacagaactatttgatacgaaaatgttcattgacttacacacaagagaagcacaaaataaaaaattaataattaatttaatgtctttgaaaatgtaccatttatttttacatttggggtcataagaattgtattacacttaagaatgcaatacaatttgaagatcagatttttctccctttgtgagaatttctcagtatgtgtgatgactaccaagaaatcatagccagtcataaattcagtgagttactcataaacgaacaagaaccacctacttcttggggaggtaggtctgcttcccttcaactcaggatacaactgctttcaactgctttcttcacattagctgactaattagctagaagcctgtcgtaaacaattttatggttgactccttccctgggctcagggttccctagaacagaggtccccaaatcccggtctgtggcctgtccgcctaagctctgcctcctgccagatcagcaggcagcattagattctcataggagctggacgcctattgtgaactgcgcatgtgcgggatccagattgtgcactctttatgagaatctaactaatgcttgatgatctatctgaaccagaacaatttcatcctgaaaccatcccccaccaatccatagaaatactgtcttccacaaaaatgatccctggtgccaaaaatgttagagaccactcccctaaaactctcttcttagctctcacctcctgtattactatctcatctcagtacattgaagcccccatcttttccccatggatgcctcatttcctattagggaggcatttttttattttttgtttttatttttttccgagacggagtctcgctctgtcgccaaggctggagtgcagtggcgcgatctcggctcactgcaagctccgcctcccgggttcacgccattctcctgcctcagcctcccgagtagctgggactacaggcgcccgccactacgcccggctaattttttgtatttttagtagagacggggtttcaccgtggtagccaggatggtctcgatctcctgacctcgtgatccgcccgccttggcctcccaaagtgctgggattacaggcgtgagccaccgcgcccggccgtcatttggtatgtcttaatgtgcctcaggacctagcacagtccctggtacccagtagagacctatgtaatgttcattattcaattaataaatacatgaattaaagagtgagagtggattttgtaatgttacgactgatagagaaatactcagtgattctaagggatggggaagaacggttggagctagaggttgtgctcaggaaactattaaatagacgttccgcaggaagggattgacgaagtgtgaggttaatgaggaagggaaaatagaatataaaatttggtggtggaaaagatctgattcatgatgccgtgtcagagagcaaagctcctgtccttttggcctaatttggtgatgctgttcttgggtctaccacacctccttttgccctccgcaggagcctgtgttggaagagatggtgatgggcctgggcgttttgttgttggtcttcgtgctgggtctgggtctgaccccaccgaccctggctcaggataactccaggtacacacacttcctgacccagcactatgatgccaaaccacagggccgggatgacagatactgtgaaagcatcatgaggagacggggcctgacctcaccctgcaaagacatcaacacatttattcatggcaacaagcgcagcatcaaggccatctgtgaaaacaagaatggaaaccctcacagagaaaacctaagaataagcaagtcttctttccaggtcaccacttgcaagctacatggaggttccccctggcctccatgccagtaccgagccacagcggggttcagaaacgttgttgttgcttgtgaaaatggcttacctgtccacttggatcagtcaattttccgtcgtccgtaaccagcgggcccctggtcaagtgctggctctgctgtccttgccttccatttcccctctgcacccagaacagtggtggcaacattcattgccaagggcccaaagaaagagctacctggaccttttgttttctgtttgacaacatgtttaataaataaaaatgtcttgatatcagtaagaatcagagtcttctcactgattctgggcatattgatctttcccccattttctctacttggctgctccctgagaggactgcataggatagaaatgcctttttcttttcttttcgttttttttttttttttttttgagatggagtctcactctgtcgcccaggcttaagtgcaatggcacaatctcggctcactgcaacctctctctcctgggttcaagtgattctcctgcctcagcctcccaaatagctgagattacaggcatgcaccaccacacctggctaatttttgtgtttttagtagagacagggtttcaccgttttggccaggttggtcttgaactcctgacctcgggagatccgcccaccttggcctctcaaagtgctgggattacaggcatgagccactgagccgggccactttttccttatcagtcagtttttacaagtcattagggaggtagactttacctctctgtgaaggaaagtatggtatgttgatctacagagagagatggaaaaattccagggctcgtagctactaagcagaatttccaagataggcaaattgttttttctgtcaaataataagctaatattacttctacaaatatgagaccttggagagaagtttccaaggaccaagtaccaacataccaacagattattatagtttctctcactcttacacacacacacacacatatacacatatgtaatccagcatgaataccaaaattcattcagggtagccaccttttgtcttaagcgagagataattttgatgtttgaatggaatgctcccaggatattctcttgtcatggttattttatataaaattcaaaaaccaattacattatttcctctgtaatcttttactttatcaactaatgtctggcaagtgtgatgttttggggaagttatagaagattccggccaggcgctgtggctcacgcctgtaatccagcactttgggaagctgaggcggacagatcacgaggtcaagagatcaagaccatcctggacaacatggtgaaaccttgtctctactaaaaatgtgaaaattagctgggcgtggtggcacacacctatagtcccagctactcgggaggctgaggcaggagaatcgcttgaacctaggaggcggaggttgcactgagccgagatcacgccactgcactccagcctgggcgacagagcgagactccatctcaaaaaaaaaaaaaaaagaaagatcccagtttatcccagtttatcccttattcttcctcaattctcaagatttgtttttaagttaacataacttaggttaacacactctttgtaaaatacactgttcaatctacagactcagtggttagcttcctgttaactaatttctgttgacaggtacttggatattttatttagaaagtggttgccaataaattagttataagtcgccagtttcactgccttgtgaacacataattattgtggtctcagtattccctatggtggcttctcctgctcctggtattgccctgaaatgggccaaaagccgtggctccccaatactcaggttatagaacattgtccaggtaccacctaggagagcccagcctcactgaaagtattcaaatttaggaatgggtttgagaagtaggtagctggtatgtgcttagcacaagaatctctcttccttgggttagtctgtttcaaaactgaaaacactgtcattccttaagaaaataggaaaaagtattccaaacctctgtcactagaaaatttgccatattaccaaatctcaaaaacctctcaggaaatgagaaagtcccagtttctggtaaactatttgggcccttttctcaagttctccaaccagtgctatttccttgaggtgaggcaaagttactcaagatcatcgctgccactcaaggccttgatagggcaagtgaaaggcatggaccattattatattgatcacagcataagctgtgaaaacccacatcttctccaaacatctgcttggagcattatcatcgcatagtttgctctggtgttcagggaaatcgctgtttcataggaaatcacatggcagtgggatgggagtgtttcctgacctgccgatggtactggcacctgagcaagcattcctagtcctttttggtctgggcctcttgttctatcacaaccacaagctgtttaaaataaaacgtcaagtcacaggcaggtcattttatcctgcgtgaatcaattgaagaattgaagtcctggtcagctgtgattgttggctttgcaaggaaaaagaaggaacactccaaaatgctaaatgctttgaagaatagtgacatgcaaggaagaaaattccatgcaatttggagaagaaacatttttctaggaatctagtagctgagatattttatagtttaaaaacagacttccaatatcttgagaatacagttagctctacgaaataaagaggagtcaataatatatgcaactctgtagtcattcattattttatccagattgacagaagcagaggaaaccaagaaaaatggaagtggtaaaaaaaaaaaaatgcagtaattgccaatatttactgagcatttaccctgtgctaggccttgttataggcgcttagattgattttcttttcataacatccctaagaggtaggtattacatgctcattttgcttatgaggaaatctgctgagagagattaagtaattttctcatggtcacagcctcagtcttttagaccacggtgtcaaaagaagttcttactgtgagtcagggtggtgcagtggagaaagcgtttggggaaaatcctgacccaaaagtgtgcgttgggatgatgtggagatcattcaattttctcttgtagctacagctttgagatgcacccattggtatccacactggcctgctgtcctcaaatatttctctatgtcttgtttacttgagtagtgcagaaaagctgacgtcctgggtctgctctcgcatgcataggtgaggttttctctgtagctagtcatcttgctaaaccatatttttggtcacaaagaatcaggatgagagaaagtagataaaaccatggaaactcgaggtgccaccggaagagcaaaccaaagtatattgatctgctgaagctataatccaagtactaagtataccttcagaagttgtatgcattgttcttggggcaaaaaggaaatcaaacatctgcccttcctcccacaacctctttacttccctctcttcctaacactgtatctcacatctacagctgcaggcacacagatatgacatacagcttgatatctttatgtgcaaaggcaaaagatggctatggatataggaggtggcatttaaaagaacaatatccatgtttgatactgctttcattatcaataatgaaaaggtagcctttcagcagaacaaagtaaggtacaggtcgtttggaagccatttcagtcattcacacagtgtgatccaccattacgaagcagaatttgaaatcaaaagacaatagtacccatatacatcaaactaggagttcaactggggtcagtccttggactgcacatacagagttgggctgcctgactctcaaaacattgcccttgctaatttgcgttgctcacttcctattgaatcagggatgatttttctttgctgtagtgaatcagccctgctacactgcaagaatcaagccgattcacaattcacatcaattccaggagtgacggccagatggcagcatctggctagtcagttggtgagtgagtccacctccagtcttgattaaactctgctgctgggaagtagcaattctgagcataaatgtctttgatatcctgaatattttgggtgtgggatgagagaggcagtagatagtggaaagagcacagggtgtagactaagataggcctggattcctttctttaatctgtcaatgaaagcaatgaatgttctgaaactacatggcaatgctttttttgttgccactctggcttttttttttaatgctataaaatatatgcctatttttcactgccaagaggtgacaacacaatgatgctgcaaccagagaagacagacagctaccaacaactattgacaagacagaaggaacatcaagaatagatgcaagacaaagcaatgtatctccatggcctagattttttaggttatgcatagaaacagcaaggagaagaagacacaatccaagttttcttttgagaatattgtgctgtttaaatttattttttaaacatttcttagcttttcttattagtcaatttcataagcctgggaaacatctttactctatccatttaattgcatttgttcattagcttactcatttcacttattagttcattcaaacagtagctattatcaactgtcttctaagaactgtaactgggtattctggttttgcttttttttaaaaaatagaattaaaaggatttccaaaaggacctgattttacattagaattagaataatacattttaaattaatgtataacatatattattctagaattagaatagattttaaatcacattagaataatagccacatgacaaaaaagaaaagaaaaataataaaattaatgacaaagaaaaacaatagtcatgtgagaggaaaggcaaagcctaaatattcctctgtcctcttcctccacctagattcccagatgtaaggtattaaaagtagtttgtaaactttctgtgacgtatgctgagtctatccaaactgtcgggaattggagcctcccgggaaccaaacactccccaaagatcatacacctacggagttcccccaagagacacacaactgcagtttcatgtaggttcaccttttctctctttttatactctgtccacacaaacctggcagccacgcagtcagtccctgccctgtgtcattgtactggccacagcaacttctctctgtaaatgagaaaggtcttgctttcagaaaagatcatgtttgcaaactactcactcttgaggaagaagggtgggaaaagtagcagagatgagtggggagatggtgcatataagaaggaggaaagaggagactatggagtcaggatgccggcttgctattctcaacacctttttccagtgcccagttcttaccttatttctcctgccccttgctttcttttctaggcacctctaagatactgatggctctgcagaggacccattcattgcttctgcttttgctgctgaccctgctggggctggggctggtccagccctcctatggccaggatggcatgtaccagcgattcctgcggcaacacgtgcaccctgaggagacaggtggcagtgatcgctactgcaacttgatgatgcaaagacggaagatgactttgtatcactgcaagcgcttcaacaccttcatccatgaagatatctggaacattcgtagtatctgcagcaccaccaatatccaatgcaagaacggcaagatgaactgccatgagggtgtagtgaaggtcacagattgcagggacacaggaagttccagggcacccaactgcagatatcgggccatagcgagcactagacgtgttgtcattgcctgtgagggtaacccacaggtgcctgtgcactttgacggttagatgccaccatgtagggattatcgcgagtggttgaccttacacttactccttaaatagcagtgagtaatgcatttgagctgtcccaggctctgtctcctcagctcatttcctactctttttctctatataactcattctattaaatacattgcaccaaagagatatggagacataaacctgtaatgaatgaggctgggcttttctgtaataagcttccttttataatactggtcagcttagctctctcagatcctatcctgtggaatttagttattatgtgtatttatgtagtatttcaaacatttcaaaatgctttcatctatgtttatcacattttaataccacagcacttataatgatgtcactacatatagaagctcaaagttaagggatttgctgaagactgtaaagttaatggaagaattgagacaaaaatccagtgtagctggccacttatccagggctttttctacttcatcacaaggaatgttttgaaagtgtctgctttttttatccttaaaattcacctgtcagggaggcattaaaaatttggaaatgtatgccagcaaaatgtgagctctgtattttttggcattcttatgtttgggtttaataagattaagaaaatgatactgggaattttctttttcctgaaactttgaatcaccctagtaagtcaaagtactaaaaaatgtactagatcattaagacttatgtgctcttactgattgaaagattttttatgttttccttgtaataaaggacctaaaccgaaggtacctgagaagactgtgctatggtattattttttttctctgacttttaaattcttgtttagtttataaacatgcatgcaccttaataacctcataaactctggtcaaagactaatgcctatcagatccatgaccacaacacagaagatttgtctcatttactccagagagaatgattcctctcaaagacaattctcacagctcttccttctcccttagaatatttagaagcaaattagggagctgtcaggtctctgagcccaagcctgcacgtatacatccagatggcctgaagcaactgaagaaccacaaaagaagtgaaaataaccaattcctgccttaactgatgacattccaccactgtgatttgttcctgcccc

SEQ ID NO: 8 - DNA, 1268 bp, Homo sapiens
atgtctgaatatattcgggtaaccgaagatgagaacgatgagcccattgaaataccatcggaagacgatgggacggtgctgctctccacggttacagcccagtttccaggggcgtgtgggcttcgctacaggaatccagtgtctcagtgtatgagaggtgtccggctggtagaaggaattctgcatgccccagatgctggctggggaaatctggtgtatgttgtcaactatccaaaagataacaaaagaaaaatggatgagacagatgcttcatcagcagtgaaagtgaaaagagcagtccagaaaacatccgatttaatagtgttgggtctcccatggaaaacaaccgaacaggacctgaaagagtattttagtacctttggagaagttcttatggtgcaggtcaagaaagatcttaagactggtcattcaaaggggtttggctttgttcgttttacggaatatgaaacacaagtgaaagtaatgtcacagcgacatatgatagatggacgatggtgtgactgcaaacttcctaattctaagcaaagccaagatgagcctttgagaagcagaaaagtgtttgtggggcgctgtacagaggacatgactgaggatgagctgcgggagttcttctctcagtacggggatgtgatggatgtcttcatccccaagccattcagggcctttgcctttgttacatttgcagatgatcagattgcgcagtctctttgtggagaggacttgatcattaaaggaatcagcgttcatatatccaatgccgaacctaagcacaatagcaatagacagttagaaagaagtggaagatttggtggtaatccaggtggctttgggaatcagggtggatttggtaatagcagagggggtggagctggtttgggaaacaatcaaggtagtaatatgggtggtgggatgaactttggtgcgttcagcattaatccagccatgatggctgccgcccaggcagcactacagagcagttggggtatgatgggcatgttagccagccagcagaaccagtcaggcccatcgggtaataaccaaaaccaaggcaacatgcagagggagccaaaccaggccttcggttctggaaataactcttatagtggctctaattctggtgcagcaattggttggggatcagcatccaatgcagggtcgggcagtggttttaatggaggctttggctcaagcatggattctaagtcttctggctggggaatgtagacagtggggttgtggttggttgg

SEQ ID NO: 9 - DNA, 3123 bp, Homo sapiens
acgtcctccagccccgctcccgacgtgaggggcggggcttgcctggaggcggggcgcagggatccggaaacacctgatcatctataggtttagtgcctaatgggtgttgttcctggctggacttgatgtccagggcctgaggggttttctcgccgagtctcctggggcggtccggaggctcgtgccctgttgtggggcccccatttgccgccgccatgcccacggccgccgcccccatcatcagctcggtccagaagctggttctgtatgagactagagctagatactttctagttgggagcaataatgcagaaacgaaatatcgtgtcttgaagattgatagaacagaaccaaaagatttggtcataattgatgacaggcatgtctatactcaacaagaagtaagggaacttcttggccgcttggatcttggaaatagaacaaagatgggacagaaaggatcctcgggcttatttcgagcggtttcagcttttggtgttgtgggttttgtcaggttcttagaaggctattatattgtgttaataactaaaaggaggaagatggcggatattggaggtcatgcaatctataaggtcgaagatacaaatatgatctatatacccaatgattctgtacgggttactcatcctgatgaagctaggtatctacgaatatttcaaaatgtggacctatctagcaatttttactttagttacagctatgatttgtcccactcacttcaatataatctcactgtcttgcgaatgcccctggagatgttaaagtcagaaatgacccagaatcgccaagagagctttgacatctttgaagatgaaggattaattacacaaggtggaagcggggtatttgggatctgtagtgagccttatatgaaatatgtatggaatggtgaacttctggatataattaaaagtactgtgcatcgtgactggcttttgtatattattcatgggttctgtgggcagtcaaagctgttgatctatggacgaccagtgtatgtcactctaatagctagaagatccagtaaatttgctggcacccgttttcttaaaagaggtgcaaactgtgagggtgatgttgcaaatgaagtggagactgaacaaatactctgcgatgcttctgtgatgtctttcactgcaggaagttattcttcatatgtacaagttagaggatctgtgcccttatactggtctcaggacatttcaactatgatgcctaaaccacctattacattggatcaggcagatccatttgcacatgtggctgcccttcactttgaccagatgttccagaggtttggctctcccatcatcatcttgaatttagtgaaggaacgagagaaaagaaagcatgaaagaattctgagtgaagaacttgttgctgctgtgacctatctcaaccaatttttgcctcctgagcacactattgtttatattccctgggacatggccaagtataccaaaagcaagctgtgtaatgttcttgatcgactaaatgtgattgcagaaagtgtggtgaagaaaacaggtttctttgtaaaccgccctgattcttactgtagcattttgcggccagatgaaaagtggaatgaactaggaggatgtgtgattcccactggtcgcctgcagactggcatccttcgaaccaactgtgtggactgtttagatcgcaccaacacagcacagtttatggtgggaaaatgtgctctggcctatcagctgtattcactgggactgattgacaaacctaatctacagtttgatacagatgcagttaggttatttgaggaactctatgaagatcatggtgataccctatcccttcagtatggtggttctcaacttgttcatcgtgtgaaaacctacagaaagatagcaccatggacccagcactccaaagacatcatgcaaaccctgtctagatattacagcaatgctttttcagatgccgatagacaagattccattaatctcttcctgggagttttccatcccactgaagggaaacctcatctctgggagctcccaacagatttttatttgcatcacaaaaataccatgagacttttgccaacaagaagaagttatacttactggtggacaccagaggtgataaagcatttaccattgccctatgatgaagttatctgtgctgtgaacttaaagaagttgatagtgaagaaattccacaaatatgaagaagagattgatatccacaatgagttctttcggccatatgagttgagcagctttgatgataccttttgcttggctatgacaagctcagcacgtgactttatgcctaagaccgttggaattgatccaagtccatttactgtgcgtaaaccagatgaaactggaaaatcagtattgggaaacaaaagcaatagagaagaagctgtattacagcggaaaacggcagccagcgccccgccgccccccagcgaggaggctgtgtccagcagctctgaggatgactctgggactgatcgggaagaagagggctctgtgtctcagcgctccactcccgtgaagatgactgatgcaggagacagtgccaaagtgaccgagaatgtggtccaacccatgaaggagctatatggaattaacctctcagatggcctctcagaagaagatttctccatttattcaagatttgttcagctggggcagagtcaacataaacaagacaagaatagccagcagccctgttctaggtgctcagatggagttataaaactaacacccatctcggctttctcgcaagataacatctatgaagttcagcccccaagagtagacagaaaatctacagagatcttccaagcccacatccaggccagccaaggtatcatgcagcccctaggaaaagaggactcctccatgtaccgagagtacatcaggaaccgctacctgtgaaaagagcgcaggtccacctggtggacacgtctgattagcttagaacctgtcttgtctcatcttcaaaaggtaacttattaaaagtcctttgcgtctgaagcctttctccttttctgtcacttgcaaattccaaattatagctaataaagatgactagataatttgcaaaaaaaaaaaaaaaaaa

SEQ ID NO: 10 - DNA, 1863 bp, Homo sapiens
gttcgttgcaacaaattgatgagcaatgcttttttataatgccaactttgtacaaaaaagttggcatgtcccatcaacctctcagctgcctcactgaaaaggaggacagccccagtgaaagcacaggaaatggacccccccacctggcccacccaaacctggacacgtttaccccggaggagctgctgcagcagatgaaagagctcctgaccgagaaccaccagctgaaagaagccatgaagctaaataatcaagccatgaaagggagatttgaggagctttcggcctggacagagaaacagaaggaagaacgccagttttttgagatacagagcaaagaagcaaaagagcgtctaatggccttgagtcatgagaatgagaaattgaaggaagagcttggaaaactaaaagggaaatcagaaaggtcatctgaggaccccactgatgactccaggcttcccagggccgaagcggagcaggaaaaggaccagctcaggacccaggtggtgaggctacaagcagagaaggcagacctgttgggcatcgtgtctgaactgcagctcaagctgaactccagcggctcctcagaagattcctttgttgaaattaggatggctgaaggagaagcagaagggtcagtaaaagaaatcaagcatagtcctgggcccacgagaacagtctccactggcacggcattgtctaaatataggagcagatctgcagatggggccaagaattacttcgaacatgaggagttaactgtgagccagctcctgctgtgcctaagggaagggaatcagaaggtggagagacttgaagttgcactcaaggaggccaaagaaagagtttcagattttgaaaagaaaacaagtaatcgttctgagattgaaacccagacagaggggagcacagagaaagagaatgatgaagagaaaggcccggagactgttggaagcgaagtggaagcactgaacctccaggtgacatctctgtttaaggagcttcaagaggctcatacaaaactcagcgaagctgagctaatgaagaagagacttcaagaaaagtgtcaggcccttgaaaggaaaaattctgcaattccatcagagttgaatgaaaagcaagagcttgtttatactaacaaaaagttagagctacaagtggaaagcatgctatcagaaatcaaaatggaacaggctaaaacagaggatgaaaagtccaaattaactgtgctacagatgacacacaacaagcttcttcaagaacataataatgcattgaaaacaattgaggaactaacaagaaaagagtcagaaaaagtggacagggcagtgctgaaggaactgagtgaaaaactggaactggcagagaaggctctggcttccaaacagctgcaaatggatgaaatgaagcaaaccattgccaagcaggaagaggacctggaaaccatgaccatcctcagggctcagatggaagtttactgttctgattttcatgctgaaagagcagcgagagagaaaattcatgaggaaaaggagcaactggcattgcagctggcagttctgctgaaagagaatgatgctttcgaagacggaggcaggcagtccttgatggagatgcagagtcgtcatggggcgagaacaagtgactctgaccagcaggcttaccttgttcaaagaggagctgaggacagggactggcggcaacagcggaatattccgattcattcctgccccaagtgtggagaggttctgcctgacatagacacgttacagattcacgtgatggattgcatcatttacccaactttcttgtacaaagttggcattataagaaagcattgcttatcaatttgttgcaacgaac

SEQ ID NO: 11 - DNA, 23675 bp, Homo sapiens
agggctcttgtgatcttgctcaagtcagctctcagccttagtctccttattagtaactcaaatatggtccttgccctgagtgaggaggcacgctggaatagatcatcactgccccaggtttttcttccttttttttaattcttgcctcatagctatctatctcttagaattgggggaaactacaggcattatgcatagcttcttgctttgtaagggtagctgaggcctgtgtcctcaaggatcaagaggacccttgagctcaaggtctagccaggatagatggagataggagaagacgaggaggttgtaggcttggagcttctggttgtccttctttccccagagtggagcctctaaggatctgctgttacttctgaaaacttggagtcccccagctgaggaattagatgctccattgaccctgcaggatgcccagggattgaaggatgtcctcctgacagcatttgcctaccgccaaggtcagctagcaacctgactcatctcttctacctttaagtggtcctccagactcatgccctctctcttgtcccattcagttccatgggcttcttagacccttgttccccatcatgacccctgctctatagccctctccaagctcacaccctgactgtcctcaggtctccaggagctgatcacagggaacccagacaaggcactaagcagccttcatgaagcggcctcaggcctgtgtccacggcctgtgttggtccaggtgtacacagcactggggtcctgtcaccgtaagatggtaaagacagtcctagggtggattggggaaaccacaagcattatgtatagctttttgctttgtaagggtagctgaggcccgtgtcctcctatccctttagggaaatccacagagagcactgttgtacttggttgcagccctgaaagagggatcagcctggggtcctccacttctggaggcctctaggctctatcagcaactgggggacacaacagcagagctggagagtctggagctgctagttgaggtagggaactgccaggtgaagatgtggggtggaggattcctgaggtgcttgtgatggggtgacttgactagtcaagatccaagtataaggaagtatggctcccaaagattatagatacaatttttttctttctcttttaggccttgaatgtcccatgcagttccaaagccccgcagtttctcattgaggtagaattactactgccaccacctgacctagcctcaccccttcattgtggcactcagagccagaccaagcacatactagcaagcaggtgcctacagacggggaggtgaggttccccttgctcacctgagcttctcttcccacttctgatgcctcccctggggtgtgactctgcttttgttcttgtgacttgggggatgaatgaggcagtggcaggacataggtcgccactcctcagcctcttaccttgcttctagacctgaactgaggactcttctctaagacattaaccttctctatctttgacactctgaagtctcttccccagtccattctgtaactggggtagatccatactgagccaaaattggagccctaagaccttccctccctttctcctcctcagggcccatgaacatccatctgttagctaagagagaacattctcaggacccttgcaggttccctgctagggcctagggtgaggcttatgggcttttactcctcagggcaggagacgctgcagagcattacttggacctgctggccctgttgctggatagctcggagccaagggtgggtgtgtcttcaagcttctctgcaatggggtagacgggttggtgtcccctttgaaatacagcaagactgcaattttttgttttttcctctattctgtctgtccagtctgggggcccaggactctgcatggtaccagagcctggtgaagaggttggggatggtggctcatgcctttcaagacactgcttcttcttcccactccagttctccccacccccctcccctccagggccctgtatgcctgaggtgtttttggaggcagcggtagcactgatccaggcaggcagagcccaagatgccttgactctatgtgaggagttgctcagccgcacatcatctctgctacccaagatgtcccggctgtgggaagatgccagaaaaggaaccaaggaactgccatactgcccactctgggtctctgccacccacctgcttcagggccaggcctgggttcaactgggtgcccaaaaagtggcaattagtgaatttagcaggtgagcccggggtcctagagggggtgtggagggatgattttctgattgggacctgagttggtgagctccatgttcacctacttaccctagagtttctgaaattgattcctcttcacccctgttctgtgtcctacttcaggtgcctcgagctgctcttccgggccacacctgaggaaaaagaacaaggtgatctaaactttcagttttcctcttacctcctggaagtggtagtggtggaagtgggttccctttaatgtgtcccagcccaccaggagacttaagagaaagggactgtggacacaagaagaaatagaggaggatttggggttttggtgactgtgaggctgggaagacacttcttgactttgggagtggtccccaggggcagctttcaactgtgagcagggatgtaagtcagatgcggcactgcagcagcttcgggcagccgccctaattagtcgtggactggaatgggtagccagcggccaggataccaaagccttacaggacttcctcctcagtgtgcagatgtgcccaggtatgtatacttgcatgacgtcggctatgcatagatgtgcaaggacagagaggcccatgggaaatacttgggcgtaagtgtaagagtggtgttctcagacaggaggggtgattggggttgtgctatatatacctagggatatatccaggtgttcaaatatacacagtgtgtatgcagggctcccaagagttccctggtttccccgatatcctcagacacaaagaccatgactctgctgttgtgggttgcacaggagtagggggcagatgatggacactgggatgggaatggttgtgaatgatctgcccccgcttccatatgtgagtgtaggcaagatacttcctctctctaggcctcagtttccctttttataaaacaatgcctaagactatgtcaagttctaagactctgtactctgggctggggctacaggtaatcgagacacttactttcacctgcttcagactctgaagaggctagatcggagggatgaggccactgcactctggtggaggctggaggcccaaactaaggggtcacatgaagatgctctgtggtgaggctcaggcctgctggcttggggctgaggggcagttggtgtcctagagtccttggacctattgtgggtggtgaacactgaaagcgggggtatggctgcccagctttctgccaatctttcacccttaggcatcatccttctttctctccaggtctctccccctgtacctagaaagctatttgagctggatccgtccctctgatcgtgacgccttccttgaagaatttcggacatctctgccaaagtcttgtgacctgtagctgccacgttttgaagagcttgagctgggtccccagtgggctgtctctctgtggggagggctttctgcttcaccatcattaggaatgtgaccattcctatataattcctggactggtgagattggtggtaggcctgtgaaatttgccctagttactaccattctcgttttggaggaaacaatctctgccaccaccaagtcattgactttgctcgaggcaccttttttcctgtttctccttttctgttgtcgagtaaaatttcatatttatctcgtctgcctactacatatacttgcgtgggaaggcttgtgaccgtctagttgagtgctagttgtctatgatgccctttcctgacatcgtctaccttatgaacttgaagtctgggtatgaggaaagggctacaggaatcctcactttggaggcagtggctttagtccttcacattgcctgaatgcagagccccaaagcccaggacagtgacaatgagtggagttgggtctgaagggtggtctagataggcctctcaggacaagggcaggtacctgttacttagagacaggcaggtttctctcagagtaactcttcactcccttcctaattccaaattctagaaattcgactcgtcttagtcaaatgtgtctgtgtgggtacagggtgaaaggtgaattgagtcagcatatcaccaacagctgcctgttgactactaacagggacccctgttatttggggcccaaggattttcaagtcccagcatatgcagttttcttgctaatttgggttctactgaattggctgtgtgttctgtggttgccctttgacttctctggatttgagtgccttattataaggtattaatattaatgacgggaggaagaaaggcacaagcgctgagaaactgcaaaggctgtgggattggggttaccagagggtgcagtgacaatggggtaggagtggggtgctagtcagaaagttcagagtaggggtggttggaggcagcgttgagaaggagcaagaagtgtcagggtggtgatggcgtcttagaagatctccaggttttgggagaagatgggtgtatgggttaccacaataggactagactaatacaagggttcagtctattgtgccacgttcttccaggaggataaactccggagccaatgaactggagccaagtgggagagtccgcctctaagggattaaagggtctcctttcactaatcgatctcgccctattcctttcgttgattggctgagaattccaatccgtcgaggaagcgtagcgttgcggccaattgacgtggcgttactaggcgtgtcgcatcactgaggcgggagccaggccgcaagcgaatttcctgattggctgtgatctgcgggttgctggggagaggcgcggagaggcgggcgagagtccgcagggcaggcgctgattggctgaggtgggagcagcttcccttccgatgattcggctcttctcggctcagtctcagcgaagcgtctgcgaccgtcgtttgagtcgtcgctgccgctgccgctgccactgccactgccacctcgcggatcaggagccagcgttgttcgcccgacgcctcgctgccggtgggaggaagcgagagggaagccgcttgcgggtttgtcgccgctgctcgcccaccgcctggaagagccgagccccggcccagtcggtcgcttgccaccgctcgtagccgttacccgcgggccgccacagccgccggccgggagaggcgcgcgccatggcttctggagccgagtgagtgtgcgcgcgccgttcgcttgctgcggcggcgcgcaccgggaccagccgcgcagggtaggcccggcggggcctggccgtgggcgcgtcagagaggtggagaccaggaaagagggaagggaagctagggggcgagtgaggggccgtggacccggcaggcccggctggggccagctgcgcacccgcgcgccccctaggcggggcttgcgttgggtccgggtcggagcctgggccggatcgtgctgtgtcatgcaggccccggcccgcccgattggctcccttagaacggacgtctgggcctggccaggtcttccggcccactccgccggcgcggcgccccggggctttggggcgccagtctgccgtccggcctaccacccgccgaaagcctttggtccccggagagagcaggccccgcgagcccgaggccccagccgggcccggtgggcgtggactttgcgccatgtgaaggcctcaggagctctgccaccgaggcggagcccggggtcggggaaggcccgccgagctcagggaagctcacagccgcctttttggagccgggtcggcggggccggaggtgggcatcctctgtgtgtgacctgagtcgtgaaggactgttagggagagggcgagccctacctttcgcttctggtcttcttactttaggcctgttgttcatactagaaagtttgagccagttttccttaatgttatgactcgtgggtggggagaaaggaatttttctttaacattttaggttcttgctaaagttggaatctcagttcgtgttttgtgcgcctttccagatttccaggtttcgtggtgtgcagtttagagttgcactaactcttcaaaaacacaaacgtgcgcgcactcctttccccaaccagctatattgagaaattacccgggctctggggctttctgaccccacctccatccttaggaaagcgttagataaaacttggctacctcagcccattcaatttagaatagagagcttttagaggcaaaaaaaaaaaaaaaaaaaaaaaaaaaaacacagccagcccaaggaaactctatgcaaattaccttccttccttgaagagattagttttttgaggtgtgttcctttttctcaagtttctgaattcggataaagtattactcagttgcttatttaaaagagtcacttattctgaaagtattcttttggtttatatgcagtcagcctcttattttcaaaagtaaaaatctaaaattcttggaacctggcatcatcagctgtttctaacctcttggtcaccttgatgactcttagctgaaacccttccaagtcccgtggagtcccctgtgcagttctggaaagggattgacttatacagaagacaatggaggattgccccaaagagtgtccagaactgctgttggcccatggtcaggccaaattcagtcttttagagattaccacatctgatctcagtaggtagatggggaattagaggctttaccttaagaaaagggcataccactcatctccagagccagagtccgggatggtaggcagaatggaggttttcctaggatacagctttgagattaaaatacaaactattgaccgggcacagtggctcaggcctgtaatcccagcattttgggaggccgaggcaggcagatcacttgaggtcaggagtttgagaccagcccgggcaacatggagaaaccccatctctattaaaatgcaaaaagtagccaggcatggtgatggtgcacacctgtagtcccagccactctggaggctgagacaggagaatcacttgaacccagggaggcagagattgcagtgagggagatcgcgccactgcactccagcctgggcaacagagggagactccgtttcaaaaaataaaaaactatggtgactagaggcatctggcgttttatttttctccagtcccagttctataagtcaagcaagaagatgggcagtccctggaagagtattttgatgggacaggagtgggagagctcgtgggtttacattgctctcaactgtcacattgagcatgcttggcctctagtgtgttgataagcattggaagagtctgcctactcagcagcattgtgcctggagtggcagacttttggaatgggggaagcaaatttgagcagaggaaactgtcgttagaaactagtttagaggcagtggttaaaaatgcagcctatgtgtgagggttagcagaaggcctaccattttgttagatgaatggggtttgtttttctcttgggtgtatcaggacccaaagatgtaagaaccccatggcttcctagctgagcacagcatttttctttgtctcttgcaaattgtgaggatatttccaatgggaatactattttgtcttgttttgttgacttcagtatccccagcccttagaacaatgcctggtacataatagagactcatgaatttgttgaatgaagaaattcgttttaaaaatttattttctttgcttccttcattgtctggccttcctactttggttaatgcttatgttttcctgagccttactaacacgaggccgctcttaaaaaagagagagcgctgggtgctgtggctcacgcctgtaatcccagtactttgggaggccgaggtgggcagatcacctgaggtcaggagttcgagaccagcctgaccaccatggtgaaaccccatctctagtaaaaatgcaaaattagccaggtgtggtggcgcatgcctgtaatcccagctactcgggaggctgaggcaggagaatcacttgaacccgggaggtggaggttgcagttagctgagatcgcagcattgcattccagcctggacaacaaaagcgaaactccatctcaaaaaaaaaaaaaaaaaaaaaaaaagggagagggagcttgctgagtctagtaagtgacagctggaaacgggctaggtaataagttggtgtcactgtctggtgaatgatcctagcttctaggaaataacactgagtgtagacccagtcgactttgatttgggtgagagggatttggatttgccccatgtctcagcatttcttggttttgattttttgagccaactttatggaattgtgtacttttgcagatattactgtgaagttccttttgaccttgagcctctttctggcggtttgatgtctgttagtgttttttccaaatatgatggtctttatgcttggcattcctttggtactatggaatgccctggcatcagtaggtttagcctataaggagggtagcaccaatgattctgcttcgttgtccccaggcttgcttggtagaacttaaggcctccctgagcctcaagggaggttgtctggcttacttaattccctggaaagttagcttatgctttactctatactgttaatcatggtgaccattaataccatgtgccaggtattatgttaagcattttacattcattatctcccttaagacaataagcctctgagataagtattatattccttcttttatagaggaggacatcaaagttcagaggttaggtaacttgcccaagatcacaattaagcagtggcataattggaattcagacccaatgggtctgactatagagttcctgctcttaaccactcttctgtaagtctaagactatttttatttctctaacaactattcagcctccatttctattatgtcttcttcttaggccattattttctgaccctggggcaggatcactcacttggggccttatagctgggacactgacgctcaaaataccaggagctgctggaatgggtattgtaatatgtatggtagatactgctcctcgtgaccttggctgcctttccttcatctgagctttctggtctagggacagcttcatctattcactgtttctttcctaagtatgagttttagagactggcgaggcgcttggggcaggagtatctactgactccatttcctccttctagttcaaaaggtgatgacctatcaacagccattctcaaacagaagaaccgtcccaatcggttaattgttgatgaagccatcaatgaggacaacagtgtggtgtccttgtcccaggtaagctgtggccacagactagtctttccttactgcacttacttgagggattttcccaggtttcttttctcatttttcttgcagtgactgcagatagagtggggtttactgggaatcccaatctccagggctgctgcttactccccgtcagcccaatgacccaaaggccttaactttctttcctcagcccaagatggatgaattgcagttgttccgaggtgacacagtgttgctgaaaggaaagaagagacgagaagctgtttgcatcgtcctttctgatgatacttgttctgatgagaagattcggatgaatagagttgttcggaataaccttcgtgtacgcctaggggatgtcatcaggtgtgtgtggggtttttggcttcacagggatgggaggccagagatagcctgcattacaggcaggacccatgtattacaggcaggaccaagttcttggcacctgtcgatgcaggaagcctcctggtcatgggaagacttatgcttcagggttgtctttaggttttggttctgcctccctgggacttcaaaatccatttctgcaggtcccttgagacaaattggcgttcctgtaactttcttgatggctttattttttcctactagaggtgtaatttatcatacttatatcctggcttaggataccactcgagggtgtgtatgtatatgccaaataatctcctcactactctcactagtatgtctaattgatggcttgtgttggggttggaatgaggtgggggtatgggcatggaaggtgagctgctagcaggtcttttaagcccctaagttaacccgggagagaggaatagttggagccagacctgggatagctctcaatgtgagtgattttgcttgttcttgcataattttaggcaagaggttaccccatcttggagtcatctctagccactcctacccaaccaaccatcacctggccaggatcatctcaggcttttgattcttttgaatggagtctaagtttcatgtagctttcttcttgggagtgcttagtcacttcctcaaggtgttctgaccacctggctgagataattttgtttttttcacttctgtttcactgaccctgacaattgttagcttaagaccttcccttgtaatattgggtcaccagtattagctagaaggggatcatccttggatatctccctgaagaccctgcatgtctttgtggggtttctaaatgtgtggctcttgattttggctcactgattaggagtgagtggggctgttccttcgccctcacttccaccctgttctccttcctctcttcgcctaaagccatcctgccttttctttttcacttactatcagctatctgtgccaggcccttttggacacccagtgcttgggcccgaagtgtggttggtaatatggagtctgcttgtcatcctcagcatccagccatgccctgatgtgaagtacggcaaacgtatccatgtgctgcccattgatgacacagtggaaggcattactggtaatctcttcgaggtataccttaagccgtacttcctggaagcgtatcgacccatccggaaaggtgagagctaattctgagcttaaggattattgactgtagggaataaaccttggaacatctttatctcattttctttttctttttttttttttaaatcttttatgcttttcccctgtatttatttattcattttttaagagatggggtcagctgggcaccgtggctcacacctataatcccagcaatttgggaggctgaggcgggtggatcacttgaggccaggagtttgagaccagcctggccaacatggcgaaaccccatcgtgggcacctgtaatcccagctacctgggaggctgaggcatgagaattgcttgaacccaagaggtggaggttgcagtgagccaagattgggccacagtactgcagcctgggtgacagagcaagactctgtctcaaaaaaaaaaaaaaagagacagggtctcactatgatgcccaggctggtctcaaactgctgggctcaagtgatccatctgccatggcctcccaaagtgctgggattacaggcatgagccatcaagcctagtctcattttcttttcttttttttttgagacagagtgtcgcgctgtcccccaggctggagcgcagtggtgcaatctcggctcactgcaacctccgcctcctgggttcaagcaattctcctgcctcagcctcccaagtagctgggattacaggcgtctgccaccacgcccggctaatttttgtgtttttagtagagacggggtttcaccatgttggtcaggctcgtctcgaactcttgacctcaggtgagccactgtgcccggccgctagactcattttcatatatttgtatacacacacatgcaaaccctgcacacatattcatatgtcttaccctctttttttcctccatccttcctttgctccatctctccccttctctgttccaggagagtaagctatctttatggatctctgaaggagaaagtggtccattttggctgggtcagggtccagagtgcacagttctaccattggtggttgtagtgaaaacttgggctacctatatggcagaagtcagaacttgatgggcttctgacatgtcaggttttgttcactgacctcttgtcagagggactcttcacagtttacctttctcatcttgcctgctgcttattaagacaggtggggtggagttggggagaggtagggcaatatctaatgaagggcactatctaatgagcttggcattttgaccccagggtctgatgagttctcactttgtcttgtagttgacacctctaactgtgcttgtactgtttgctctcgcaggagacatttttcttgtccgtggtgggatgcgtgctgtggagttcaaagtggtggaaacagatcctagcccttattgcattgttgctccagacacagtgatccactgcgaaggggagcctatcaaacgagaggtgagttttctccctgattccagtatccgattttatgattactcagtgtggcatcatgtggtaactgtcaggactgggtgctcggccggctgcggtggctgacacctgtaatcccagtactttgggagactgagatgggcagatcacttgaggtcaggtgttcaagaccagcctgggcaacatggtgaaatcccatctctactaaaaatacaaaaattagccaggcatggtggtacacatctgtaatcccagctactcaggaggctgaggcaggagaatcggttgaacccaggagtcggaggttgcagtgagctgagattgtgccactgcactccagcctgggtgacagagtgagactctgtctcaaaaaagaaaaagactgggtgttctttggagaactaaccatctttcagggatgagaaacctgccagctattcatttctgggcctaattgtttcttggatttacctaatgccaggaatttcaaaaaactagactgaacccaaaatatataagtgattgaaatcatttttgaagtaaagctgatggtggcttcaggcctctgcccattcccagggtttccagcttcagattttagagaccccttctcagtaagactacgagtaatgtgagaggcaaggactgtgctagaaatctttgccttgggatttttgtagttgttctttgaggccggatccctttagaggagaatcttttttaaatttaatttaatttttaatgagatggagtcttgctgtattgcccaggaactcctggactcaagcattcctcccacctctgcctcccaaagtgctgggattacagatgtgagccaccatgccgggttgagaatcttcttatacggtaggtttttgcacactaggtagtggaatgatttagagaaactcagcttttgctgatataatattcttgccttctcctttctttatctcctccatattcaggatgaggaagagtccttgaatgaagtagggtatgatgacattggtggctgcaggaagcagctagctcagataaaggagatggtggaactgcccctgagacatcctgccctctttaaggcaattggtgtgaaggtgagcatcctgggctctggaatcaagtctaaagtggtgccaatgtctaatcctgtcccaatgtctaatcctgggactgttttcatgcatggctttcattattgccttggattagaggggcaataacgtatcctttagtttacctaaggctctaaattcattagagctgatggtctaaaaccagagtaggctaatcaaattgtctgttgtgtgcgtgtgcgcacaaaacacacacacatatatatatgggtttttctttacaactcttagaatataaaagccattcttgtatcaatggaccctgtaaaaacaaatctcaccatagtttgccagcctgtctagagcaatgtcacccagtagaagtaaggaagttaaggaaattttcagagtgttaaagggttctgagtctaaaacatttgagaactattggtctagagtgtagcttctcaatcttttcctagtgggaaagtgtttccatggaacacactgaagatgaagttactcattttcctagtgggtggcacacaaataatttcattttctatgtggacagtttacatgttctgcttgtggatgaggccatagaaagggtagtgttgaagaagaaaaatgatgattgtaaggaacagcattccagtgtgataaattctggagggcatgattactggagtgagtgatcctctggcaatgaagaaaatagaccctgctctcttaaatggcttagctagtctttggcccttggtctgtctaaaattgagcccttagtgtaatggcctcttgcctttccctagtcatgtatcttcaaacgcatttggactacagtttctctgcccttagtctcctatgcaagttgcaatcataaatgttgcccactttctagcagtattttccctgctagtaatagaaatgagtgtggcctaaagtaattgtcttcttagcatttactgcggagggcttattcttaatattgtcagggttgaagcctgattctcaccctctctggagcgctagtcaagccattttagggtttgggagaaggtgggaacctaatcacactctgcattggtccacagcctcctagaggaatcctgctttacggacctcctggaacaggaaagaccctgattgctcgagctgtagcaaatgagactggagccttcttcttcttgatcaatggtgagatatttggttcatcttatgtctagctagacccaattttgaactgggcttatgagctggagcacttatgaacacatcctttttgcacccatgccctccttcatgtttatagcatatttcttatgctggggtatgttacagacagaagagcaataaagggaagatattttacattggtgctccctgtcctgccccctttgagaaagattgtggacagactgcagagcgggagcaagctagaatgagaaatcaaagggtgaatggttagtgatttgagagggtttggggcaaatgaactttgatcactggctcttggagaatgctgtttagtggtgtgccatctggtgtgccatctctcttgctctagccagaggtcctagagcatttgctgtcacctttacagttcaactgtgagaagagtatagtgagtccctgggcttctctccagccttgcctggtggctgtcctgggataatggctggtagaggatgtgagaagtaggcagaggttaccaccttctcacccaggacctgtctctgggccaaacaagcaagataactgatttttgggaggaattgggaaagactatcattttgttattgtctccattctgtatcctttcaggtcctgagatcatgagcaaattggctggtgagtctgagagcaaccttcgtaaagcctttgaggaggctgagaagaatgctcctgccatcatcttcattgatgagctagatgccatcgctcccaaaagagagaaagtaggagcttacctgaggggatagaggggggttgaaaggccctgacttcacttctgaccagacatcctgttctggcagactcatggcgaggtggagcggcgcattgtatcacagttgttgaccctcatggatggcctaaagcagagggcacatgtgattgttatggcagcaaccaacagacccaacagcattgacccagctctacggcgatttggtaaggactccagatacttttgaccccgtccttgcttaggtcctacttctctccttcatctaagtcacctaatcctcttgaagcccttcacagtgattgggtccaggggtctttttcctttaccctacgtcctgtctagagtgaccaaccaccctggttttcctgagactgaaaggtttcccagagcttgagacttttttagtgctggcattaggacaatcctgtgctggctgagatggttggtcaccctaggcctgtctcttacctctggactagagatgagccctgtttatgtttgtgtactgtcccacaggtcgctttgacagggaggtagatattggaattcctgatgctacaggacgcttagagattcttcagatccataccaagaacatgaagctggcagatgatgtggacctggaacaggtgaagtgatgatgatggctgaccaggcgttacagtgtctctaggcagttgctgggaactggctagagacataaggttaagatgtgaggagatgggttttgatttctggacaggggaaaggaagtaatctgagattgaatccaggaaatgggagttggcatttttcatagttgacgctgcatttagagtaaatcagaattgttggagcagccttatttctaggtcccaagtccagaattaagtacttaaaacccagcccataaaggtattgatagtatatattcaaggaaatgagaggacccagggatagcagtcaggggaaggattctattgtctctgagcctcctgcagcagctgggtctttgaggcagcatagtaagtagatctttctctgcaggtagccaatgagactcacgggcatgtgggtgctgacttagcagccctgtgctcagaggctgctctgcaagccatccgcaagaagatggatctcattgacctagaggatgagaccattgatgccgaggtcatgaactctctagcagttactatggatgacttccgggtaaggaccacacccgtgcctcaggtacacacatacgtgctttgacccctcccttgataagtctcatccccagttttccctccttttctagtgggccttgagccagagtaacccatcagcactgcgggaaaccgtggtagaggtgccacaggtaacctgggaagacatcgggggcctagaggatgtcaaacgtgagctacaggagctggtccaggtagggcaacttggtccagggtgagtcactgtctcagtacattgtaattgatctgggtgatctcagggtgtcaacacatttgctgcaagagttgtgagagcacgacttaggaacctactgttcttaggtttgaggcactaaggagtcttcttctagagaacctggatctgataccattgggtacaccatgaaataatggaggggatgcttctgtttagttaggtttctttcaaaatgtggaggtagccttgaaccctctttccttttcctcctagtatcctgtggagcacccagacaaattcctgaagtttggcatgacaccttccaagggagttctgttctatggacctcctggctgtgggaaaactttgttggccaaagccattgctaatgaatgccaggccaacttcatctccatcaagggtcctgagctgctcaccatgtggtttggggagtctgaggccaatgtcagagaaatctttgacaaggtgagctacaataggctgaactatgtattgatttgcctgagggcaaggaatagaggctgtttttctttaagagggttgaaatattcttagtgctggctgctcaactgcacagtaagtcacttgattttctttctgaggtctgagagacctagtgttattttttttttctctctctctctcttgagacagggtctggctctgttgcccaggttggagggcagtggtacagtcatggctcactgtaaccttgaaacctgggcttaagcaattctcctacttcagcctcctgagtagctgggactataggcatgcgtcaccacatctggctaattttttattttttgtagagacaaagtctcagtatgttgcccatgctggtttcggatttctggcctcaagtgatcctcccaccttggcctcccaaagtgctgggaatacaggtgtgagccaccacgtttgcctagagacatctagttttgttagtgcttgaatcaatccattcctcctacaggcccgccaagctgccccctgtgtgctattctttgatgagctggattcgattgccaaggctcgtggaggtaacattggagatggtggtggggctgctgaccgagtcatcaaccagatcctgacagaaatggatggcatgtccacaaaaaaaaatgtgttcatcattggcgctaccaaccggcctgacatcattgatcctgccatcctcagacctggccgtcttgatcagctcatctacatcccacttcctgatgagaagtcccgtgttgccatcctcaaggctaacctgcgcaagtccccagttgccaaggcaggtgcaagatcatgggctgtgggagacttgcatgagtcctcaggctggtacggagtgctctttagtttctggacaagattccactggggttagggttggtctaaagggaaggtagaatttttgaggatatcaagataatctagaatcaggaataaaatggggtggccaaagaaggggcaaactgtagttgggagtgctcgggtagcccaaagatctgcgtatctcgagaggagaggctaaatgctaaggtacctctgctgctgcttttaggatgtggacttggagttcctggctaaaatgactaatggcttctctggagctgacctgacagagatttgccagcgtgcttgcaagctggccatccgtgaatccatcgagagtgagattaggcgagaacgagagaggcagacaaacccatcagccatggtgagtctgcatcctttccccagatgtgccaatcatggagagccaggcagcagccaccaccatgccctggagttgagagtagaagctgttggaaagatcatctaactgagaagaattttaatagggcatcaaagataaagaatgctgaggtgaatccattcaatttggaataaggccgagaagagatggtcaggctccattctcagtctgaaccaagctccatgagggaaatcaaagtatgagagtgcagcaaacacagcaaggttttttttgttttttgttttttgttttttttttgagacgaagtctcactctgttgcccagactggagtgcagtggcacgatcttggctcactgcaacttctgcctcccaggttcaagcgattctcctgcctcagcctcccgagtagctgggactacaggcacatgccaccatgtccggctagttttttgtattttttttttagtagaaacgtggtttcaccacgttagccaggatggtctcgatctcctgaccttgtgatgtgcccacctcggcctcccaaagtgctgggattacaggcgtgaaccacagagcaaggttttgagctgagatgagactcatatacttatccctgatggttggggaagggatagggtccacagacctcccaaaatgaaaaggcaaattcatgtgtttgtaagttccataaaggtaagatctctgtcatctcacttgttttccactatgtcttgtgttacccttaattaattcattaagttccaaacatgggacttaatgagcaaataaatggctttctttcccttttgaagggtctgtgacatcccttctctctcccataaaagcttaacaactactgatgaactaatcctaggaggtagtcacataagtcacagaaattggcctctcaatggaagagataggttttgagctgggctgtgaagagagtagaatttgaataaagggaataagcagcccaaataatgtgctctagtagtaggattgcaatcattgggaaaccctgggtagatttaagagtatatatgtcactggaagtgagaccgctaggtaggatgtaatccaaatgtggtaagcactgaaagccattggcatttccttttaaagtattaaggtttattaaggtatgatataaatacaataaaattcactctttctatataccatttccatgctttatgacaagtgtgtgtaagttctataactactaccacagttgagacttaaaatttctactatctcaaaaagtttccttagccacttcagtcaacatctcccctccttaagccccatcactgatgtgatttctgtccctacagttttcccttttccagagtgcattgacaagtttttaagcagagcagtgactcaattttaggaagcatggcctagcatcttacctcaggttggattggaagggcaaggagaccaataaactgcagtaatgggaggcctgggatgaaatccaggctgggctttaactagccctagtgatctgtgtttaccaactataggaggtagaagaggatgatccagtgcctgagatccgtcgagatcactttgaagaagccatgcgctttgcgcgccgttctgtcagtgacaatgacattcggaagtatgagatgtttgcccagacccttcagcagagtcggggctttggcagcttcaggtaagttggttgggagcattagacagtgcttaagttactttggggacctacaccaaaagggatgggagtcctaaggaagctagaggggtagttgtggaaatcttacacaggccctgtcctaaccctcttttttggctttgctcttgtacacagattcccttcagggaaccagggtggagctggccccagtcagggcagtggaggcggcacaggtggcagtgtatacacagaagacaatgatgatgacctgtatggctaagtggtggtggccagcgtgcagtgagctggcctgcctggaccttgttccctgggggtgggggcgcttgcccaggagagggaccaggggtgcgcccacagcctgctccattctccagtctgaacagttcagctacagtctgactctggacagggggtttctgttgcaaaaatacaaaacaaaagcgataaaataaaagcgattttcatttggtaggcggagagtgaattaccaacagggaattgggccttgggcctatgccatttctgttgtagtttggggcagtgcaggggacctgtgtggggtgtgaaccaaggcactactgccacctgccacagtaaagcatctgcacttgactcaatgctgcccgagccctcccttccccctatccaacctgggtaggtgggtaggggccacagttgctggatgtttatatagagagtaggttgatttattttacatgcttttgagttaatgttggaaaactaatcacaagcagtttctaaaccaaaaaatgacatgttgtaaaaggacaataaacgttgggtcaaaatggagcctgagtcctgggccctgtgcctgcttcttttcctgggaacagccttgggctacccaccactcccaaggcattcttccaaatgtgaaatcctggaagtaagattgcaccttcttcctctcctgatcaacatcggtatgatgtctcctgttgcctcaccctttgtctgcagtatcactggataggactggtggaaagggagcagcctgacagagctccaaatgtggagaatatggcatccctccacctatatttgatgtggacggtaaggctaggcctgcaggatcccttatcctgaccaaagactgtgttggggtgccatttgaaaatcgcagggttgcaaaagaatacaatcttacttgcaggtggatattctctatactctcttttaatgcatctaaaaatcccaaacatcccctggttggtgatcacttacagttgtgtccacctttattttatgtactttgattaaaaaaaaaaaactttttgttaatataaaattttagtattgaattttttttttttccaaacagaaaatagactatcctcttcaacagtaatcacttagtgcttctagggtcagtacagtgatgccttacccagagagagagtagtgcagagaaaataaattactaaattaaatatatgttgattggctttgggacataatctcaaaagacagtcctgaacaccgtaattttgaataaaatactgtaatctccaaagatcaaaatccctaaagtctaaaattctgaaaatcacaatcccaaaaggtcaaaatcccaaaatacaattctggaagaaatactaaacattcttcgaaaatttacttacatttttaaaagcgtatttatttgagaaacaacacaacagaacgtttcataggccactacacgataaaatagggaatagtaacatttttgcaagataaacactcaggtataccaatgacagttgcacggatataacggtgatgagcagatgaaacattcataaagaaataggtcaaaaagtgaaatgtataaatgctttgtcactatgcttggtaattgtgggcacctagctttatataactggtcatctgtaatactgtgacagaaaacctaagtcttgatgagatggttcaaaaactgttgcgttaccactgcattgtctcccaaagagacgaggtcttgagaaattttatccttcacaaatgcacgtgtacaaaaaacagatgtctcttcgtttattgaggaactttcaacatttttatgcacacatataatgcttacacacagagtcaacattgcggtcatggagtcaaatttttaatgtccaaggcaccagaagaaaaatctgtcctaggctgggcatggtggctcatgcctgtaatcccagcactttgggatgctgaggcgggcagatcacctgaggtcgggagctcaagaccagcctgaccaaaatggagaaaccccatctctactacaaatacaaaattagccaggcgtggtggcgcttgcctgtactcccagctacttgggaggctgaggcagagaatagcttgaacccgggaggcggaggttgtcgtgagctgagatggagccattgcactccagcctgggcaacaagagcaaaactcaatcttaaaaaagaagaaaaatctatcctacctcttagagaccaatttgccttctgtatttgttctctctgggcccccgcctgttggatggtacccaccaacattgaaggcagatcttccccactcagtccactcagacttacaaactaatccccggaaacaacctcacagacacacccagataatgctttaccaggtttctaaatactccttaatccagtaaaattgacacctaaaattaagtccacaattctgctcattggcaacttggcacccttatgtgtctaaaccatacttaagacaataacaaggtaatacttctgtataacatgatgcaactgtcttgtttacaacgaaaaacatactaatcctttccccagaatttgaaattttattttgtccaggttgtgattttaaggattttgatcttttgggacttcaacatttggggttatggtgtttgggattgtgtccttcaggattatgatcaacactggtattagtcatcctagactttatgctaggcaagaccaggctccgttttggtctacatagataatcttcaaggatgcatgatacttacactggttgactaaactggagcagctctggaactcatgctgtgaaatgggtatttttgcacttttggtatttgttatttcaggaggtttttagggaacaggtggtggttggttacgtgaataagttctttagtggtgatttctaagattttggtgcacctgtcatctgagcggtgtacaccgtacccaatgtgtagtcttttatccctcaccgcccccccaccttttccctgagtccccagagtccgttgtatcattcttacgcctttgcattctcatagcttagctcccatg

SEQ ID NO: 12 - DNA, 3418 bp, Homo sapiens
tgccagagttgctgggagtgcgcgcggtcggatcacaaggcggcggcggaggaggcccagagaccggagcgcggagacctcagccagcggcctacgcccaggcctttctccaccggaggaccagggaaccgcagtcttcatcacagaggtaccgtgctccgcgctccccgcctgacccggcccagcccgctgcggcggtgcctccttccttcctccttccctcgcgctctctctttcgcccgcccgcgccttccctgcccgcctgcgtcaccgcggccgccatggctgagaatggcgagagcagcggccccccgcgcccctcccgcggccctgctgcggcccaaggctcggctgctgccccggctgagcctaaaatcatcaaagtcacggtgaagactcccaaagagaaagaggagttcgcggtgcccgagaacagctcggttcagcagtttaaggaagcgatttcgaaacgcttcaaatcccaaaccgatcagctagtgctgatttttgccggaaaaatcttaaaagatcaagataccttgatccagcatggcatccatgatgggctgactgttcaccttgtcatcaaaagccagaaccgacctcagggccagtccacgcagcctagcaatgccgcgggaactaacactacctcggcgtcgactcccaggagtaactccacacctatttccacaaatagcaacccgtttgggttggggagcctgggaggacttgcaggccttagcagcctgggcttgagctcgaccaacttctctgagctccagagccagatgcagcagcagcttatggccagccctgagatgatgatccaaataatggaaaatccctttgttcagagcatgctttcgaatcccgatctgatgaggcagctcattatggctaatccacagatgcagcaattgattcagagaaacccagaaatcagtcacctgctcaacaacccagacataatgaggcagacactcgaaattgccaggaatccagccatgatgcaagagatgatgagaaatcaagacctggctcttagcaatctagaaagcatcccaggtggctataatgctttacggcgcatgtacactgacattcaagagccgatgctgaatgccgcacaagagcagtttgggggtaatccatttgcctccgtggggagtagttcctcctctggggaaggtacgcagccttcccgcacagaaaatcgcgatccactacccaatccatgggcaccaccgccagctacccagagttctgcaactaccagcacgaccacaagcactggtagtgggtctggcaatagttccagcaatgctactgggaacaccgttgctgccgctaattatgtcgccagcatctttagtaccccaggcatgcagagcctgctgcaacagataactgaaaacccccagctgattcagaatatgctgtcggcgccctacatgagaagcatgatgcagtcgctgagccagaatccagatttggctgcacagatgatgctgaatagcccgctgtttactgcaaatcctcagctgcaggagcagatgcggccacagctcccagccttcctgcagcagatgcagaatccagacacactatcagccatgtcaaacccaagagcaatgcaggctttaatgcagatccagcaggggctacagacattagccactgaagcacctggcctgattccgagcttcactccaggtgtgggggtgggggtgctgggaaccgctataggccctgtaggcccagtcacccccataggccccataggccctatagtcccttttacccccataggccccattgggcccataggacccactggccctgcagccccccctggctccaccggctctggtggccccacggggcctactgtgtccagcgctgcacctagtgaaaccacgagtcctacatcagaatctggacccaaccagcagttcattcagcaaatggtgcaggccctggctggagcaaatgctccacagctgccgaatccagaagtcagatttcagcaacaactggaacagctcaacgcaatggggttcttaaaccgtgaagcaaacttgcaggccctaatagcaacaggaggcgacatcaatgcagccattgaaaggctgctgggctcccagccatcgtaatcacatttctgtacctggaaaaaaaatgtatcttatttttgataatggctcttaaatctttaaacacacacacaaaatcgttctttactttcattttgattcttttaaatctgtctagttgtaagtctaatatgatgcattttaagatggagtccctccctcctacttccctcactccctttctcctttgcttatttttcctaccttcccttcctcttgtctccccactccctccctctttgtttccttccttccttatttcctttagtttccttccttagccgttttgagtggtgggaatcaatgctgtttcactcaaaagtgttgcatgcaaacacttctctttattctgcatttattgtgatttttggaaacaggtatcaaccttcacagttgggtgaacaagtgttgtcctacagatgtccaatttatttgcatttttaaacattagcctatgatagtaatttaatgtagaatgaagatattaaaaacagaagcaaattatttgaagctctctaatttgtggtacgatattgcttattgtgactttggcatgtatttttgctagcaaaatgctgtaagatttataccattgatcttttttgctatatttgtatacagtacagtaagcacaattggcactgtacatctaaaaatattacagtagaatctgagtgtaatatgtgtaaccaaaatgagaaagaatacaagaaatgtttctggagctagttatgtctcacaattttgtagaatcttacagcatctttgataaacttctcagtgaaaatgttggctaggcaagttcagttaaaatatagtagaaatgtttatcctggtatctctaagtatacatttaattgtacagaaaatttacagtgtaacattgtgtcaacatttgcagattgactgtatatgaccttaatctttgtgcagcctgaaggatcagtgtagtaatgccaggaaagtgctttttacctaagacttccttctcagcttctcccataaagagaccctaatatgcattttgatttgtaattggaaatgtaactttcactgaaagtgtcatgtgatgtttgcattacttttaactgctatgtataaaggaaagtgtgtcttttgacttcatcagttatttctcttgtgcacagagaaaaatgcattaaaaatgactaaaaaaaataaaaaattaaaaaatggataaatcttttctttttgccttttggccctaggatcgtgtttaggaggattatcccaccccgaga

SEQ ID NO: 13 - DNA, 2814 bp, Homo sapiens
ggtggcgagtggatatctccggagcatttagataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattcaaaaaaaaaaaaaaaaaaaaaaaaaaaaaaaaaaaaaaaaacaaaaaaaaaaaaaaaa

SEQ ID NO: 14 - protein, 1278 aa, Homo sapiens
MAQSKRHVYSRTPSGSRMSAEASARPLRVGSRVEVIGKGHRGTVAYVGATLFATGKWVGVILDEAKGKNDGTVQGRKYFTCDEGHGIFVRQSQIQVFEDGADTTSPETPDSSASKVLKREGTDTTAKTSKLRGLKPKKAPTARKTTTRRPKPTRPASTGVAGASSSLGPSGSASAGELSSSEPSTPAQTPLAAPIIPTPVLTSPGAVPPLPSPSKEEEGLRAQVRDLEEKLETLRLKRAEDKAKLKELEKHKIQLEQVQEWKSKMQEQQADLQRRLKEARKEAKEALEAKERYMEEMADTADAIEMATLDKEMAEERAESLQQEVEALKERVDELTTDLEILKAEIEEKGSDGAASSYQLKQLEEQNARLKDALVRMRDLSSSEKQEHVKLQKLMEKKNQELEVVRQQRERLQEELSQAESTIDELKEQVDAALGAEEMVEMLTDRNLNLEEKVRELRETVGDLEAMNEMNDELQENARETELELREQLDMAGARVREAQKRVEAAQETVADYQQTIKKYRQLTAHLQDVNRELTNQQEASVERQQQPPPETFDFKIKFAETKAHAKAIEMELRQMEVAQANRHMSLLTAFMPDSFLRPGGDHDCVLVLLLMPRLICKAELIRKQAQEKFELSENCSERPGLRGAAGEQLSFAAGLVYSLSLLQATLHRYEHALSQCSVDVYKKVGSLYPEMSAHERSLDFLIELLHKDQLDETVNVEPLTKAIKYYQHLYSIHLAEQPEDCTMQLADHIKFTQSALDCMSVEVGRLRAFLQGGQEATDIALLLRDLETSCSDIRQFCKKIRRRMPGTDAPGIPAALAFGPQVSDTLLDCRKHLTWVVAVLQEVAAAAAQLIAPLAENEGLLVAALEELAFKASEQIYGTPSSSPYECLRQSCNILISTMNKLATAMQEGEYDAERPPSKPPPVELRAAALRAEITDAEGLGLKLEDRETVIKELKKSLKIKGEELSEANVRLSLLEKKLDSAAKDADERIEKVQTRLEETQALLRKKEKEFEETMDALQADIDQLEAEKAELKQRLNSQSKRTIEGLRGPPPSGIATLVSGIAGEEQQRGAIPGQAPGSVPGPGLVKDSPLLLQQISAMRLHISQLQHENSILKGAQMKASLASLPPLHVAKLSHEGPGSELPAGALYRKTSQLLETLNQLSTHTHVVDITRTSPAAKSPSAQLMEQVAQLKSLSDTVEKLKDEVLKETVSQRPGATVPTDFATFPSSAFLRAKEEQQDDTVYMGKVTFSCAAGFGQRHRLVLTQEQLHQLHSRLIS

SEQ ID NO: 15 - protein, 3144 aa, Homo sapiens
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 16 - protein, 2527 aa, Homo sapiens
MASGSCQGCEEDEETLKKLIVRLNNVQEGKQIETLVQILEDLLVFTYSEHASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 17 - DNA, 979 bp, Homo sapiens
tgagtctgcgcagtgtggggctgagggaggccggacggcgcgcgtgcgtgctggcgtgcgttcattttcagcctggtgtggggtgagtggtacccaacgggccggggcgccgcgtccgcaggaagaggcgcggggtgcaggcttgtaaacatataacataaaaatggcttccaaaagagctctggtcatcctggctaaaggagcagaggaaatggagacggtcatccctgtagatgtcatgaggcgagctgggattaaggtcaccgttgcaggcctggctggaaaagacccagtacagtgtagccgtgatgtggtcatttgtcctgatgccagccttgaagatgcaaaaaaagagggaccatatgatgtggtggttctaccaggaggtaatctgggcgcacagaatttatctgagtctgctgctgtgaaggagatactgaaggagcaggaaaaccggaagggcctgatagccgccatctgtgcaggtcctactgctctgttggctcatgaaataggttttggaagtaaagttacaacacaccctcttgctaaagacaaaatgatgaatggaggtcattacacctactctgagaatcgtgtggaaaaagacggcctgattcttacaagccgggggcctgggaccagcttcgagtttgcgcttgcaattgttgaagccctgaatggcaaggaggtggcggctcaagtgaaggctccacttgttcttaaagactagagcagcgaactgcgacgatcacttagagaaacaggccgttaggaatccattctcactgtgttcgctctaaacaaaacagtggtaggttaatgtgttcagaagtcgctgtccttactacttttgcggaagtatggaagtcacaactacacagagatttctcagcctacaaattgtgtctatacatttctaagccttgtttgcagaataaacagggcatttagcaaactaaaaaaaaaaaaaaaaaaa

SEQ ID NO: 18 - DNA, 2680 bp, Homo sapiens
cgcagaggcaccgccccaagtttgttgtgaccggcgggggacgccggtggtggcggcagcggcggctgcgggggcaccgggccgcggcgccaccatggcggtgcgacaggcgctgggccgcggcctgcagctgggtcgagcgctgctgctgcgcttcacgggcaagcccggccgggcctacggcttggggcggccgggcccggcggcgggctgtgtccgcggggagcgtccaggctgggccgcaggaccgggcgcggagcctcgcagggtcgggctcgggctccctaaccgtctccgcttcttccgccagtcggtggccgggctggcggcgcggttgcagcggcagttcgtggtgcgggcctggggctgcgcgggcccttgcggccgggcagtctttctggccttcgggctagggctgggcctcatcgaggaaaaacaggcggagagccggcgggcggtctcggcctgtcaggagatccaggcaatttttacccagaaaagcaagccggggcctgacccgttggacacgagacgcttgcagggctttcggctggaggagtatctgatagggcagtccattggtaagggctgcagtgctgctgtgtatgaagccaccatgcctacattgccccagaacctggaggtgacaaagagcaccgggttgcttccagggagaggcccaggtaccagtgcaccaggagaagggcaggagcgagctccgggggcccctgccttccccttggccatcaagatgatgtggaacatctcggcaggttcctccagcgaagccatcttgaacacaatgagccaggagctggtcccagcgagccgagtggccttggctggggagtatggagcagtcacttacagaaaatccaagagaggtcccaagcaactagcccctcaccccaacatcatccgggttctccgcgccttcacctcttccgtgccgctgctgccaggggccctggtcgactaccctgatgtgctgccctcacgcctccaccctgaaggcctgggccatggccggacgctgttcctcgttatgaagaactatccctgtaccctgcgccagtacctttgtgtgaacacacccagcccccgcctcgccgccatgatgctgctgcagctgctggaaggcgtggaccatctggttcaacagggcatcgcgcacagagacctgaaatccgacaacatccttgtggagctggacccagacggctgcccctggctggtgatcgcagattttggctgctgcctggctgatgagagcatcggcctgcagttgcccttcagcagctggtacgtggatcggggcggaaacggctgtctgatggccccagaggtgtccacggcccgtcctggccccagggcagtgattgactacagcaaggctgatgcctgggcagtgggagccatcgcctatgaaatcttcgggcttgtcaatcccttctacggccagggcaaggcccaccttgaaagccgcagctaccaagaggctcagctacctgcactgcccgagtcagtgcctccagacgtgagacagttggtgagggcactgctccagcgagaggccagcaagagaccatctgcccgagtagccgcaaatgtgcttcatctaagcctctggggtgaacatattctagccctgaagaatctgaagttagacaagatggttggctggctcctccaacaatcggccgccactttgttggccaacaggctcacagagaagtgttgtgtggaaacaaaaatgaagatgctctttctggctaacctggagtgtgaaacgctctgccaggcagccctcctcctctgctcatggagggcagccctgtgatgtccctgcatggagctggtgaattactaaaagaacatggcatcctctgtgtcgtgatggtctgtgaatggtgagggtgggagtcaggagacaagacagcgcagagagggctggttagccggaaaaggcctcgggcttggcaaatggaagaacttgagtgagagttcagtctgcagtcctctgctcacagacatctgaaaagtgaatggccaagctggtctagtagatgaggctggactgaggaggggtaggcctgcatccacagagaggatccaggccaaggcactggctgtcagtggcagagtttggctgtgacctttgcccctaacacgaggaactcgtttgaagggggcagcgtagcatgtctgatttgccacctggatgaaggcagacatcaacatgggtcagcacgttcagttacgggagtgggaaattacatgaggcctgggcctctgcgttcccaagctgtgcgttctggaccagctactgaattattaatctcacttagcgaaagtgacggatgagcagtaagtaagtaagtgtggggatttaaacttgagggtttccctcctgactagcctctcttacaggaattgtgaaatattaaatgcaaatttacaactgcagatgacgtatgtgccttgaactgaatatttggctttaagaatgattcttatactctgaaggtgagaatattttgtgggcaggtatcaacattggggaagagatttcatgtctaactaactaactttatacatgatttttaggaagctattgcctaaatcagcgtcaacatgcagtaaaggttgtcttcaactgaaaaaaaaaaaaaaaaaaaaa

SEQ ID NO: 19 - DNA, 4073 bp, Homo sapiens
attcctagggccgggcgcgggggcggggaggcctggaggatttaacccaggagagccgctggtgggaggcgcggctggcgccgctgcgcgcatgggcctgttcctggcccgcagccgccacctacccagtgaccatgatagtgtttgtcaggttcaactccagccatggtttcccagtggaggtcgattctgacaccagcatcttccagctcaaggaggtggttgctaagcgacagggggttccggctgaccagttgcgtgtgattttcgcagggaaggagctgaggaatgactggactgtgcagaattgtgacctggatcagcagagcattgttcacattgtgcagagaccgtggagaaaaggtcaagaaatgaatgcaactggaggcgacgaccccagaaacgcggcgggaggctgtgagcgggagccccagagcttgactcgggtggacctcagcagctcagtcctcccaggagactctgtggggctggctgtcattctgcacactgacagcaggaaggactcaccaccagctggaagtccagcaggtagatcaatctacaacagcttttatgtgtattgcaaaggcccctgtcaaagagtgcagccgggaaaactcagggtacagtgcagcacctgcaggcaggcaacgctcaccttgacccagggtccatcttgctgggatgatgttttaattccaaaccggatgagtggtgaatgccaatccccacactgccctgggactagtgcagaatttttctttaaatgtggagcacaccccacctctgacaaggaaacatcagtagctttgcacctgatcgcaacaaatagtcggaacatcacttgcattacgtgcacagacgtcaggagccccgtcctggttttccagtgcaactcccgccacgtgatttgcttagactgtttccacttatactgtgtgacaagactcaatgatcggcagtttgttcacgaccctcaacttggctactccctgccttgtgtggctggctgtcccaactccttgattaaagagctccatcacttcaggattctgggagaagagcagtacaaccggtaccagcagtatggtgcagaggagtgtgtcctgcagatggggggcgtgttatgcccccgccctggctgtggagcggggctgctgccggagcctgaccagaggaaagtcacctgcgaagggggcaatggcctgggctgtgggtttgccttctgccgggaatgtaaagaagcgtaccatgaaggggagtgcagtgccgtatttgaagcctcaggaacaactactcaggcctacagagtcgatgaaagagccgccgagcaggctcgttgggaagcagcctccaaagaaaccatcaagaaaaccaccaagccctgtccccgctgccatgtaccagtggaaaaaaatggaggctgcatgcacatgaagtgtccgcagccccagtgcaggctcgagtggtgctggaactgtggctgcgagtggaaccgcgtctgcatgggggaccactggttcgacgtgtagccagggcggccgggcgccccatcgccacatcctgggggagcatacccagtgtctaccttcattttctaattctcttttcaaacacacacacacacgcgcgcgcgcgcacacacactcttcaagtttttttcaaagtccaactacagccaaattgcagaagaaactcctggatccctttcactatgtccatgaaaaacagcagagtaaaattacagaagaagctcctgaatccctttcagtttgtccacacaagacagcagagccatctgcgacaccaccaacaggcgttctcagcctccggatgacacaaataccagagcacagattcaagtgcaatccatgtatctgtatgggtcattctcacctgaattcgagacaggcagaatcagtagctggagagagagttctcacatttaatatcctgccttttaccttcagtaaacaccatgaagatgccattgacaaggtgtttctctgtaaaatgaactgcagtgggttctccaaactagattcatggctttaacagtaatgttcttatttaaattttcagaaagcatctattcccaaagaaccccaggcaatagtcaaaaacatttgtttatccttaagaattccatctatataaatcgcattaatgaaataccaactatgcgtaaatcaacttgtcacaaagtgagaaattatgaaagttaatttgaatgttgaatgtttgaattacagggaagaaatcaagttaatgtactttcattccctttcatgatttgcaactttagaaagaaattgtttttctgaaagtatcaccaaaaaatctatagtttgattctgagtattcattttgcaacttggagattttgctaatacatttggctccactgtaaatttaatagataaagtgcctataaaggaaacacgtttagaaatgatttcaaaatgatattcaatcttaacaaaagtgaacattattaaatcagaatctttaaagaggagcctttccagaactaccaaaatgaagacacgcccgactctctccatcagaagggtttatacccctttggcacaccctctctgtccaatctgcaagtcccagggagctctgcataccaggggttccccaggagagaccttctcttaggacagtaaactcactagaatattccttatgttgacatggattggatttcagttcaatcaaactttcagcttttttttcagccattcacaacacaatcaaaagattaacaacactgcatgcggcaaaccgcatgctcttacccacactacgcagaagagaaagtacaaccactatcttttgttctacctgtattgtctgacttctcaggaagatcgtgaacataactgagggcatgagtctcactagcacatggaggcccttttggatttagagactgtaaattattaaatcggcaacagggcttctctttttagatgtagcactgaaatccttgctggagggaagagaggggatgaactcaagttttccacatcctgggacacctgtccctcttttcctaactgcctaagataacccatttcttccaaccatctgaggacagtcccgtcgtctcagaggccctgcaccggggagagactgggctctgcagcagccacatcagcattcacagcttcatgtggcttcactgtctgaaaatctaccgactccaacatggccccacggtgacaacagacctgtgacaggaagcccaaagctcacatagaaatggtggacagatcaaagtctctatagtaagggaaaaaaagagaggtggcaggcatgagccccctgcacccagtggctcgtgtccatactgagtccagaccctgatcaaggcctgacttagtgtcactggcagtcccactaaattacacttccttacactggcccgatgcgacaaatcaggtggctcccttctgtcacgtggagcacacagtgttttccatcatccatagctttcttcctgatggtgtttgcattattgcgccttcccaatctgcatgctgcgttgggcttgcggtgcctgaacaaggtttgctcccatgagctcaggcaccctaggatcccctgttagactattaggctgtccagcatggtctcctttcccttcttggtggtggtcttttccctttccagaatagaacagtgattcttaaaataagttagagcaggccgggcgcggtggctcatgcctgtaatcccagcactttgggaggccgaggtgggtggatcacgaggtcaggagttcaagaccagcctggccaagatgatgaaaccccgtctctattaaaaatacaaaaattagctgggcgtggtggcaggcacctgtaatcccagcttcctgggaggctgaggcaggagaatcacttgaacccggggggcagaggttgcagtgagccgagatcacgccactgaactccagcctgggcaacagagtgagactctgtctcaaaaaaaaaaaaaaaacaaaaacaaaaaagcaagatcatccactacacatgaacatgaatcacagtattatttgcaca

SEQ ID NO: 20 - DNA, 3215 bp, Homo sapiens
aggagaaggagaaggaggaggactaggaggaggaggacggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 21 - DNA, 6176 bp, Homo sapiens
tgagtgtcatttcttcaacgggacggagcgggtgcggttcctggacagatacttctatcaccaagaggagtacgtgcgcttcgacagcgacgtgggggagtaccgggcggtgacggagctggggcggcctagcgccgagtactggaacagccagaaggacctcctggagcagaggcgggccgcggtggacacctactgcagacacaactacggggttgtggagagcttcacagtgcagcggcgaggtgagcgcggcgcggggcggggcctgagtccctgtgagcggagaatctgagtgtgtgtgtgtgtgtgtgtgtgtgtgtgtgtgtgtgtgtgtgagagagagagagagagagagagagagagagagagagagagagagagcgccatctgtgagcatttagaatcctctctatcctgagcaaggagttctgcgggcacaggtgtgtgtgtagagtgtggatttgtccgtgtctgtgaggctgttgtgggaggggaggcaggagggggctgcttcttattcttggagacttctgtggggaggtgacaagggaggtgggtgctgggggctggagagagaggcgaccttgattgtctcgggtccttagagatgcaaggaagggaaatgtatggggtgtgtggttggggtgaaggtttaggggaggagagctgaggggtaaggaaggtttgggataatgtgaagaggccagtttcagactgtccctggcacacacccttcatgtaatctctgaaataaaagtgtgtgctgtttgtttgtaaaagcattagattaacttctaggggaattgagtagacctctgaggcacctctgaagcttctttaggtataaatttcttgctagttttttgttttcttagtgttatatttttacatagttgaaatgactgtgaaactaactttttgaattaaagtttgaaaacactgttactattttattataatgctaataatttcatagttactttttaaatatataatagttgtgacacaaattacctcactttctttgtttttttttcttacactttaagttttagggtacatgtgcacaacgtgcaggtttgttacatatgtatacatgtgccatgttggtgtgctgcacccattaactcgtcatttaacattaggtatatctcctaatgctatccctccccaccccccaccccacaacaggccccagtgtgtgatgttccccttcctgtgtccatgtgttctcactgttcaattcccacctatgagtgagaacatgcggtgttcggttttttgtccttgccatagtttgctgagaatgatggtttccagcttcatccatgtccctacaaaggacatgaactcattcttttttgtggctgcatagtattccatagtgtatatgtgccacattttcttaatccagtctatcattgttggacatttgggttggttccaagtctttgctattgtgaatagtgccgcaataaacatacatgtgcatatgtctttatagcagcatgatttataatccttgggttatatacccagtaatgggatggctgggtcaaatggtatttctagttctagatccctgaggaatcgccacactgacttccacaatggttgaactagtttagagtcccaccaacagggtaaaagtgttcctatttctccacatcctctccagcacctgttgcttcctgactttttaatgatcgccattctaactggtgtgagatggtatctcattgtggttttgatttgcaattctctgatggccagtgatgatgagcattttttcatgtgtcttttggctgcataaatgtcttcttttgagaagtgtctgttcatgtcctttgcccactttttgatggggtattttgttttttcttgtaaatttgtttgagttcattgcagattctggatattagccctttgtcatatgagtagattgcaaaaattttctcccattctgtaggttgcctcttcactctgatggtagtttcttttgctgtgcagaagctctttagtttaattagatcccatttgtccattttggcttttgttgccattgcttttggtgttttagacatgaagttcttgcccatgcctatgtcctgaatggtattgcctaggttttcttctagggtttttatggtttcaggtctaacatttaagtctttaatccatcttgaattaatttttgtataagcaaattacgtcactttccccattgatgacctttattatgacattcaccaatagttgaaaatgtatgtttctggttaatttttgatttatattttttgatttgtaattattttgaattattttgacctatttattggccagttgtaattactgctctgctctacgaattacctgttgtatttggtaggtaatggacaatgatctattgtctcttatctttagggcttagtatttttctcagtgactttgtgggtttgttgtactgtaagattattaacactttattgatatttgattcagtattttctccagtttgtggtatgtatattttgaaaattcttttccatgttaagaatttgaacatttttatttaataaaatatattgcaaaatgttaattaatgattcacaaactagctcaagtctaccattttgtggtattgatgtctccaggtttctccttccttcttaaaaaaaaatgtatttattgagagtatgctagtgtcagggatttccctaggcataagcactccaagtaatgagtcccagacactgccttgatccaaatgtcattctggaaagaaaaatcattttacagtgataagcctaataatagttatacttgttttgcctgggagatgcattgatcagctaaatgtaaatataagaactttcaaaactaaaatgacgttccttaatctttctctctgctttaggaatcatgctttcttaggaacttaaagatttggagaatcatttctgtctgtcccaccttcccaggagcataaccatttctgtggtgttctaaggtgtgagtgcatggcagtagtattcctaaaaatccatattcagtttcctcatgtgccctactccgtccctttctctatccacattgctttaaatcatatttttctctcaaggtgtacaaggatgataaataggtgccaagtggagaacccaagtgtgacgagccctctcacagtagaatggagtgagaagctttctgacctcataaattgaaggctatcgtaattcattcttttatatattttacttgcattaatcctcatataacctcaagaggtaaattaatataattatcctccattattggagagaaagttgagacacaaaagaatcaaaaactcttccaggatcaaccagtaaaaggcagaccttggatttgaaccaggcaacctggctcagaagtcagttttaattaccacactctgtactttcaaagatttgtaaacgctttgacaatgcatgtcaatttcaagctatgaagagccaaacataatttttcacaatatctctcaaatctaatgggtccccactataaagattaaattccaggctgatgacactgtgaggccacatggccagctgtgctggaggcctgctcaaggccagagcctaggtttacagagaagcagacaaaaagctaaacaaggagacttactctgtctgcatgacttattccctctaccttgttttctcctagtctatcctgaggtgactgtgtatcctgcaaagacccagcccctgcagcaccacaacctcctggtctgctctgtgaatggtttctatccaggcagcattgaagtcaggtggttccggaacggccaggaagagaagactggggtggtgtccacaggcctgatccagaatggagactggaccttccagaccctggtgatgctggaaacagttcctcggagtggagaggtttacacctgccaagtggagcacccaagcctgacgagccctctcacagtggaatggagtgagcagctttctgacttcataaatttctcacccaccaagacgcgaactttactaatccctgagtatcaggcttctcctatcccacatcctattttcatttgctccacgttctcatctccatcagcacaggtcactggggggtagccctgtaatactttctagaaacacctgtaccccctggggaagcagtcatgcctgccaggcaggagaggctgtccctcttttgaacctccccatgatgtcacaagtcggggtcacctgctgtctgtgggctccaggccctgcctctgggtctgagactgagtttctggtactgttgctctgagtcgtttgttgtaatctgagaagaggagaagtatagggaccttcctgacatgaggggagtccaatctcagctccgccttttattagatctgtcactctaggcaactacttaacctcattgggtctcaggctttctgttcatcagatgttgaagtcctgtcttacatcaaggctgtaatatttgaatgagtttgatgactgaaccttgtaactgttcagtgtgatttgaaaacctttctcaagaaatggtcagttattttagttcttgcagagcagccttctttctcattttcaaagctctgaatctcaaggtgtcaattaaagaggttccatttgggataaaaatcactaaacctggcttcctctctcaggagcacggtctgaatctgcacagagcaagatgctgagtggagtcgggggctttgtgctgggcctgctcttccttggggccgggctgttcatctacttcaggaatcagaaaggtgaggagcctttggtagctggctgtctccatacgcttttctggaggaggaactatggctttgctgaagttggttctcagcatatgaatggccctggataaagcctctctactcccaaatgacctccaatgttctgcaaatccagaaatcatcagtgcatggttgctatgtcaaagcataatagcttgtggcctacagagataacagaaagattaacaggtataggtgctttggttgagatcgtggagcaaattaaggaagagcaactaaagctaatacaattacactggatcctgtgacagacacttcacacttcatgggtcacatggtctgtttctgctcctctctgccctggctggtgtgggttgtggtgtcagagaactctcaggtgggagatctggagctgggacattgtgttggaggacagatttgcttccatatcctttaagtgtatatcttctctttttcctaggacactctggacttcagccaacaggtaataccttttcatcctctttaagaaacagatttggaggccaggcgcagtggctcacgcctgtaatcccagcactttgggaggccgaggcgggcgaatcatgaggtcaggagttcgagaccagcctgaccaacgtggtgaaaccccgtctctactaaaaatacaaaaaaaaatcagtcgggcgtggtggtgtgcgcctgtaatcccagctactcaggaggccaaggcaggagaatcgctggaacccgggaggcagaggttgcagtgagccgagattgggccactgcactccagcctaggtgacagagtgagaccccatctcaaaaaaacaaaaaaagaaagaaagaaacagatttcctttccctagaatgatggtagaggtaataaggcatgagacagaagtaatagcaaagacattggatccaaatttctgatcaggcaatttacaccagaactcctcctctccacttagaaaaggcctgtgctctgcaggagtattgactcatggagacttcagaacttgtttttcttcttcctgcagtgctctcatctgagtccttgaaagagggcaaaataaactgttagtagagccaggtctgaaaacaacactttcttgcgtctctgcaggattcctgagctgaagtgaagatgaccacattcaaggaagaaccttctgccccagctttgcaggatgaaacacttccccgcttggctctcattcttccacaagagagacctttctccggacctggttgctactggttcagcagctctgcagaaaatgtcctcccttgtggctgcctcagctcgtacctttggcctgaagtcccagcattaatggcagcccctcatcttccaagttttgtgctcccctttacctaa

SEQ ID NO: 22 - DNA, 5859 bp, Homo sapiens
  misc_feature (1534)..(1633): n is a, c, g, or t
  misc_feature (2121)..(2220): n is a, c, g, or t
  misc_feature (2554)..(2554): n is a, c, g, or t
  misc_feature (2660)..(2759): n is a, c, g, or t
  misc_feature (3089)..(3188): n is a, c, g, or t
  misc_feature (3592)..(3691): n is a, c, g, or t
  misc_feature (4046)..(4145): n is a, c, g, or t
atacctaggcactaatttagttccatatgtactatgtgtacctgaaaagttgtgtggcaatcaaattttcacaaatagaatcctgttttaaatacactaagaaagtacctactttatcctttaaacaagaggtcagcagactttttctacaaagggtcagatagtaaagattttacaccttttgtacaatacaatctctatctcatctacttagctctgccattgttgcataaaagcagctgtagatgatacacaaatgggtgaggctgtattccaaatgaaacgttatttgcaaaaacaggtggtagattaaatttggtcccaaggcttacttgggaaaaaaaaagatcttttgaaaaagaaaaaataaatgaataatttttttaaaaaattgttccctaggtcatagtttgccagcccctgccctaaacaaataattcttgaatgcctactgtggtgtgtaagatatgagtaaataccagggatacacagagaacaaaagagaaaaactgctattcttgtgaaacttggaagttggaggtaagctatttaaaataaacccacaataaagtacttcacatagtgcagactgtttctttaaatcaaaactcactccaaacaaccaattgattcactttgtaagtttgaatttttgtcttcagattcttttaaagtgggcccttagtcaggagcggtggctcatgcctgtagtcctagcactttgggaggctgaggcaggcagatcacttgaggtcaggagttcgagacaagcctggccaacatggcgaaaccccgtctccactgaaaacacaaaaattaggctggcatagtggcatttgcctgtagtcctagctactcaggaggctgaggcaggagaattgcttgaacctgggaggtgaaaattgcagtgagccgagatcatgctattgtactccagcctgggcaacaaagcaagactcgtctcaaaaaaataaaaattaaaaaaataaagtagcctctagcctaagatagcttgagcctaggtgtgaatctactgccttactctgatgtaagcacagtaagtgtgggggctgcagggaatatccaggaggaacaataatttcagaggctctgtctcttcatgtccttgacctctgcttacagcagcaatacttttactcagacttcctgtttctggaacttgccttcttttttgctgtgtttatacttcccttgtctgtggttagataagtataaagccctagatctaagcttctctgtcttcctccctccctcccttcctcttactctcattcatttcatacacactggctcacacatctactctctctctctatctctctcagaatgacaattctaggtacaacttttggcatggttttttctttacttcaagtcgtttctggagaaagtggctatgctcaaaatggtgagtcatttctaacttttcttatggattttggattatctgtagcatggtttcaggttattcagttccctaagagacctgagtcaggcactgggtttgagtgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngagcgtttttgttttttgttttttgttggttggttggttttgaagcatttttcttgtctttgcccttcccattttcttccttgaatactacataatccattactatttcatgtctgccacagagtctgctattttattaaggtcatgccatatttcaaaaggatgcatttatttgtttcattaacagctgcatgtttgttcctccccaggagacttggaagatgcagaactggatgactactcattctcatgctatagccagttggaagtgaatggatcgcagcactcactgacctgtgcttttgaggacccagatgtcaacaccaccaatctggaatttgaaatatggtgagggatggtggttttaatggttgcttagacatcctctgtctctcttttcatatgctgtttttaatagccacaaaagaaagaatatgtggcctaattaacaaatgttaacatctaaggaattcccaaaggcctcctgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncctacctgaatcaagacatacccccttttattcctacagtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactataggtaagaagttgtatataaaagtatggttgtcacttttgggctacctgaaaacactgtgtctggacattctgtaggttaaaagtagacaaatagtggaaagaactggcaatagataatagataattccctactgtaantttttataataaatgaaaagcttgaaatttatactttcctgcagttgaaagaattctgaggatcttcaaacccaggtgtgaaagatagtgtttgtgcaacctacannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcaaagtgacttgcagaggagatgaattttaaatactataattatttccttggctgccctttagacagaatttatttctttttcttttccagttaaacctgaggctccttttgacctgagtgtcatctatcgggaaggagccaatgactttgtggtgacatttaatacatcacacttgcaaaagaagtatgtaaaagttttaatgcacgatgtagcttaccgccaggaaaaggatgaaaacaaatggacggtatgtacttcaactacattaataaaataaaaacttatgaatgttttctattttgttggcctagtagtgcatttcccctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngagctccttattctaacaaatacgagacaacttcagagaatgcttatgggactaaaggaatcccaattgaaatgatttgggagatttaggcaacacctcttttcccatcctaagaatgtaactgcactctactctctagcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggtaaggaatggtggtagagtttttgttccctcagagtgctttgcatgtcaaagtgtgggagcaagtgagaggaagattgttgaaactaacctgcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatacttcaagtggcagatgctctgggcctggtcacccaagtcaatgccttttaaaccaaatccctccataaagctgtcaaatatgtctcttaactgaaaagcaactttcaggaaataataagtgggcccacattactaagtaaatgcaaatcaccctgagaccctacccccactgcatggctactgaatgctcaccacaatctattcttgctttccaggggagatggatcctatcttactaatcatcagcattttgagttttttctctgtcgctctgttggtcatcttggcctgtgtgttatggaaaaaaaggtgaccttcttcaacgtaataaagagggtgattgtgtgggtcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttgtcatgtcttgttcacagaatggattgatatctgtggtctctggtccaacccctccttgaattgatagggccccgaggcccagagaaagccagtctcttgaccatggtcacccacctaattgtgttagagccaagactagaaatctgttcttctgattccaagctcagaataagtgggaagactcagtgtgcctgtgccctctgccattcacttcatctttcaatgttctctgatttcaggattaagcctatcgtatggcccagtctccccgatcataagaagactctggaacatctttgtaagaaaccaagaaaagtgagtgtttttggtgcttaaaaagtgttgtgttggcaacatcccagtggccaagaatgatattccaggacaaggaacagttgaacctcaccttttggtatttgattcatcctgtaactagggtccctcctaagaccctagctgcagtagggaactgaaataagatacacatctcagaacttctgggctccctggggctggagggcacagccagtggtcacttcaagtcttgaagtgtctcagaagctccagaagcaaagagtccattgaggaacatgctggcaattctgtgacattccctgtcagaaaactctatagacctactcctgaactgaacatttgatggtgtgtctctctggtgccatcttaataccctttctcctttttctgtgcagaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtgaagtgtaagaaacccagactgaacttaccgtgagcgacaaagatgatttaaaagggaagtctagagttcctagtctccctcacagcacagagaagacaaaattagcaaaaccccactacacagtctgcaagattctgaaacattgctttgaccactcttcctgagttcagtggcactcaacatgagtcaagagcatcctgcttctaccatgtggatttggtcacaaggtttaaggtgacccaatgattcagctatttaaaaaaaaaagaggaaagaatgaaagagtaaaggaaatgattgaggagtgaggaaggcaggaagagagcatgagaggaaataaataaaggaaaataaaaatgatagttgccattattaggatttaatatatatccagtgctttgcaagtgctctgcgcaccttgtctcactccatcctgacaataatcctgggaggtgtgtgcaattactacgactactctcttttttatagatcgagctc

SEQ ID NO: 23 - DNA, 10241 bp, Homo sapiens
ccggcgtcggcggcgcgcgcgctccctcctctcggagagagggctgtggtaaaagccgtccggaaaatggccgccgccgccgccgccgcgccgagcggaggaggaggaggaggcgaggaggagagactgctccataaaaatacagactcaccagttcctgctttgatgtgacatgtgactccccagaatacaccttgcttctgtagaccagctccaacaggattccatggtagctgggatgttagggctcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccgtcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaattt

SEQ ID NO: 24 - DNA, 630 bp, Homo sapiens
atgtggactctcgggcgccgcgcagtagccggcctcctggcgtcacccagcccagcccaggcccagaccctcacccgggtcccgcggccggcagagttggccccactctgcggccgccgtggcctgcgcaccgacatcgatgcgacctgcacgccccgccgcgcaagttcgaaccaacgtggcctcaaccagatttggaatgtcaaaaagcagagtgtctatttgatgaatttgaggaaatctggaactttgggccacccaggctctctagatgagaccacctatgaaagactagcagaggaaacgctggactctttagcagagttttttgaagaccttgcagacaagccatacacgtttgaggactatgatgtctcctttgggagtggtgtcttaactgtcaaactgggtggagatctaggaacctatgtgatcaacaagcagacgccaaacaagcaaatctggctatcttctccatccagtggacctaagcgttatgactggactgggaaaaactgggtgtactcccacgacggcgtgtccctccatgagctgctggccgcagagctcactaaagccttaaaaaccaaactggacttgtcttccttggcctattccggaaaagatgct

SEQ ID NO: 25 - DNA, 858 bp, Homo sapiens
atggcgaaccttggctgctggatgctggttctctttgtggccacatggagtgacctgggcctctgcaagaagcgcccgaagcctggaggatggaacactgggggcagccgatacccggggcagggcagccctggaggcaaccgctacccacctcagggcggtggtggctgggggcagcctcatggtggtggctgggggcagcctcatggtggtggctgggggcagccccatggtggtggctgggggcagcctcatggtggtggctgggggcagcctcatggtggtggctgggggcagcctcatggtggtggctgggggcagccccatggtggtggctggggacagcctcatggtggtggctggggtcaaggaggtggcacccacagtcagtggaacaagccgagtaagccaaaaaccaacatgaagcacatggctggtgctgcagcagctggggcagtggtggggggccttggcggctacatgctgggaagtgccatgagcaggcccatcatacatttcggcagtgactatgaggaccgttactatcgtgaaaacatgcaccgttaccccaaccaagtgtactacaggcccatggatgagtacagcaaccagaacaactttgtgcacgactgcgtcaatatcacaatcaagcagcacacggtcaccacaaccaccaagggggagaacttcaccgagaccgacgttaagatgatggagcgcgtggttgagcagatgtgtatcacccagtacgagagggaatctcaggcctattaccagagaggatcgagcatggtcctcttctcctctccacctgtgatcctcctgatctctttcctcatcttcctgatagtgggatga

SEQ ID NO: 26 - DNA, 1571 bp, Homo sapiens
ccacaaatgtgggagggcgataaccactcgtagaaagcgtgagaagttactacaagcggtcctcccggccaccgtactgttccgctcccagaagccccgggcggcggaagtcgtcactcttaagaagggacggggccccacgctgcgcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatggaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactggggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaatttttttttaaa

SEQ ID NO: 27 - DNA, 1689 bp, Homo sapiens
cccctagacaagccggagctgggaccggcaatcgggcgttgatccttgtcacctgtcgcagaccctcatccctcccgtgggagccccctttggacactctatgaccctggaccctcgggggacctgaacttgatgcgatgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtattcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctcctgatactcgggatcctcaggacgcaggtcacattcacctgtgggcagagggacaggtcagacacccaggcccaccccagagaccctccatgaactgtgctcccagccttcccggcaggtctgggagtagggaagggctgaagccttgtttccttgcaggggggccagccattgtctcccacttggggagtttcttcctggcatcatgccttctgaataaatgccgattttgtccatgg

SEQ ID NO: 28 - DNA, 2347 bp, Homo sapiens
caggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcacccctgtctgcgccgctgtgctgttgtctgccgt